(19) United States
(12) Patent Application Publication
(10) Pub. No.: US 2015/0374767 A1
(43) Pub. Date: Dec. 31, 2015

(54) Treatment of Cancer

(71) Applicant: VIRTTU BIOLOGICS LIMITED, Glasgow (GB)

(72) Inventor: Joe CONNER, Glasgow (GB)

(21) Appl. No.: 14/768,549

(22) PCT Filed: Feb. 21, 2014
(86) PCT No.: PCT/GB2014/050522

§ 371 Date: Aug. 18, 2015

(30) Foreign Application Priority Data
Feb. 22, 2013 (GB) 1303184.4

(51) Int. Cl. A61K 35/763; G01N 33/50; A61K 45/06

(52) U.S. Cl. CPC A61K 35/763; A61K 45/06; G01N 33/5011; G01N 2500/10; G01N 2500/04

(57) ABSTRACT

A method of treating cancer is disclosed, the method comprising simultaneous or sequential administration of a virus and a receptor tyrosine kinase (RTK) inhibitor.

### FIELD OF THE INVENTION

The present invention relates to the use of a virus and a receptor tyrosine kinase inhibitor in the treatment of cancer.

### BACKGROUND TO THE INVENTION

Oncolytic virotherapy concerns the use of lytic viruses which selectively infect and kill cancer cells. Some oncolytic viruses are promising therapies as they display exquisite selection for replication in cancer cells and their self-limiting propagation within tumors results in fewer toxic side effects. Several oncolytic viruses have shown great promise in the clinic (Bell, J., “Oncolytic Viruses: An Approved Product on the Horizon?” Mol Ther. 2010; 18(2): 233-234).

Kulu et al (Concurrent chemotherapy inhibits herpes simplex virus-1 replication and oncolysis. Cancer Gene Therapy (2013) 20; 133-140) indicate that cellular responses to chemotherapeutic agents provide an unfavorable environment for HSV-1-mediated oncolysis and indicates that this observation is relevant to preclinical and clinical studies of HSV-1 oncolysis.

### Receptor Tyrosine Kinase (RTKs)

Receptor tyrosine kinases are cell-surface receptors which are key regulators of critical cellular processes such as proliferation and differentiation, cell survival and metabolism, cell migration and cell-cycle control (Blume-Jensen and Hunter, “Oncogenic kinase signalling” Nature (2001) 411, 355-365; Ullrich and Schlessinger “Signal transduction by receptors with tyrosine kinase activity” Cell (1990) 61, 203-212).

Humans have 58 known RTKs, which fall into 20 subfamilies. All RTKs have a similar molecular architecture, with ligand binding domains in the extracellular region, a single transmembrane helix and a cytoplasmic region that contains the protein tyrosine kinase (TK) domain plus additional carboxy (C-) terminal and juxtamembrane regulatory regions.

The overall topology of RTKs, their mechanism of activation and key components of the intracellular signalling pathways that they trigger are highly conserved in evolution from the nematode Caenorhabditis elegans to humans, which is consistent with the key regulatory roles they play. Furthermore, numerous diseases result from genetic changes or abnormalities that alter their activity, abundance or regulation of RTKs. Mutations in RTKs and aberrant activation of their intracellular signalling pathways have been causally linked to cancers, diabetes, inflammation, severe bone disorders, arteriosclerosis and angiogenesis. These connections have driven the development of a new generation of drugs that block or attenuate RTK activity (Lemmon and Schlessinger “Cell Signalling by Receptor Tyrosine Kinasese” (2010) Cell 141, 1117-1134).

Several drugs have been developed and approved by the US Food and Drug Administration (FDA) for treating cancers and other diseases caused by activated RTKs.

Many of these drugs are small molecule inhibitors that target the ATP binding site of the intracellular TK domain.

Many small-molecule RTK inhibitors act at multiple tyrosine kinases. For example, imatinib (Gleevec; 4-[(4-methylpiperazin-1-yl)methyl]-N-(4-methyl-3-{[4-(pyridin-3-yl)pyrimidin-2-yl]amino}phenyl)benzamide) was initially identified in a program to develop PDGFR inhibitors, but it also potently inhibits KIT and the non-receptor tryrosine kinase Abl. Imatinib has shown clinical activity in CML, which arises from constitutive tyrosine kinase activity of the aberrant Bcr-Abl fusion protein. Imatinib has also been successfully applied to the treatment of GISTs, cancers that are primarily driven by constitutively activated KIT. Sunitinib (Sutent; N-(2-diethylaminoethyl)-5-[(Z)-(5-fluoro-2-oxo-1H-indol-3-ylidene)methyl]-2,4-dimethyl-1H-pyrrole-3-carboxamide) also blocks the tyrosine kinase activities of several RTKs, including KIT, VEGFR2, PDGFR, Flt3 and Ret and it has been successfully applied in the treatment of GIST and renal cell carcinoma. On the other hand, the EGFR inhibitors erlotinib (Tarceva; N-(3-ethynylphenyl)-6,7-bis(2-methoxyethoxy)quinazolin-4-amine) and gefitinib (Iressa; N-(3-chloro-4-fluoro-phenyl)-7-methoxy-6-(3-morpholin-4-ylpropoxy)quinazolin-4-amine) show much greater specificity and are capable of selectively inhibiting EGFR under conditions where the closely related ErbB2 TK domain is unaffected.

Although several tyrosine kinase inhibitors have been successfully applied to treat cancers, key difficulties encountered during treatment include side effects that arise from a lack of selectivity toward an individual target and the acquisition of drug resistance.

It is now clear that drug resistance almost invariably develops in cancer patients treated with tyrosine kinase inhibitors. Selective pressure leads to the emergence of drug-resistant variants of the targets or to compensations in the signalling networks that overcome the need for the inhibited RTK to continue growth.

Mutations that abrogate the inhibitory activity of tyrosine kinase inhibitors are frequently seen in the TK domain of the targeted RTK of tumor cells. For example, resistance of EGFR to inhibition by gefitinib is caused by T766M or T830A mutations (mature EGFR numbering) in the EGFR kinase domains of patients' lung adenocarcinomas. Drug resistance in tumors treated with tyrosine kinase inhibitors has also emerged from compensatory activation or overexpresssion of a different RTK or other cell signalling proteins. For example, upregulation of ErbB3 signalling via amplification of the Met gene or through other growth mechanisms overcomes growth inhibition by EGFR inhibitors. It is anticipated that resistance will eventually develop toward every inhibitor that is targeted against RTKs (Lemmon and Schlessinger 2010).

A problem in the art is therefore how to expand the clinical utility of tyrosine kinase inhibitors to provide greater therapeutic benefit.

### Receptor Tyrosine Kinase Families

It is currently known in the art that there are some 58 human receptor tyrosine kinases (RTKs) which fall into 20 subfamilies (FIG. 1 of Lemmon and Schlessinger p 1118).

Without limiting the scope of the present invention, of particular clinical relevance are the RTK subfamilies: ErbB (which includes the family member EGFR), VEGFR, PDGFR, ALK and Insulin-like growth factor receptors. As acknowledged above, however, many tyrosine kinase inhibitors act at multiple tyrosine kinases.

### ErbB Receptors

The ErbB protein family consists of 4 members; ErbB-1 (also called epidermal growth factor receptor [EGFR]), ErbB-2 (also named HER2 in humans and neu in rodents), ErbB-3 (also named HER3) and ErbB-4 (also named HER4) (Bubil E M & Yarden Y 2007 Curr. Opin. Cell. Biol. Vol. 19, pp. 124-134). ErbB proteins are cell surface receptors for a variety of ligands including epidermal growth factor (EGF-family) transforming growth factor α (TGF-α), heparin binding (HB-EGF), amphiregulin, beta-cellulin and epiregulin family proteins (Shawver L K et al., 2002 Cancer Cell. Vol. 1, pp. 117-123). ErbB receptors are made up of an extracellular region of approximately 620 amino acids, a single transmembrane spanning region and a cytoplasmic tyrosine kinase domain (Bubil E M & Yarden Y 2007 Curr. Opin. Cell. Biol, Vol. 19, pp. 124-134).

### ErbB-1/EGFR

EGFR signalling modulates cell migration, cell adhesion, cell proliferation and has downstream effects in the innate immune response. EGFR is a 170-kd glycoprotein consisting of an extracellular receptor domain, a transmembrane region and an intracellular domain with tyrosine kinase function (Herbst R S. 2004 I. J. Radiation Oncology Vol. 59, pp. 21). Upon ligand binding, EGFR dimerizes and autophosphorylation of tyrosine residues (e.g. Tyr992, Tyr1045, Tyr1068, Tyr1148 and Tyr1173) in the C-terminal domain of EGFR occurs (Downward J et al., 1984 Nature Vol. 311, pp. 483-485). Receptor dimerization promotes the activation of the intrinsic kinase, leading to phosphorylation of specific tyrosine residues located in the cytoplasmic domain. These phosphorylated residues serve as docking sites for a variety of signalling molecules, such a Ras, stimulating diverse downstream signalling cascades (Holbro T & Hynes N E 2004 Ann. Rev. Pharm. Tox. Vol. 44, pp. 195-217). Downstream signalling cascades include MAPK, Akt and JNK pathways, leading to DNA synthesis and cell proliferation (Oda K et al. 2004 Mol. Sys. Biol. Vol. 1, pp. 0010).

The Ras-Raf mitogen-activated protein kinase pathway and the phosphatidyl inositol 3′ kinase/Akt pathway are the two major signalling routes for the EGFR family. These pathways regulate multiple biological processes such as gene expression, cellular proliferation, angiogenesis and inhibition of apoptosis, which contribute to the development of malignancy (Chan T O et al., 1999 Ann. Rev. Biochem. Vol. 68, pp, 965-1014). Furthermore stimulation of EGFR pathways has also been shown to promote tumor cell motility, adhesion and metastasis (Shibata T et al., 1996 Tumor Biol. Vol. 17 pp. 168-175).

Overexpression of EGFR is associated with a number of cancers and appears to promote solid tumor growth (Nicholson R I et al. 1990 Euro. J. Cancer. Vol. 37 Supp4, pp. S9-15). For example, some breast cancers may express up to 2×10⁶ EGFR molecules per cell (Ennis B W et al., 1991 Cancer Invest Vol. 9, pp. 553-562).

Overexpression of HER2 occurs in 25-30% of breast cancers, and is associated with poor prognosis and shorter survival.

Its pivotal role in governing cellular proliferation, survival and metastasis makes EGFR an attractive molecular target for cancer therapeutics.

### VEGFR

Vascular endothelial growth-factor receptors (VEGFRs) regulate the cardiovascular system; specifically regulating vascular development during embryogenesis (vasculogenesis) and blood-vessel formation (angiogenesis) in adults (Olsson A-K et al., 2006 “VEGF receptor signalling-in control of vascular function”, Nature Reviews: Mol. Cell Biol. Vol 7, pp 359-371). VEGFRs are members of the receptor tyrosine kinase (RTK) superfamily, capable of binding to VEGF ligands, of which there are several members in mammals (Hoeben A et al., 2004 “Vascular endothelial growth factor and angiogenesis” Pharma. Rev._Vol. 56, pp. 549-580).

There are three members of the VEGFR tyrosine kinase family, VEGFR1, VEGFR2 and VEGFR3 (encoded by the genes FLT1, KDR and FLT4 respectively).

VEGFR1 is expressed on haematopoietic stem cells, monocytes and vascular endothelial cells. VEGFR1 is a positive regulator of monocyte and macrophage migration, and a positive and negative regulator of VEGFR2 signalling capacity. VEGFR2 is expressed on vascular endothelial cells and lymphatic endothelial cells and mediates angiogenesis. VEGFR3 is only expressed on lymphatic endothelial cells, and mediates lymphangiogenesis.

All three VEGFRs have an approximately 750-amino-acid-residue extracellular domain, organised into seven immunoglobulin (Ig)-like folds, a single transmembrane region and a split tyrosine kinase intracellular domain (Cross M J et al., 2003 “VEGF-receptor signal transduction”, Trends Biochem. Sci. Vol. 28, pp. 488-494). VEGFR1 expression is directly regulated by hypoxia-inducible factors (HIFs), and VEGFR2 is also up-regulated during hypoxia. VEGFR1 and VEGFR2 are expressed predominately by vascular endothelial cells, and the receptors are both up-regulated during angiogenesis. VEGFR3 is found mainly in venous endothelium during early embryonic development, together with VEGFR2, but in later development it becomes mainly confined to lymphatic endothelial cells, and VEGFR3 is up-regulated in lymphangiogenic vessels but not angiogenic vessels.

VEGFR1 and VEGFR2 are mainly expressed in endothelial cells, but are also expressed by a wide variety of cancer cell lines (Su J-L et al. 2007 “The role of the VEGF-C/VEGFR-3 axis in cancer progression” Br, J. Cancer Vol. 96, pp. 541-545), and knockout studies suggest both VEGFR1 and VEGFR2 are crucial for normal development of embryonic vasculature. However, while VEGFR2 mediates differentiation, migration and proliferation of endothelial cells, VEGFR1 regulates maintenance of blood vessels in later development stages. VEGFR3 is expressed in lymphatic endothelial cells, and in a variety of human malignancies. VEGFR3 expression in colon cancer has been associated with poorer survival, and expression of VEGFR3 is significantly correlated with cervical carcinogenesis. It is believed that VEGFR3 enhances cancer cell motility and invasion capabilities promoting cancer cell metastasis (Su J L et al. 2007 “The role of the VEGF-C/VEGFR-3 axis in cancer progression” Br. J. Cancer Vol. 96, pp. 541-545).

The term “angiogenesis” describes the formation of new blood-vessels from a pre-existing vascular net, and is fundamental to the progression of many pathological diseases. For example angiogenesis is a critical step in tumor growth and the progression to metastasis of many cancer types (Domingues I et al., 2011 “VEGFR2 translocates to the nucleus to regulate its own transcription”, PLOs One, Vol 6, pp. e25668).

### VEGFR1

VEGFR1 (Flt1) plays a negative role in angiogenesis at embryogenesis. In adulthood, it is expressed not only on endothelial cells but also on macrophages, and promotes the function of macrophages, inflammatory diseases, cancer metastasis and atherosclerosis via its kinase activity. (Shibuya M (2006) “Vascular endothelial growth factor receptor-1 (VEGFR-1/Flt-1): a dual regulator for angiogenesis” Angiogenesis Vol. 9, pp. 225-230). VEGFR1 binds the ligands VEGFA, PIGF and VEGFB. The binding affinity of VEGFR1 for VEGFA is one order of magnitude higher than that of VEGFR2, whereas the kinase activity of VEGFR1 is about 10-fold weaker than that of VEGFR2. A naturally occurring soluble form of VEGFR1 is known; this is a spliced variant of VEGFR1 with a unique C-terminal extension of 31 residues which binds to the ligand VEGF with high affinity possibly sequestering VEGF. The physiological role of sequestration of VEGF is as yet unknown (Garvin S et al. “Effects of oestradiol and tamoxifen on VEGF, soluble BEGFR1 and VEGFR2 in breast cancer and endothelial cells” Br. J, Cancer Vol 93, pp. 1005-1010).

VEGF-induced up-regulation of VEGFR1 is dependent on Protein Kinase B (Akt) and Extra-cellular signal-regulated kinase (ERK) signalling. Binding partners of VEGFR1 include the p85 subunit of phosphatidylinositol 3 kinase (PI3K), phospholipase Cγ (PLC-γ), the tyrosine protein phosphatase SHP-2, growth factor receptor bound 2 (GRB2) protein and the adaptor cytoplasmic protein NCK (non-catalytic region of tyrosine kinase adaptor protein 1) (Ivy et al. 2009 “An overview of small-molecule inhibitors of VEGFR signalling” Nature Rev. Olin. Oncol. Vol. 6, pp. 569-579).

### VEGFR2

VEGFR2 (KDR) binds the ligand VEGF-A, upon binding and subsequent dimerization, VEGFR2 is autophosphorylated at the carboxy terminal tail and kinase insert region, and several tyrosine residues are phosphorylated (residues 951, 1054, 1059, 1175 and 124) (Shibuya et al., 1990 “Nucleotide sequence and expression of a novel human receptor-type kinase gene closely related to the fms family,” Oncogene Vol 5, pp, 519-524).

Of these phosphorylated residues Tyr1054 and Tyr1059 are required for maximal kinase activity. PLCγ interacts with Tyr1175 and mediates the activation of mitogen-activated protein kinase (MAPK)/extracellular-signal-regulated kinase-1/2 (ERK1/2) cascade and proliferation of endothelial cells. The adaptor molecule Shb also binds to Tyr1175, and VEGF-induced migration and activation of P13K is inhibited by siRNA-mediated knockdown of Shb in endothelial cells. The serine/threonine kinase Akt/PKB is activated downstream of P13k and mediates survival of endothelial cells.

The significance of the classic Ras-Raf-MEK-MAPK pathway downstream of VEGFR2 is unclear. Raf is a ubiquitous serine/threonine kinase. Raf kinase isoforms are overexpressed in a variety of solid tumor types including renal cell carcinoma (RCC), hepatocellular carcinoma (HCC), non-small cell lung carcinoma (NSCLC), melanoma and papillary thyroid carcinoma (Gollob J A et al., 2006 “Role of Raf kinase in cancer: Therapeutic potential of targeting the Raf/MEK/ERK signal transduction pathway” Seminars in Oncology Vol. 33, pp. 3892-406). Ras activation has been coupled to an angiogenic phenotype of endothelial cells (Meadows K N et al., 2001 J. Biol. Chem. Vol. 276, pp. 49289-29298), but Ras-independent induction of the Raf-MEK-MAPK pathway, after VGEF stimulation, through PLCγ-activated PKC, has been shown in primary liver sinusoidal endothelial cells (Takahashi T et al., 1999 Oncogene Vo. 18, pp. 2221-2230).

VEGFR2 also has a crucial role in cell migration via TSAd (T-cell specific adaptor) which binds at Tyr951, and stimulates the phosphorylated Tyr951-TSAd pathway regulating endothelial cell migration.

### VEGFR3

VEGFR3 (Flt4) is regulated by phosphorylation of conserved residues in the kinase domain (Tyr1063 and Tyr1068). VEGFR3 forms homodimers or heterodimers with VEGFR2 in response to processed VEGFC. It is reported that VEGFR3 mediates activation of the ERK1/2 in a protein kinase-C (PKC)-dependent manner. It also activates the P13K-Akt/PKB pathway, important during embryonic development, when VEFGC guides migration and sprouting of lymphendothelial precursor cells from restricted regions of the cardinal vein. Other signal transducers that are potentially used by VEGFR3 include PLCγ, SHO2 and the transcription factor STAT3 (signal transducer and activator of transcription-3) and STAT5.

### PDGFR

Platelet-derived growth factor receptors (PDGFRs) bind platelet-derived growth factor (PDGF) which is a major mitogen for connective tissue cells, fibroblasts, smooth muscle cells and certain other cell types (Heldin C-H & Westermark 1999 Physiol. Rev. Vol. 79, pp. 1283-1316). There are four PDGF proteins (PDGF-A, PDGF-B, PDGF-C, PDGF-D) which exist as homo- and hetero-dimers. PDGFRs are important factors regulating cell proliferation, cellular differentiation, cell growth and development. Additionally, they are implicated in the pathology of numerous diseases including cancers (Williams L T 1989 Science. Vol. 242, pp. 1564-1570).

There are two members of the PDGFR family; PDGFRα and PDGFRβ. The PDGFRα binds both A and B chains of PDGF, whilst PDGFRβ binds only the B-chain. Both receptors dimerise on binding PDGF. PDGF-AA, -AB, -BB, and -CC dimers bind to the PDGFRα with high affinity, whereas PDGF-BB and DD dimers bind to PDGFRβ. Activation of PDGFR leads to stimulation of cell growth, but also changes to cell shape and motility. Both PDGFRα and PDGFRβ have five extracellular Ig-like domains and an intracellular tyrosine kinase domain.

Both α- and β-PDGFR homodimers produce potent mitogenic signals, but there are differences regarding their effects on the actin filament system and chemotaxis in certain cell types. Fibroblasts and smooth muscle cells express both α- and β-PDGFRs, but generally higher PDGFRβ (Heldin C-H & Westermark 1999 Physiol. Rev. Vol. 79, pp. 1283-1316). O-2A glial precursor cells, human platelets and rat liver endothelial cells express only PDGFRα, whilst mouse capillary cells express only PDGFRβ (Smits et al. 1989 Growth Factors. Vol. 2, pp. 1-8).

Binding PDGF and subsequent dimerization of PDGFRs autophosphorylates conserved tyrosine residues (Tyr849 in PDGFRα and Tyr857 in PDGFRβ), which creates docking sites for further signal transduction molecules containing SH2 domains (Heldin C-H & Westermark 1999 Physiol. Rev. Vol. 79, pp. 1283-1316). Downstream signalling cascades involving PI3 kinase, PLCγ, Src and SH2, and there is thought to be extensive cross talk with other signalling pathways, in particular with Integrin signalling.

### PDGFRα

PDGFRα homodimers stimulate edge ruffling and loss of stress fiber actin filaments and inhibit chemotaxis of certain cell types including fibroblasts and smooth muscle cells (Yokote K et al. 1996 J. Biol. Chem. Vol. 271, pp. 5101-5111).

### PDGFRβ

PDGFRβ homodimers also stimulate edge ruffling and loss of stress fiber actin filaments, but also mediate the formation of circular actin structures on the dorsal surface of the cell (Eriksson A et al., 1992 EMBO J. Vol. 11, pp. 543-550). They additionally stimulate chemotaxis.

Aberrant PDGF signalling leads to increased interstitial fluid pressure, stromal cell recruitment and neo-angiogenesis in many cancer types, including breast and brain cancer. It additionally may lead to deregulated autocrine/paracrine signalling (Badruddoja M A et al., 2010 Letters in Drug Design and Discovery. Vol. 7, pp. 290-299). PDGFR expression has been demonstrated in malignant breast tissue and surrounding stromal cells (Ostman A. 2004 Cytokine Growth Factor Rev. Vol. 15, pp. 275-286).

Other members of the PDGFR family include Colony stimulating factor 1 receptor (CSF1R) [also known as macrophage colony-stimulating factor receptor (M-CSFR), and CD115 (Cluster of Differentiation 115)], Mast/stem cell growth factor receptor (SCFR) [also known as proto-oncogene c-Kit or tyrosine-protein kinase Kit or CD117], and Cluster of differentiation antigen 135 (CD135) [also known as Fms-like tyrosine kinase 3 (FLT-3), receptor-type tyrosine-protein kinase FLT3, or fetal liver kinase-2 (Flk2)].

### Anaplastic Lymphoma Kinase (ALK)

Anaplastic lymphoma kinase (ALK) is also known as ALK tyrosine kinase receptor or CD246 (cluster of differentiation 256) and is encoded by the ALK gene. ALK is preferentially expressed in neurons of the central and peripheral nervous systems at late embryonic stages (Motegi A et al. 2004 J. Cell Sci. Vol. 117, pp. 3319-3329).

The ALK is a member of the insulin receptor subfamily, and the ALK protein comprises an extracellular domain, a single pass transmembrane region, and an intracellular kinase domain. It plays an important role in the development of the brain and exerts its effects on specific neurons in the nervous system. This gene has been found to be rearranged, mutated, or amplified in a series of tumours including anaplastic large cell lymphomas, neuroblastoma, and non-small cell lung cancer.

ALK was originally identified in a gene fusion with nucleophosmin (NPM1) in anaplastic large cell lymphomas (ALCL) and is also implicated in non-Hodgkin's lymphoma. (Shiota M et al., 1994 Oncogene Vol. 9, pp. 1567-1574; Morris S et al., 1994 Science Vol. 263, pp. 1281-1284). ALK exerts oncogenic effects by forming fusion genes. In approximately 60% of ALCLs, ALK creates a fusion gene with NPM; fusing the 3′ half of ALK to the 5′ portion of NPM. About 4% of patients with non-small cell lung carcinoma have a chromosomal rearrangement that generates a fusion gene between EML4 (‘echinoderm microtubule-associated protein-like 4’) and ALK, which results in constitutive kinase activity that contributes to carcinogenesis and seems to drive the malignant phenotype.

Despite the potential importance of ALK in the development of the nervous system, little is known about its ligand; it is proposed that pleiotrophin (PTN) and midkine (MK) are ligands for ALK (Stoica G E. et al., 2001 J. Biol. Chem. Vol. 226, pp. 16772-16779; Stoica G E. et al., 2002 J. Biol. Chem. Vol. 227, pp. 35990-35998). PTN and MK are secretory heparin-binding growth and differentiation factors affecting roles in mitogenic, neuritogenic, angiogenic and morphogenetic activities (Muramatsu T 2002 J. Biochem. Vol. 132, pp. 359-371). It is currently unknown whether PTN and MK directly or indirectly interact with ALK (Palmer R H., et al. 2009 Biochem. Vol. 420, pp. 345-361).

ALK has an intracellular tyrosine kinase domain with three phosphorylation sites (Y1278, Y1282, and Y1283), followed by a C-terminal domain with interaction sites for phospholipase C-gamma and Src homology 2 domain-containing SHC (Shaw A T & Solomon B 2011 Clin. Cancer Res. Vol. 17, pp. 2081-2086). As for most RTKs, ALK activation results in the recruitment of adaptor proteins, such as IRS-1, Shc and FRS2 and initiation of intracellular signaling pathways, including the canonical Ras/ERK cascade (Palmer R H., et al. 2009 Biochem. Vol. 420, pp. 345-361; Degoutin J et al., 2007 FEBS Lett. Vol. 581, pp. 727-734). Aberrant activation of the ALK kinase by chromosomal translocations or point mutations has been causally implicated in anaplastic large cell lymphoma, non-small cell lung cancer, and neuroblastoma (Gouzi J Y et al., 2011 PLOS One). ALK signaling may also be a rate limiting factor controlling the growth of glioblastoma cells (Powers C et al., 2002 J Biol Chem. Vol. 277, pp. 14153-14158) and non-synonymous polymorphisms in the gene may be associated with schizophrenia (Kunugi H., et al. 2006 J Neural Transm. Vol. 113, pp. 1569-1573).

### Insulin-Like Growth Factor Receptors

Insulin-like growth factor receptors bind to insulin-like growth factors (IGFs) which are proteins with high sequence similarity to insulin. They form part of a complex “IGF axis” (also referred to as the Growth Hormone/IFG1 Axis) comprising two cell surface insulin-like growth factor receptors (IGF-1R and IGF-2R), two ligands (IGF-1 and IGF-2), a family of six high-affinity IGF-binding proteins (IGFBP-1 to IGFBP-6) and associated IGFBP degrading enzymes.

The IGF axis plays a crucial role in the promotion of cell proliferation and the inhibition of cell death. IGF-2 is considered the primary growth factor, whilst IGF-1 is important for achieving maximal growth.

### IGF-1R

Insulin-like growth factor receptor 1 (IGF-1R) is expressed predominately in the central nervous system, hematopoietic cells, buffy coat leukocytes, Esptein-Barr virus transformed lymphocytes, foetal tissues and various malignant cells. The IGF-1R and insulin receptor (IR) share approximately 70% amino acid sequence homology (Ridemann J & Macaulay V M 2006 Endocrine-Related Cancer Vol. 13, S33-S43). IGF-1R binds IGF-1 with high affinity and IGF-2 and insulin (INS) with a lower affinity. The activated IGF-1R is involved in cell growth and survival control. IGF-1R is crucial for tumor transformation and survival of malignant cells.

The IGF-1R is a tetrameric receptor tyrosine kinase consisting of two ligand-binding extracellular a-subunits and two β-subunits composing a transmembrane domain, an intracellular tyrosine kinase domain and a C-terminal domain (Sehat B et al., 2007 PloS ONE Vol. 2, pp. e340). Ligand-receptor interaction results in phosphorylation of tyrosine residues in the tyrosine kinase (TK) domain (spanning from amino acid 973-1229) of the β-subunit. The crystal structure of the inactive and phosphorylated kinase domain has provided a molecular model of the IGF-1R catalytic activity (Favelyukis S et al. 2001 Nat. Struc. Biol. Vol. 8, pp. 1058-1063). In unstimulated state, the activation loop, containing the critical tyrosine (Y) residues 1131, 1135 and 1136, behaves as a pseudosubstrate that blocks the active site. Upon ligand binding the three tyrosines of the activation loop are transphosphorylated by the dimeric subunit partner. Phosphorylation of Y1135 and Y1131 destabilizes the auto-inhibitory conformation of the activation loop, whereas phosphorylation of Y1136 stabilizes the catalytically optimized conformation (Sehat B et al., 2007 PloS ONE Vol. 2, pp. e340), allowing substrate and ATP access. The phosphorylated tyrosine residues serve as docking sites for other signaling molecules such as insulin receptor substrate 1-4 (IRS-1-4) and Shc, leading to the subsequent activation of the phosphatidyl inositol-3 kinase (PI3K), the mitogen-activated protein kinase (MAPK), and the 14-3-3 pathways (Sehat B et al., 2007 PloS ONE Vol. 2, pp. e340; Baserga R2000 Growth Harm IGF Res 10 Suppl AS43-44; Yu H. & Rohan T 2000 J Natl Cancer Inst. Vol. 92, pp. 1472-1489).

Although IGF-1R axis components can be highly altered in cancer, little is known about the molecular mechanisms involved in this process. Chromosome 15q26, where IGF-1R is located, was found to be amplified in basal-like breast cancer (Adelaide J et al., 2007 Cancer Res. Vol. 67, pp. 11565-11575; Gombos A et al., 2012 Invest. New. Drugs Vol. 30, pp. 2433-2442). Low levels of IGF-1R copy number gain were also shown in lung cancer (Dziadziuszko R et al., 2010 J. Clin. Oncol. Vol. 28, pp. 2174-2180), pancreatic adenocarcinoma and colon cancer (Pitts T M et al., 2010 Clin. Cancer res. Vol. 16, pp. 3193-3204). A more recent study has demonstrated that KIT and PDGFR-α wild type GIST have a significantly higher level of IGF-1R amplification than mutated ones (Tam C et al., 2008 Proc. Natl. Acad. Sci. USA Vol. 105, pp. 8387-8392). Whereas no mutation of IGF-1R was described to date, there are some reports of gene polymorphism encoding IGF-1 or IGFBP-3 (Gombos A et al., 2012 Invest. New. Drugs Vol. 30, pp. 2433-2442).

### IGF-2R

Insulin-like growth factor receptor 2 (IGF-2R) is expressed predominately in adipose tissue, liver and muscle and IGF-2R binds only IGF-2 and activates no known intracellular signalling pathways (Nolan et al. 1990).

c-KIT (CD117)

Mast/stem cell growth factor receptor (SCFR), also known as proto-oncogene c-Kit, tyrosine-protein kinase Kit or CD117, is a 145-kd transmembrane glycoprotein, and is the normal cellular homologue of the viral oncogene v-kit and a member of the receptor tyrosine kinase subclass III family that includes receptors for platelet-derived growth factor (PDGF), macrophage colony-stimulating factor, and flt3 ligand. All members of this family have an extracellular domain containing 5 immunoglobulin-like domains, a single transmembrane domain, and a cytoplasmic domain with a split kinase domain and a hydrophilic kinase insert sequence. The juxtamembrane and kinase domains of these receptors are strongly conserved.

The c-kit gene product is expressed by hematopoietic progenitor cells, mast cells, germ cells, interstitial cells of Cajal (ICC), and some human tumours. Studies of mice with inactivating mutations of c-kit or its ligand, stem cell factor (SCF), demonstrated that normal functional activity of the c-kit gene product is absolutely essential for maintenance of normal hematopoiesis, melanogenesis, gametogenesis and growth and differentiation of mast cells and ICC. SCF is produced by human and murine hematopoietic stromal cells, including endothelial cells, fibroblasts, and bone marrow-derived stromal cells. The biologic features of SCF and c-kit are reviewed by Broudy, (Stem cell factor and hematopoiesis. Blood. 1997; 90: 1345-1364) and Lyman and Jacobsen. (c-kit ligand and flt3 ligand: stem/progenitor cell factors with overlapping yet distinct activities. Blood. 1998; 91: 1101-1134).

In addition to its importance in normal cellular physiologic activities, c-kit plays a role in the biologic aspects of certain human cancers, including germ cell tumours, mast cell tumours, gastrointestinal stromal tumours (GIST), small-cell lung cancer, melanoma, breast cancer, acute myelogenous leukemia (AML), and neuroblastoma. Proliferation of tumour cell growth mediated by c-kit occurs either by a specific mutation of the c-kit polypeptide that results in ligand-independent activation or by autocrine stimulation of the receptor. In some types of tumours, inhibition of c-kit activity reduces cellular proliferation, suggesting a role for use of pharmacologic inhibitors of c-kit in the treatment of c-kit-dependent malignancies. (Heinrich et al. (2000) Blood; vol. 96 no. 3: 925-932).

c-MET (HGFR)

c-Met (MET or MNNG HOS Transforming gene) is a proto-oncogene that encodes a membrane receptor protein called hepatocyte growth factor receptor (HGFR) [Bottaro D P, Rubin J S, Faletto D L, Chan A M, Kmiecik T E, Vande Woude G F, Aaronson S A (February 1991). “Identification of the hepatocyte growth factor receptor as the met proto-oncogene product”. Science 251 (4995): 802-4]. The hepatocyte growth factor receptor protein possesses tyrosine-kinase activity [Cooper C S (January 1992). “The met oncogene: from detection by transfection to transmembrane receptor for hepatocyte growth factor”. Oncogene 7 (1): 3-7.].

Hepatocyte growth factor (HGF) is the only known ligand of the MET receptor. Abnormal MET activation in cancer correlates with poor prognosis, where aberrantly active MET triggers tumor growth, formation of new blood vessels that supply the tumor with nutrients, and cancer spread to other organs. MET is deregulated in many types of human malignancies, including cancers of kidney, liver, stomach, breast, and brain.

WO2008/043576A1 describes the use of a broad class of oncolytic viruses and a broad class of anti-angiogenic agents in the treatment of cancer. The authors present a single case report from one patient with metastatic colorectal cancer. Following extensive pre-treatment, the patient received oncolytic HSV (NV1020) and, after completion of the NV1020 course, follow-on combination therapy of irinotecan (CPT11) and cetuximab, an anti-EGFR monoclonal antibody. The patient demonstrated stabilised disease at 3 and 6 months. The authors state that this case reports shows a lasting radiological benefit to second line treatment using NV1020/CPT11/cetuximab in a patient with progressive metastatic colorectal cancer. Irinotecan (CPT11) has shown efficacy in metastatic colon cancer and is approved by the FDA for such use. Furthermore, cetuximab has shown efficacy in metastatic colon cancer and is approved by the FDA for such use in combination with chemotherapy. Without any comparator data, it is not possible to attribute the disease stabilisation to the regimen of NV1020/CPT11/Cetuximab or the CPT11/Cetuximab. In contrast to the present invention, no data is presented as to how or why disease stabilisation was achieved or whether the sequential administration regimen of NV1020 followed by CPT11/cetuximab exhibits any combinatorial effect. Consistent with the disclosure of the use of an anti-angiogenic agent, the authors place emphasis on providing treatment without directly treating the tumor tissue, e.g. by inhibiting angiogenesis (page 28, lines 24-30 and page 29, lines 15-19) so that the blood supply to the tumor is cut off and the tumor is treated without directly treating the tumor cells in any manner. The mechanism of treatment thus does not involve combined action of the virus and anti-angiogenic agent to directly kill cancer cells (page 30, lines 25-28).

In contrast with the disclosure of WO2008/043576 A1, the inventors have developed a screen for the systematic assessment of combinations of oncolytic viruses and receptor tyrosine kinase inhibitors. Surprisingly, this has found that successful combinations of oncolytic viruses and receptor tyrosine kinase trigger an apoptotic cell death which is not seen when each agent is used as a monotherapy.

Furthermore, and in contrast with the disclosure of WO2008/043576 A1, the inventors have discovered that EGFR inhibitors work antagonistically with oncolytic viruses. This finding demonstrates the care that is required in postulating combinations for clinical usage and the need for a screen of the kind disclosed herein to avoid the risk of the agents working antagonistically to the detriment of a patient.

Further work elucidating the mechanism of action of cell death when combining oncolytic virus and receptor tyrosine kinase inhibitors has shown that caspase activation correlates with synergistic interactions and thus the inventors also present here a method of selecting patients for combination therapy.

This finding has application in assessing whether oncolytic virus therapy can be combined with standard of care treatments with receptor tyrosine kinase inhibitors and in expanding the therapeutic utility of the said inhibitors.

### Apoptosis

Apoptosis is a highly ordered and heavily orchestrated natural cellular process that is often dysfunctional in many pathological conditions. In cancer, many tumour cells are resistant to apoptosis and the resulting malignant cells will not die. Apoptosis is complex and involves many pathways and routes and the defects leading to apoptotic resistance can occur at many different nodes in these pathways. In addition to their acquired avoidance of programmed cell death, the malignant cell can also gain a resistance to anticancer drugs, many of which function by inducing apoptosis. Consequently, inducing apoptosis in the resistant cell is an important target for the treatment of cancer.

Caspases are central to all routes to apoptotic death, they function as both initiators and executioners and there are three pathways which lead to their activation. Initiation can either be intrinsic via the mitochondria or extrinsic, mediated by cellular death receptors, and both pathways converge on the same execution phase of apoptosis. A third, less well understood initiation pathway is via the endoplasmic reticulum.

The extrinsic death receptor pathway is initiated when death ligands, such as TNFalpha or Fas ligand, bind to their death receptors, type 1 TNF receptor (TNFR1) and a related protein called Fas (CD95) respectively. These death receptors have an intracellular death domain that recruits adapter proteins such as TNF receptor-associated death domain (TRADD) and Fas-associated death domain (FADD), as well as cysteine proteases like caspase 8. The binding of the death ligand to the death receptor results in the formation of death-inducing signalling complex (DISC) comprising the death receptor and its adaptor protein. DISC then initiates the assembly and activation of pro-caspase 8 and the resultant caspase 8 initiates apoptosis by cleaving other downstream or executioner caspases such as caspase 3.

The intrinsic mitochondrial pathway is initiated within the cell by internal stimuli such as irreparable DNA damage, hypoxia, extremely high concentrations of cytosolic Ca² and severe oxidative stress. Regardless of the stimuli, this pathway is initiated via increased mitochondrial permeability and consequent release of pro-apoptotic molecules such as cytochrome-c into the cytoplasm. The intrinsic pathway is heavily regulated by proteins of the Bcl-2 family, named after the BCL2 gene originally identified at the chromosomal breakpoint of the translocation of chromosome 18 to 14 in follicular non-Hodgkin lymphoma. There are two main groups of Bcl-2 proteins, the pro-apoptotic proteins such as Bax, Bak, Bad, Bcl-Xs, Bid, Bik, Bim and Hrk and the anti-apoptotic proteins such Bcl-2, Bcl-X_{L}, Bcl-W, Bfl-1 and Mcl-1). The anti-apoptotic proteins regulate apoptosis by blocking the mitochondrial release of cytochrome-c whereas the pro-apoptotic proteins act by promoting its release and the balance between the pro- and anti-apoptotic proteins governs whether apoptosis will be initiated. Other apoptotic factors can be released from the mitochondrial intermembrane space into the cytoplasm include apoptosis inducing factor (AIF), second mitochondria-derived activator of caspase (Smac), direct IAP Binding protein with Low pI (DIABLO) and Omi/high temperature requirement protein A (HtrA2). Cytoplasmic cytochrome c combines with Apaf-1 and caspase 9 to form the apoptosome whereas Smac/DIABLO or Omi/HtrA2 promotes caspase activation by releasing sequestered caspase 3 or 9 from inhibitor of apoptosis proteins (IAPs).

Both extrinsic and intrinsic pathways converge on the execution phase of apoptosis which involves a series of caspases. Activated caspase 9 initiates the executioner phase for the intrinsic pathway whereas caspase 8 is the central node for the extrinsic pathway. Both activate caspase 3 which then cleaves the inhibitor of the caspase-activated deoxyribonuclease, which is responsible for nuclear apoptosis and other downstream cleavage targets are protein kinases, cytoskeletal proteins, DNA repair proteins and inhibitory subunits of endonucleases family.

The intrinsic endoplasmic reticulum (ER) pathway is less well understood and involves caspase 12 and is independent of the mitochondria. Briefly, ER injury via hypoxia, free radicals or glucose starvation, causes unfolding of proteins and reduced protein synthesis. Consequently, the adaptor protein TNF receptor associated factor 2 (TRAF2) dissociates from procaspase-12, resulting in its activation.

Evasion of cell death is one of the essential changes in a cell that causes malignant transformation and reduced apoptosis or its resistance plays a vital role in carcinogenesis. There are many ways a malignant cell can acquire diminished apoptosis or gain apoptosis resistance and the principal mechanisms for evasion are disrupted balance of pro-apoptotic and anti-apoptotic proteins, reduced caspase function and impaired death receptor signalling (Wong, R S Y. Apoptosis in cancer: from pathogenesis to treatment. Journal of Experimental & Clinical Cancer Research 2011 30:87).

### Apoptosis and HSV

The apoptotic pathway is often triggered as a consequence of viral infection and almost all DNA viruses including herpes simplex virus type 1 encode anti-apoptotic proteins to modulate the apoptotic pathway. Apoptosis is triggered principally as an intrinsic defense against infections. During HSV replication there is an intricate balance between pro- and anti-apoptotic factors that delays apoptotic death until the virus replication cycle is complete (for review see Nguyen and Blaho, Viruses 2009, 1(3), 965-978). Perturbations in the apoptotic balance can cause premature cell death and have the potential to dramatically alter the outcome of infection.

HSV infection triggers the apoptotic pathway early in infection, however, as viral infection progresses, anti-apoptotic proteins expressed by early and late herpes viral genes prevent apoptosis. Therefore, there is an intricate balance between the pro- and antiapoptotic factors in the infected cell and, when there is a defect in any anti-apoptotic factors, the HSV-infected cells die through Herpes Simplex Virus-Dependent Apoptosis (HDAP). A number of viral proteins which act to block apoptosis during infection have been identified and they include the immediate early proteins ICP27 and ICP4, that most likely act as upstream regulators of crucial later anti-apoptotic viral genes. Deletion of either the ICP27 or ICP4 genes results in a replication defective and pro-apoptotic virus. Late viral genes shown to possess anti-apoptotic properties include US3, gD, gJ, and the latency associated transcripts. Single deletions of these late genes generate viruses that fail to cause apoptosis to the same extent as ICP27- and ICP4-null viruses, suggesting that the late viral genes act in concert to prevent apoptosis during a wild type HSV infection. Crucially, if the proteins responsible for preventing apoptosis are not efficiently produced, as is the case when protein synthesis is blocked during HSV-1 infection, then infected cells die via apoptosis. For review, see Nguyen and Blaho, Viruses 2009, 1(3), 965-978.

### SUMMARY OF THE INVENTION

The present invention concerns the use of a virus to treat cancer, wherein the subject receives the virus and a chemotherapeutic agent as part of the programme of treatment. The virus is preferably an oncolytic virus. The chemotherapeutic agent is preferably a receptor tyrosine kinase (RTK) inhibitor. In preferred embodiments the RTK inhibitor is an inhibitor of one or more of an ErbB receptor, and/or a vascular endothelial growth-factor receptor (VEGFR), and/or a platelet derived growth factor receptor (PDGFR) and/or an ALK inhibitor and/or an insulin-like growth factor receptor inhibitor and/or c-KIT (CD117) inhibitor and/or c-MET inhibitor. In preferred embodiments the RTK inhibited is a human RTK.

The inventors have investigated a virus that will not normally induce apoptosis in infected cells, in combination with RTK inhibitors that normally lead to inhibition of cell growth (cytostasis) but do not normally cause cell death, or apoptotic cell death. The inventors identified that combinations of virus and inhibitors of certain classes of RTK induce apoptotic cell death in cells contacted with the virus and RTK inhibitor. As such, the inventors have identified an effect caused by the combined action of these agents, which is not caused by either agent alone. This effect has also been found to correspond with the provision of a synergistic level of improvement of cell killing compared to that provided by either agent alone.

In some aspects and embodiments the present invention concerns the selection of specific combinations of virus and RTK inhibitor that provide a synergistic cell killing effect. In some embodiments these combinations may be identified and selected by assaying the effect of the combination on the induction of apoptosis in cancer/tumor cells. Such assays may be performed in vitro.

The virus and chemotherapeutic agent are administered as part of a method of treating cancer in the subject. They may be administered simultaneously, e.g. as a combined preparation or as separate preparations, one administered immediately after the other.

Alternatively, they may be administered separately and sequentially, where one agent is administered and then the other administered later after a predetermined time interval.

In one aspect of the present invention an oncolytic herpes simplex virus is provided for use in a method of treating cancer, the method comprising simultaneous or sequential administration of an oncolytic herpes simplex virus and a receptor tyrosine kinase (RTK) inhibitor, wherein the RTK inhibitor is an inhibitor of at least one of a vascular endothelial growth factor receptor (VEGFR) and/or a platelet derived growth factor receptor (PDGFR) and/or c-KIT (CD117) and/or c-MET and/or, optionally, ALK, and wherein the combination of oncolytic herpes simplex virus and RTK inhibitor induces apoptosis in cells of the cancer.

In another aspect of the present invention the use of an oncolytic herpes simplex virus in the manufacture of a medicament for use in a method of treatment of cancer is provided, the method of treatment comprising simultaneous or sequential administration of an oncolytic herpes simplex virus and a receptor tyrosine kinase (RTK) inhibitor, wherein the RTK inhibitor is an inhibitor of at least one of a vascular endothelial growth factor receptor (VEGFR) and/or a platelet derived growth factor receptor (PDGFR) and/or c-KIT (CD117) and/or c-MET and/or, optionally, ALK, and wherein the combination of oncolytic herpes simplex virus and RTK inhibitor induces apoptosis in cells of the cancer.

In another aspect of the present invention a method of treating cancer in a subject, the method comprising simultaneous or sequential administration to said subject of an oncolytic herpes simplex virus and a receptor tyrosine kinase (RTK) inhibitor, wherein the RTK inhibitor is an inhibitor of at least one of a vascular endothelial growth factor receptor (VEGFR) and/or a platelet derived growth factor receptor (PDGFR) and/or c-KIT (CD117) and/or c-MET and/or, optionally, ALK, and wherein the combination of oncolytic herpes simplex virus and RTK inhibitor induces apoptosis in cells of the cancer.

In another aspect of the present invention a receptor tyrosine kinase (RTK) inhibitor for use in a method of treating cancer is provided, the method comprising simultaneous or sequential administration of a receptor tyrosine kinase (RTK) inhibitor and an oncolytic herpes simplex virus, wherein the RTK inhibitor is an inhibitor of at least one of a vascular endothelial growth factor receptor (VEGFR) and/or a platelet derived growth factor receptor (PDGFR) and/or c-KIT (CD117) and/or c-MET and/or, optionally, ALK, and wherein the combination of RTK inhibitor and oncolytic herpes simplex virus induces apoptosis in cells of the cancer.

In another aspect of the present invention use of a receptor tyrosine kinase (RTK) inhibitor in the manufacture of a medicament for use in a method of treatment of cancer is provided, the method of treatment comprising simultaneous or sequential administration of a receptor tyrosine kinase (RTK) inhibitor and an oncolytic herpes simplex virus, wherein the RTK inhibitor is an inhibitor of at least one of a vascular endothelial growth factor receptor (VEGFR) and/or a platelet derived growth factor receptor (PDGFR) and/or c-KIT (CD117) and/or c-MET and/or, optionally, ALK, and wherein the combination of RTK inhibitor and oncolytic herpes simplex virus induces apoptosis in cells of the cancer.

In another aspect of the present invention a method of treating cancer in a subject is provided, the method comprising simultaneous or sequential administration to said subject of an oncolytic herpes simplex virus and a receptor tyrosine kinase (RTK) inhibitor, wherein the RTK inhibitor is an inhibitor of at least one of a vascular endothelial growth factor receptor (VEGFR) and/or a platelet derived growth factor receptor (PDGFR) and/or c-KIT (CD117) and/or c-MET and/or, optionally, ALK, and wherein the combination of oncolytic herpes simplex virus and RTK inhibitor induces apoptosis in cells of the cancer.

In some embodiments, the oncolytic herpes simplex virus and RTK inhibitor are selected such that their combined action induces apoptosis in cells of the subject's cancer. Subjects having cancers suitable for treatment with the combination may also be selected by selecting for cancers that exhibit an apoptotic response to the combined treatment. Such selections may be determined by in vitro assay of cancer cells where the assay indicates the apoptotic response of cells to the combination treatment.

In some embodiments, the combination of oncolytic herpes simplex virus and RTK inhibitor provides a synergistic level of cell killing. In some embodiments the oncolytic herpes simplex virus and RTK inhibitor are selected such that their combined action provides a synergistic level of cell killing in cells of the subject's cancer. Subjects having cancers suitable for treatment with the combination may also be selected by selecting for cancers that exhibit a synergistic level of cell killing in response to the combined treatment. Such selections may be determined by in vitro assay of cancer cells where the assay indicates the level of cell killing in response to the combination treatment, and the assay result is analysed for synergistic effect.

In some embodiments the subject is selected for treatment by a method comprising:
infecting, in vitro, at least one cell of a subject's cancer with an oncolytic herpes simplex virus,
contacting said at least one cell with an RTK inhibitor that is an inhibitor of at least one of a vascular endothelial growth factor receptor (VEGFR) and/or a platelet derived growth factor receptor (PDGFR) and/or c-KIT (CD117) and/or c-MET and/or, optionally, ALK,
determining whether (i) the combined action of the oncolytic herpes simplex virus and RTK inhibitor induces apoptosis said at least one cell and/or (ii) the combined action of the oncolytic herpes simplex virus and RTK inhibitor provides a synergistic level of cell killing.

In one aspect of the present invention a virus is provided for use in a method of treating cancer, the method comprising simultaneous or sequential administration of a virus and an RTK inhibitor.

In another aspect of the present invention the use of a virus in the manufacture of a medicament for use in a method of treatment of cancer is provided, wherein the method of treatment comprises administering an RTK inhibitor to the patient in need of treatment.

In another aspect of the present invention a method of treating cancer is provided, the method comprising administration of a virus and an RTK inhibitor to a patient in need of treatment, thereby treating the cancer.

In another aspect of the present invention a virus is provided for use in a method of treating cancer, wherein the method of treatment comprises administering an RTK inhibitor to the patient in need of treatment.

In another aspect of the present invention an RTK inhibitor is provided for use in a method of treating cancer, wherein the method of treatment comprises administering a virus to the patient in need of treatment.

In another aspect of the present invention the use of an RTK inhibitor in the manufacture of a medicament for use in a method of treatment of cancer is provided, wherein the method of treatment comprises administering a virus to the patient in need of treatment.

In a further aspect of the present invention a pharmaceutical composition or medicament is provided comprising a virus and an RTK inhibitor.

In preferred embodiments the RTK inhibitor is an inhibitor of at least one of a vascular endothelial growth factor receptor (VEGFR) and/or a platelet derived growth factor receptor (PDGFR) and/or c-KIT (CD117) and/or c-MET, and/or, optionally, ALK. The RTK inhibitor may be selected from the group consisting of Sorafenib, Pazopanib, Cabozantinib, Sunitinib, Crizitonib, Regorafenib, Dovitinib. Where the cancer is a glioma the RTK inhibitor may be selected from the group consisting of Sorafenib, Pazopanib, Cabozantinib, Sunitinib, Crizitonib, Regorafenib. Where the cancer is a liver cancer, e.g. hepatocellular carcinoma, the RTK inhibitor may be selected from the group consisting of Sorafenib, Pazopanib, Cabozantinib, Sunitinib, Crizitonib, Regorafenib, Dovitinib. Where the cancer is a lung cancer, e.g. mesothelioma, the RTK inhibitor may be selected from the group consisting of Sorafenib, Pazopanib, Sunitinib, Crizitonib, Regorafenib. Where the cancer is an ovarian cancer the RTK inhibitor may be selected from the group consisting of Sorafenib, Pazopanib, Cabozantinib, Sunitinib, Crizitonib, Regorafenib.

In some embodiments administration of the RTK inhibitor (and optionally also virus) is direct administration to the cancer. The direct administration may be directly to cancer cells or to tissue immediately surrounding the cancer. In some embodiments, intratumoural administration of RTK inhibitor (and optionally also virus) [e.g. intratumoural injection] is preferred. In some other embodiments administration of RTK inhibitor (and optionally also virus) is to the blood being supplied directly to the cancer. Such administration may be intravenous or intra-arterial. Optionally, the RTK inhibitor is administered orally.

The RTK inhibitor may be an inhibitor of one or more of an ErbB receptor, and/or a vascular endothelial growth factor receptor (VEGFR) and/or a platelet derived growth factor receptor (PDGFR) and/or ALK and/or an insulin-like growth factor receptor.

In some embodiments the RTK inhibitor may be an ErbB inhibitor. The ErbB inhibitor may be selected from the group consisting of Erlotinib, Gefitinib, or Lapatinib. In some embodiments the ErbB inhibitor may be another ErbB inhibitor, e.g. as described herein.

In some embodiments the RTK inhibitor is not Erlotinib, Cetuximab or Getifinib, or an anti-EGFR antibody, or a salt, solvate or prodrug thereof.

In some embodiments, the RTK inhibitor is Erlotinib, Cetuximab or Getifinib, or an anti-EGFR antibody, or a salt, solvate or prodrug thereof, and the cancer is not a liver cancer (optionally not a cancer originating in the liver, or not a primary liver cancer, or not a hepatocellular carcinoma, or not a cancer occurring in the liver, or not a cancer that has metastasised to the liver).

In some embodiments of the present invention the RTK inhibitor is not Erlotinib, Cetuximab or Getifinib, or an anti-EGFR antibody or a salt, solvate or prodrug thereof, and the cancer may be a liver cancer (optionally a cancer originating in the liver, or a primary liver cancer, or a hepatocellular carcinoma, or a cancer occurring in the liver, or a cancer that has metastasised to the liver).

In some embodiments the RTK inhibitor may be a VEGFR inhibitor. The VEGFR inhibitor may be selected from the group consisting of Sorafenib, Pazopanib, or Cabozantinib. In some embodiments the VEGFR inhibitor may be another VEGFR inhibitor, e.g. as described herein.

In some embodiments the RTK inhibitor is not Sorafenib, or a salt, solvate or prodrug thereof.

In some embodiments the VEGFR inhibitor is an inhibitor of one or more of VEGFR1, VEGFR2 or VEGFR3. In some embodiments the VEGFR inhibitor may be an inhibitor of VEGFR-2, optionally having combined inhibitory action at VEGFR-1 as well and/or optionally not having combined inhibitory action at VEGFR-3.

In some embodiments, the RTK inhibitor is Sorafenib, or a salt, solvate or prodrug thereof, and the cancer is not a liver cancer (optionally not a cancer originating in the liver, or not a primary liver cancer, or not a hepatocellular carcinoma, or not a cancer occurring in the liver, or not a cancer that has metastasised to the liver).

In some embodiments of the present invention the RTK inhibitor is not Sorafenib, or a salt, solvate or prodrug thereof (for example it may be Pazopanib, or Cabozantinib), and the cancer may be a liver cancer (optionally a cancer originating in the liver, or a primary liver cancer, or a hepatocellular carcinoma, or a cancer occurring in the liver, or a cancer that has metastasised to the liver).

In some embodiments of the present invention the RTK inhibitor may be Pazopanib, or a salt, solvate or prodrug thereof, and the cancer, may be an ovarian cancer, e.g. epithelial ovarian cancer. The ovarian cancer may be a primary ovarian cancer.

In some embodiments of the present invention the RTK inhibitor may be Pazopanib, or a salt, solvate or prodrug thereof, and the cancer may be a central nervous system cancer or brain cancer, e.g. glioma, glioblastoma multiforme, astrocytoma, oligodendroglioma, or ependymoma. The central nervous system or brain cancer may be a primary central nervous system or brain cancer.

In some embodiments of the present invention the RTK inhibitor may be Cabozantinib, or a salt, solvate or prodrug thereof, and the cancer may be a central nervous system cancer or brain cancer, e.g. glioma, glioblastoma multiforme, astrocytoma, oligodendroglioma, or ependymoma. The central nervous system or brain cancer may be a primary central nervous system or brain cancer.

In some embodiments the RTK inhibitor may be a PDGFR inhibitor. The PDGFR inhibitor may be selected from the group consisting of Dasatinib, Amuvatinib. In some embodiments the PDGFR inhibitor may be another PDGFR inhibitor, e.g. as described herein.

In some embodiments the RTK inhibitor may be an ALK inhibitor. The ALK inhibitor may be selected from the group consisting of Crizotinib or GSK1838705A. In some embodiments the ALK inhibitor may be another ALK inhibitor, e.g. as described herein.

In some embodiments the RTK inhibitor may be an insulin-like growth factor receptor inhibitor. The insulin-like growth factor receptor inhibitor may be GSK1838705A. In some embodiments the insulin-like growth factor receptor inhibitor may be another insulin-like growth factor receptor inhibitor, e.g. as described herein.

An RTK inhibitor may have selective inhibitor (or antagonist) action at a given class of RTK (e.g. an ErbB receptor, VEGFR, PDGFR, ALK, c-KIT, c-MET or insulin-like growth factor receptor). However, in some embodiments the RTK may exhibit inhibitor (or antagonist) action at more than one classes of RTK. As such, an RTK inhibitor may be an inhibitor of an ErbB receptor and/or a VEGFR and/or a PDGFR and/or ALK and/or c-KIT and/or c-MET and/or insulin-like growth factor receptor, and/or may be an inhibitor of another RTK.

The cancer may be any type of cancer but in some optional embodiments the cancer is not a liver cancer, e.g. not a liver cancer in a human patient. In an optional embodiment the cancer is not a cancer originating in the liver. In another optional embodiment the cancer is not a primary liver cancer. In another optional embodiment the cancer is not a hepatocellular carcinoma. In another optional embodiment the cancer is not a cancer occurring in the liver. In another optional embodiment the cancer is not a cancer that has metastasised to the liver.

In some embodiments the virus is an oncolytic virus. In some embodiments the oncolytic virus is an oncolytic herpes simplex virus. In some embodiments all copies of the ICP34.5 gene in the genome of the oncolytic herpes simplex virus are modified such that the ICP34.5 gene is incapable of expressing a functional ICP34.5 gene product. As such the oncolytic herpes simplex virus may be an ICP34.5 null mutant.

In some embodiments one or both of the ICP34.5 genes in the genome of the oncolytic herpes simplex virus are modified such that the ICP34.5 gene is incapable of expressing a functional ICP34.5 gene product.

In some embodiments the oncolytic herpes simplex virus is a mutant of HSV-1 strain 17. In preferred embodiments the oncolytic herpes simplex virus is HSV1716 (ECACC Accession No. V92012803), In some embodiments the herpes simplex virus is a mutant of HSV-1 strain 17 mutant 1716.

In other embodiments the oncolytic virus is selected from one of: an oncolytic reovirus, an oncolytic vaccinia virus, an oncolytic adenovirus, an oncolytic Coxsackie virus, an oncolytic Newcastle Disease Virus, an oncolytic parvovirus, an oncolytic poxvirus, an oncolytic paramyxovirus.

In another aspect of the present invention a kit comprising a predetermined amount of virus and a predetermined amount of chemotherapeutic agent is provided, wherein the chemotherapeutic agent is an RTK inhibitor. The kit may be provided together with instructions for the administration of the virus and RTK inhibitor sequentially or simultaneously in order to provide a treatment for cancer.

In another aspect of the present invention products containing therapeutically effective amounts of:
(i) a virus, preferably HSV1716, and
(ii) an RTK inhibitor
   for simultaneous or sequential use in a method of medical treatment, preferably treatment of cancer, are provided. The products may be pharmaceutically acceptable formulations and may optionally be formulated as a combined preparation for coadministration.

In another aspect of the present invention a method of selecting a subject for treatment with an oncolytic herpes simplex virus and a receptor tyrosine kinase (RTK) inhibitor is provided, the method comprising:
infecting, in vitro, at least one cell of a subject's cancer with an oncolytic herpes simplex virus,
contacting said at least one cell with an RTK inhibitor that is an inhibitor of at least one of a vascular endothelial growth factor receptor (VEGFR) and/or a platelet derived growth factor receptor (PDGFR) and/or c-KIT (CD117) and/or c-MET and/or, optionally, ALK,
determining whether (i) the combined action of the oncolytic herpes simplex virus and RTK inhibitor induces apoptosis in said at least one cell and/or (ii) the combined action of the oncolytic herpes simplex virus and RTK inhibitor provides a synergistic level of cell killing in said at least one cell.

In another aspect of the present invention a method of determining the ability of a combination of an oncolytic herpes simplex virus (HSV) and a receptor tyrosine kinase (RTK) inhibitor to provide a synergistic level of cell killing in cells of or from a cancer is provided, the method comprising:
infecting, in vitro, at least one cancer cell with an oncolytic herpes simplex virus,
contacting said at least one cancer cell with an RTK inhibitor that is an inhibitor of at least one of a vascular endothelial growth factor receptor (VEGFR) and/or a platelet derived growth factor receptor (PDGFR) and/or c-KIT (CD117) and/or c-MET and/or, optionally, ALK,
determining whether the combined action of the oncolytic herpes simplex virus and RTK inhibitor induces apoptosis in said at least one cancer cell.

The following numbered paragraphs disclose statements of inventive features:

1. A virus for use in a method of treating cancer, the method comprising simultaneous or sequential administration of a virus and a receptor tyrosine kinase (RTK) inhibitor.
2. A virus for use in a method of treating cancer according to paragraph 1, wherein the RTK inhibitor is an inhibitor of one or more of an ErbB receptor, a vascular endothelial growth factor receptor (VEGFR) and/or a platelet derived growth factor receptor (PDGFR) and/or ALK and/or an insulin-like growth factor receptor.
3. A virus for use in a method of treating cancer according to paragraph 1, wherein the RTK inhibitor is selected from Sorafenib, Pazopanib, or Cabozantinib.
4. A virus for use in a method of treating cancer according to any one of paragraphs 1 to 3, wherein the RTK inhibitor is a VEGFR inhibitor, and optionally is an inhibitor of one or more of VEGFR1, VEGFR2 or VEGFR3.
5. A virus for use in a method of treating cancer according to paragraph 1 or 2, wherein the RTK inhibitor is selected from Erlotinib, Gefitinib, Lapatinib, Dasatinib, Dovitinib, Crizotinib, Ninetedinib, Ponatinib, Amuvatinib, GSK1838705A, or Sunitinib
6. A virus for use in a method of treating cancer according to any one of paragraphs 1 to 5, wherein the virus is an oncolytic virus.
7. An oncolytic virus for use in a method of treating cancer according to paragraph 6, wherein the oncolytic virus is an oncolytic herpes simplex virus.
8. An oncolytic virus for use in a method of treating cancer according to paragraph 7, wherein all copies of the ICP34.5 gene in the genome of the oncolytic herpes simplex virus are modified such that the ICP34.5 gene is incapable of expressing a functional ICP34.5 gene product.
9. An oncolytic virus for use in a method of treating cancer according to paragraph 7 or 8, wherein the oncolytic herpes simplex virus is a mutant of HSV-1 strain 17.
10. An oncolytic virus for use in a method of treating cancer according to paragraph 7 or 8, wherein the oncolytic herpes simplex virus is HSV1716.
11. An oncolytic virus for use in a method of treating cancer according to paragraph 6, wherein the oncolytic virus is selected from one of: an oncolytic reovirus, an oncolytic vaccinia virus, an oncolytic adenovirus, an oncolytic Coxsackie virus, an oncolytic Newcastle Disease Virus, an oncolytic parvovirus, an oncolytic poxvirus, an oncolytic paramyxovirus.
12. A pharmaceutical composition comprising a virus and an RTK inhibitor.
13. A pharmaceutical composition according to paragraph 12, wherein the RTK inhibitor is selected from Sorafenib, Pazopanib, or Cabozantinib.
14. A pharmaceutical composition according to paragraph 12 or 13, wherein the RTK inhibitor is a VEGFR inhibitor, and optionally is an inhibitor of one or more of VEGFR1, VEGFR2 or VEGFR3.
15. A pharmaceutical composition according to paragraph 12, wherein the RTK inhibitor is selected from Erlotinib, Gefitinib, Lapatinib, Dasatinib, Dovitinib, Crizotinib, Ninetedinib, Ponatinib, Amuvatinib, GSK1838705A, or Sunitinib
16. A pharmaceutical composition according to any one of paragraphs 12 to 15, wherein the virus is an oncolytic virus.
17. A pharmaceutical composition according to paragraph 16, wherein the oncolytic virus is an oncolytic herpes simplex virus.
18. A pharmaceutical composition according to paragraph 17, wherein the oncolytic herpes simplex virus is a mutant of HSV-1 strain 17.
19. A pharmaceutical composition according to paragraph 17, wherein the oncolytic herpes simplex virus is HSV1716.
20. A kit comprising a predetermined amount of a virus and a predetermined amount of chemotherapeutic agent, wherein the chemotherapeutic agent is an RTK inhibitor.
21. A kit according to paragraph 20, wherein the RTK inhibitor is selected from Sorafenib, Pazopanib, or Cabozantinib.
22. A kit according to paragraph 20 or 21, wherein the RTK inhibitor is a VEGFR inhibitor and optionally is an inhibitor of one or more of VEGFR1, VEGFR2 or VEGFR3.
23. A kit according to paragraph 20, wherein the RTK inhibitor is selected from Erlotinib, Gefitinib, Lapatinib, Dasatinib, Dovitinib, Crizotinib, Ninetedinib, Ponatinib, Amuvatinib, GSK1838705A, or Sunitinib

### DESCRIPTION

### Viruses

As described herein, an improvement in cell killing identified by combination of RTK inhibition and cellular viral infection has been identified that is indicated to be independent of enhancing the cell lysis property of the virus. As such, the present invention may be put into practice using the combination of any suitable virus and RTK inhibitor.

Because oncolytic viruses are generally pre-selected because of their favourable properties for use in treatment of cancer they are preferred in several embodiments of the present invention.

However, in other embodiments viruses that are suitably attenuated and/or made replication deficient or incompetent may be selected.

Attenuated viruses are well known, e.g. providing the basis of many vaccines. Replication deficient viruses are also well known, e.g. providing the basis of viral vectors for use in gene therapy. Attenuated or replication deficient viruses can be generated through modification of the viral genome, commonly to modify part of the viral genome that is required for viral replication. Such viruses will normally retain the ability to infect cells but not replicate to produce viral progeny. A number of viral types have been used for the generation of vaccines or for gene therapy, including retrovirus, adenovirus, lentivirus, herpes simplex virus, vaccinia, pox virus, and adeno-associated virus. For a discussion of suitable viruses and the modifications required to provide for attenuation and/or replication deficiency see Fields Virology 5^{th} Edition Edited by David M Knipe and Peter M Howley (2007 Lippincott Williams & Wilkins).

Viruses used in the present invention are preferably viruses that do not cause apoptotic cell death following infection of cells, particularly cancer cells, alone, e.g. in the absence of other active agents.

### Oncolytic Viruses

In several preferred embodiments of the present invention the virus is an oncolytic virus.

The oncolytic virus may be any oncolytic virus. Preferably it is a replication-competent virus, being replication-competent at least in the target tumor cells. In some embodiments the oncolytic virus is selected from one of an oncolytic herpes simplex virus, an oncolytic reovirus, an oncolytic vaccinia virus, an oncolytic adenovirus, an oncolytic Newcastle Disease Virus, an oncolytic Coxsackie virus, an oncolytic measles virus. An oncolytic virus is a virus that will lyse cancer cells (oncolysis), preferably in a selective manner. Viruses that selectively replicate in dividing cells over non-dividing cells are often oncolytic. Oncolytic viruses are well known in the art and are reviewed in Molecular Therapy Vol. 18 No. 2 Feb. 2010 pg 233-234.

In some embodiments the oncolytic virus is a herpes simplex virus. The herpes simplex virus (HSV) genome comprises two covalently linked segments, designated long (L) and short (S). Each segment contains a unique sequence flanked by a pair of inverted terminal repeat sequences. The long repeat (RL or R_{L}) and the short repeat (RS or R_{S}) are distinct.

The HSV ICP34.5 (also called γ34.5) gene, which has been extensively studied, has been sequenced in HSV-1 strains F and syn17+ and in HSV-2 strain HG52. One copy of the ICP34.5 gene is located within each of the RL repeat regions. Mutants inactivating one or both copies of the ICP34.5 gene are known to lack neurovirulence, Le, be avirulent/non-neurovirulent (non-neurovirulence is defined by the ability to introduce a high titre of virus (approx 10⁶ plaque forming units (pfu)) to an animal or patient without causing a lethal encephalitis such that the LD₅₀ in animals, e.g. mice, or human patients is in the approximate range of ≧10⁶ pfu), and be oncolytic.

Oncolytic HSV that may be used in the present invention include HSV in which one or both of the γ34.5 (also called ICP34.5) genes are modified (e.g. by mutation which may be a deletion, insertion, addition or substitution) such that the respective gene is incapable of expressing, e.g. encoding, a functional ICP34.5 protein. Preferably, in HSV according to the invention both copies of the γ34.5 gene are modified such that the modified HSV is not capable of expressing, e.g. producing, a functional ICP34.5 protein.

In some embodiments the oncolytic herpes simplex virus may be an ICP34.5 null mutant where all copies of the ICP34.5 gene present in the herpes simplex virus genome (two copies are normally present) are disrupted such that the herpes simplex virus is incapable of producing a functional ICP34.5 gene product. In other embodiments the oncolytic herpes simplex virus may lack at least one expressible ICP34.5 gene. In some embodiments the herpes simplex virus may lack only one expressible ICP34.5 gene. In other embodiments the herpes simplex virus may lack both expressible ICP34.5 genes. In still other embodiments each ICP34.5 gene present in the herpes simplex virus may not be expressible. Lack of an expressible ICP34.5 gene means, for example, that expression of the ICP34.5 gene does not result in a functional ICP34.5 gene product.

Oncolytic herpes simplex virus may be derived from any HSV including any laboratory strain or clinical isolate (non-laboratory strain) of HSV. In some preferred embodiments the HSV is a mutant of HSV-1 or HSV-2. Alternatively the HSV may be an intertypic recombinant of HSV-1 and HSV-2. The mutant may be of one of laboratory strains HSV-1 strain 17, HSV-1 strain F or HSV-2 strain HG52. The mutant may be of the non-laboratory strain JS-1. Preferably the mutant is a mutant of HSV-1 strain 17. The herpes simplex virus may be one of HSV-1 strain 17 mutant 1716, HSV-1 strain F mutant R3616, HSV-1 strain F mutant G207, HSV-1 mutant NV1020, or a further mutant thereof in which the HSV genome contains additional mutations and/or one or more heterologous nucleotide sequences. Additional mutations may include disabling mutations, which may affect the virulence of the virus or its ability to replicate. For example, mutations may be made in any one or more of ICP6, ICP0, ICP4, ICP27. Preferably, a mutation in one of these genes (optionally in both copies of the gene where appropriate) leads to an inability (or reduction of the ability) of the HSV to express the corresponding functional polypeptide. By way of example, the additional mutation of the HSV genome may be accomplished by addition, deletion, insertion or substitution of nucleotides.

A number of oncolytic herpes simplex viruses are known in the art. Examples include HSV1716, R3616 (e.g. see Chou & Roizman, Proc. Natl. Acad. Sci. Vol. 89, pp. 3266-3270, April 1992), G207 (Toda et al, Human Gene Therapy 9:2177-2185, Oct. 10, 1995), NV1020 (Geevarghese et al, Human Gene Therapy 2010 September; 21(9):1119-28), RE6 (Thompson et al, Virology 131, 171-179 (1983)), and Oncovex™ (Simpson et al, Cancer Res 2006; 66:(9) 4835-4842 May 1, 2006; Liu et al, Gene Therapy (2003): 10, 292-303), dlsptk, hrR3, R4009, MGH-1, MGH-2, G47Δ, Myb34.5, DF3γ34.5, HF10, NV1042, RAMBO, rQNestin34.5, R5111, R-LM113, CEAICP4, CEAγ34.5, DF3γ34.5, KeM34.5 (Manservigi et al, The Open Virology Journal 2010; 4:123-156), rRp450, M032 (Campadelli-Fiume et al, Rev Med. Viral 2011; 21:213-226), Baco1 (Fu et al, Int. J. Cancer 2011; 129(6):1503-10) and M032 and C134 (Cassady et al, The Open Virology Journal 2010; 4:103-108).

In some preferred embodiments the herpes simplex virus is HSV-1 strain 17 mutant 1716 (HSV1716). HSV 1716 is an oncolytic, non-neurovirulent HSV and is described in EP 0571410, WO 92/13943, Brown et al (Journal of General Virology (1994), 75, 2367-2377) and MacLean et al (Journal of General Virology (1991), 72, 631-639). HSV 1716 has been deposited on 28 Jan. 1992 at the European Collection of Animal Cell Cultures, Vaccine Research and Production Laboratories, Public Health Laboratory Services, Porton Down, Salisbury, Wiltshire, SP4 0JG, United Kingdom under accession number V92012803 in accordance with the provisions of the Budapest Treaty on the International Recognition of the Deposit of Microorganisms for the Purposes of Patent Procedure (herein referred to as the ‘Budapest Treaty’).

In some embodiments the herpes simplex virus is a mutant of HSV-1 strain 17 modified such that both ICP34.5 genes do not express a functional gene product, e.g. by mutation (e.g. insertion, deletion, addition, substitution) of the ICP34.5 gene, but otherwise resembling or substantially resembling the genome of the wild type parent virus HSV-1 strain 17+. That is, the virus may be a variant of HSV1716, having a genome mutated so as to inactivate both copies of the ICP34.5 gene of HSV-1 strain 17+ but not otherwise altered to insert or delete/modify other protein coding sequences.

Other types of oncolytic virus are also known in the art. These include oncolytic poxvirus (e.g. orthopoxviruses) such as vaccinia virus JX-954 and GLV-1 h68 and myoma (Park, B H et al. (2008) Lancet Oncol 9:533-542; Kelly et al. Human Gene Therapy 19:744-782 (August 2008); Wennier et al. Expert Rev Mol Med. 13 e18 5 Dec. 2011, Chan, Rahman, McFadden Oncolytic myxoma virus: the path to the clinic (2013) Vaccine 31(39); 4252-4258) oncolytic reovirus such as oncolytic reovirus type 3 Dearing (Pandha, H S, et al. (2009) Clin Cancer Res 15:6158-6166; Vidal, L et al. (2008) Clin Cancer Res 14:7127 oncolytic adenovirus such as Onyx-015 (Cohen and Rudin. Curr Opin Investig Drugs 2001 December; 2(12):1770-5), oncolytic paramyxovirus such as oncolytic measles virus MV-Edm (Nakamura, T, et al. (2005) Nat Biotechnol 23: 209-214; Wennier et al. Expert Rev Mol Med. 13 e18 5 Dec. 2011), oncolytic Coxsackie virus such as A13, A15, A18, A21 (Au et al, Virology Journal 2011, 8:22), oncolytic Newcastle Disease Virus (Mansour et al, J Virol 2011, June; 85(12):6015-23), and oncolytic parvoviruses such as H-1 PV and MVM (Wennier et al. Expert Rev Mol Med. 13 e18 5 Dec. 2011), oncolytic rhabdoviruses such as Maraba virus (Pol et al. Maraba virus as a potent oncolytic vaccine vector, (2014) Mol Ther. 22(2); 420-429.

In some embodiments the genome of an oncolytic virus according to the present invention may be further modified to contain nucleic acid encoding at least one copy of a polypeptide that is heterologous to the virus (i.e. is not normally found in wild type virus) such that the polypeptide can be expressed from the nucleic acid. As such, the oncolytic virus may also be an expression vector from which the polypeptide may be expressed. Examples of such viruses are described in WO2005/049846, WO2005/049845 and WO2008/099189. In some embodiments of the present invention, the oncolytic virus encodes ING4. HSV capable of expressing ING4 are described in PCT/GB2008/000527; WO2008/099189. HSV1716ING4 has been deposited at the European Collection of Animal Cell Cultures, Vaccine Research and Production Laboratories, Public Health Laboratory Services, Porton Down, Salisbury, Wiltshire, SP4 0JG, United Kingdom under accession number 08021501 in accordance with the provisions of the Budapest Treaty.

In order to effect expression of the polypeptide, nucleic acid encoding the polypeptide is preferably operably linked to a regulatory sequence, e.g. a promoter, capable of effecting transcription of the nucleic acid encoding the polypeptide. A regulatory sequence (e.g. promoter) that is operably linked to a nucleotide sequence may be located adjacent to that sequence or in close proximity such that the regulatory sequence can effect and/or control expression of a product of the nucleotide sequence. The encoded product of the nucleotide sequence may therefore be expressible from that regulatory sequence.

Oncolytic viruses may be formulated as medicaments and pharmaceutical compositions for clinical use and in such formulations may be combined with a pharmaceutically acceptable carrier, diluent or adjuvant. The composition may be formulated for topical, parenteral, systemic, intracavitary, intravenous, intra-arterial, intramuscular, intrathecal, intraocular, intratumoral, subcutaneous, oral or transdermal routes of administration which may include injection. Suitable formulations may comprise the virus in a sterile or isotonic medium. Medicaments and pharmaceutical compositions may be formulated in fluid (including gel) or solid (e.g. tablet) form. Fluid formulations may be formulated for administration by injection or via catheter to a selected region of the human or animal body.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Targeting therapies may be used to deliver the oncolytic virus to certain types of cell, e.g. by the use of targeting systems such as antibody or cell specific ligands. Targeting may be desirable for a variety of reasons; for example if the virus is unacceptably toxic in high dose, or if it would otherwise require too high a dosage, or if it would not otherwise be able to enter the target cells.

HSV capable of targeting cells and tissues are described in (PCT/GB2003/000603; WO 03/068809).

An oncolytic virus may be administered alone or in combination with other treatments, either simultaneously or sequentially dependent upon the condition to be treated. Such other treatments may include chemotherapy (including either systemic treatment with a chemotherapeutic agent or targeted therapy using small molecule or biological molecule (e.g. antibody) based agents that target key pathways in tumor development, maintenance or progression) or radiotherapy provided to the subject as a standard of care for treatment of the cancer.

### Chemotherapy

Chemotherapy refers to treatment of a tumor with a drug. For example, the drug may be a chemical entity, e.g. small molecule pharmaceutical. The drug may be formulated as a pharmaceutical composition or medicament. The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs/agents, one or more of which may be intended to treat the tumor. In the present invention a virus and chemotherapeutic may be administered simultaneously, separately, or sequentially which may allow the two agents to be present in the tumor requiring treatment at the same time and thereby provide a combined therapeutic effect, which may be additive or synergistic.

The chemotherapy may be administered by one or more routes of administration, e.g. parenteral, intra-arterial injection or infusion, intravenous injection or infusion, intraperitoneal, intratumoral or oral. Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug; the time interval between administrations; the length of each treatment; the number and nature of any treatment holidays, if any etc. For a co-therapy a single treatment regime may be provided which indicates how each drug/agent is to be administered.

RTK inhibitors

Receptor tyrosine kinases (RTK) are cell-surface receptors which are key regulators of critical cellular processes such as proliferation and differentiation, cell survival and metabolism, cell migration and cell-cycle control (Blume-Jensen and Hunter, “Oncogenic kinase signalling” Nature (2001) 411, 355-365; Ullrich and Schlessinger “Signal transduction by receptors with tyrosine kinase activity” Cell (1990) 61, 203-212).

Mutations in RTKs and aberrant activation of their intracellular signalling pathways have been causally linked to cancers, diabetes, inflammation, severe bone disorders, arteriosclerosis and angiogenesis. These connections have driven the development of a new generation of drugs that block or attenuate RTK activity (Lemmon and Schlessinger “Cell Signalling by Receptor Tyrosine Kinasese” (2010) Cell 141, 1117-1134).

Several drugs have been developed and approved by the US Food and Drug Administration (FDA) for treating cancers and other diseases caused by activated RTKs. Many of these drugs are small molecule inhibitors that target the ATP binding site of the intracellular TK domain. As such, preferred RTK inhibitors may be inhibitors that act at, e.g. bind to, an intracellular portion or domain of the RTK. In some embodiments the RTK inhibitor may act at, bind to, or inhibit the ATP binding site of the RTK, preferably an ATP binding site present in an intracellular portion or domain of the RTK.

Many small-molecule RTK inhibitors act at multiple tyrosine kinases. For example, Imatinib (Gleevec) was initially identified in a program to develop PDGFR inhibitors, but it also potently inhibits KIT and the non-receptor tryrosine kinase Abl. Imatinib has shown clinical activity in CML, which arises from constitutive tyrosine kinase activity of the aberrant Bcr-Abl fusion protein. Imatinib has also been successfully applied to the treatment of GISTs, cancers that are primarily driven by constitutively activated KIT. Sunitinib (Sutent) also blocks the tyrosine kinase activities of several RTKs, including KIT, VEGFR2, PDGFR, Flt3 and Ret and it has been successfully applied in the treatment of GIST and renal cell carcinoma. On the other hand, the EGFR inhibitors Erlotinib (Tarceva) and Gefitinib (Iressa) show much greater specificity and are capable of selectively inhibiting EGFR under conditions where the closely related ErbB2 TK domain is unaffected.

A number of small molecule inhibitors RTK inhibitors are described below, by way of example. It will be appreciated that a number of the known RTK inhibitors exhibit inhibition at more than one type of RTK. Any indication given below that an agent is an inhibitor of a particular RTK, or class of RTK is not an indication that it does not have inhibitory effects against a different RTK or different class of RTK. It is not unusual for an RTK inhibitor to have action at more than one RTK, either within an RTK family or between RTK families.

Whilst an RTK inhibitor may exhibit inhibition at a number of RTKs in some embodiments of the present invention it is preferred that the RTK inhibitor exhibits a threshold level of inhibition at at least one of a vascular endothelial growth factor receptor (VEGFR) and/or a platelet derived growth factor receptor (PDGFR) and/or c-KIT (CD117) and/or c-MET, and/or, optionally ALK.

As such, the RTK inhibitor may exhibit specificity of inhibition for at least one of these RTKs, and in some instances for two, three or four of them but may not exhibit a threshold level of inhibition at other RTKs. For example, the RTK inhibitor may exhibit a lower 1050 for a given RTK than for another RTK.

The RTK for which the RTK inhibitor exhibits specificity may exhibit a percentage residual activity below a given threshold, at a given concentration of RTK inhibitor. For example, Anastassiadis et al. 2011 (Comprehensive assay of kinase catalytic activity reveals features of kinase inhibitor selectivity, Nature Biotechnology 29, 1039-1045) provides percentage residual catalytic activity of kinases at a concentration of 500 nM inhibitor, and describes methods for determining such.

In accordance with the present invention, the RTK for which the RTK inhibitor exhibits specificity may exhibit residual catalytic activity of one of <100%, <99%, <98%, <97%, <96%, <95%, <94%, <93%, <92%, <91%, <90%, <89%, <88%, <87%, <86%, <85%, <84%, <83%, <82%, <81%, <80%, <79%, <78%, <77%, <76%, <75%, <74%, <73%, <72%, <71%, <70%, <69%, <68%, <67%, <66%, <65%, <64%, <63%, <62%, <61%, <60%, <59%, <58%, <57%, <56%, <55%, <54%, <53%, <52%, <51%, <50%, <49%, <48%, <47%, <46%, <45%, <44%, <43%, <42%, <41%, <40%, <39%, <38%, <37%, <36%, <35%, <34%, <33%, <32%, <31%, <30%, <29%, <28%, <27%, <26%, <25%, <24%, <23%, <22%, <21%, <20%, <19%, <18%, <17%, <16%, <15%, <14%, <13%, <12%, <11%, <10%, <9%, <8%, <7%, <6%, <5%, <4%, <3%, <2%, <1% or 0% at 500 nM RTK inhibitor. The RTK for which the RTK inhibitor does not exhibit specificity may exhibit residual catalytic activity of >0%, >1%, >2%, >3%, >4%, >5%, >6%, >7%, >8%, >9%, >10%, >11%, >12%, >13%, >14%, >15%, >16%, >17%, >18%, >19%, >20%, >21%, >22%, >23%, >24%, >25%, >26%, >27%, >28%, >29%, >30%, >31%, >32%, >33%, >34%, >35%, >36%, >37%, >38%, >39%, >40%, >41%, >42%, >43%, >44%, >45%, >46%, >47%, >48%, >49%, >50%, >51%, >52%, >53%, >54%, >55%, >56%, >57%, >58%, >59%, >60%, >61%, >62%, >63%, >64%, >65%, >66%, >67%, >68%, >69%, >70%, >71%, >72%, >73%, >74%, >75%, >76%, >77%, >78%, >79%, >80%, >81%, >82%, >83%, >84%, >85%, >86%, >87%, >88%, >89%, >90%, >91%, >92%, >93%, >94%, >95%, >96%, >97%, >98%, >99%, >100% at 500 nM RTK inhibitor.

In some embodiments the RTK inhibitor has an IC50 for a specific RTK (which may be selected from one of a vascular endothelial growth factor receptor (VEGFR) and/or a platelet derived growth factor receptor (PDGFR) and/or c-KIT (CD117) and/or c-MET, and/or, optionally ALK), e.g. as measured according to the description of measurement of respective RTK activity provided herein, which is less than one of 50 μM, 10 μM, 500 nM, 250 nM, 200 nM, 100 nM, 50 nM, 40 nM, 30 nM, 20 nM, 10 nM, 5 nM, 4 nM, 3 nM, 2 nM or 1 nM.

In this specification an RTK inhibitor is preferably a small molecule inhibitor and not a biologic such as a peptide, protein or antibody.

The RTK inhibitor may be one that has a cytostatic effect, i.e. inhibition of cell growth. It may also induce cell death but in some preferred embodiments it does not act alone to cause cell death, or apoptotic cell death.

### ErbB Inhibitors.

ErbB inhibitors are examples of chemotherapeutic agents that may be used together with a virus to treat a cancer in accordance with the present invention

As such, an ErbB inhibitor is an agent capable of inhibiting the activity of any one of ErbB-1, ErbB-2, ErbB-3, and/or ErbB-4 individually or in combination, preferably mammalian ErbB-1, ErbB-2, ErbB-3, and/or ErbB-4, more preferably human. In some preferred embodiments the ErbB inhibitor is capable of inhibiting ErbB-1 (EGFR), alone or in addition to being capable of inhibiting one or more of ErbB-2, ErbB-3, and/or ErbB-4.

An ErbB inhibitor will typically be a chemical entity, e.g. small molecule pharmaceutical.

Inhibition of ErbB activity can be tested using routine procedures known to those of ordinary skill in the art, thus allowing one to confirm whether a given agent is an ErbB inhibitor. For example, the skilled person would readily be able to identify suitable ErbB inhibitors for use in the present invention by ELISA assay and detection of phosphorylated tyrosine as described in Wakeling A E et al. 2002 Cancer Res. Vol. 62, pp. 5749 or Hennequin L F et al. 1999 J. Med. Chem. Vol. 42, pp. 5369-5389.

A number of ErbB inhibitors are well known, and are reviewed Hynes N E & Lane H A 2005 Nature Reviews Cancer Vol. 5, pp. 341-354. Several are discussed below.

### Erlotinib (Tarceva; N-(3-ethynylphenyl)-6,7-bis(2-methoxyethoxy)quinazolin-4-amine)

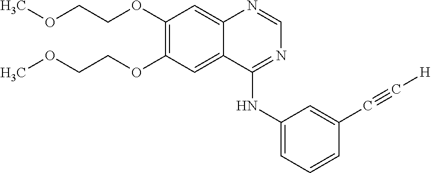 [SMILES: C#Cc1cccc(Nc2ncnc3cc(OCCOC)c(OCCOC)cc23)c1]

Erlotinib is a potent EGFR inhibitor with an IC50 of 2 nmol/L. It acts by inducing the expression of the cell-cycle inhibitor p27, and suppressing the expression of the cell-cycle promoter cyclin D1, thereby blocking cell-cycle progression at the G1 phase. Assessment of the effect of various oral doses of Erlotinib on tumor growth in the HN5 head and neck tumor xenograft model indicated a marked improvement in antitumor effect between doses of 1.6 and 12.5 mg/kg; since the 12.5 mg/kg dose resulted in no substantive tumor growth, relative improvements in antitumor effect at higher doses are not readily apparent (Cancer Res. 1997; 57:4838-48; Oncology; Vol. 17 No. 11 12).

### Gefintinib (Iressa; N-(3-chloro-4-fluoro-phenyl)-7-methoxy-6-(3-morpholin-4-ylpropoxy)quinazolin-4-amine)

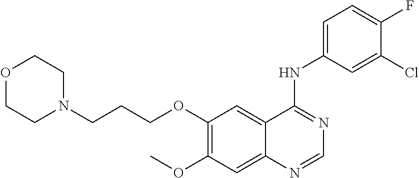 [SMILES: COc1cc2ncnc(Nc3ccc(F)c(Cl)c3)c2cc1OCCCN1CCOCC1]

Gefintinib is a novel potent EGFR tyrosine kinase and Akt phosphorylation inhibitor with IC50 of 37, 26 and 57 nM for Tyr1173, Tyr1173 and Tyr992 in, respectively, low and high EGFR expressing cell lines (Int. J. Cancer. 2001; 774-782), The FDA approved Gefitinib for use in NSCLC in May 2003.

Lapatinib (Tyverb; N-[3-chloro-4-[(3-fluorophenyl)methoxy]phenyl]-6-[5-[(2-methylsulfonylethylamino)methyl]-2-furyl]quinazolin-4-amine): is dual tyrosine kinase inhibitor which inhibits the oncogenes epidermal growth factor receptor (EGFR) and HER2/ERB2 and is most commonly used to treat breast cancer. Lapatinib acts by blocking the receptor signal process by binding to the ATP-binding pocket of EGFR/HER2 protein kinase domain, inhibiting self-phospholylation and consequent activation of the signal cascade (Moreira C, Kaklamani V. Lapatinib and breast cancer: current indications and outlook for the future. Expert Review of Anticancer Therapy. 2010; 10(8):1171-1182). Lapatinib was found to have 50% inhibitory concentration (IC50) values against purified EGFR and HER2 of 10.2 and 9.8 nM, respectively (Hinohara K, et al. Proc Natl Acad Sci USA 2012; 109(17), 6584-6589).

Afantinib (BIBW2992; N-[4-[(3-Chloro-4-fluorophenyl)amino]-7-[[(3S)-tetrahydro-3-furanyl]oxy]-6-quinazolinyl]-4(dimethylamino)-2-butenamide): is an irreversible EGFR/HER2 inhibitor with an 1050 of 14 nM for in vitro potency against HER2. In cell-free in vitro kinase assays, BIBW2992 shows potent activity against wild-type and mutant forms of EGFR and HER2, similar to Gefitinib in potency for L858R EGFR, but about 100-fold more active against the Gefitinib resistant L858R-T790M EGFR double mutant, with an IC50 of 10 nM. Afatinib (BIBW2992) was effective in inhibiting survival of lung cancer cell lines harboring wild-type (H1666) or L858R/T790M (NCI-H1975) EGFR, with IC50s below 100 nM for these isoforms resistant to first-generation inhibitors and a subnanomolar IC50 for the Gefitinib sensitive L858R expressed by H3255 (Oncogene 2008; 27:4702-4711).

CI-1033 (Canertinib;_N-{4-[(3-Chloro-4-fluorophenyl)amino]-7-[3-(morpholin-4-yl)propoxy]quinazolin-6-yl}prop-2-enamide): is an orally bioavailable irreversible Pan-ErbB tyrosine kinase inhibitor, targeting EGFR with IC50 of 0.8, 19 and 7 nM for EGFR, HER-2 and ErbB-4, respectively. It effectively inhibits the growth of esophageal squamous cell carcinoma which co-expresses both EGFR and HER2 with the inhibition of phosphorylation of both MAPK and AKT (Clin Cancer Res 2004 Nov. 1; 10(21):7112-20).

Neratinib (HKI-272; (2E)-N-[4-[[3-chloro-4-[(pyridin-2-yl)methoxy]phenyl]amino]-3-cyano-7-ethoxyquinolin-6-yl]-4-(dimethylamino)but-2-enamide): is an orally available, irreversible tyrosine kinase inhibitor with 1050 of 59 nM and 92 nM for HER2 and EGFR, respectively. Neratinib binds to the HER-2 receptor irreversibly, thereby reducing autophosphorylation in cells, apparently by targeting a cysteine residue in the ATP-binding pocket of the receptor. Neratinib also inhibits the epidermal growth factor receptor (EGFR) kinase and the proliferation of EGFR-dependent cells (J. Med. Chem 2005; 48, 1107-1131).

TAK-285 (N-(2-(4-((3-chloro-4-(3-(trifluoromethyl)phenoxy)phenyl)amino)-5H-pyrrolo[3,2-d]pyrimidin-5-yl)ethyl)-3-hydroxy-3-methylbutanamide): is a novel dual HER2 and EGFR inhibitor with IC50 of 17 nM and 23 nM, respectively (J Med Chem 2011; 54(23), 8030-8050).

AST-1306 (N-[4-[[3-Chloro-4-[(3-fluorobenzyl)oxy]phenyl]amino]quinazolin-6-yl]acrylamide): is a novel irreversible epidermal growth factor receptor inhibitor for EGFR and ErbB4 with IC50 of 0.5 and 0.8 nM, respectively (PLoS One. 2011; 6(7), e21487).

ARRY334543 (4-N-[3-chloro-4-(1,3-thiazol-2-ylmethoxy)phenyl]-6-N-[(4R)-4-methyl-4,5-dihydro-1,3-oxazol-2-yl]quinazoline-4,6-diamine): is a selective and potent kinase ErbB-1 and ErbB-2 inhibitor with IC50 of 7 and 2 nM, respectively.

Tyrphostin 9 (SF 6847; 2-[[3,5-bis(1,1-dimethylethyl)-4-hydroxyphenyl]methylene]-propanedinitrile); is an inhibitor of EGFR with IC50 of 460 μM.

### VEGFR Inhibitors

VEGFR inhibitors are examples of chemotherapeutic agents that may be used together with a virus to treat a cancer in accordance with the present invention.

There are three VEGFR receptors in humans, VEGFR1, VEGFR2 and VEGFR3, which have distinct physiological functions. Deregulation of multiple elements of the VEGFR signal transduction cascades have been reported in many types of cancers.

The present invention concerns the use of any VEGFR inhibitor, including any VEGFR inhibitor in either of these classes.

As such, a VEGFR inhibitor is an agent capable of inhibiting the activity of any one of VEGFR1, VEGFR2 and/or VEGFR3 individually or in combination, preferably mammalian VEGFR1, VEGFR2 or VEGFR3, more preferably human. In some preferred embodiments the VEGFR inhibitor is capable of inhibiting VEGFR2 (alone or in addition to being capable of inhibiting VEGFR1 and/or VEGFR3).

A VEGFR inhibitor will typically be a chemical entity, e.g. small molecule pharmaceutical.

Inhibition of VEGFR activity can be tested using routine procedures known to those of ordinary skill in the art, thus allowing one to confirm whether a given agent is a VEGFR inhibitor. For example, the skilled person would readily be able to identify suitable VEGFR inhibitors for use in the present invention by ELISA assay and detection of phosphorylated tyrosine as described in Wedge S R et al., (20000 Cancer Res. Vol. 60, pp. 970 or Hennequin L F (1999) J. Med. Chem., Vol. 42 pp. 5369-5389). Alternatively in vitro kinase assays as described in Wood et al. (2000) Cancer Res. Vol, 60, pp, 2178-2189 and Traxler P (2004) Cancer Res. Vol. 64, pp. 4931, would enable the determination of VEGFR inhibitors using recombinant GST-fused kinase domains expressed in baculovirus using γ-[³³P]ATP as a phosphate donor and polyGluTyr-(4:1) peptide as an acceptor, and detecting phosphorylation by scintillation counting. Alternatively, a commercially available kit designed for screening of VEGFR inhibitors may be selected such as the MSD® 384-well MULTI-ARRAY® Phospho-VEGFR-2 Assay (Meso Scale Diagnostics, LLC, Rockvill, Md., USA).

A number of VEGFR inhibitors are well known, and are reviewed in Ivy S P et al., “An overview of small molecule inhibitors of VEGFR signalling” Nature Rev. Clin. Oncol. Vol 6, pp/569-579. Several are discussed below.

### VEFR1 Inhibitors:

ZM 306416 (4-[(4′-Chloro-2′-fluoro)phenylamino]-6,7-dimethoxyquinazoline hydrochloride) is a VEGFR1 inhibitor with an IC50 of 33 μM (Antczak C et al., J. Biomol, Screen Vol. 17, pp. 885-898).

Regorafenib (BAY 73-4506, Stivarga™) (4-[4-({[4-Chloro-3-(trifluoromethyl)phenyl]carbamoyl}amino)-3-fluorophenoxy]-N-methylpyridine-2-carboxamide hydrate) is an oral multi-kinase inhibitor of VEGFR 1, 2 and 3, PDGFRβ, c-KIT, RET and Ra-1. IC50 against VEGFR 1/2/3 of 13/4, 2/46 nM respectively. 1050 against PDGFRβ of 22 nM. IC50 against c-KIT of 7 nM. IC50 against RET of 1.5 nM. IC50 against Raf-1 of 2.5 nM.

### VEGFR2 Inhibitors:

### Sorafenib

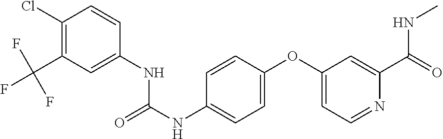 [SMILES: CNC(=O)c1cc(Oc2ccc(NC(=O)Nc3ccc(Cl)c(C(F)(F)F)c3)cc2)ccn1]

Sorafenib (4-[4-[[4-chloro-3-(trifluoromethyl)phenyl]carbamoylamino]phenoxy]-N-methyl-pyridine-2-carboxamide), marketed under the trade name Nexavar™ is a small molecule VEGFR2 inhibitor, thought to inhibit almost all known cellular responses to VEGF and therefore inhibiting angiogenesis. Sorafenib also inhibits PDGFR, Raf and c-Kit and inhibition of the Raf/MEK/ERK signalling axis abrogated tumour growth (Wilhelm et al., 2008 Mol Cancer Ther Vol. 7, pp. 3129-3140). Sorafenib was co-developed and co-marketed by Bayer and Onyx as Nexavar™ and was approved by the FDA/EMA in 2005/2006 for use in the treatment of advanced RCC, and by EMA/FDA in 2007 for advanced HCC. Monotherapy of HCC with Sorafenib reduces the risk of death during year 1 by 31% and prolonged median survival and the time to progression by nearly 3 months (reviewed in Liovet et al., 2008 N Engl J Med, Vol. 359, pp. 378-390).

### Cabozantinib (Cometriq (Formerly Known as XL1814); N-(4-((6,7-Dimethoxyquinolin-4-yl)oxy)phenyl)-N-(4-fluorophenyl)cyclopropane-1,1-dicarboxamide; Exelixis Inc,)

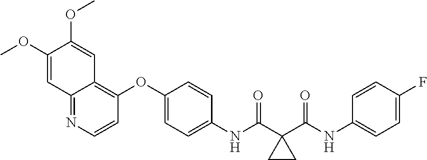 [SMILES: COc1cc2nccc(Oc3ccc(NC(=O)C4(C(=O)Nc5ccc(F)cc5)CC4)cc3)c2cc1OC]

Cabozantinib is a potent, orally bioavailable tyrosine kinase inhibitor that targets multiple pathways including VEGFR2, the rearranged during transfection (RET) and mesenchymal-epithelial transition factor (MET) receptor. Hepatocyte growth factor (HGF) is the ligand for c-MET and is a potent motility factor and mitogen. HGF and c-MET are upregulated in many human cancers in which they contribute to tumour growth, angiogenesis, invasiveness and metastasis and Cabozantinib can reduce tumour growth, angiogenesis and metastases in preclinical models (You et al Cancer Res 2011; 71: 4758-4768, Yakes et al Mol Cancer Ther (2011); 10: 2298-2308).

Cabozantinib was approved by the FDA in November 2012 for the treatment of medullary thyroid cancer and is undergoing other extensive trials for the treatment of prostate, ovarian, brain, melanoma, breast, non-small cell lung, HCC and RCC with positive trial data indicating that Cabozantinib is particularly beneficial in metastatic advanced prostate cancer (Smith et al J Clin Oncol (2013); 31: 412-419).

Sunitinib (Sutent; N-(2-diethylaminoethyl)-5-[(Z)-(5-fluoro-2-oxo-1H-indol-3-ylidene)methyl]-2,4-dimethyl-1H-pyrrole-3-carboxamide) is a multi-targeted inhibitor targeting VEGFR2 and PDGFRβ with IC50 of 80 nM and 2 nM, and also inhibits c-Kit. Sunitinib also potently inhibits Kit and FLT-3 (Sun L, et al. 2008 J. Med. Chem Vol. 46, pp. 1116-1119). Sunitinib inhibits VEGF-dependent VEGFR2 phosphorylation and PDGF-dependent PDGFRβ phosphorylation with IC50 of 10 nM and 10 nM, respectively (Mendel D B et al., 2003 Clin. Cancer Res. Vol. 9, pp. 327-337).

Foretinib (GSK1363089, XL880, EXEL-2880; N1′-[3-fluoro-4-[[6-methoxy-7-(3-morpholinopropoxy)-4-quinolyl]oxy]phenyl]-N1-(4-fluorophenyl)cyclopropane-1,1-dicarboxamide) is an ATP-competitive inhibitor of MET and VEGFR2 with 1050 of 0.4 nM and 0.9 nM, respectively (Qian F et al., 2009 Cancer Res Vol. 69, pp. 8009-8016).

Cediranib (AZD2171; 4-[(4-fluoro-2-methyl-1H-indol-5-yl)oxy]-6-methoxy-7-[3-(pyrrolidin-1-yl)propoxy]quinazoline) is a highly potent VEGFR2 inhibitor with IC50 of 0.5 nM.

Golvatinib (E7050; N-(2-fluoro-4-((2-(4-(4-methylpiperazin-1-yl)piperidine-1-carboxamido)pyridin-4-yl)oxy)phenyl)-N-(4-fluorophenyl)cyclopropane-1,1-dicarboxamide) is a dual c-Met and VEGFR2 inhibitor with an 1050 of 14 nM and 16 nM, respectively, and potently represses the growth of both c-met amplified tumor cells and endothelial cells stimulated with either HGF or VEGF (Nagagawa T et al., 2010 Cancer Sci Vol. 101, pp. 210-215).

ZM 323881 HCl (5-((7-Benzyloxyquinazolin-4-yl)amino)-4-fluoro-2-methylphenol hydrochloride) is a potent and selective inhibitor of VGEFR2 with an IC50<2 nM (Whittles C E, et al., 2002 Microcirculation, Vol. 9, pp. 513-522.)

Apatinib (YN968D1; N-[4-(1-cyanocyclopentyl) phenyl-2-(4-picolyl)amino-3-Nicotinamide methanesulphonate) in a VEGFR2 inhibitor with an 1050 of 2.34 nM.

BMS 794833 (N-(4-((2-amino-3-chloropyridin-4-yl)oxy)-3-fluorophenyl)-5-(4-fluorophenyl)-4-oxo-1,4-dihydropyridine-3-carboxamide) is a potent inhibitor of Met and VEGFR2 with an IC50 of 1.7 nM and 15 nM, respectively (WO/2009094417).

RAF265 (CHIR-265; 1-methyl-5-(2-(5-(trifluoromethyl)-1H-imidazol-2-yl)pyridin-4-yloxy)-N-(4-(trifluoromethyl)phenyl)-1H-benzo[d]imidazol-2-amine) is a selective and potent inhibitor of B-Raf and VEGFR2 with IC50 of 3-60 nM and EC50 of 30 nM, respectively (Amiri P et al., US20070299039)

AEE788 (NVP-AEE788; 6-[4-[(4-Ethylpiperazin-1-yl)methyl]phenyl]-N-[(1R)-1-phenylethyl]-7H-pyrrolo[2,3-d]pyrimidin-4-amine) inhibits VEGFR2 with an IC50 of 77 nM. It also inhibits EGFR, HER2/ErbB2 ad Fit (Traxler P et al., 2004 Cancer Res, Vol. 64, pp. 4931-4941).

Ponatinib (AP24534; 3-(2-Imidazo[1,2-b]pyridazin-3-ylethynyl)-4-methyl-N-[4-[(4-methylpiperazin-1-yl)methyl]-3-(trifluoromethyl)phenyl]benzamide) is a multi-target inhibitor of VEGFR2 with an IC50 of 1.5 nM. It also inhibits Abl, PDGFRα, FGFR1 and Src (O'Hare T et al., 2009 Cancer Cell Vol. 16, pp. 401-412).

TSU-68 (SU6668, Orantinib; (Z)-3-(2,4-dimethyl-5-((2-oxoindolin-3-ylidene)methyl)-1H-pyrrol-3-yl)propanoic acid) is a potent inhibitor of VEGFR2 with Ki of 2.1 μM. It also inhibits FGFR1 and PDGFRβ (Laird A M et al., 2000 Cancer Res Vol. 60, pp. 4152-4160).

Vandetanib (Zactima, ZD6474; N-(4-bromo-2-fluorophenyl)-6-methoxy-7-[(1-methylpiperidin-4-yl)methoxy]quinazolin-4-amine) is an inhibitor of VEGFR2 with an 1050 of 40 nM (Inoue K et al., 2012 Clin. Can. Res.). It also inhibits VEGFR3 and EGFR with IC50 of 110 nM and 500 nM, respectively.

Regorafenib (BAY 73-4506, Stivarga™) (4-[4-({[4-Chloro-3-(trifluoromethyl)phenyl]carbamoyl}amino)-3-fluorophenoxy]-N-methylpyridine-2-carboxamide hydrate)—see above.

Other small molecule VEGFR2 inhibitors include: Ki8751 (N-(2,4-Difluorophenyl)-N′-[4-[(6,7-dimethoxy-4-guinolinyl)oxy]-2-fluorophenyl]urea), OSI-930 (3-((guinolin-4-ylmethyl)amino)-N-(4-(trifluoromethoxy)phenyl)thiophene-2-carboxamide), TSU-68 (5-[1,2-Dihydro-2-oxo-3H-indol-3-ylidene)methyl]-2,4-dimethyl-1H-pyrrole-3-propanoic acid), PD173074 (N-[2-[[4-(Diethylamino)butyl]amino]-6-(3,5-dimethoxyphenyl)pyrido[2,3-d]pyrimidin-7-yl]-N′-(1,1-dimethylethyl)urea), and Brivanib (BMS-540215; (S)-(R)-1-((4-((4-fluoro-2-methyl-1H-indol-5-yl)oxy)-5-methylpyrrolo[2,1-f][1,2,4]triazin-6-yl)oxy)propan-2-yl 2-aminopropanoate).

### VEGFR3 Inhibitors:

SAR131675 ((R)-2-amino-1-ethyl-7-(3-hydroxy-4-methoxy-3-methylbut-1-ynyl)-N-methyl-4-oxo-1,4-dihydro-1,8-naphthyridine-3-carboxamide) is a VEGFR3 inhibitor with an 1050 of 20 nM (Alam A., 2012 Mol. Cancer Ther. Vol. 11, pp. 1637-1649).

### Inhibitors Targeting Multi VEGFRs:

### Pazopanib (Votrient, GW786034, GSK; 5-[[4-[(2,3-Dimethyl-2H-indazol-6-yl)methylamino]-2-pyrimidinyl]amino]-2-methylbenzolsulfonamide)

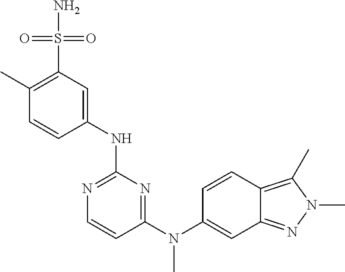

Pazopanib is a second-generation multi-targeted tyrosine kinase inhibitor against VEGFR-1, -2, and -3, platelet-derived growth factor receptor-α, platelet-derived growth factor receptor-β, and c-kit (Sonpavde and Hutson, Curr Oncol Rep. 2007; 9:115-119). Preclinical evaluation has revealed excellent antiangiogenic and antitumor activity (Kumar et al., Mol Cancer Ther. (2007); 6: 2012-2021). Some activity against B-Raf has also been reported in xenograft tumour models (Gril et al PLoS ONE (2011); 6:1-11). Pazopanib has demonstrated significantly improved progression-free survival and tumour responses (Sternberg et al., J Clin Oncol (2010); 28: 1061-1068) and was approved for use in advanced renal cell carcinoma (RCC) by the Food and Drug Administration in the United States in October 2009.

Axitinib (AG-013736; N-Methyl-2-[[3-[(E)-2-pyridin-2-ylethenyl]-1H-indazol-6-yl]sulfanyl]benzamide) is a second generation multi-target inhibitor of VEGFR1, BEGFR2, VGFR3, PDGFRβ and c-Kit with an IC50 0.1 nM, 0.2 nM, 0.1-0.3 nM, 1.6 nM and 1.7 nM, respectively. It is thought that it inhibits the proliferation of variable cell lines, and blocks the autophosphorylation of BEGFR mediated endothelial cell viability tude formation and downstream signalling. Axitinib has shown activity in variable tumors including RCC, thyroid cancer, non-small lunch cancer, colorectal cancer and melanoma (Hu-Lowe D D et al., 2008 Clin. Cancer Res. Vol. 14, pp. 7272-7283).

Ninetedinib (BIBF1120, Vargatef; methyl (3Z)-3-{[(4-{methyl[(4-methylpiperazin-1-yl)acetyl]amino}phenyl)amino](phenyl)methylidene}-2-oxo-2,3-dihydro-1H-indole-6-carboxylate) is a is a potent inhibitor of VEGFR1, VEGFR2, VEGFR3, FGFR1/2/3 and PDGFRα/β with IC50 of 34 nM/13 nM/13 nM, 69 nM/37 nM/108 nM and 59 nM/65 nM, respectively (Hilberg F et al., 2008 Cancer Res. Vol. 68, pp. 4774-4782).

Dovitinib (TKI-258, CHIR258; 1-amino-5-fluoro-3-(6-(4-methylpiperazin-1-yl)-1H-benzo[d]imidazol-2-yl)quinolin-2(1H)-one) is a multi-target inhibitor for VEGFR1, VEGFR2 and VEGFR3 with an IC50 of 10 nM, 13 nM and 8 nM respectively. It also inhibits Flt3, c-Kit and PDGFRα/β (Huynh H et al., 2012 J. Hepatol Vol. 56, pp. 595-601).

Telatinib (BAY 57-9352; (4E,6E,8S,9S,10E,12S,13R,14S,16R)-19-(allylamino)-13-hydroxy-8,14-dimethoxy-4,10,12,16-tetramethyl-3,20,22-trioxo-2-azabicyclo[16.3.1]docosa-1(21),4,6,10,18-pentaen-9-ylcarbamate) is a potent inhibitor of VEGFR2 and VEGFR3, c-KIT and PCGFRβ with an IC50 of 6 nM/4 nM, 1 nM and 15 nM, respectively (Steeghs N et al., 2008 Clin. Cancer Res. Vol. 14, pp. 3470-3476).

KRN 633 (N-[2-Chloro-4-[(6,7-dimethoxy-4-quinazolinyl)oxy]phenyl]-N′-propylurea) is an ATP-competitive inhibitor of VEGFR1, VEGFR2 and VEGFR3 with IC50 of 170 nM/160 nM/125 nM and also inhibits PDGFRα/β with IC50 of 965 nM/9850 nM (Nakamura K et al., 2004 Mol. Cancer Ther. Vol. 3, pp. 1639-1649).

Regorafenib (BAY 73-4506, Stivarga™) (4-[4-({[4-Chloro-3-(trifluoromethyl)phenyl]carbamoyl}amino)-3-fluorophenoxy]-N-methylpyridine-2-carboxamide hydrate)—see above.

Other multi-VEGFR inhibitors include: MGCD-265, Tivozanib (AV-951), ENMD-2076, Regorafenib (BAY 73-4506), E7080 (Levantinib), Brivanib alaninate (BMS-582664) and Motesanib diphosphate (AMG-706), Linifanib (ABT-869).

### PDGFR Inhibitors

PDGFR inhibitors are examples of chemotherapeutic agents that may be used together with a virus to treat a cancer in accordance with the present invention

There are two PDGFR receptors in humans, PDGFRα and PDGFRβ, which have distinct physiological functions. Deregulation of multiple elements of the PDGFR signal transduction cascades have been reported in many types of cancers. Other members of the PDGFR family include CSF1R, SCFR/c-Kit, FLT3/Flk2.

The present invention concerns the use of any PDGFR inhibitor, including any PDGFR inhibitor in any of these classes.

As such, a PDGFR inhibitor may be an agent capable of inhibiting the activity of any one of PDGFRα, PDGFRβ, CSF1R, SCFR/c-Kit, and/or FLT3/FIk2 individually or in combination, preferably mammalian PDGFRα, PDGFRβ, CSF1R, SCFR/c-Kit, and/or FLT3/Flk2, more preferably human. In some preferred embodiments the PDGFR inhibitor is capable of inhibiting PDGFRα (alone or in addition to being capable of inhibiting PDGFRβ).

A PDGFR inhibitor will typically be a chemical entity, e.g. small molecule pharmaceutical.

Inhibition of PDGFR activity can be tested using routine procedures known to those of ordinary skill in the art, thus allowing one to confirm whether a given agent is a PDGFR inhibitor. For example, the skilled person would readily be able to identify suitable PDGFR inhibitors for use in the present invention by methods described in Iwata H et al. (2011) Biochemistry Vol. 50, pp. 738-751.

A number of PDGFR inhibitors are well known, and are reviewed in Arora A & Scholar E M 2005 J. Pharm. Expr. Ther. Vol. 315, pp. 971-979. Several are discussed below.

### Imatinib (INN, 511571, Gleevec, Glivec; 4-[(4-methylpiperazin-1-yl)methyl]-N-(4-methyl-3-{[4-(pyridin-3-yl)pyrimidin-2-yl]amino}phenyl)benzamide)

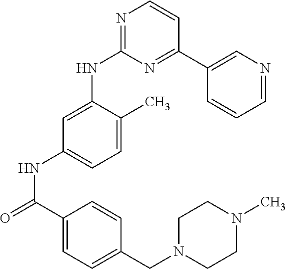 [SMILES: Cc1ccc(NC(=O)c2ccc(CN3CCN(C)CC3)cc2)cc1Nc1nccc(-c2cccnc2)n1]

Imatinib is a multi-target inhibitor of v-Abl, c-Kit and PDGFR with IC50 of 0.6 μM, 0.1 μM and 0.1 μM, respectively. Imatinib is used in chronic myelogenous leukemia (CML), gastrointestinal stromal tumors (GISTs) amongst other malignancies. It is FDA approved for use in adult patients with relapsed or refractory Ph-positive ALL, myelodysplastic/myeloproliferative diseases associated with platelet-derived growth factor receptor gene re-arrangements, aggressive systemic mastocytosis (ASM) without or an unknown D816V c-KIT mutation, hypereosinophilic syndrome (HES) and/or chronic eosinophilic leukemia (CEL) who have the FIP1L1-PDGFRα fusion kinase (CHIC2 allele deletion) or FIP1L1-PDGFRα fusion kinase negative or unknown, unresectable, recurrent and/or metastatic dermatofibrosarcoma protuberans

Imatinib Mesylate (4-[(4-Methyl-1-piperazinyl)methyl]-N-(4-methyl-3-{[4-(3-pyridinyl)-2-pyrimidinyl]amino}phenyl)benzamide methanesulfonate (1:1)): a mesylate salt of Imatinib, which is a multi-target inhibitor of v-Abl, c-Kit and PDGFR with IC50 of 0.6 μM, 0.1 μM and 0.1 μM, respectively.

Linifanib (ABT-869; see above): is a potent ATP-competitive inhibitor of KDR, CSF-1R, Flt-1, Flt-3 and PDGFRβ with IC50 of 4 nM, 3 nM, 3 nM, 3 nM, and 66 nM respectively.

Dasatinib (BMS-354825, Sprycel; N-(2-chloro-6-methylphenyl)-2-[[6-[4-(2-hydroxyethyl)-1-piperazinyl]-2-methyl-4-pyrimidinyl]amino]-5-thiazole carboxamide monohydrate): is a novel, potent and multi-targeted inhibitor that targets Abl, Src and c-Kit, with IC50 of <1 nM, 0.5 nM and 79 nM, respectively.

Dovitinib (TKI-258, CHIR258; see above): is a multi-targeted inhibitor for Flt3, c-Kit, FGFR1/3, VEGFR1/2/3, PDGFRα/β with IC50 of 1 nM, 2 nM, 8 nM/9 nM and 10 nM/13 nM/8 nM, 210 nM/27 nM respectively (Trudel S, et al. 2005 Blood. Vol. 105, pp. 2941-2948).

Masitinib (AB1010; 4-[(4-Methylpiperazin-1-yl)methyl]-N-(4-methyl-3-{[4-(pyridin-3-yl)-1,3-thiazol-2-yl]amino}phenyl)benzamide): is an inhibitor for Kit and PDGFRα/β with IC50 of 200 nM and 540 nM/800 nM, respectively. Masitinib is a competitive inhibitor against ATP at concentrations≦500 nM. Masitinib also potently inhibits recombinant PDGFR and the intracellular kinase Lyn, and to a lesser extent, fibroblast growth factor receptor 3. In contrast, Masitinib demonstrates weak inhibition of Abl and c-Fms. Masitinib more strongly inhibits degranulation, cytokine production, and bone marrow mast cell migration than Imatinib (Dubreuil P, et al. PLoS One, 2009, 4(9), e7258.)

Tivozanib (AV-1951, KRN-951; see above): AV-951 is a novel quinoline-urea derivative, AV-951 inhibits phosphorylation of PDGFRβ and c-Kit with IC50 of 1.72 and 1.63 nM, respectively (Nakamura K, et al. Cancer Res, 2006, 66(18), 9134-9142).

Amuvatinib (MP-470; N-(benzo[d][1,3]dioxol-5-ylmethyl)-4-(benzofuro[3,2-d]pyrimidin-4-yl)piperazine-1-carbothioamide): is a potent and multi-targeted inhibitor of C-Kit^{D816H} PDGFα^{V561D} and Flt3^{D835Y} with IC50 of 10 nM, 40 nM and 81 nM, respectively (Mahadevan D, et al. Oncogene, 2007, 26(27), 3909-3919).

TSU-68 (SU6668, Orantinib; see above): is a potent inhibitor of Flk-1/KDR, FGFR1 and PDGFRβ with K, of 2.1 μM, 1.2 μM, and 8 nM, respectively (Laird A D, et al. Cancer Res, 2000, 60(15), 4152-4160).

CP 673451 (1-(2-(5-(2-methoxyethoxy)-1H-benzo[d]imidazol-1-yl)quinolin-8-yl)piperidin-4-amine): is a potent PDGFR-β inhibitor with an IC50 of 1 nM.

MK-2461 (N-((2R)-1,4-Dioxan-2-ylmethyl)-N-methyl-N′-[3-(1-methyl-1H-pyrazol-4-yl)-5-oxo-5H-benzo[4,5]cyclohepta[1,2-b]pyridin-7-yl]sulfamide): is a potent inhibitor of c-Met, Ron, Flt1/3, PDGFRβ, Mer and FGFR1/2/3 with IC50 of 2.5 nM, 7 nM, 10 nM/22 nM, 22 nM, 24 nM and 65 nM/39 nM/50 nM, respectively (Pan B S, et al. Cancer Res, 2010, 70(4), 1524-1533).

Sorafenib (4-[4-[[4-chloro-3-(trifluoromethyl)phenyl]carbamoylamino]phenoxy]-N-methyl-pyridine-2-carboxamide)—see above.

Sunitinib (N-(2-diethylaminoethyl)-5-[(Z)-(5-fluoro-2-oxo-1H-indol-3-ylidene)methyl]-2,4-dimethyl-1H-pyrrole-3-carboxamide)—see above.

Pazopanib (5-[[4-[(2,3-Dimethyl-2H-indazol-6-yl)methylamino]-2-pyrimidinyl]amino]-2-methylbenzolsulfonamide)—see above.

Regorafenib (4-[4-({[4-Chloro-3-(trifluoromethyl)phenyl]carbamoyl}amino)-3-fluorophenoxy]-N-methylpyridine-2-carboxamide hydrate)—see above.

### ALK Inhibitors

ALK inhibitors are examples of chemotherapeutic agents that may be used together with a virus to treat a cancer in accordance with the present invention.

Deregulation of multiple elements of the ALK signal transduction cascades have been reported in many types of cancers.

An ALK inhibitor is an agent capable of inhibiting the activity of ALK individually, preferably mammalian ALK, more preferably human.

An ALK inhibitor will typically be a chemical entity, e.g. small molecule pharmaceutical.

Inhibition of ALK activity can be tested using routine procedures known to those of ordinary skill in the art, thus allowing one to confirm whether a given agent is an ALK inhibitor. For example, the skilled person would readily be able to identify suitable ALK inhibitors for use in the present invention by using a cellular ALK kinase phosphorylation ELISA as described in Christensen J G et al. (2007) “Cytoreductive antitumor activity of PF-2341066, a novel inhibitor of anaplastic lymphoma kinase and c-Met, in experimental models of anaplastic large-cell lymphoma” Mol. Cancer Ther. Vol. 6, pp. 3314-3322 or alternatively by time-resolved fluorescence resonance energy transfer (TR-FRET) assay or fluorescence polarization assay (FP) as described in Sakamoto H et al., (2011) “CH5424802, a Selective ALK Inhibitor Capable of Blocking the Resistant Gatekeeper Mutant” Cancer Cell. Vol. 19, pp. 679-690. Alternatively a commercial assay may be used such as the phosphor-ALK (Tyr1604) assay (Cell Signalling Technologies).

A number of ALK inhibitors are well known, and are reviewed in Hallberg B & Palmer R H (2011) ALK and NSCLC: Targeted therapy with ALK inhibitors. Med Rep. Vol. 3, pp. 21. Several are discussed below.

### Crizotinib (PF-2341066; Xalkori; 3-[(1R)-1-(2,6-dichloro-3-fluorophenyl)ethoxy]-5-(1-piperidin-4-ylpyrazol-4-yl)pyridin-2-amine)

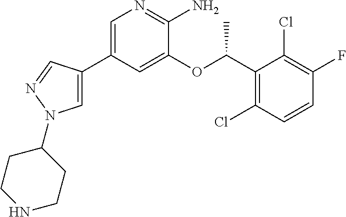 [SMILES: C[C@@H](Oc1cc(-c2cnn(C3CCNCC3)c2)cnc1N)c1c(Cl)ccc(F)c1Cl]

Crizotinib is an anti-cancer drug acting as an ALK (anaplastic lymphoma kinase) and ROS1(c-ros ongogene1) inhibitor. Crizotinib functions as a protein kinase inhibitor by competitively binding with the ATP-binding pocket of target kinases. Crizotinib inhibits the c-Met/Hepatocyte Growth Factor Receptor (HGFR) tyrosine kinase, which is involved in the oncogenesis several histological forms of malignant neoplasms (Rodig, S L and Shaprio G I. Curr Opin Investig Drugs, December 2010: 11(12):1477-90). Crizotinib is thought to exert its effects through modulation of the growth, migration, and invasion of malignant cells. Crizotinib may also act via inhibition of angiogenesis in malignant tumours.

Crizotinib is approved for the treatment of some non-small cell lung carcinoma (NSCLC) in the US and some other countries, and undergoing clinical trials testing its safety and efficacy in anaplastic large cell lymphomas, neuroblastoma and other advanced solid tumours in both adults and children.

Crizotinib is a potent ATP-competitive inhibitor of c-Met and ALK with IC50 of 11 nM and 24 nM, respectively (Blood 2005; 105: 2640-2653). Approved by the FDA for the treatment of late stage lung cancer.

GSK1838705A (2-(2-(1-(2-(dimethylamino)acetyl)-5-nnethoxyindolin-6-ylamino)-7H-pyrrolo[2,3-d]pyrimidin-4-ylamino)-6-fluoro-N-methylbenzamide): GSK1838705A is a potent inhibitor of IGF-1R, IR and ALK with IC50 of 2.0 nM, 1.6 nM and 0.5 nM, respectively. GSK1838705A prevents the in vitro proliferation of cell lines derived from solid and hematologic malignancies, including multiple myeloma and Ewing's sarcoma, and retards the growth of human tumor xenografts in vivo (Mol Cancer Ther 2009 October; 8(10):2811-20).

TAE684 (NVP-TAE684; 5-chloro-N4-(2-(isopropylsulfonyl)phenyl)-N2-(2-methoxy-4-(4-(4-methylpiperazin-1-yl)piperidin-1-yl)phenyl)pyrimidine-2,4-diamine): is a highly potent and selective small molecule ALK inhibitor, which blocked the growth of ALCL-derived and ALK-dependent cell lines with IC50 values between 2 and 10 nM (PNAS Jan. 2, 2007; 104:270-275).

CH5424802 (9-ethyl-6,6-dimethyl-8-(4-morpholinopiperidin-1-yl)-11-oxo-6,11-dihydro-5H-benzo[b]carbazole-3-carbonitrile): is a potent and selective ALK inhibitor with an IC50 of 1.9 nM (Cancer Cell. 2011; 19(5), 679-690).

### Insulin-Like Growth Factor Receptor Inhibitors

Insulin-like growth factor receptor inhibitors are examples of chemotherapeutic agents that may be used together with a virus to treat a cancer in accordance with the present invention.

There are two insulin-like growth factor receptors in humans, IGF-1R and IGF-2R, which have distinct physiological functions. Deregulation of multiple elements of the IGF-1R signal transduction cascades have been reported in many types of cancers.

The present invention concerns the use of any insulin-like growth factor receptor inhibitor, including any insulin-like growth factor receptor inhibitor in either of these classes.

As such, an insulin-like growth factor receptor is an agent capable of inhibiting the activity of any one of IGF-1R and/or IGF-2R individually or in combination, preferably mammalian IGF-1R and/or IGF-2R, more preferably human. In some preferred embodiments the insulin-like growth factor receptor inhibitor is capable of inhibiting IGF-1R (alone or in addition to being capable of inhibiting IGF-2R).

An insulin-like growth factor receptor inhibitor will typically be a chemical entity, e.g. small molecule pharmaceutical.

Inhibition of insulin-like growth factor receptor activity can be tested using routine procedures known to those of ordinary skill in the art, thus allowing one to confirm whether a given agent is an insulin-like growth factor receptor inhibitor. For example, the skilled person would readily be able to identify suitable insulin-like growth factor receptor inhibitors for use in the present invention by receptor phosphorylation assays using NIH-3T3 cells overexpressing human IGR-IR or IR as described in Kaleko M et al., (1990) “Overexpression of the human insulin growth factor 1 receptor promotes ligands-dependent neoplastic transformation. Mol. Cell. Biol. Vol. 10, pp. 464-473 and Sabbatini P et al., (2009) “GSK1838705A inhibits the insulin-like growth factor-1 receptor and anaplastic lymphoma kinase and shows antitumor activity in experimental models of human cancers” Mol. Cancer Ther. Vol. 8, pp. 2811.

A number of IGF-1R inhibitors are well known, and are reviewed in Gombos A et al., (2012) “Clinical development of insulin-like growth factor receptor-1 (IGF-1R) inhibitors: At the crossroad?” Invest. New. Drugs Vol. 30, pp. 2433-2442. Several are discussed below.

GSK1838705A (see above):

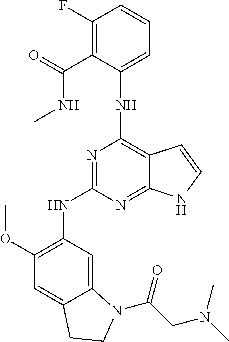 [SMILES: CNC(=O)c1c(F)cccc1Nc1nc(Nc2cc3c(cc2OC)CCN3C(=O)CN(C)C)nc2[nH]ccc12]

GSK1838705A is a potent ATP-competitive inhibitor of IGF-1R, IR and ALK with IC50 of 2.0 nM, 1.6 nM and 0.5 nM, respectively (Sabbatini P, et al. Mol Cancer Ther. 2009, 8(10), 2811-2820).

GSK1904529A (N-(2,6-difluorophenyl)-5-(3-(2-(5-ethyl-2-methoxy-4-(4-(4-(methylsulfonyl)piperazin-1-yl)piperidin-1-yl)phenylamino)pyrimidin-4-yl)H-imidazo[1,2-a]pyridin-2-yl)-2-methoxybenzamide): is a selective inhibitor of IGF-1R and IR with IC50 of 27 nM and 25 nM, respectively (Sabbatini P, et al. Mol Cancer Ther. 2009, 8(10), 2811-2820).

NVP-AEW541 (7-((1s,3s)-3-(azetidin-1-ylmethyl)cyclobutyl)-5-(3-(benzyloxy)phenyl)-7H-pyrrolo[2,3-d]pyrimidin-4-amine): is a potent inhibitor of IGF-1R with IC50 of 86 nM (García-Echeverría C, et al. Cancer Cell. 2004, 5(3), 231-239).

Linsitinib (OSI-906; (1s,3s)-3-(8-amino-1-(2-phenylquinolin-7-yl)imidazo[1,5-a]pyrazin-3-yl)-1-methylcyclobutanol): is a selective inhibitor of IGF-1R and IR with IC50 of 35 nM and 75 nM, respectively (Mulvihill M J, et al. Future Med Chem, 2009, 1(6), 1153-1171).

BMS-536924 (4-[[(2S)-2-(3-Chlorophenyl)-2-hydroxyethyl]amino]-3-[7-methyl-5-(4-morpholinyl)-1H-benzimidazol-2-yl]-2(1H)-pyridinone): is an ATP-competitive IGF-1R and IR inhibitor with IC50 of 100 nM and 73 nM, respectively (Wittman M, et al. J Med Chem, 2005, 48(18), 5639-5643).

NVP-ADW742 (5-(3-(benzyloxy)phenyl)-7-((1r,3r)-3-(pyrrolidin-1-ylmethyl)cyclobutyl)-7H-pyrrolo[2,3-d]pyrimidin-4-amine): is an IGF-1R inhibitor with IC50 of 0.17 μM (Mitsiades C S, et al. Cancer Cell, 2004, 5(3), 221-230).

BMS-754807 ((S)-1-(4-(5-cyclopropyl-1H-pyrazol-3-ylamino)pyrrolo[1,2-f][1,2,4]triazin-2-yl)-N-(6-fluoropyridin-3-yl)-2-methylpyrrolidine-2-carboxamide): is an IGF-1R inhibitor with an IC50 of 13 nM.

Tyrphostin (AG-1024; 2-(3-bromo-5-tert-butyl-4-hydroxybenzylidene)malononitrile): is a specific IGF-1R and IR inhibitor with IC50 of 0.4 μM and 0.1 μM, respectively (Parrizas M, et al. Endocrinology, 1997, 138(4), 1427-1433).

c-KIT (cD117) Inhibitors

Mast/stem cell growth factor receptor (SCFR), also known as proto-oncogene c-Kit, tyrosine-protein kinase Kit or CD117, is a 145-kd transmembrane glycoprotein, and is the normal cellular homologue of the viral oncogene v-kit and a member of the receptor tyrosine kinase subclass III family that includes receptors for platelet-derived growth factor (PDGF), macrophage colony-stimulating factor, and flt3 ligand.

A number of c-KIT inhibitors are well known. Examples include:

Sorafenib (4-[4-[[4-chloro-3-(trifluoromethyl)phenyl]carbamoylamino]phenoxy]-N-methyl-pyridine-2-carboxamide)—see above.

Sunitinib (N-(2-diethylaminoethyl)-5-[(Z)-(5-fluoro-2-oxo-1H-indol-3-ylidene)methyl]-2,4-dimethyl-1H-pyrrole-3-carboxamide)—see above.

Pazopanib (5-[[4-[(2,3-Dimethyl-2H-indazol-6-yl)methylamino]-2-pyrimidinyl]amino]-2-methylbenzolsulfonamide)—see above.

Dovitinib (TKI-258, CHIR258) (1-amino-5-fluoro-3-(6-(4-methylpiperazin-1-yl)-1H-benzo[d]imidazol-2-yl)quinolin-2(1H)-one)—see above.

c-MET (HGFR) Inhibitors

c-Met (MET or MNNG HOS Transforming gene) is a proto-oncogene that encodes a membrane receptor protein called hepatocyte growth factor receptor (HGFR) [Bottaro D P, Rubin J S, Faletto D L, Chan A M, Kmiecik T E, Vande Woude G F, Aaronson S A (February 1991). “Identification of the hepatocyte growth factor receptor as the met proto-oncogene product”. Science 251 (4995): 802-4]. The hepatocyte growth factor receptor protein possesses tyrosine-kinase activity [Cooper C S (January 1992). “The met oncogene: from detection by transfection to transmembrane receptor for hepatocyte growth factor”. Oncogene 7 (1): 3-7.].

A number of c-MET inhibitors are well known. Examples include:

Cabozantinib (N-(4-(6,7-dimethoxyquinolin-4-yloxy)phenyl)-N-(4-fluorophenyl)cyclopropane-1,1-dicarboxamide)—see above.

Crizotinib (3-[(1R)-1-(2,6-dichloro-3-fluorophenyl)ethoxy]-5-(1-piperidin-4-ylpyrazol-4-yl)pyridin-2-amine)—see above.

Regorafenib (4-[4-({[4-Chloro-3-(trifluoromethyl)phenyl]carbamoyl}amino)-3-fluorophenoxy]-N-methylpyridine-2-carboxamide hydrate)—see above.

### Forms of Chemotherapeutic Agent

The active compound of a given chemotherapeutic agent may be provided in the form of a corresponding salt, solvate, or prodrug. In this specification reference to the chemotherapeutic agent includes reference to such forms.

### Salts

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also include salt forms thereof.

### Solvates

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

Unless otherwise specified, a reference to a particular compound also include solvate forms thereof.

### Prodrugs

It may be convenient or desirable to prepare, purify, and/or handle the active compound in the form of a prodrug. The term “prodrug,” as used herein, pertains to a compound which, when metabolised (e.g., in vivo), yields the desired active compound. Typically, the prodrug is inactive, or less active than the active compound, but may provide advantageous handling, administration, or metabolic properties.

Unless otherwise specified, a reference to a particular compound also include prodrugs thereof.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is: C₁₋₇alkyl (e.g., -Me, -Et, -nPr, -iPr, -nBu, -sBu, -iBu, -tBu); C₁₋₇aminoalkyl (e.g., aminoethyl; 2-(N,N-diethylamino)ethyl; 2-(4-morpholino)ethyl); and acyloxy-C₁₋₇alkyl (e.g., acyloxymethyl; acyloxyethyl; pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxyl)carbonyloxyethyl; (4-tetrahydropyranyl)carbonyloxymethyl; and 1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Simultaneous or Sequential Administration

Compositions may be administered alone or in combination with other treatments, either simultaneously or sequentially dependent upon the condition to be treated.

In this specification a virus and chemotherapeutic agent may be administered simultaneously or sequentially.

Simultaneous administration refers to administration of the virus and chemotherapeutic agent together, for example as a pharmaceutical composition containing both agents, or immediately after each other and optionally via the same route of administration, e.g. to the same artery, vein or other blood vessel.

Sequential administration refers to administration of one of the virus or chemotherapeutic agent followed after a given time interval by separate administration of the other agent. It is not required that the two agents are administered by the same route, although this is the case in some embodiments. The time interval may be any time interval.

Whilst simultaneous or sequential administration is intended such that both the virus and chemotherapeutic agent are delivered to the same tumor tissue to effect treatment it is not essential for both agents to be present in the tumor tissue in active form at the same time.

However, in some embodiments of sequential administration the time interval is selected such that the virus and chemotherapeutic agent are expected to be present in the tumor tissue in active form at the same time, thereby allowing for a combined, additive or synergistic effect of the two agents in treating the tumor. In such embodiments the time interval selected may be any one of 5 minutes or less, 10 minutes or less, 15 minutes or less, 20 minutes or less, 25 minutes or less, 30 minutes or less, 45 minutes or less, 60 minutes or less, 90 minutes or less, 120 minutes or less, 180 minutes or less, 240 minutes or less, 300 minutes or less, 360 minutes or less, or 720 minutes or less, or 1 day or less, or 2 days or less.

### Synergy

In the present application synergy has been measured using the CompuSyn software (ComboSyn Incorporated, USA) [Chou T C and Martin N. CompuSyn for Drug Combinations: PC Software and User's Guide: A Computer Program for Quantitation of Synergism and Antagonism in Drug Combinations, and the Determination of IC50 and ED50 and LD50 Values, ComboSyn Inc, Paramus, (NJ), 2005].

The existence of a synergism between an RTK inhibitor and virus is not essential to all aspects of the present invention, although may be an advantageous characteristic. It may be an essential feature of some aspects of the present invention.

Intuitively, synergism between an oncolytic virus and an RTK inhibitor would suggest an unexpected level of improvement of enhancement of cell killing. Although this may in fact be the outcome, the combination may not lead to improvement in cell lysis by the oncolytic virus—the means by which the virus would be expected to contribute to cell killing. Evidence obtained by the inventors suggests that the effect of the RTK inhibitor can be to depress viral replication in the cell, and therefore cell lysis by viral progeny, such that an additive or synergistic effect would not be expected at all and the presence of such an effect is therefore not dependent on the cell lysis property of the virus and is unexpected in view of the effect of the RTK inhibitor on viral replication.

Synergism in cell killing may result from the induction of apoptosis by the combination of HSV and RTK inhibitor.

### Cancer

A cancer may be any unwanted cell proliferation (or any disease manifesting itself by unwanted cell proliferation), neoplasm or tumor or increased risk of or predisposition to the unwanted cell proliferation, neoplasm or tumor. The cancer may be benign or malignant and may be primary or secondary (metastatic). A neoplasm or tumor may be any abnormal growth or proliferation of cells and may be located in any tissue. Examples of tissues include the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentume, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, haematologic cancer and sarcoma.

The cancer may be one in which cells of the cancer are resistant (e.g. non- or semi-permissive) to viral (e.g. HSV-1, oncolytic virus, oncolytic HSV or HSV1716) infection (e.g. as discussed in Fu et al., supra).

In some optional embodiments the cancer is not a liver cancer, e.g. not a liver cancer in a human patient. In an optional embodiment the cancer is not a cancer originating in the liver. In another optional embodiment the cancer is not a primary liver cancer. In another optional embodiment the cancer is not a hepatocellular carcinoma. In another optional embodiment the cancer is not a cancer occurring in the liver. In another optional embodiment the cancer is not a cancer that has metastasised to the liver.

### Apoptosis

Results with tyrosine kinase inhibitors that target one or more of a VEGFR, a PDGFR, c-KIT (CD117) or c-MET signalling axes indicate that they combine with HSV1716 to enhance cell death in most of the cell lines tested even though the drugs themselves have limited toxicity (FIGS. 67 and 72).

The mechanism for this combinatorial effect was investigated by measuring the levels of apoptosis in the cell lines when infected with HSV1716, treated with selected tyrosine kinase inhibitors or infected with HSV1716 and treated with tyrosine kinase inhibitors simultaneously.

Results indicate that where the combination of drug and virus significantly stimulated apoptosis (compared to either drug or virus alone) the stimulation of apoptosis corresponded with virus/drug combinatorial synergies since, in cell lines in which the tyrosine kinase inhibitors were not synergistic, there was no significant increase in the activity of the apoptotic marker caspase 3/7.

If the late stage inhibition of apoptosis by HSV can be prevented by the action of an extrinsic agent, e.g. a drug, apoptosis might be initiated resulting in enhanced cell death. The results obtained indicate that certain classes of receptor tyrosine kinase inhibitor act to interfere with HSV anti-apoptotic gene expression such that the apoptosis process is allowed to continue irreversibly, with resulting caspase 3/7 and/or 9 activation.

As such, the inventors have established that some combinations of HSV and RTK inhibitor act in synergy to enhance cell death in cancer cells, i.e. enhancing cell death beyond a level that could have been predicted. These combinations have been found to generate synergy via induction of apoptosis in the cancer cells.

The results indicate that synergistic enhancement of cell death via apoptosis is induced when an oncolytic HSV is administered in combination with an RTK inhibitor that inhibits one or more of a VEGFR (e.g. one or more of VEGFR1, VEGFR2, VEGFR3), a PDGFR (e.g. PDGFRα and/or PDGFRβ), c-KIT (CD117) or c-MET, whereas a synergistic effect is not obtained by induction of apoptosis for RTK inhibitors that inhibit EGFR but do not exhibit a significant inhibition at one of a VEGFR, PDGFR, c-KIT (CD117) or c-MET.

This observation allows for the selection of specific combinations of HSV and RTK inhibitor that will provide synergistic enhancement of cell death. These selections can be made by determining (i) the presence or absence of a synergistic level of enhancement of cell killing by the combined administration of HSV and RTK inhibitor, and/or (ii) the induction of apoptosis by the combined administration of HSV and RTK inhibitor.

The presence or absence of a synergistic level of cell killing may be determined using Chou-Talalay analysis of induction of cell death by combination of HSV and RTK inhibitor, e.g. as described herein.

The induction of apoptotic cell death may be determined by assaying for activation of (i) caspase 3 (and/or caspase 7), and/or (ii) caspase 9.

### Subjects

The subject to be treated may be any animal or human. The subject is preferably mammalian, more preferably human. The subject may be a non-human mammal, but is more preferably human. The subject may be male or female. The subject may be a patient. A subject may have been diagnosed with a cancer, or be suspected of having a cancer.

### Other Chemotherapeutic Agents

In addition to treating a cancer by using a virus with a RTK inhibitor, subjects being treated may also receive treatment with other chemotherapeutic agents. For example, other chemotherapeutic agents may be selected from:
(i) alkylating agents such as cisplatin ((SP-4-2)-diaminedichloroplatinum(II)), carboplatin (cis-diammine(cyclobutane-1,1-dicarboxylate-O,O′)platinum(II)), mechlorethamine (Bis(2-chloroethyl)methylamine), cyclophosphamide ((RS)-N,N-bis(2-chloroethyl)-1,3,2-oxazaphosphinan-2-amine 2-oxide), chlorambucil (4-[bis(2-chlorethyl)amino]benzenebutanoic acid), ifosfamide (N-3-bis(2-chloroethyl)-1,3,2-oxazaphosphinan-2-amide-2-oxide);
(ii) purine or pyrimidine anti-metabolites such as azathiopurine (6-[(1-methyl-4-nitro-1-imidazol-5-yl)sulfanyl]-7H-purine) or mercaptopurine (3,7-dihydropurine-6-thione);
(iii) alkaloids and terpenoids, such as vinca alkaloids (e.g. vincristine ((3aR,3a1R,4R,5S,5aR,10bR)-methyl 4-acetoxy-3a-ethyl-9-((5S,7S,9S)-5-ethyl-5-hydroxy-9-(methoxycarbonyl)-2,4,5,6,7,8,9,10-octahydro-1H-3,7-methano[1]azacycloundecino[5,4-b]indol-9-yl)-6-formyl-5-hydroxy-8-methoxy-3a,3a1,4,5,5a,6,11,12-octahydro-1H-indolizino[8,1-cd]carbazole-5-carboxylate), vinblastine (dimethyl (2β,3β,4β,5α,12β,19α)-15-[(5S,9S)-5-ethyl-5-hydroxy-9-(methoxycarbonyl)-1,4,5,6,7,8,9,10-octahydro-2H-3,7-methanoazacycloundecino[5,4-b]indol-9-yl]-3-hydroxy-16-methoxy-1-methyl-6,7-didehydroaspidospermidine-3,4-dicarboxylate), vinorelbine (4-(acetyloxy)-6,7-didehydro-15-((2R,6R,8S)-4-ethyl-1,3,6,7,8,9-hexahydro-8-(methoxycarbonyl)-2,6-methano-2H-azecino(4,3-b)indol-8-yl)-3-hydroxy-16-methoxy-1-methyl-methyl ester), vindesine (methyl (5S,7S,9S)-9-[(2β,3β,4β,5α,12β,19α)-3-(aminocarbonyl)-3,4-dihydroxy-16-methoxy-1-methyl-6,7-didehydroaspidospermidin-15-yl]-5-ethyl-5-hydroxy-1,4,5,6,7,8,9,10-octahydro-2H-3,7-methanoazacycloundecino[5,4-b]indole-9-carboxylate)), podophyllotoxin (10R,11R,15R,16R)-16-hydroxy-10-(3,4,5-trimethoxyphenyl)-4,6,13-trioxatetracyclo[7.7.0.0^{3,7}0.0^{11,15}]hexadeca-1,3(7),8-trien-12-one), etoposide (4′-demethyl-epipodophyllotoxin 9-[4,6-O-(R)-ethylidene-beta-D-glucopyranoside], 4′-(dihydrogen phosphate)), teniposide ((5R,5aR,8aR,9S)-5,8,8a,9-Tetrahydro-5-(4-hydroxy-3,5-dimethoxyphenyl)-9-({4,6-O-[(R)-2-thienylmethylene]-β-D-glucopyranosyl}oxy)furo[3′,4′:6,7]naphtho[2,3-d]-1,3-dioxol-6(5aH)-one), taxanes such as paclitaxel (Taxol™; (2α,4α,5β,7β,10β,13α)-4,10-bis(acetyloxy)-13-{[(2R,3S)-3-(benzoylamino)-2-hydroxy-3-phenylpropanoyl]oxy}-1,7-dihydroxy-9-oxo-5,20-epoxytax-11-en-2-yl benzoate), docetaxel (1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate});
(iv) topoisomerase inhibitors such as the type I topoisomerase inhibitors camptothecins irinotecan ((S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate) and topotecan ((S)-10-[(dimethylamino)methyl]-4-ethyl-4,9-dihydroxy-1H-pyrano[3′,4′:6,7]indolizino[1,2-b]quinoline-3,14(4H,12H)-dione monohydrochloride), or the type 11 topoisomerase inhibitors amsacrine (N-(4-(acridin-9-ylamino)-3-methoxyphenyl)methanesulfonamide), etoposide (see above), etoposide phosphate, teniposide (see above);
(v) antitumor antibiotics (e.g. anthracyline antibiotics) such as dactinomycin (2-Amino-N,N′-bis[(6S,9R,10S,13R,18aS)-6,13-diisopropyl-2,5,9-trimethyl-1,4,7,11,14-pentaoxohexadecahydro-1H-pyrrolo[2,1-i][1,4,7,10,13]oxatetraazacyclohexadecin-10-0]-4,6-dimethyl-3-oxo-3H-phenoxazine-1,9-dicarboxamide), doxorubicin (Adriamycin™; (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione), epirubicin ((8R,10S)-10-((2S,4S,5R,6S)-4-amino-5-hydroxy-6-methyltetrahydro-2H-pyran-2-yl)-6,8,11-trihydroxy-8-(2-hydroxyacetyl)-1-methoxy-7,8,9,10-tetrahydrotetracene-5,12-dione), bleomycin (3-{[(2′-{(5S,8S,9S,10R,13S)-15-{6-amino-2-[(1S)-3-amino-1-{[(2S)-2,3-diamino-3-oxopropyl]amino}-3-oxopropyl]-5-methylpyrimidin-4-yl}-13-[{[(2R,3S,4S,5S,6S)-3-{[(2R,3S,4S,5R,6R)-4-(carbamoyloxy)-3,5-dihydroxy-6-(hydroxymethyl)tetrahydro-2H-pyran-2-yl]oxy}-4,5-dihydroxy-6-(hydroxymethyl)tetrahydro-2H-pyran-2-yl]oxy}(1H-imidazol-5-yl)methyl]-9-hydroxy-5-[(1R)-1-hydroxyethyl]-8,10-dimethyl-4,7,12,15-tetraoxo-3,6,11,14-tetraazapentadec-1-yl}-2,4′-bi-1,3-thiazol-4-yl)carbonyl]amino}propyl)(dimethyl)sulfonium), rapamycin ((3S,6R,7E,9R,10R,12R,14S,15E,17E,19E,21S,23S,26R,27R,34aS)-9,10,12,13,14,21,22,23,24,25,26,27,32,33,34,34a-hexadecahydro-9,27-dihydroxy-3-[(1R)-2-[(1 S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]-oxaazacyclohentriacontine-1,5,11,28,29(4H,6H,31H)-pentone).

In some embodiments of the present invention the only chemotherapeutic agent for simultaneous, separate or sequential administration with the virus is an RTK inhibitor, e.g. an inhibitor of one or more of an ErbB receptor, VEGFR and/or PDGFR and/or ALK and/or insulin-like growth factor receptor inhibitor. Optionally, further chemotherapeutic agents (e.g. as described above) are not administered as part of the treatment.

### Routes of Administration

Viruses, chemotherapeutic agents, medicaments and pharmaceutical compositions according to aspects of the present invention may be formulated for administration by a number of routes, including but not limited to, parenteral, intravenous, intra-arterial, intramuscular, intratumoural and oral. Viruses, chemotherapeutic agents, medicaments and compositions may be formulated in fluid or solid form. Fluid formulations may be formulated for administration by injection to a selected region of the human or animal body.

Optionally, the present invention does not involve or rely on an antiangiogenic effect of the RTK inhibitor or virus. Whereas an anti-angiogenic agent may be administered to non-cancer tissues in order to influence the blood supply to a cancer and not directly treat the cancer, the present invention is preferably concerned with the effect of each of the RTK inhibitor and virus on cells of the cancer (i.e. tumor cells, cancerous cells).

Preferably this effect is a direct cell killing effect. As such, direct administration to the cancer or tumor or to tissue immediately surrounding the cancer/tumor may be preferred. In some embodiments, intratumoural administration of RTK inhibitor and/or virus (e.g. intratumoural injection) is preferred. In some other embodiments administration of RTK inhibitor and/or virus is to the blood being supplied directly to the cancer/tumor is preferred.

### Dosage Regime

Multiple doses of the virus may be provided. One or more, or each, of the doses may be accompanied by simultaneous or sequential administration of a chemotherapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months.

By way of example, doses may be given once every 7, 14, 21 or 28 days (plus or minus 3, 2, or 1 days). The dose of virus given at each dosing point may be the same, but this is not essential. For example, it may be appropriate to give a higher priming dose at the first, second and/or third dosing points.

### Kits

In some aspects of the present invention a kit of parts is provided. In some embodiments the kit may have at least one container having a predetermined quantity of virus, e.g. predetermined viral dose or number/quantity/concentration of viral particles. The virus may be formulated so as to be suitable for injection or infusion to a tumor or to the blood. In some embodiments the kit may further comprise at least one container having a predetermined quantity of chemotherapeutic agent. The chemotherapeutic agent may also be formulated so as to be suitable for injection or infusion to the tumor or to the blood, or alternatively may be formulated for oral administration. In some embodiments a container having a mixture of a predetermined quantity of virus and predetermined quantity of chemotherapeutic agent is provided, which may optionally be formulated so as to be suitable for injection or infusion to the tumor or to the blood.

In some embodiments the kit may also contain apparatus suitable to administer one or more doses of the virus and/or chemotherapeutic agent. Such apparatus may include one or more of a catheter and/or needle and/or syringe, such apparatus preferably being provided in sterile form.

The kit may further comprise instructions for the administration of a therapeutically effective dose of the virus and/or chemotherapeutic agent.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

Embodiments and experiments illustrating the principles of the invention will now be discussed with reference to the accompanying figures in which:

FIG. 1, Graphs showing toxicity profiles for Sorafenib. Cells were incubated with Sorafenib at concentrations 50 μM, 20 μM, 10 μM, 5 μM, 2 μM, 1 μM, 0.5 μM, 0.2 μM, 0.1 μM, 0.05 μM and 0.01 μM and DCP leakage determined after 72 hrs. Each concentration was assayed in at least triplicate and values are graphed as % of control (cells alone with no drug) for Sorafenib IC50 determinations in the Virttu cell line panel. FIG. 1(a) Hep3B; 1(b) HuH7; 1(c) HepG2-luc; 1(d) CP70; 1(e) Ovcar3; 1(f) SKOV3; 1(g) U87; 1(h) UVW; 1(i) One58; 1(j) SPC111.

FIG. 2. Table 1 showing Sorafenib IC50 values as estimated from the curves shown in FIG. 1. Data marked with * are reported by Chang & Wang, 2013, “In-vitro growth inhibition of chemotherapy and molecular targeted agents in hepatocellular carcinoma,” Anti-Cancer Drugs Vol. 24, pp. 251-259.

FIG. 3. Graphs showing effects of Sorafenib on GFP expression during infection with HSV1716gfp. Sorafenib was incubated with cells plus virus at moi 0.5/0.05 and at 2.5, 1, 0.5 and 0.25 μM for 72 hrs and GFP fluorescence measured. Drug-mediated virus toxicities are presented as percentage of control cells (virus alone and no drug). FIG. 3(a) Hep3B; 3(b) HuH7; 3(c) CP70; 3(d) Ovcar3; 3(e) SKOV3; 3(f) U87; 3(g) UVW; 3(h) One58 and 3(i) SPC-111.

FIG. 4. Table 2 showing summary of Sorafenib effects on HSV1716 replication from FIG. 3. nd=not determined, +/−=control value+/−<5%, −=some inhibition (5%< but >10% of control), −− inhibition (>10% of control).

FIG. 5. Chou/Talalay plots for combination of Sorafenib with HSV1716 in Hep3B, HuH7, HepG2-luc, CP70, Ovcar3, SKOV3, U87, UVW, One58 and SPC-111 cells. The relevant tables of Fa and Cl values for the individual Sorafenib concentrations and HSV1716 moi accompany each Chou/Talalay plot. If the Fa value was negative then the corresponding Cl value could not be determined and Cl values above 4 are not presented in the graphs. Cl values below 1 (horizontal line) indicate synergy and those above indicate antagonistic effects.

FIG. 6. Table 3 showing a summary of Chou/Talalay analysis of Sorafenib in combination with HSV1716 at moi 0.5 and 0.05 in the Virttu cell panel from FIG. 5. Numbers of synergistic combinations at the two moi are indicated and the overall synergy/antagonism was assigned on this basis with +=mostly synergy, +/−=mixed synergy/antagonism, −=mostly antagonism.

FIG. 7. Graphs showing toxicity profiles for Cabozantinib (XL-184). Cells were incubated with XL-184 at concentrations 100 μM, 75 μM, 50 μM, 25 μM, 10 μM, 7.5 μM, 5 μM, 2.5 μM, 1 μM and 0.1 μM and DCP leakage determined after 72 hrs. Each concentration was assayed in triplicate at least and values are graphed as % of control (cells alone with no drug) for XL-184 IC50 determinations in the Virttu cell line panel. FIG. 7(a) Hep3B; 7(b) HuH7; 7(c) HepG2-luc; 7(d) CP70; 7(e) Ovcar3; 7(f) SKOV3; 7(g) U87; 7(h) UVW; 7(i) One58; 7(j) SPC111.

FIG. 8. Table 4 showing Cabozantinib (XL-184) IC50 values as estimated from the curves shown in FIG. 7. IC50 values of between 1.1 and 21.7 μM are reported for Cabozantinib in various human cancer cell lines without mesenchymal-epithelial transition factor (MET) amplification (Yakes et al., 2011 “Cabozantinib (XL184), a novel MET and VEGFR2 inhibitor, simultaneously suppresses metastasis, angiogenesis, and tumor growth”_Mol. Cancer Ther. Vol 10, pp, 2298-2308).

FIG. 9. Graphs showing effects of Cabozantinib on GFP expression during infection with HSV1716gfp. Cabozantinib was incubated with cells plus virus at moi 0.5/0.05 at 10 μM, 5 μM, 2 μM, 1 μM, 0.2 μM and 0.1 μM for 72 hrs and GFP fluorescence measured. Drug-mediated virus toxicities are presented as percentage of control cells (virus alone and no drug). FIG. 9(a) Hep3B; 9(b) HuH7; 9(c) HepG2-luc; 9(d) CP70; 9(e) Ovcar3; 9(f) SKOV3; 9(g) U87; 9(h) UVW; 9(i) One58; 9(j) SPC-111.

FIG. 10. Table 5 showing a summary of Cabozantinib effects on HSV1716 replication from FIG. 9. +/−=control value+/−<5%, −=some inhibition (5%< but >10% of control), −− inhibition (>10% of control).

FIG. 11. Chou/Talalay plots for combination of Cabozantinib (XL-184) with HSV1716 in Hep3B, HuH7, HepG2-luc, CP70, Ovcar3, SKOV3, U87, UVW, One58 and SPC-111 cells. The relevant tables of Fa and Cl values for the individual XL-184 concentrations and HSV1716 moi accompany each Chou/Talalay plot. If the Fa value was negative then the corresponding Cl value could not be determined and Cl values above 4 are not presented in the graphs. Cl values below 1 (horizontal line) indicate synergy and those above indicate antagonistic effects

FIG. 12. Table 6 showing a summary of Chou/Talalay analysis of Cabozantinib in combination with HSV1716 at moi 0.5 and 0.05 in the Virttu cell panel from FIG. 11. Numbers of synergistic combinations at the two moi are indicated and the overall synergy/antagonism was assigned on this basis with +=mostly synergy, +/−=mixed synergy/antagonism, −=mostly antagonism.

FIG. 13. Graphs showing toxicity profiles for Pazopanib in the Virttu cell line panel. Cells were incubated with Pazopanib at concentrations 100 μM, 75 μM, 50 μM, 25 μM, 10 μM, 7.5 μM, 5 μM, 2.5 μM, 1 μM and 0.1 μM and DCP leakage determined after 72 hrs. Each concentration was assayed in triplicate at least and values are graphed as % of control (cells alone with no drug) for Pazopanib IC50 determinations in the Virttu cell line panel. FIG. 13(a) Hep3B; 13(b) HuH7; 13(c) HepG2-luc; 13(d) CP70; 13(e) Ovcar3; 13(f) SKOV3; 13(g) U87; 13(h) UVW; 13(i) One58; 13(j) SPC111.

FIG. 14. Table 7 showing Pazopanib IC50 values as estimated from the curves shown in FIG. 13. Pazopanib IC50s of between 1-10 μM have been reported in breast carcinoma and melanoma cell lines (Gril et al., 2011 “The B-Raf status of tumor cells may be a significant determinant of both antitumor and anti-angiogenic effects of pazopanib in xenograft tumor models,” PLoS ONE Vol, 6, pp. 1-11) and between 30-60 μM in human RCC cell lines (Canter et al., 2011 “Are all multi-targeted tyrosine kinase inhibitors created equal? An in vitro study of Sunitinib and Pazopanib in renal cell carcinoma cell lines,” Can J Urol Vol. 18, pp. 5819-5825). The range in Table 7 is in keeping with these previously reported values.

FIG. 15. Graphs showing effects of Pazopanib on GFP expression during infection with HSV1716gfp. Pazopanib was incubated with cells plus virus at moi 0.5/0.05 at 20, 10, 2, 1, 0.2 and 0.1 μM for 72 hrs and GFP fluorescence measured. Drug-mediated virus toxicities are presented as percentage of control cells (virus alone and no drug). FIG. 15(a) HuH7; 15(b) HepG2-luc; 15(c) CP70; 15(d) Ovcar3; 15(e) SKOV3; 15(f) UVW; 15(g) One58.

FIG. 16. Table 8 showing a summary of Pazopanib effects on HSV1716 replication from FIG. 15. nd=not determined, ++=stimulation>25% of control, +=stimulation>10% of control, +/−=control value+/−<5%, −=some inhibition (5%< but >10% of control), −− inhibition (>10% of control).

FIG. 17. Chou/Talalay plots for combination of Pazopanib with HSV1716 in HuH7, HepG2-luc, CP70, Ovcar3, SKOV3, U87, UVW, One58 and SPC-111 cells. The relevant tables of Fa and Cl values for the individual Pazopanib concentrations and HSV1716 moi accompany each Chou/Talalay plot. If the Fa value was negative then the corresponding Cl value could not be determined and Cl values above 4 are not presented in the graphs. Cl values below 1 (horizontal line) indicate synergy and those above indicate antagonistic effects.

FIG. 18. Table 9 showing a summary of Chou/Talalay analysis of Pazopanib in combination with HSV1716 at moi 0.5 and 0.05 in the Virttu cell panel from FIG. 17. Numbers of synergistic combinations at the two moi are indicated and the overall synergy/antagonism was assigned on this basis with +=mostly synergy, +/−=mixed synergy/antagonism, −=mostly antagonism.

FIG. 19. Graphs showing dead cell protease leakage values expressed as % control (no virus or no drug): (a) HSV1716 was added at moi 2, 1, 0.5, 0.2, 0.1, 0.05, 0.02, 0.01 and 0.005, and Sorafenib (b) was added to give final concentrations of 20 μM, 10 μM, 5 μM, 2 μM, 1 μM, 0.5 μM, 0.2 μM, 0.1 μM and 0.05 μM to Hep3B, HuH7 and HepG2-luc cells in culture.

FIG. 20. Table 10 Sorafenib and HSV1716 IC50 for Hep3B, HuH7 and HepG2-luc cells. HSV1716 and sorafenib were tested at final concentrations as described in FIG. 19. *IC50s reported by Chang and Wang (Anti-Cancer Drugs 2013; 24: 251-259).

FIG. 21. FaCl plots for the effects of combined Sorafenib and HSV1716 treatment on (a) Hep3B, (b) HuH7 and (c) HepG2-luc cells. HSV1716 was added at moi 0.5 and 0.05 and sorafenib was added at 2.5 μM, 1 μM, 0.5 μM, and 0.25 μM. The Combination Index (y-axis) was plotted against Fa (x-axis) and the line at Cl=1 divides synergy (below) from antagonism (above). Most combinations of Sorafenib with HSV1716 at both moi generate. Cl values of <1 and therefore the drug acts synergistically to enhance Hep3B, HuH7 and HepG2-luc cell death in vitro.

FIG. 22. Summary of Chou-Talalay analysis of HSV1716 in combination with Sorafenib in 3 human HCC cell lines. (+)=synergy at most moi and drug concentrations, (+/−)=mixed signals with some combinations synergistic and others antagonistic, − mostly antagonistic in combination.

FIG. 23. Comparison of HSV1716 kill on different cancer cell lines by DCP cell death assay. Cells were incubated with HSV1716 at moi 2, 1, 0.5, 0.2, 0.1, 0.05, 0.02, 0.01, 0.005, 0.001 for 23(a) Hep3B; 23(b) HuH7; 23(c) HepG2-luc; 23(d) CP70; 23(e) Ovcar3; 23(f) SKOV3; 23(g) U87; 23(h) UVW; 23(i) One58; 23(j) SPC111 at moi 10, 5, 1, 0.5, 0.1, 0.05, 0.01 and 0.001. DCP leakage was determined after 72 hrs. Each concentration was assayed in triplicate at least and values are graphed as % of control (cells alone with no virus) for HSV1716 VD50 determinations in the Virttu cell line panel.

FIG. 24. Virus VD50 values as estimated from the curves shown in FIG. 23. VD50 is the moi at which 50% of the cells are killed.

FIG. 25. Table 11 showing replication competence of HSV1716 in UVW, U87MG, HuH7, HepG2, One58, Ovcar3, SKOV3, A2780 and CP70 cells.

FIG. 26. Graphs showing toxicity profiles for Crizotinib in (a) Hep3B; (b) HuH7; (c) HepG2-luc; (d) CP70; (e) SKOV3; (f) Ovcar3; (g) U87; (h) UVW; (i) One58; (j) SPC111. Cells were incubated with Crizotinib at concentrations 50, 20, 10, 5, 2, 1, 0.5, 0.2 and 0.1 μM and DCP leakage determined after 72 hours. Each concentration was assayed in triplicate at least and values are graphed as % of control (cells alone with no drug) for Crizotinib IC50 determinations in the Virttu cell line panel.

FIG. 27. Table 12 showing Crizotinib IC50 values as estimated from the curves shown in FIG. 26.

FIG. 28. Graphs showing effects of Crizotinib on GFP expression during infection with HSV1716gfp. Crizotinib was incubated with cells plus virus at moi 0.5/0.05 at 50, 10, 5 and 1 μM for 72 hours and GFP fluorescence measured. Drug-mediated virus toxicities are presented as percentage of control cells (virus alone and no drug) in (a) Hep3B, (b) HuH7, (c) HepG2-luc, (d) CP70, (e) SKOV3, (f) Ovcar3, (g) U87, (h) UVW, (i) One58 and (j) SPC-111.

FIG. 29. Table 13 showing effects of Crizotinib on HSV1716 replication from FIG. 28.

FIG. 30. Chou/Talalay plots for HSV1716 in combination with Crizotinib in (a) HuH7, (b) HepG2-luc, (c) CP70, (d) Ovcar3, (e) U87, (f) UVW, (g) One58, (h) SPC-111 and (i) SKOV3 cells. The relevant tables of Fa and Cl values for the individual Crizotinib concentrations and HSV1716 moi accompany each Chou/Talalay plot. If the Fa value was negative then the corresponding Cl value could not be determined and Cl values above 2 are not presented in the graphs.

FIG. 31. Table 14 showing Chou/Talalay analysis of Crizotinib in combination with HSV1716 at moi 0.5 and 0.05 in the Virttu cell panel. Numbers of synergistic combinations at the two moi are indicated and the overall synergy/antagonism was assigned on this basis +++=strong synergy (6-8/8), ++=synergy (4-5/8), +=limited synergy (1-3/8) and −=antagonism (0/8), nd=not determined.

FIG. 32. Graphs showing toxicity profiles for Erlotinib in (a) Hep3B; (b) HuH7; (c) A431; (d) CP70; (e) Ovcar3; (f) SKOV3; (g) U87; (h) UVW; (i) One58; (j) SPC111. HepG2-luc cells were not available for this analysis. All cells except A431 were incubated with Erlotinib at concentrations 50, 20, 10, 5, 2, 1, 0.5, 0.2 and 0.1 μM and DCP leakage determined after 72 hours. A431 cells were incubated with 1, 0.5, 0.2, 0,1, 0.05, 0.02, 0.01, 0.005 or 0.002 μM Erlotinib. Each concentration was assayed in triplicate at least and values are graphed as % of control (cells alone with no drug) for Erlotinib 1050 determinations in the Virttu cell line panel.

FIG. 33. Table 15 showing Erlotinib IC50 values as estimated from the curves shown in FIG. 32.

FIG. 34. Chou/Talalay plots for HSV1716 in combination with Erlotinib in (a) Hep3B, (b) HuH7, (c) A431, (d) CP70, (e) SKOV3, (f) U87, (g) UVW, (h) One58; (i) SPC-111 and (j) Ovcar 3 cells. The relevant tables of Fa and Cl values for the individual Erlotinib concentrations and HSV1716 MOI accompany each Chou/Talalay plot. If the Fa value was negative then the corresponding Cl value could not be determined (nd=not determined).

FIG. 35. Table 16 showing Chou/Talalay analysis of Erlotinib in combination with HSV1716 at moi 0.5 and 0.05 in the Virttu cell panel. Numbers of synergistic combinations at the two moi are indicated and the overall synergy/antagonism was assigned on this basis +++=strong synergy (6-8/8), ++=synergy (4-5/8), +=limited synergy (1-3/8) and −=antagonism (0/8), nd=not determined.

FIG. 36. Graphs showing toxicity profiles for Gefitinib in (a) Hep3B, (b) HuH7, (c) A431, (d) CP70, (e) SKOV3, (f) Ovcar 3 (g) U87, (h) UVW, (i) One58; and (j) SPC-111 cells. HepG2-luc cells were not available for this analysis. All cells except A431 were incubated with Gefitinib at concentrations 50, 20, 10, 5, 2, 1, 0.5, 0.2 and 0.1 μM and DCP leakage determined after 72 hours. A431 cells were incubated with 1, 0.5, 0.2, 0.1, 0.05, 0.02, 0.01, 0.005 or 0.002 μM Gefitinib. Each concentration was assayed in triplicate at least and values are graphed as % of control (cells alone with no drug) for Gefitinib IC50 determinations in the Virttu cell line panel.

FIG. 37. Table 17 showing Gefitinib IC50 values as estimated from the curves shown in FIG. 36.

FIG. 38. Chou/Talalay plots for HSV1716 in combination with Gefitinib in (a) Hep3B, (b) HuH7, (c) A431, (d) CP70, (e) SKOV3, (f) Ovcar3, (g) UVW, (h) U87 and (i) SPC111 cells. The relevant tables of Fa and Cl values for the individual Gefitinib concentrations and HSV1716 moi accompany each Chou/Talalay plot. If the Fa value was negative then the corresponding Cl value could not be determined and Cl values above 2 are not presented in the graphs.

FIG. 39. Table 18 showing Chou/Talalay analysis of Gefitinib in combination with HSV1716 at moi 0.5 and 0.05 in the Virttu cell panel. Numbers of synergistic combinations at the two moi are indicated and the overall synergy/antagonism was assigned on this basis +++=strong synergy (6-8/8), ++=synergy (4-5/8), +=limited synergy (1-3/8) and −=antagonism (0/8), nd=not determined.

FIG. 40. Graphs showing toxicity profiles for Lapatinib in (a) Hep3B, (b) HuH7, (c) CP70, (d) Ovcar3, (e) SKOV3, (f) U87 (g) UVW, (h) One58, (i) SPC111 and (j) A431 cells. All cells except A431 were incubated with Lapatinib at concentrations 50, 20, 10, 5, 2, 1, 0.5, 0.2 and 0.1 μM and DCP leakage determined after 72 hours, A431 cells were incubated with 1, 0.5, 0.2, 0.1, 0.05, 0.02, 0.01, 0.005 or 0.002 μM Lapatinib. Each concentration was assayed in triplicate and values are graphed as % of control (DCP leakage from cell alone with no drug). X axis=Lapatinib concentration (μM), Y axis=% control of cell death.

FIG. 41. Table 19 showing Lapatinib IC50 values as estimated from the curves shown in FIG. 40.

FIG. 42. Chou/Talalay plots for HSV1716 in combination with Lapatinib in (a) Hep3B, (b) HuH7, (c) A431, (d) CP70, (e) SKOV3, (f) Ovcar3, (g) U87, (h) UVW, (i) One58, and (j) SPC111 cell lines. The relevant tables of Fa and Cl values for the individual Lapatinib concentrations and HSV1716 moi accompany each Chou/Talalay plot. If the Fa value was negative then the corresponding Cl value could not be determined and Cl values above 4 are not presented in the graphs.

FIG. 43. Table 20 showing Chou/Talalay analysis of Lapatinib combination with HSV1716 at moi 0.5 and 0.05 in the Virttu cell panel. Numbers of synergistic combinations at the two moi are indicated and the overall synergy/antagonism was assigned on this basis with +++=strong synergy/enhancement, ++=mostly synergy, +=limited synergy/enhancement −=antagonism.

FIG. 44. Graphs showing toxicity profiles for Sunitinib in (a) Hep3b, (b) HuH7, (c) HepG2-luc, (d) CP70, (e) SKOV3, (f) Ovcar3, (g) U87, (h) UVW, (i) One58, and (j) SPC-111. Cells were incubated with Sunitinib at concentrations 50, 20, 10, 5, 2, 1, 0.5, 0.2 and 0.1 μM and DCP leakage determined after 72 hours. Each concentration was assayed in triplicate at least and values are graphed as % of control (cells alone with no drug) for Sunitinib IC50 determinations in the Virttu cell line panel.

FIG. 45. Table 21 showing Sunitinib IC50 values as estimated from the curves shown in FIG. 44.

FIG. 46. Graphs showing Sunitinib effects on GFP expression during infection with HSV1716gfp. Sunitinib was incubated with cells plus virus at moi 0.5/0.05 at 5, 2.5, 0.625 and 0.312 μM for 72 hours and GFP fluorescence measured. Drug-mediated virus toxicities are presented as percentage of control cells (virus alone and no drug) in (a) Hep3B, (b) HuH7, (c) HepG2-luc, (d) CP70, (e) Ovcar3, (f) SPC111, (g) U87, (h) UVW, (i) One58.

FIG. 47. Table 22 showing effects of Sunitinib on HSV1716 replication from FIG. 46. +++=stimulation>30% of control value, ++=stimulation>20% of control, +=stimulation>10% of control, nd=not determined.

FIG. 48. Chou/Talalay plots for HSV1716 in combination with Sunitinib in (a) HuH7, (b) Hep3B, (c) CP70, (d) HepG2-luc, (e) Ovcar3, (f) SKOV3, (g) U87, (h) UVW, (i) One58 and (j) SPC-111 cells. The relevant tables of Fa and Cl values for the individual Sunitinib concentrations and HSV1716 moi accompany each Chou/Talalay plot. If the Fa value was negative then the corresponding Cl value could not be determined and Cl values above 2 are not presented in the graphs.

FIG. 49. Table 23 showing Chou/Talalay analysis of Sunitinib in combination with HSV1716 at moi 0.5 and 0.05 in the Virttu cell panel. Numbers of synergistic combinations at the two moi are indicated and the overall synergy/antagonism was assigned on this basis +++=strong synergy (6-8/8), ++=synergy (4-5/8), +=limited synergy (1-3/8) and −=antagonism (0/8), nd=not determined.

FIG. 50. Bar charts and Chou/Talalay plots showing caspase activity. HSV1716 combines synergistically with Sunitinib in (A) Ovcar3, (B) Hep3B, (C) One58, (D) U87, and (E) CP70 cells, and the combination significantly enhances caspase 3/7 activity compared to HSV1716 or Sunitinib alone (left panel). HSV1716 displays limited synergy with Sunitinib in (F) HuH7, (G) SKOV3 and (H) UVW cells. For HuH7 and SKOV3 cells, the combination fails to enhance caspase 3/7 activity compared to HSV1716 or Sunitinib alone. For UVW cells, the combination fails to enhance caspase 3/7 activity compared to HSV1716 alone.

FIG. 51. Bar charts and Chou/Talalay plots showing caspase activity. HSV1716 combines synergistically with Cabozantinib in (A) HuH7 and (B) Hep3B cells, and the combination significantly enhances caspase 3/7 activity compared to HSV1716 or Cabozantinib alone.

FIG. 52. Bar chart and Chou/Talalay plot showing caspase activity. HSV1716 combines synergistically with Crizotinib in HuH7 cells and the combination significantly enhances caspase 3/7 activity compared to HSV1716 alone.

FIG. 53. Bar charts and Chou/Talalay plots showing caspase activity. HSV1716 does not combine synergistically with Gefitinib in (A) Ovcar3, (B) One58, (C) CP70, (D) SKOV3 and (E) UVW cells. For Ovcar3, CP70 and SKOV3 cells, the combination fails to enhance caspase 3/7 activity compared to HSV1716 or Gefitinib alone. For One58 and UVW cells, the combination fails to enhance caspase 3/7 activity compared to HSV1716 alone.

FIG. 54. Bar charts and Chou/Talalay plots showing caspase activity. HSV1716 does not combine synergistically with Erlotinib in (A) Ovcar3, (B) Hep3, and (C) HuH7 cells, and the combination fails to enhance caspase 3/7 activity compared to HSV1716 or Erlotinib alone.

FIG. 55. Bar charts showing caspase activity. HSV1716 combines synergistically with Sunitinib to activate caspase 9 in (A) Hep3B and (B) Ovcar3 cells. However, for (C) HuH7 cells, the combination fails to enhance caspase 9 activity compared to HSV1716 or Sunitinib alone.

FIG. 56. Bar charts showing caspase activity. HSV1716 combines synergistically with Cabozantinib to activate caspase 9 in (A) Hep3B and (B) HuH7 cells.

FIG. 57. Diagram showing extrinsic and intrinsic apoptotic cascades. Extrinsic apoptosis is via caspase 8 whereas intrinsic apoptosis is mediated via the mitochondrion and caspase 9. HSV1716 in combination with Sunitinib in Ovcar3 cells results in activation of intrinsic apoptosis with caspase 9 then caspase 3/7 activation. Activated caspase 3/7 then initiates the executioner phase of apoptosis with PARP cleavage demonstrated by Western blotting. Actin acts as a loading control in the Western blotting panel.

FIG. 58. Diagram showing extrinsic and intrinsic apoptotic cascades. Extrinsic apoptosis is via caspase 8 whereas intrinsic apoptosis is mediated via the mitochondrion and caspase 9. HSV1716 in combination with Sunitinib in Hep3B cells results in activation of intrinsic apoptosis with caspase 9 then caspase 3/7 activation. Activated caspase 3/7 then initiates the executioner phase of apoptosis with PARP cleavage demonstrated by Western blotting. Actin acts as a loading control in the Western blotting panel.

FIG. 59. Diagram showing extrinsic and intrinsic apoptotic cascades. Extrinsic apoptosis is via caspase 8 whereas intrinsic apoptosis is mediated via the mitochondrion and caspase 9. HSV1716 in combination with Cabozantinib in Hep3B cells results in activation of intrinsic apoptosis with caspase 9 then caspase 3/7 activation. Activated caspase 3/7 then initiates the executioner phase of apoptosis with PARP cleavage demonstrated by Western blotting.

FIG. 60. Table 24 showing results of caspase studies. In cell lines in which HSV1716 combines synergistically with a TKi there is activation of caspases 9 and 3/7. In cell lines in which there are no combinatorial synergies, neither caspase 9 nor caspase 3/7 are activated. (nd=not determined)

FIG. 61. Graph of results from the initial study to investigate the oncolytic effect of Seprehvir in a human gastric carcinoma xenograft model when given via intratumoral (IT) or intravenous (IV) routes of administration.

FIG. 62. Graph showing inhibition of tumour growth in a human gastric carcinoma xenograft model by Cabozantinib, Regorafenib or Lapatinib.

FIG. 63. Graph showing tumour growth in a human gastric carcinoma xenograft model for untreated mice, and mice treated with control, IV Seprehvir, IV Seprehvir+Cabozantinib and IV Seprehvir+Regorafenib.

FIG. 64. Graph showing combined data from both tumour growth studies in a human gastric carcinoma xenograft model for untreated mice, and mice treated with control, IV Seprehvir (low dose), IT Seprehvir (low dose), Cabozantinib, Regorafenib, IV Seprehvir (high titer), IV Seprehvir (high titer)+Cabozantinib and IV Seprehvir (high titer)+Regorafenib.

FIG. 65. Table summarising synergy, caspas 9, and 3/7 activation and in vivo data for Sorafenib, Sunitinib, Cabozantinib, Pazopanib, Crizotinib, Regorafenib, Gefitinib, Erlotinib, Lapatinib.

Examples 1 to 4 report the in vitro experimental testing of the HSV-1 strain 17 oncolytic mutant HSV1716 in combination with the RTK inhibitors Sorafenib, Pazopanib and Cabozantinib in a range of cancer cell types.

Strong synergy signals were observed between all three RTK inhibitors and HSV1716, particularly with:—
Sorafenib in hepatocellular carcinoma (HCC) cell lines
Pazopanib in ovarian cancer cell lines
Cabozantinib in human glioma cell lines

### Example 1

The following cell lines were used in all Examples, unless stated otherwise:
High Grade Glioma (HGG): U87, UVW
Hepatocellular carcinoma (HCC): HuH7, HepG2-luc, Hep3B
Malignant Pleural Mesothelioma (MPM): One58, SPC111
Ovarian cancer: Ovcar3, SKOV3, CP70

### 1) U87 (aka U87MG, ECCAC 89081402)

Epithelial like cells derived from a malignant glioma from a female patient by explant technique and reported to produce a malignant tumour consistent with glioblastoma in nude mice. Karyotype is 2n (=46). COSMIC (Catalogue Of Somatic Mutations In Cancer) entry indicates somatic mutations in CDKN2A, CDKN2C, CDKN2a(p14) and PTEN.

U87 cells are highly permissive for HSV1716 replication in tissue culture. After 72 hrs of replication HSV-1 17+ yields approximately 43100+/−13988 pfu progeny/input virus and HSV1716 yields 8806+/−2713 pfu progeny/input virus equivalent to a replication competence ratio (HSV1716 yield/HSV-1 17+ yield) of 0.2. Thus HSV1716 replication in U87 cells is impaired 5-fold compared to wild-type HSV-1 17+.

U87 cells readily form subcutaneous xenografts in nude mice.

### 2) UVW (aka MOG-G-UVW, ECCAC-86022703)

Cell line established from an anaplastic astrocytoma of normal adult brain and forms xenografts in nude mice. No entry in COSMIC.

UVW are permissive for HSV-1 17+ and HSV1716 replication. After 72 hrs of replication HSV-1 17+ yields approximately 72234=/−18716 pfu progeny/input virus and HSV1716 yields 78369+/−19989 pfu progeny/input virus equivalent to a replication competence ratio (HSV1716 yield/HSV-1 17+ yield) of 1. Thus HSV1716 replication in UVW cells is similar to wild-type HSV-1 17+.

UVW cells readily form subcutaneous xenografts in nude mice

### 3) HuH7 (JCRB0403)

HuH-7 is a well-differentiated, hepatocyte-derived cellular carcinoma cell line that was originally taken from a liver tumor in a 57-year-old Japanese male. HuH-7 are epithelial-like tumorigenic cells which are able to form subcutaneous xenografts in nude mice. According to their entry in COSMIC HuH7 cells have mutated FAM123B and TP53 genes

HuH7 cells are highly permissive for HSV1716 replication both in tissue culture and in subcutaneous xenografts. After 72 hrs of replication HSV-1 17+ yields approximately 3250+/−814 pfu progeny/input virus and HSV1716 yields 28500+/−4815 pfu progeny/input virus equivalent to a replication competence ratio (HSV1716 yield/HSV-1 17+ yield) of 9. Thus HSV1716 replication in HuH7 cells is approximately 9-fold higher compared to wild-type HSV-1 17+.

HuH7 cells from the Virttu cell bank readily form subcutaneous xenografts in nude mice.

### 4) Hep3B (ECCAC 86062703)

Derived from an 8 year old male, cells contain integrated Hepatitis B virus genome. However there is currently no evidence that this cell line produces infectious Hepatitis B virus. No entry in COSMIC.

Hep3B cells are fully permissive for HSV-1 17+ and HSV1716 replication. After 72 hrs of replication HSV-1 17+ yields approximately 14430+/−3085 pfu progeny/input virus and HSV1716 yields 4820+/−2182pfu progeny/input virus equivalent to a replication competence ratio (HSV1716 yield/HSV-1 17+ yield) of 0.33. Thus HSV1716 replication in Hep3B cells is impaired 3-fold compared to wild-type HSV-1 17+.

5) HepG2-luc (Caliper HT1080-luc2)

The Hep G2 cell line was isolated from a liver biopsy of a male Caucasian aged 15 years, with a well differentiated hepatocellular carcinoma. The cells secrete a variety of major plasma proteins e.g. albumin, alpha2-macroglobulin, alpha 1-antitrypsin, transferrin and plasminogen but Hepatitis B virus surface antigens have not been detected. No entry in COSMIC.

HepG2-luc2 is a luciferase expressing cell line stably transfected with the firefly luciferase gene (luc2). The cell line was established by transducing lentivirus containing luciferase 2 gene under the control of human ubiquitin C promoter.

HepG2-luc are fully permissive for HSV-1 17+ and HSV1716 replication. After 72 hrs of replication HSV-1 17+ yields approximately 40670+/−5690 pfu progeny/input virus and HSV1716 yields 60030+/−9870 pfu progeny/input virus equivalent to a replication competence ratio (HSV1716 yield/HSV-1 17+ yield) of 1.5. Thus HSV1716 replication in HepG2-luc cells is slightly better compared to wild-type HSV-1 17+.

They form very slow growing xenografts in nude mice.

### 6) One58 (ECCAC 10092313)

This cell line was derived from the pleural fluid of a patient with malignant mesothelioma. The patient had known exposure to crocidolite asbestos. Cells express cytokeratin and epithelial membrane antigen (EMA) but not mucin. Cells are epithelial-like and spindle-shaped with few vacuoles. No entry in COSMIC.

One58 are permissive for HSV-1 17+ and HSV1716 replication. After 72 his of replication HSV-1 17+ yields approximately 13650 pfu progeny/input virus and HSV1716 yields 12650 pfu progeny/input virus equivalent to a replication competence ratio (HSV1716 yield/HSV-1 17+ yield) of 1. Thus HSV1716 replication in One58 cells is similar to wild-type HSV-1 17+.

Reported to form xenografts in nude mice.

### 7) SPC111 (ECCAC 11120716)

SPC111 was derived from the pleural effusion of a 55-year old male patient, prior to treatment, with a known history of exposure to asbestos. The cells are epitheloid/mesenchymal. No entry in COSMIC.

SPC111 are permissive for HSV-1 17+ and HSV1716 replication. After 72 hrs of replication HSV-1 17+ yields approximately 11920+/−1002 pfu progeny/input virus and HSV1716 yields 2710+/−1343 pfu progeny/input virus equivalent to a replication competence ratio (HSV1716 yield/HSV-1 17+ yield) of 0.23 Thus HSV1716 replication in SPC111 cells is impaired 4-fold compared to wild-type HSV-1 17+.

### 8) OVCAR3 (ATCC HTB-161)

Adherent, epithelial cells derived from the ascitic fluid from a 60 year old Caucasian female with an ovarian tumour. The cell line is aneuploid human female, with chromosome counts in the sub to near-triploid range. COSMIC entry indicates somatic mutation in TP53.

Ovcar3 are fully permissive for HSV-1 17+ and HSV1716 replication. After 72 hrs of replication HSV-1 17+ yields approximately 95543+/−25174 pfu progeny/input virus and HSV1716 yields 179009+/−20662 pfu progeny/input virus equivalent to a replication competence ratio (HSV1716 yield/HSV-1 17+ yield) of 1.9. Thus HSV1716 replication in Ovcar3 cells is slightly better compared to wild-type HSV-1 17+.

Reported by ATCC to form xenografts in nude mice.

### 9) SKOV3-(aka SK-OV3, ECCAC-91091004)

Adherent, epithelial cells derived from the ascitic fluid from a 64 year old Caucasian female with an ovarian tumour that form moderately well-differentiated adenocarcinoma consistent with ovarian primary cells. Cells have a hypodiploid to hypotetraploid karyotype. COSMIC entry indicates somatic mutations in CDKN2A, CDKN2a(p14), MLH1, PIK3CA and TP53.

SKOV3 are permissive for HSV-1 17+ and HSV1716 replication. After 72 hrs of replication HSV-1 17+ yields approximately 27849+/−7511 pfu progeny/input virus and HSV1716 yields 913+1-299 pfu progeny/input virus equivalent to a replication competence ratio (HSV1716 yield/HSV-1 17+ yield) of 0.03 Thus HSV1716 replication in SKOV3 cells is severely impaired 30-fold compared to wild-type HSV-1 17+.

### 10) CP70

CP70 is a human, ovarian cancer derived cell line. The CP70 cell line is a cisplatin-resistant derivative of A2780 cells and the cells have approximately 13-fold more resistance to cisplatin than the parental A2780 line. The A2780 human ovarian cancer cell line was established from tumour tissue from an untreated patient. According to their entry in COSMIC they have a mutated PTEN gene.

Both wild-type 17+ and HSV1716 have impaired replication kinetics in CP70 cells. After 72 hrs of replication HSV-1 17+ yields approximately 223+/−59 pfu progeny/input virus and HSV1716 yields 57+/−7pfu progeny/input virus equivalent to a replication competence ratio (HSV1716 yield/HSV-1 17+ yield) of 0.26. Thus HSV1716 replication in CP70 cells is impaired 4-fold compared to wild-type HSV-1 17+.

CP70 cells readily form subcutaneous xenografts in nude mice.

### Combination Studies

Cells were plated out in the internal 6×10 grid of a 96-well tissue culture plate at ˜5000 cells per well. Virus alone, drug alone or virus/drug combinations were added after 24 hours in culture in quadruplicate at least and, after a further 72 hours of incubation, their effects on cell death were determined using CytoTox-Glo Cytotoxicity Assay (Promega) which measures the activity of dead cell protease (DCP) released into the medium. Light emission from the DCP assay was detected using a Perkin Elmer 1420 multilabel counter Victor 3 in luminometer mode for 0.1 sec/well. Killing curves for virus alone and drug alone (GraphPad Prism 4) were used to determine IC50s and identify moi/drug concentrations for combination studies. Combination studies used 4 drug concentrations and two mois and were performed on a single plate with each plate repeated at least three times. Data was subjected to Chou-Talalay analysis to identify synergies and antagonisms using either CompuSyn software (Combosyn Inc.) or an in-house derived Chou-Talalay-based spreadsheet (a kind gift from Prof Tim Cripe).

### DCP Assay

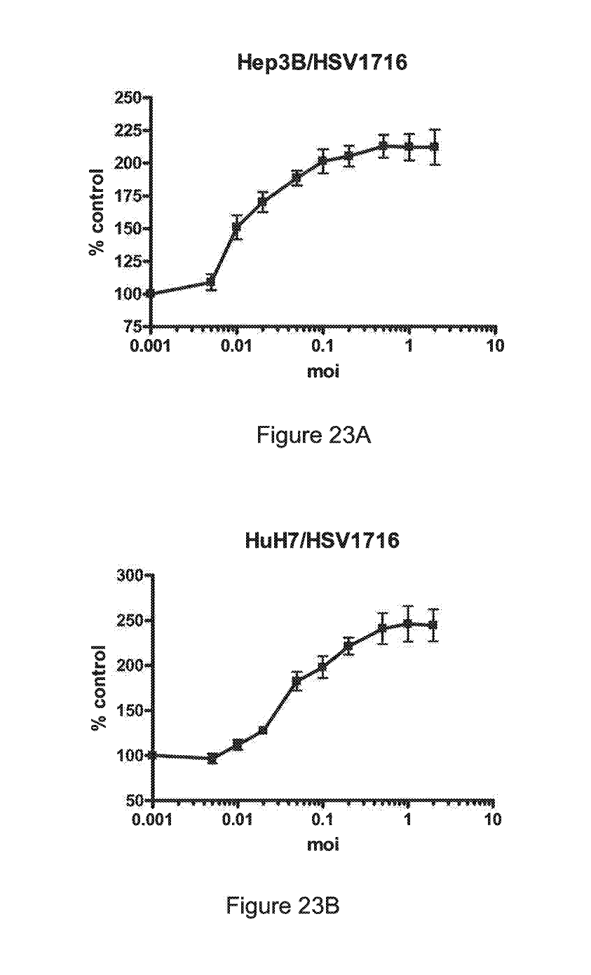

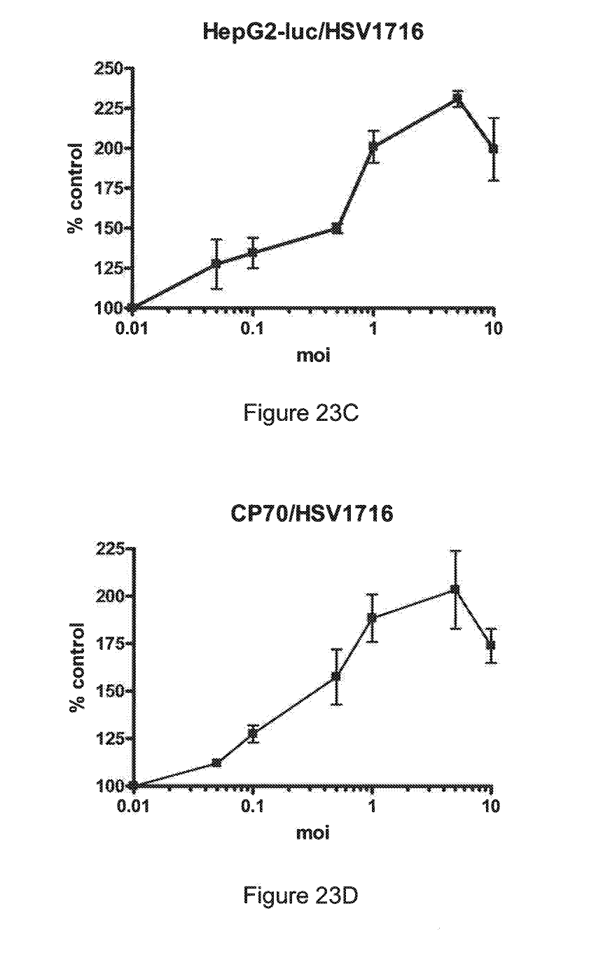

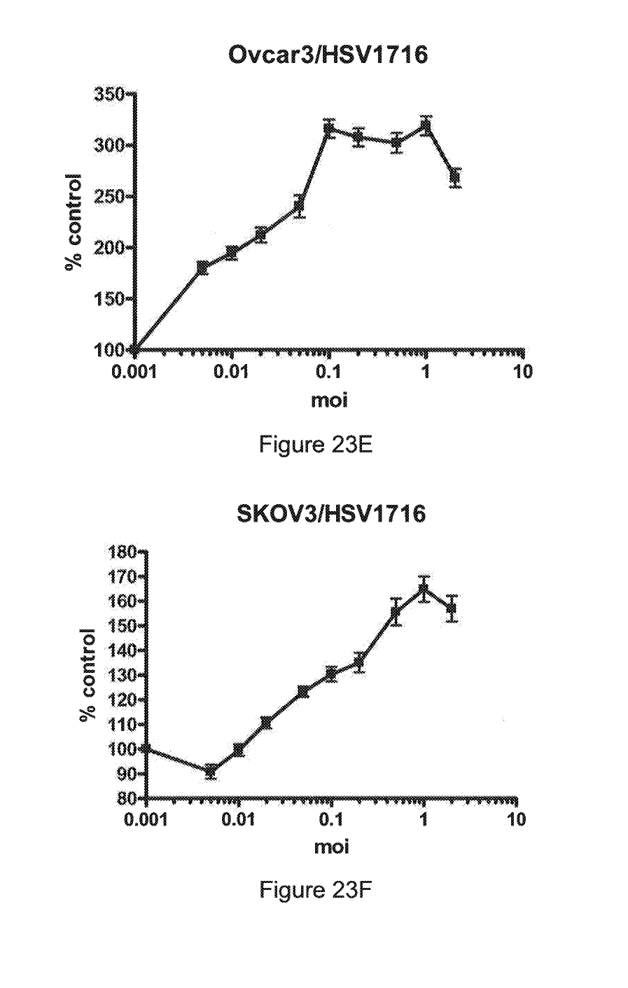

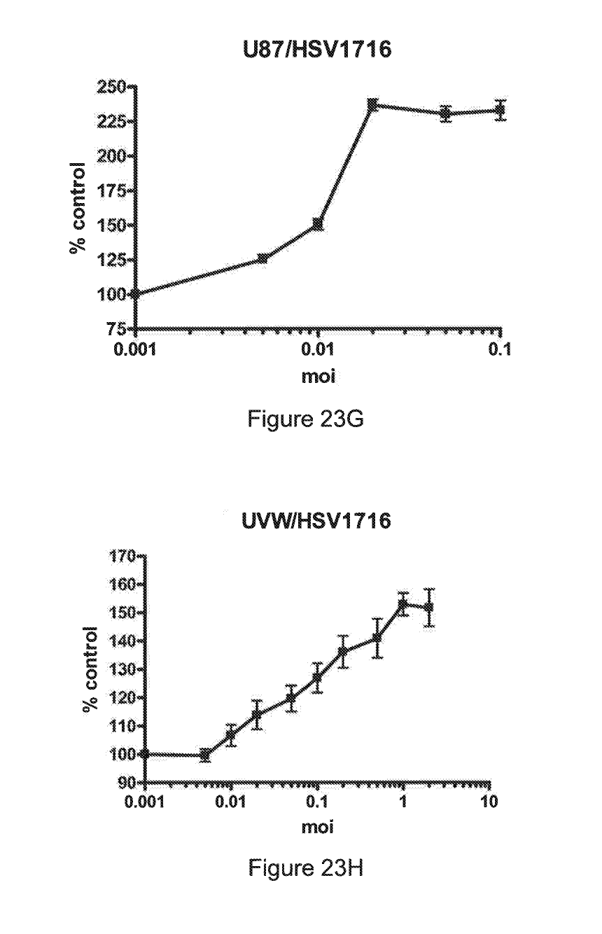

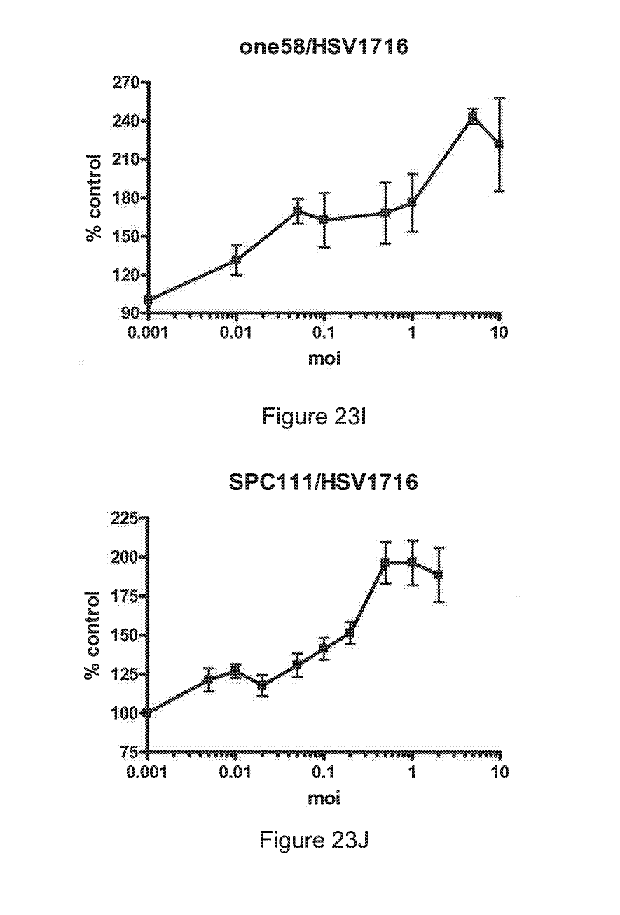

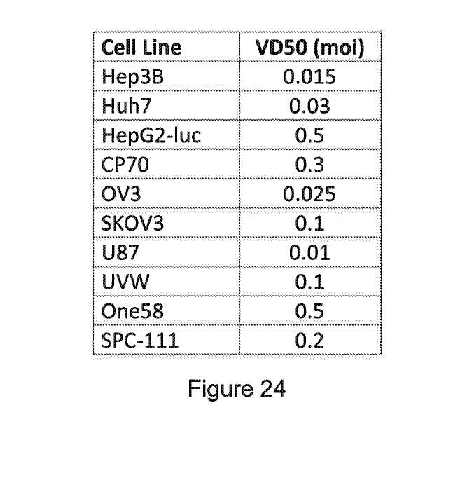

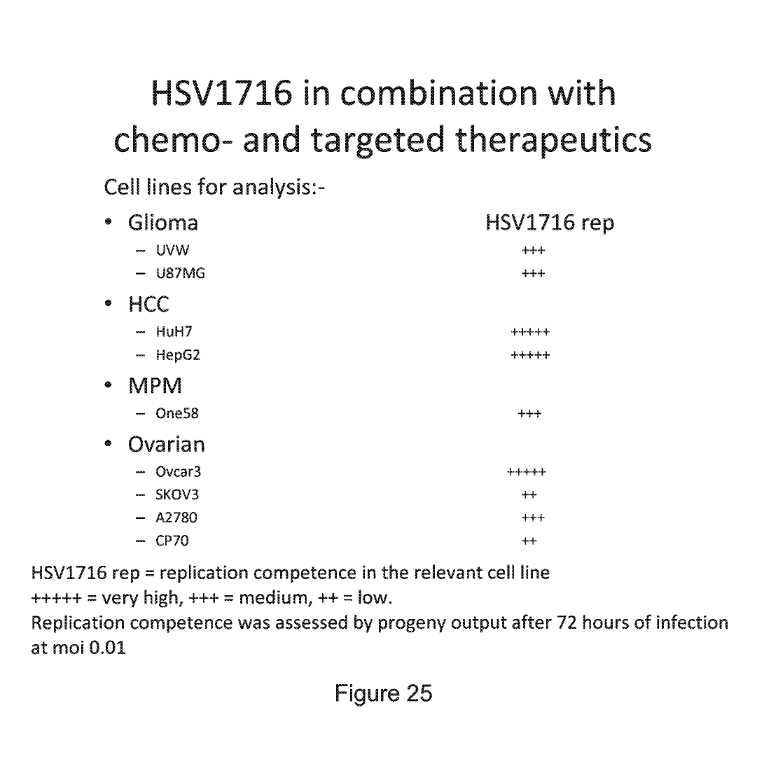

Dead cell protease was assayed using the CytoTox-Glo Cytotoxicity kit from Promega. The kit provides a lumiogenic peptide substrate, AAF-Glo, to measure dead cell protease activity in the medium. As with lactate dehydrogenase (LDH) assay, dead cell protease is released from cells which have lost membrane integrity. The peptide substrate cannot cross the intact cell membrane of a live cell and will only be cleaved when dead cell protease has been released into the medium as cells die. The assay then uses the Ultra-Glo recombinant luciferase which can use the released aminoluciferin as substrate to generate a readily detectable luminescence signal. The DCP assay is more sensitive than the LDH assay and can potentially detect as few as 200-500 dead cells/well. Use of the DCP assay to detect cell killing by HSV1716 in all cell lines of the Virttu cell panel is shown in FIG. 23 and results summarised in FIG. 24

### HSV1716 for Combination Studies

An HSV1716 variant expressing green fluorescent protein was used for combination analysis. HSV1716gfp was produced from the parental HSV1716 by insertion of a CMV-gfp expression cassette in the UL-43 gene.

The initial viral titre was 1×10⁹ pfu/ml. The stock was diluted 1:100 in medium (0.5 ml virus in 49.5 ml) to give a 1×10⁷ pfu/ml stock and dispensed in 5 ml aliquots for storage. The 5 ml of 1×10⁷ pfu/ml stock was diluted into 45 ml medium to generate a 1×10⁶ pfu/ml stock, 6 ml aliquots of which are stored for use in assays. To dilute this 6 ml to working strength (moi 0.5 and 0.05*):—
Moi 0.5: Add 5 ml of the 1×10⁶ pfu/ml stock to 45 ml medium (=1×10⁶ pfu/ml), 50 ul of this in the single well of a 96-well plate is equivalent to 5000 pfu/well.
Moi 0.05: Add 0.5 ml of the 1×10⁶ pfu/ml stock to 49.5 ml medium (=1×10⁴ pfu/ml), 50 ul of this in the single well of a 96-well plate is equivalent to 500 pfu/well.
counts suggest between 8000-10000 cells/well for most cell types at time of infection.

GFP expression was measured using a Perkin Elmer 1420 multilabel counter Victor 3 in FITC fluoremetry mode for 1 sec/well.

### Example 2

### Sorafenab

Sorafenib was analysed in combination with HSV1716 in vitro using the cell lines described in Example 1. Toxicity was assessed in each well using DCP cell death assays. Toxic effects of the virus on the drug were also assessed by using HSV1716gfp.

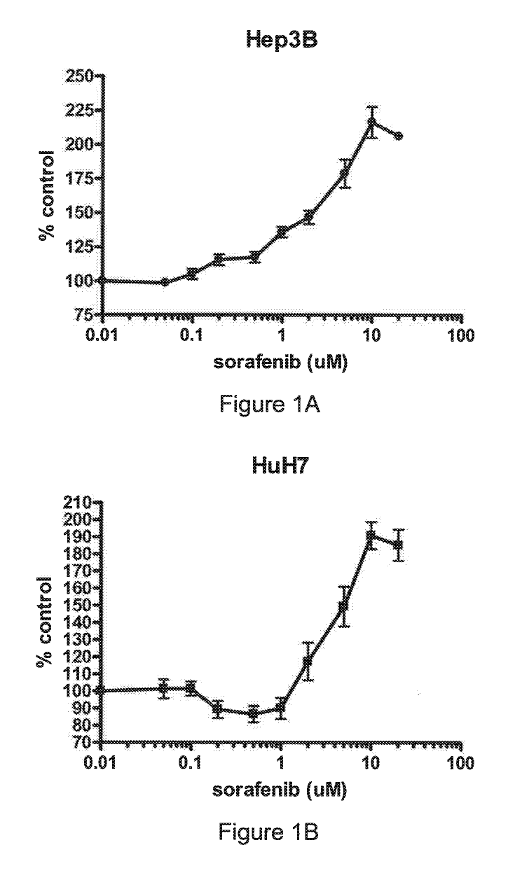

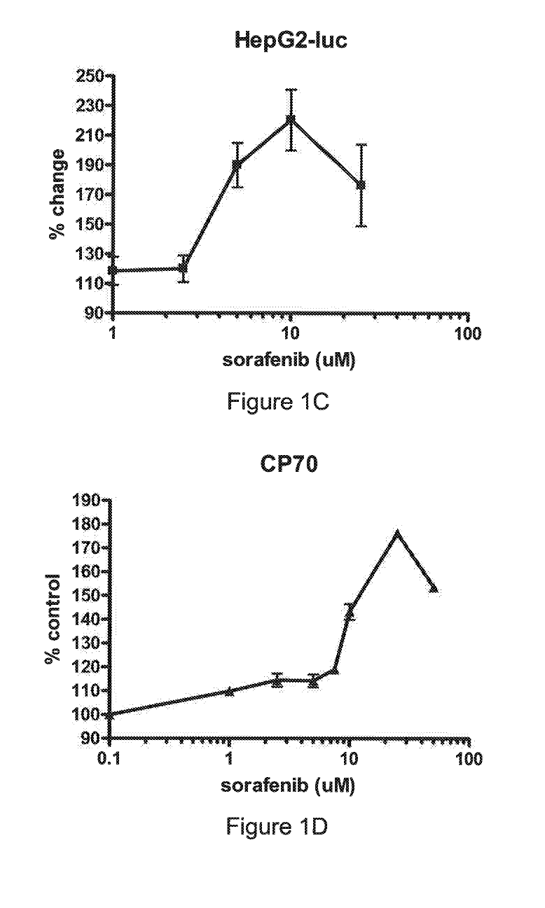

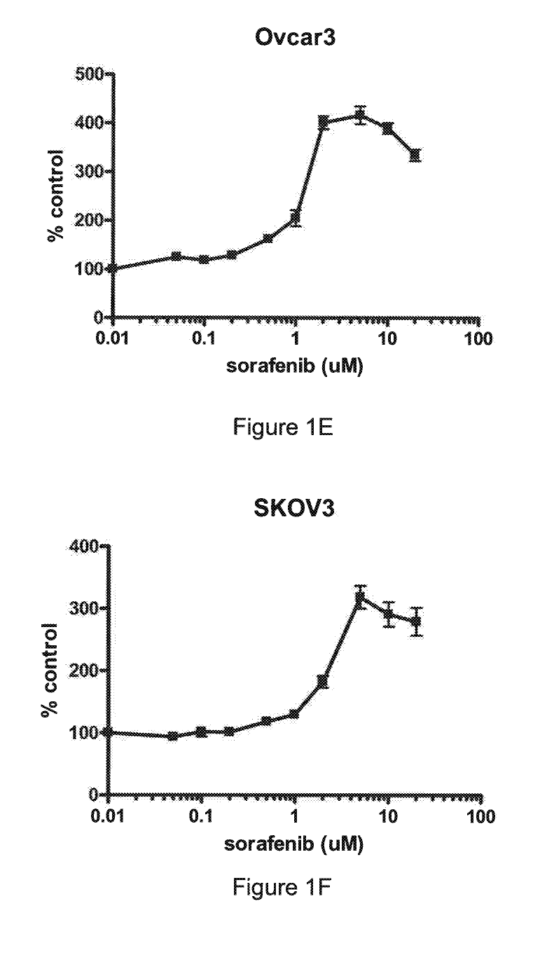

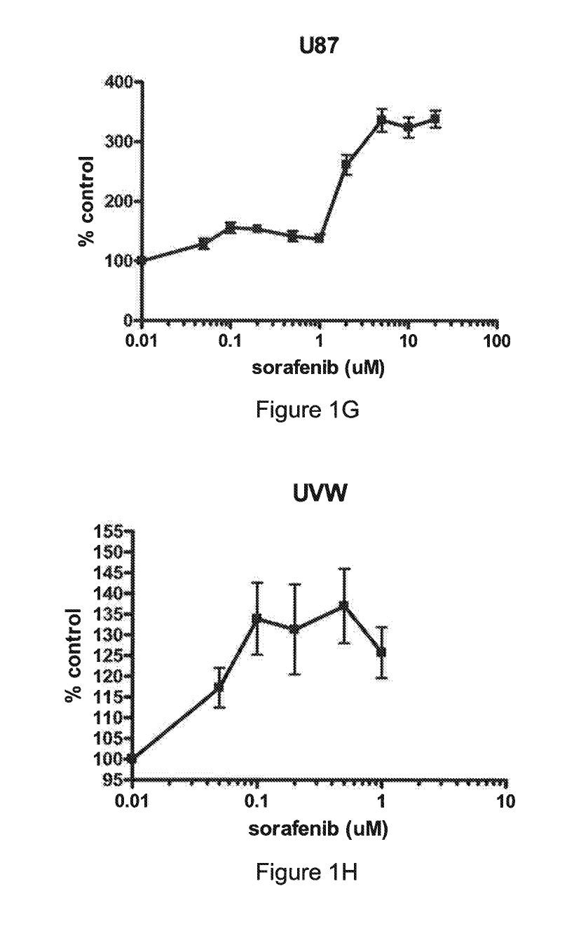

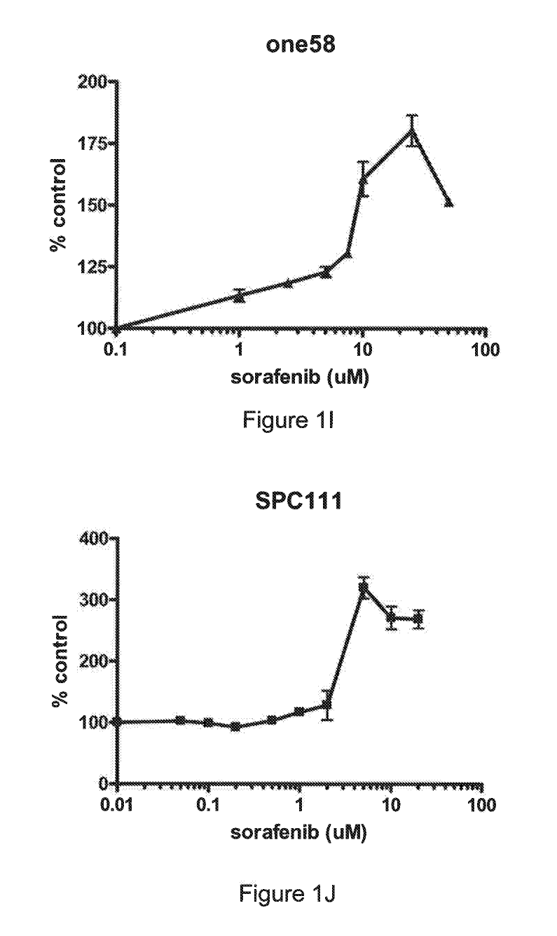

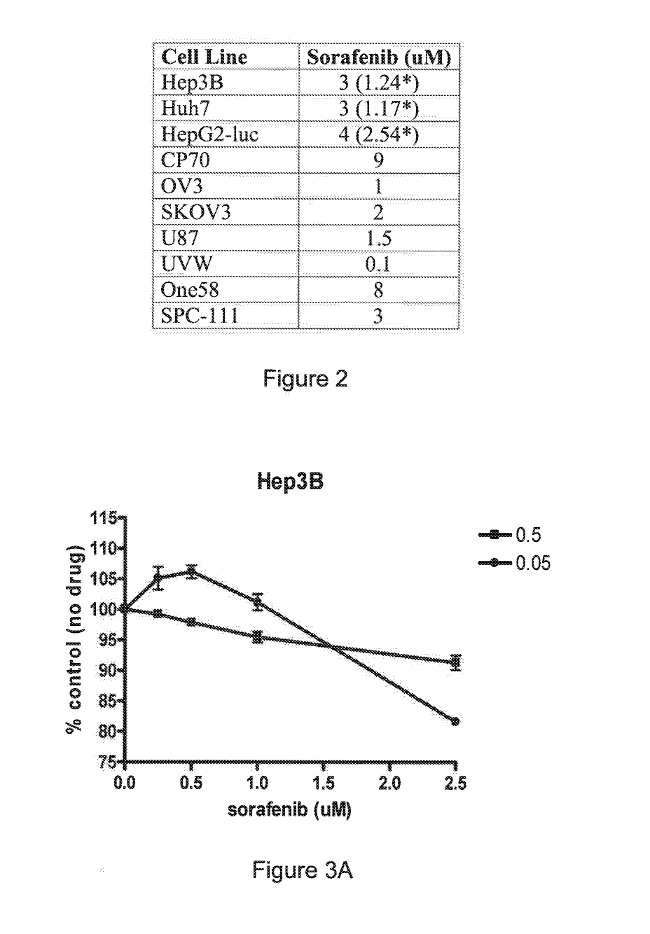

IC50 curves for each cell line tested are shown in FIG. 1 and the estimated IC50s are shown in Table 1 (FIG. 2). Sorafenib was toxic in most cell lines with IC50s for most in the range 1-10 μM with little cell line variability in toxicity. UVW were the most sensitive to this drug with an IC50 of 0.1 μM. (Table 1; FIG. 2).

### HSV1716 Toxicity

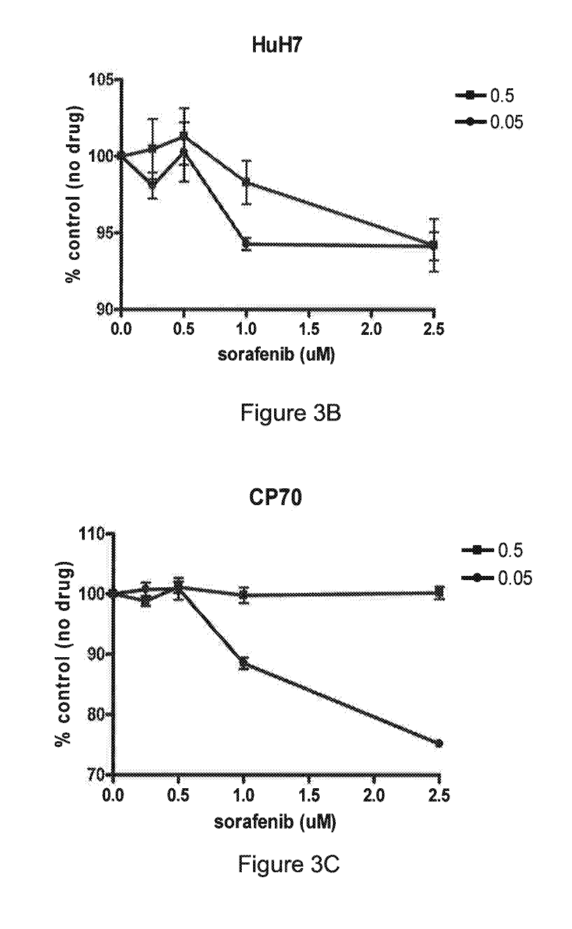

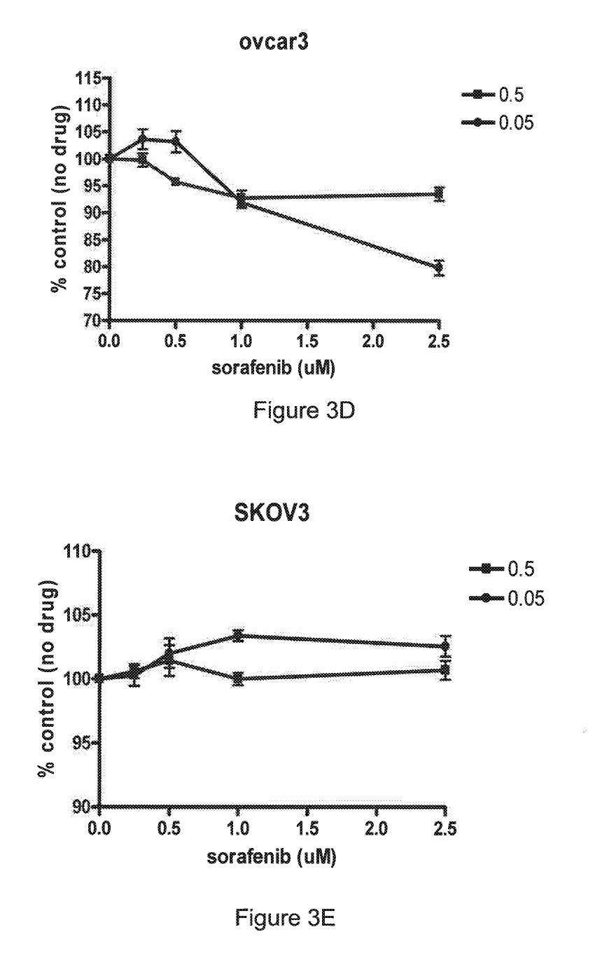

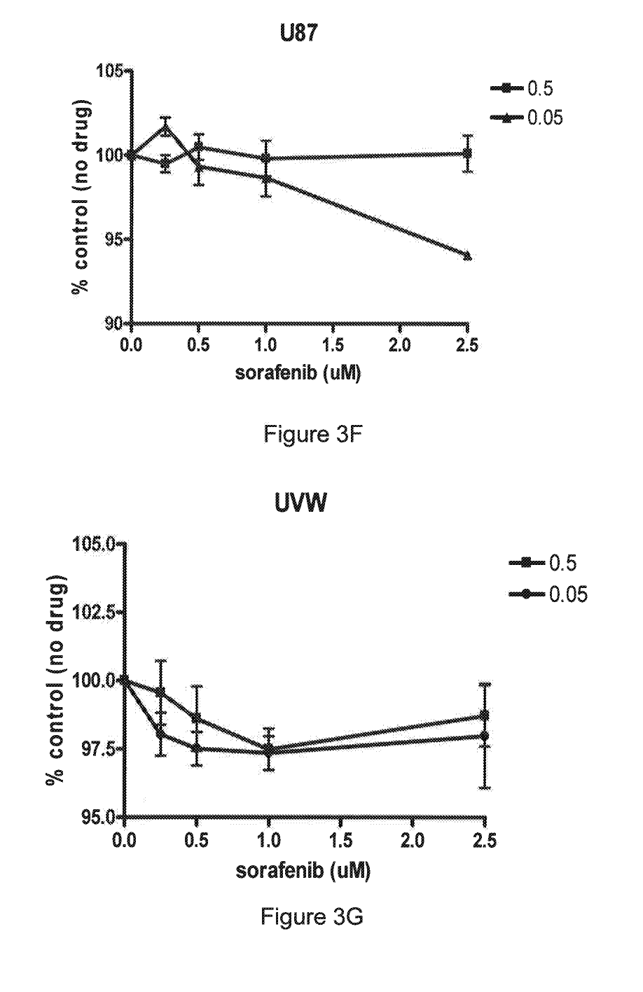

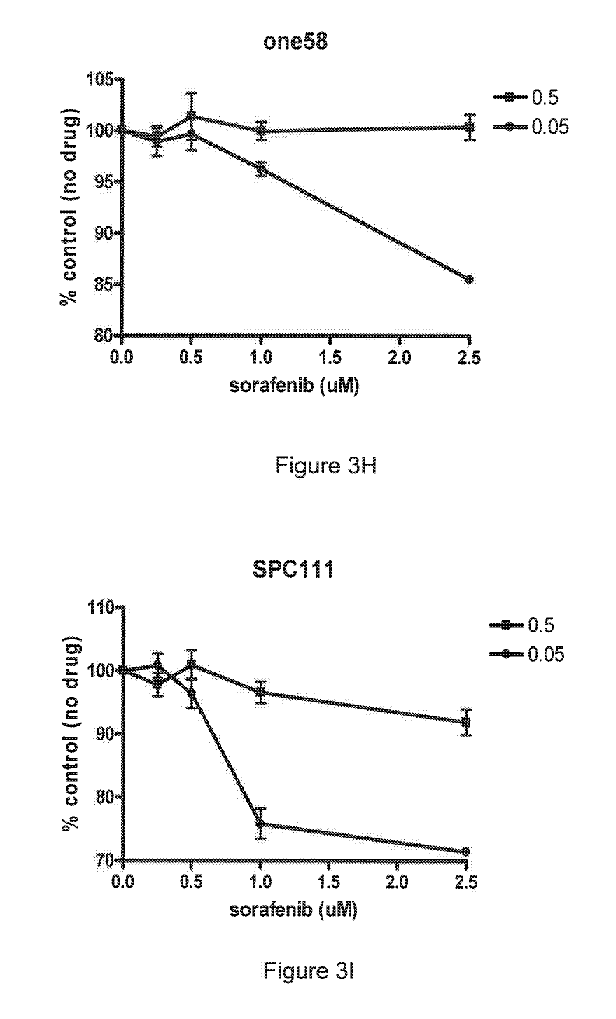

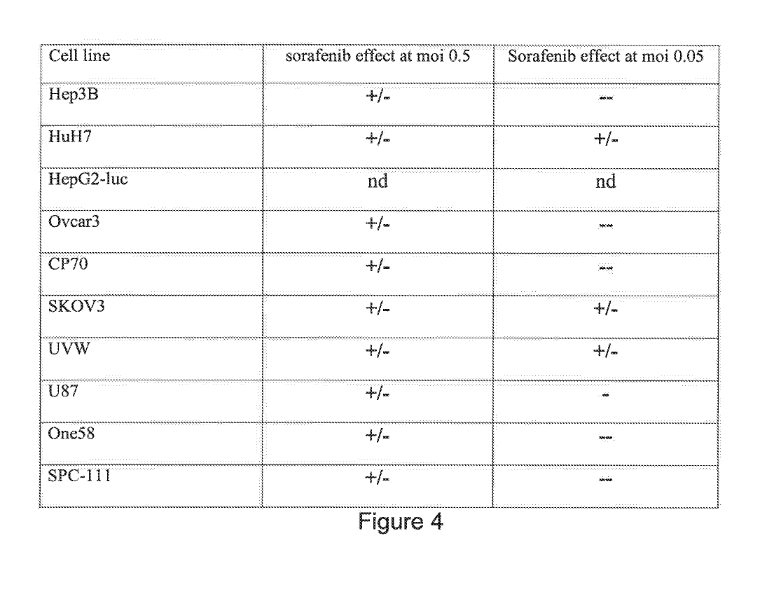

Sorafenib had limited toxicity with respect to HSV1716 as assessed by GFP expression during infection of the cell lines with HSV1716gfp. HepG2-luc cells were not available for this analysis. Sorafenib was incubated with cells plus virus at 2.5 μM, 1 μM, 0.5 μM and 0.25 μM for 72 hrs and GFP fluorescence measured. Drug-mediated virus toxicities are presented in the graphs in FIG. 3 as percentage of control cells (virus alone and no drug) in Hep3B, HuH7, CP70, Ovcar3, SKOV3, U87, UVW, One58 and SPC-111. Virus was used at moi 0.5 or moi 0.05. In all 9 of the cell lines tested, Sorafenib had little effect on GFP expression during HSV1716gfp infection at moi 0.5 (<+1-5% variation from control, Table 2 (FIG. 4)). At the lower moi 0.05, Sorafenib had no effect on GFP expression during HSV1716gfp infection in SKOV3 and UVW cells but there was a small inhibitory effect at the 2.5 μM Sorafenib concentration in HuH7 and U87 cells (>5% but <10% of control values, Table 2; FIG. 4). Sorafenib had a more marked inhibitory effect on GFP expression during HSV1716gfp infection at 2.5 μM and moi 0.05 in Hep3B, CP70, Ovcar3, One58 and SPC111 cells (>10% of control, Table 2 (FIG. 4)). Therefore Sorafenib had limited toxic effects on HSV1716 and the results are summarised in Table 2 (FIG. 4).

### Combination Analysis

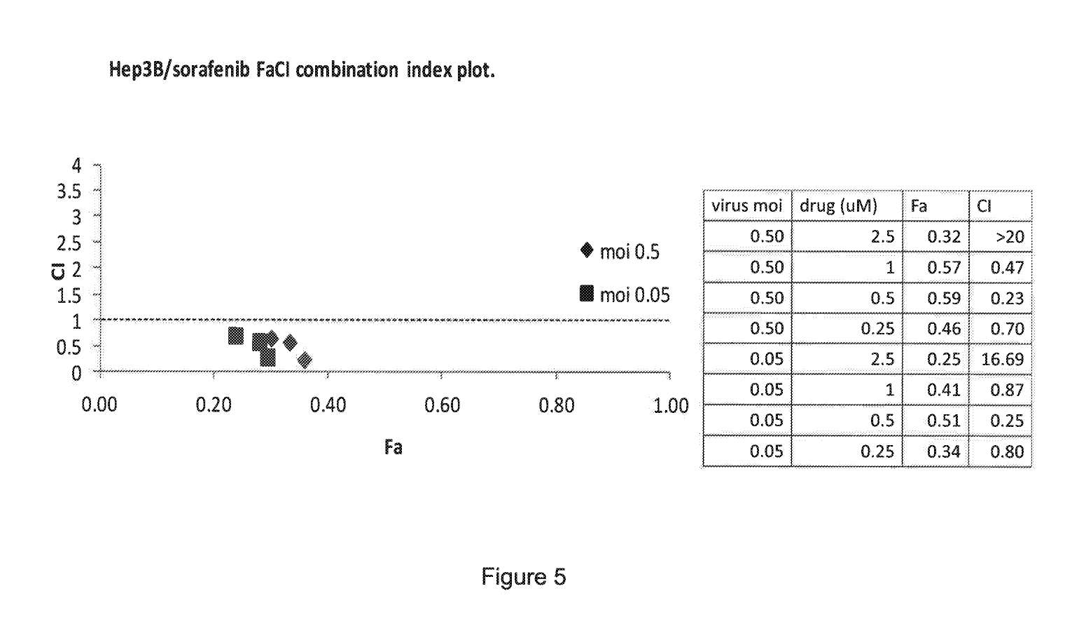

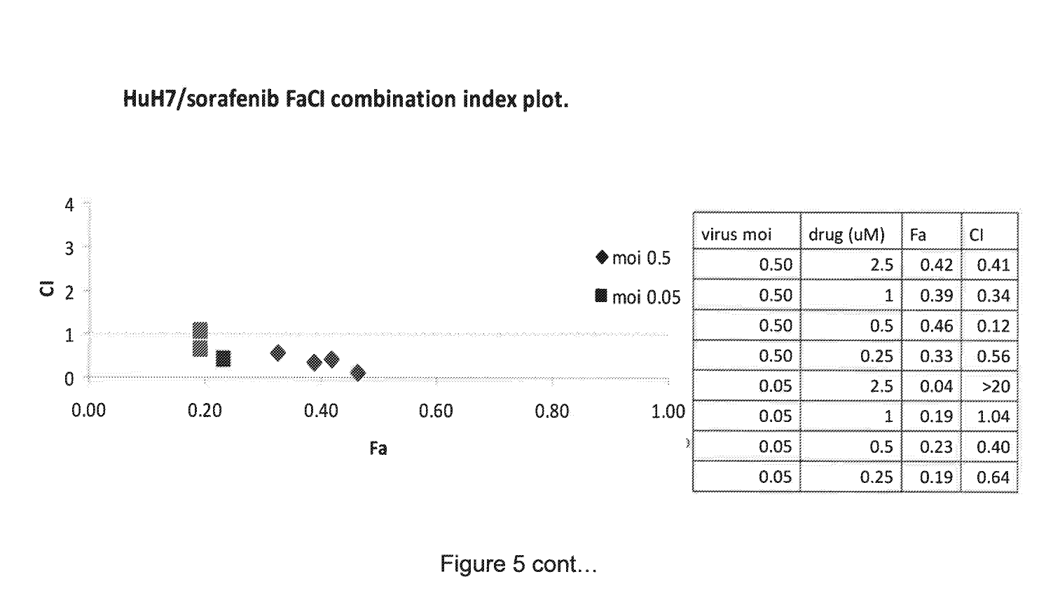

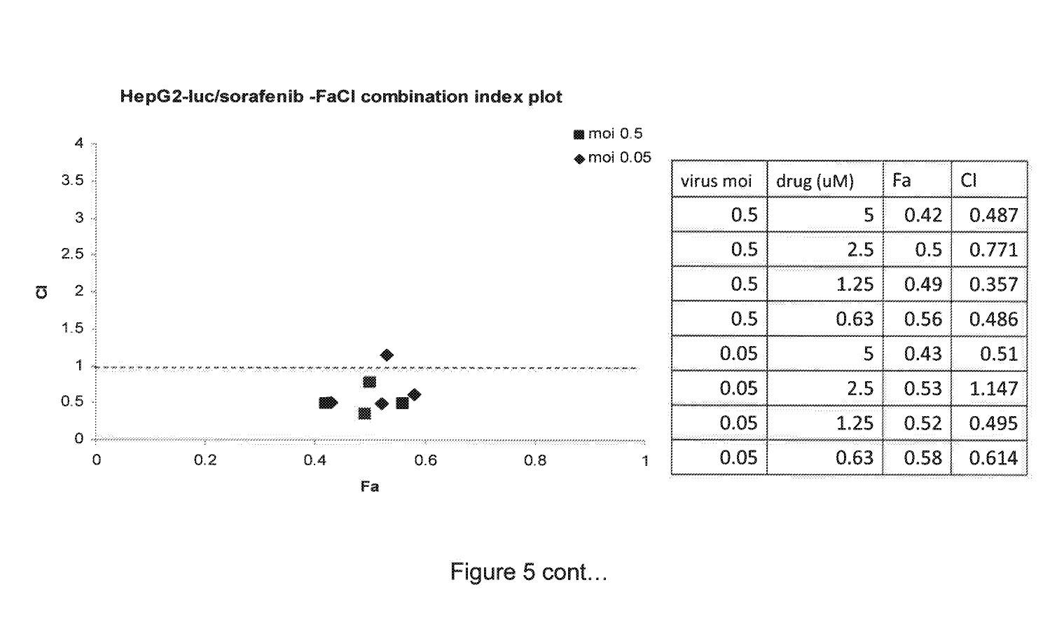

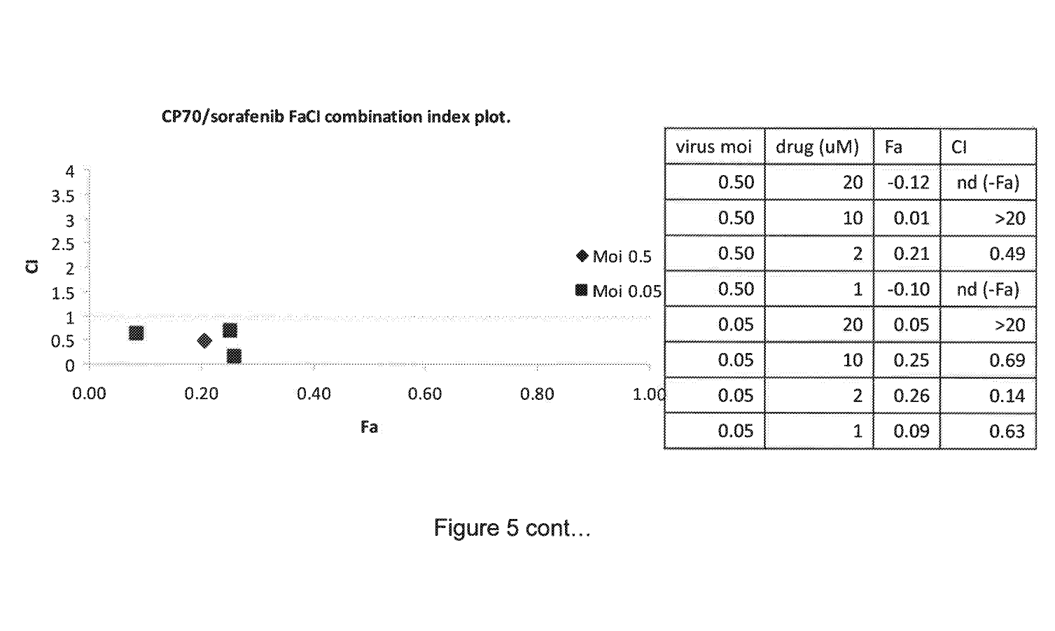

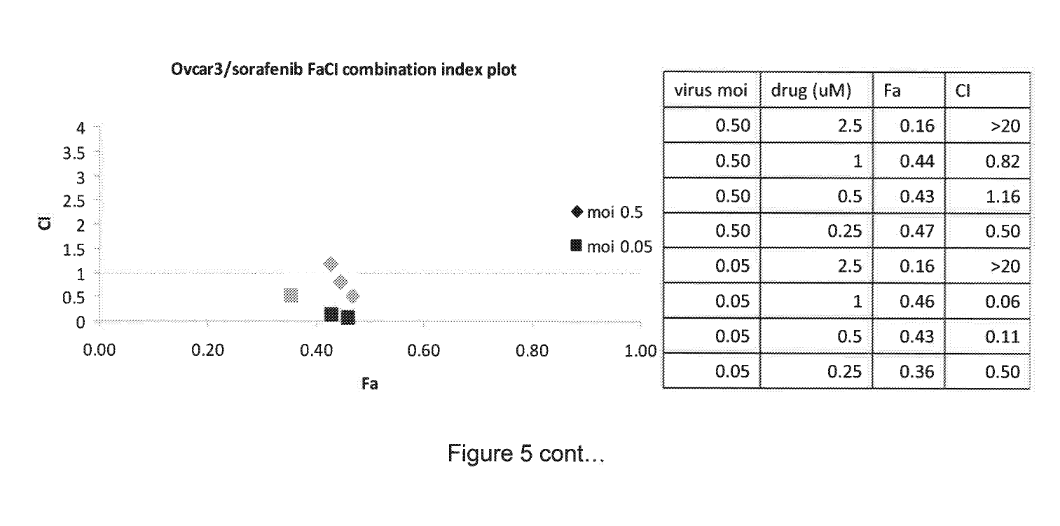

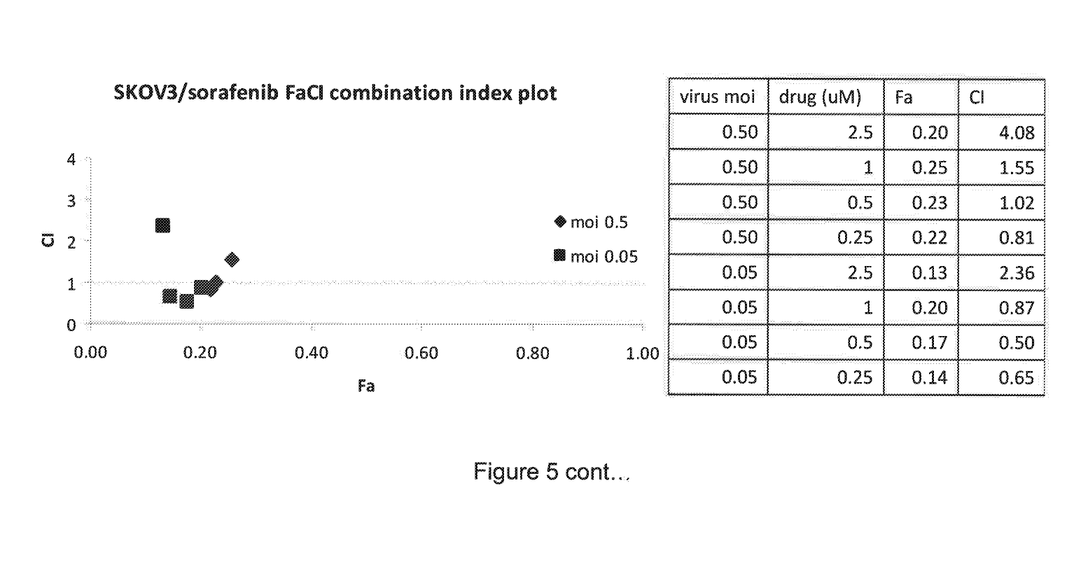

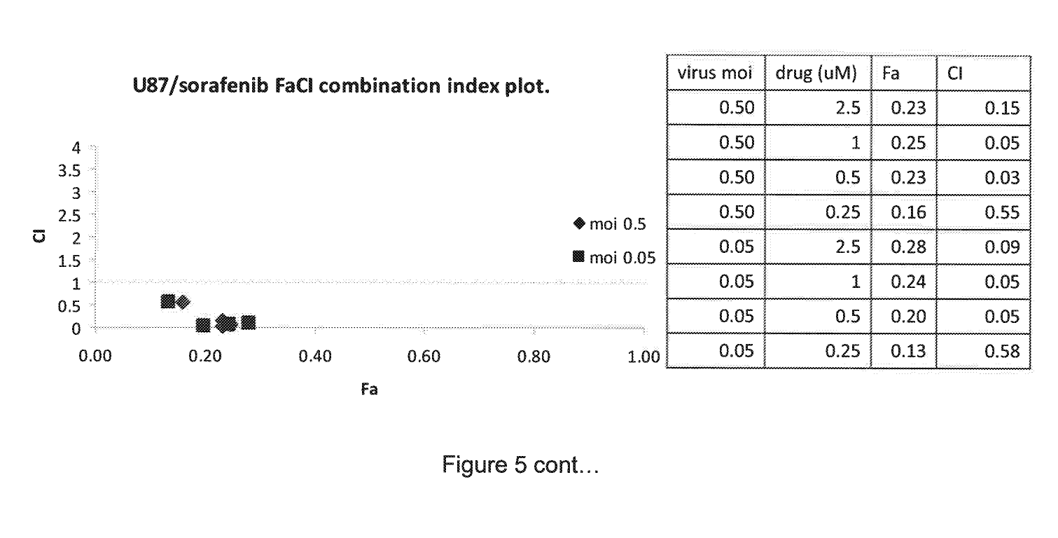

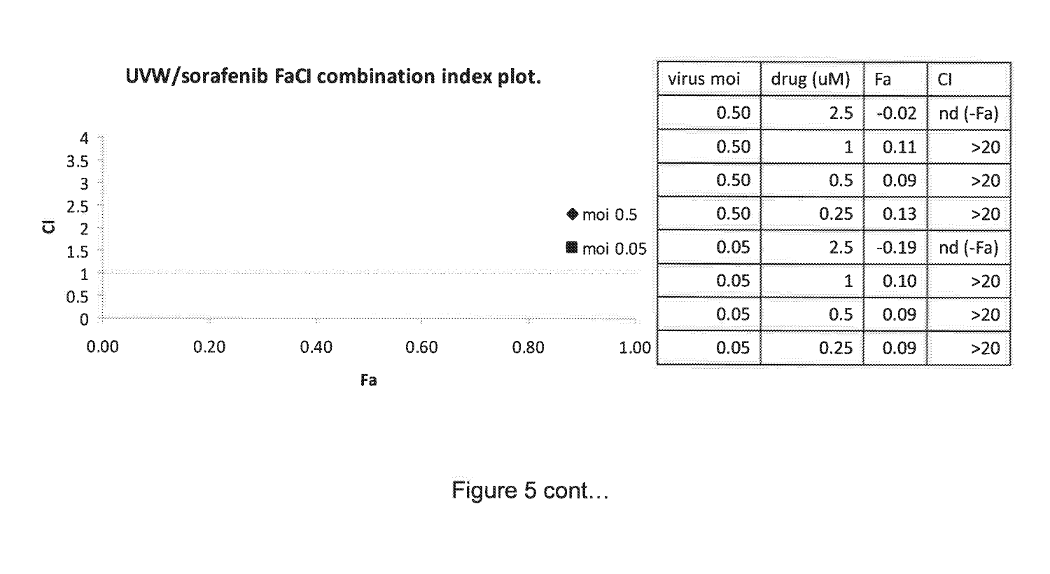

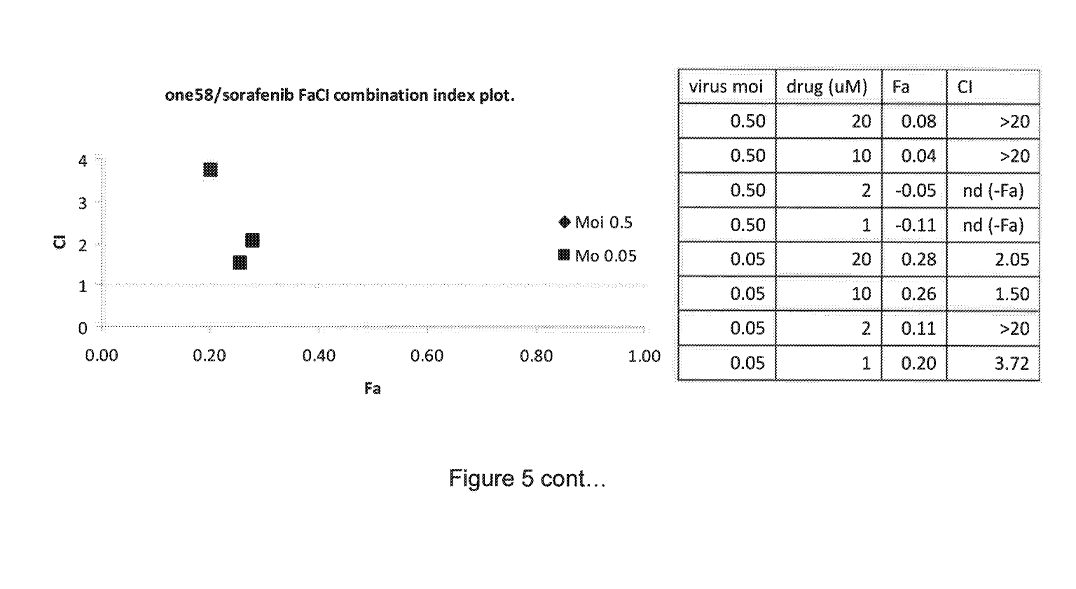

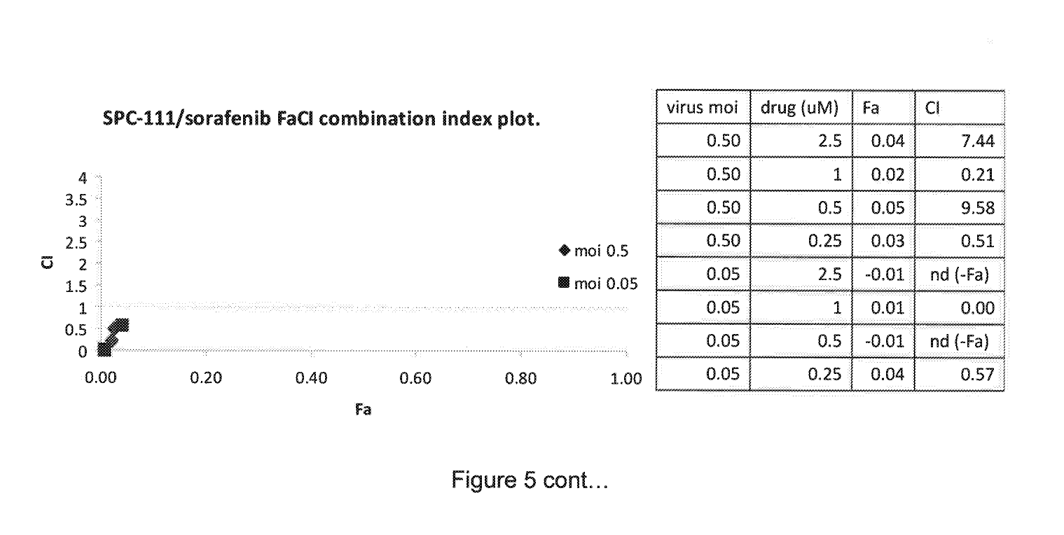

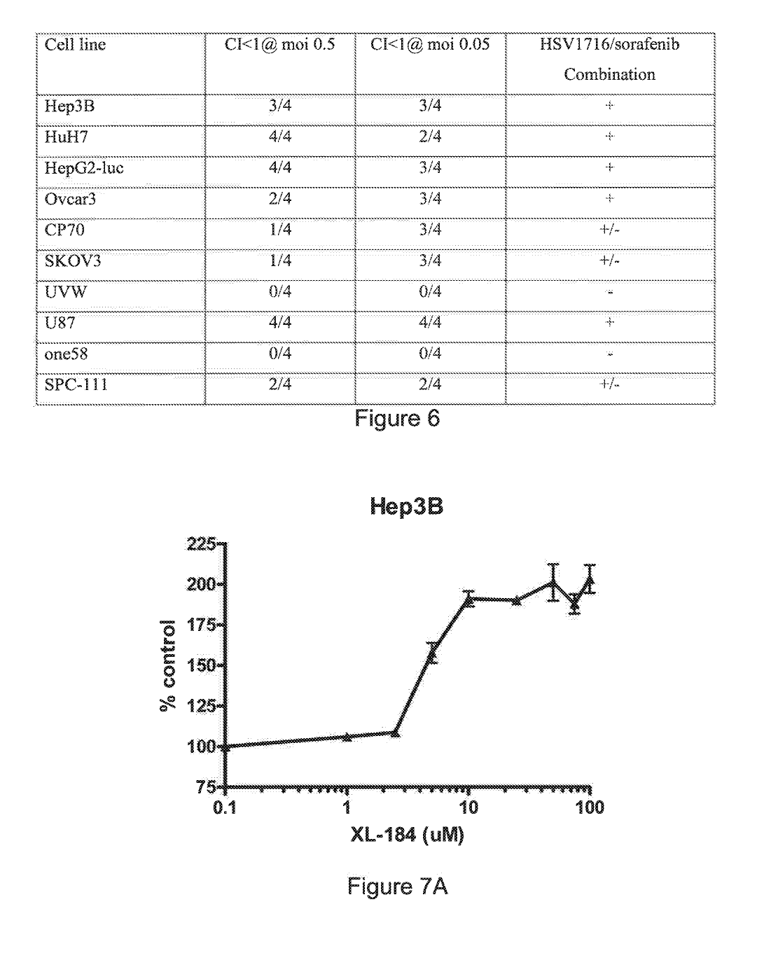

Sorafenib was tested at 4 different doses (2.5, 1, 0.5 and 0.25 μM in Hep3B, HuH7, Ovcar3, SKOV3, U87, UVW, SPC111, 5, 2.5, 1.25 and 0.63 μM in HepG2-luc and 20, 10, 2 and 1 μM in CP70 and One58) in combination with HSV1716 at moi 0.5 and 0.05 and cell death after 72 hours exposure was assessed by DCP assay. Data were analysed for synergy/antagonism by calculating combination indices and graphing them against Fa (FaCl/Chou-Talalay plots) using a spreadsheet designed to automatically calculate Fa and Cl and graph the corresponding results from the raw DCP readings. The analysis was repeated on three separate plates and representative individual plots along with their respective Fa and Cl table of values are presented in FIG. 5 with synergies/antagonisms summarised in Table 3 (FIG. 6). The Cl values for the 4 drug concentrations at the two HSV1716 moi were determined and if less than 1 then the combination of virus and drug must act synergistically to enhance cell death. If 4 or more of the 8 available combinations resulted in Cl values<1 then the drug was identified as combining synergistically with HSV1716 in that cell line. If more than 4/8 available combinations resulted in Cl values>1 then the drug was seen as antagonistic with HSV1716. A negative Fa value occurs when the test DCP value is less than the control (i.e. indicative of improved survival) and is therefore scored as antagonistic.

### Conclusions

Sorafenib is toxic in most of the cell lines tested.
There are clear synergy signals in 8/10 cell lines tested (all cell lines tested with no synergies in UVW or One58 cells).
Sorafenib was strongly synergistic with HSV1716 in 3/3 HCC cell lines,
Synergies occurred at both moi and did not appear to correlate with the inhibitory effects of the drug on the virus (i.e. synergies occurred where Sorafenib had a negative effect on GFP expression during HSV1716gfp replication in ovcar3, CP70 and Hep3B).

### Example 3

### Cabozantinib

Cabozantinib (XL-184) was analysed in combination with HSV1716 in vitro using the cell lines described. Toxicity was assessed in each well using DCP cell death assays. Toxic effects of the virus on the drug were also assessed by using HSV1716gfp.

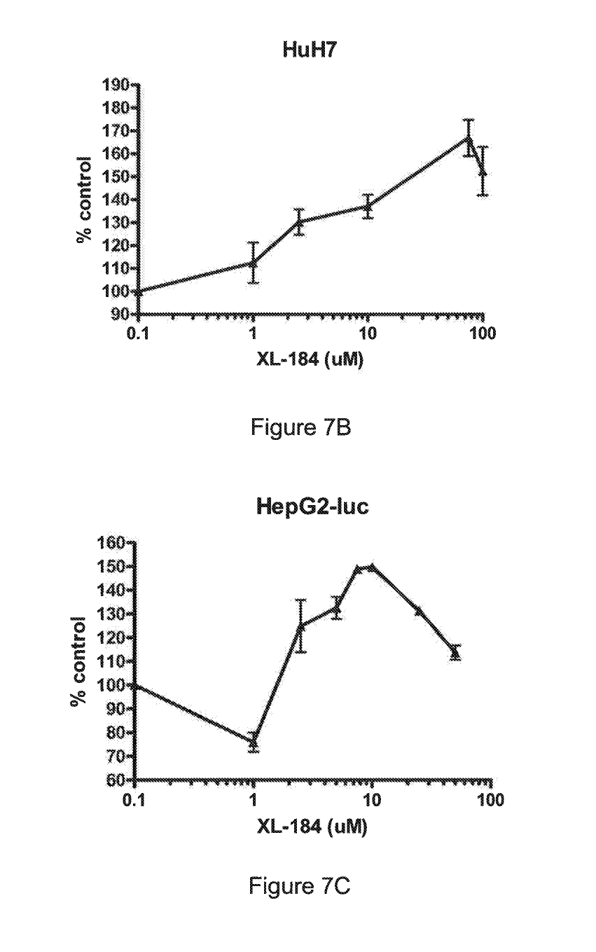

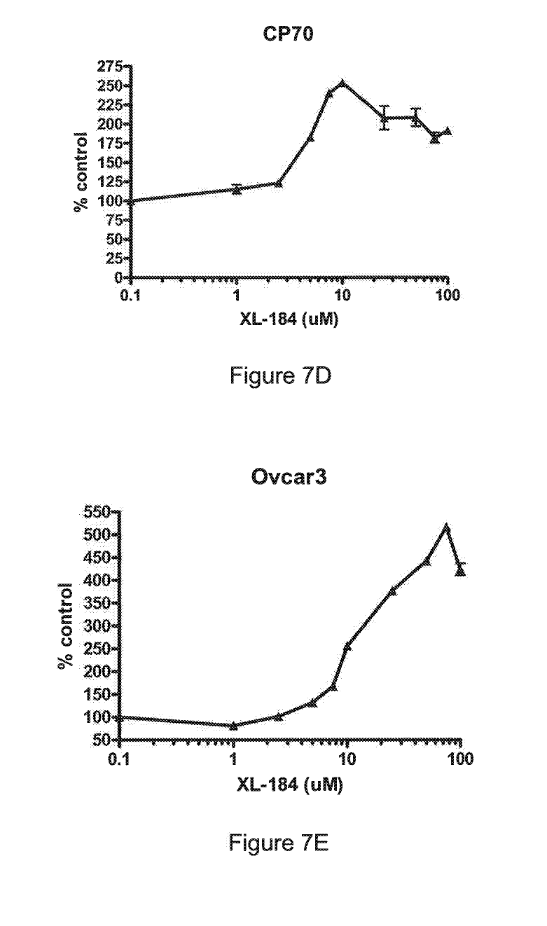

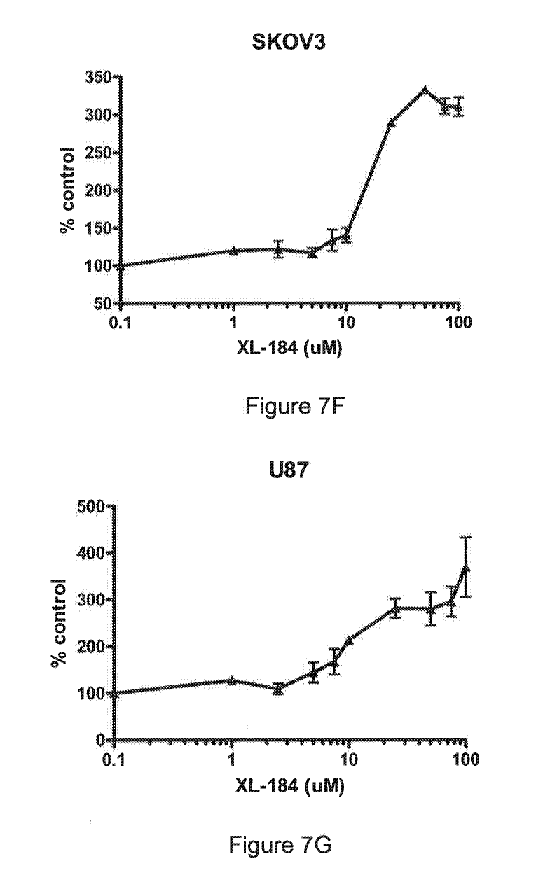

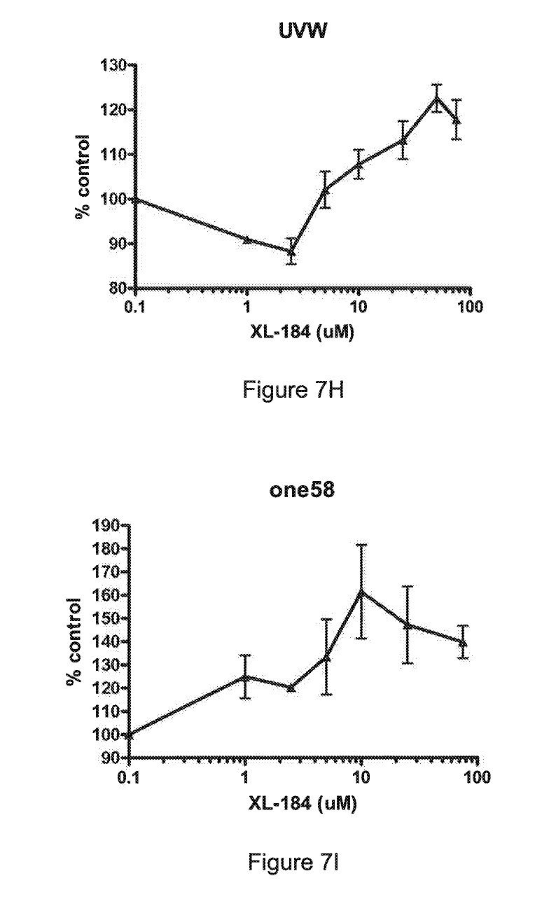

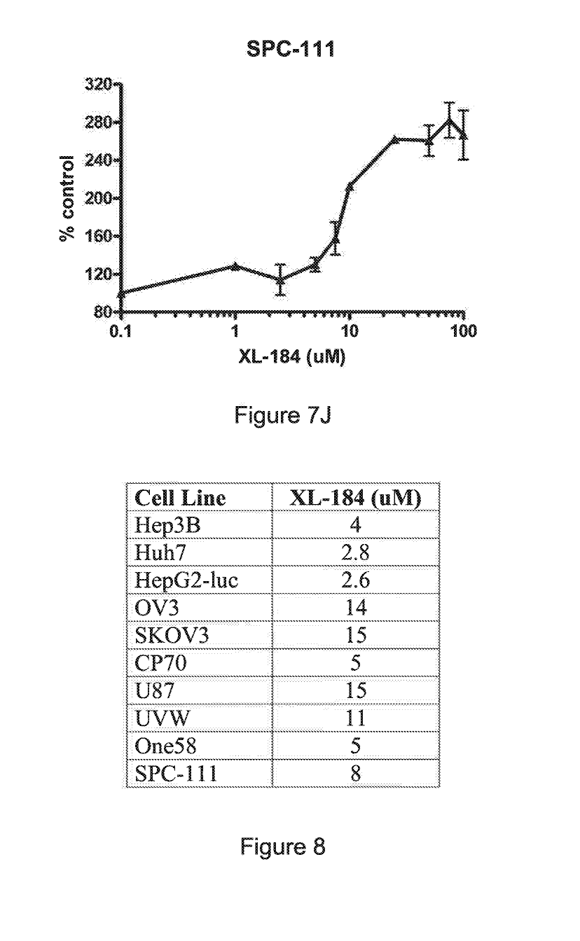

IC50 curves for each cell line tested are shown in FIG. 7 and the estimated IC50s are shown in Table 4 (FIG. 8). XL-184 was toxic in all cell lines with IC50s in the range 2.6-15 μM with limited cell line variability in toxicity (Table 4; FIG. 8).

### HSV1716 Toxicity

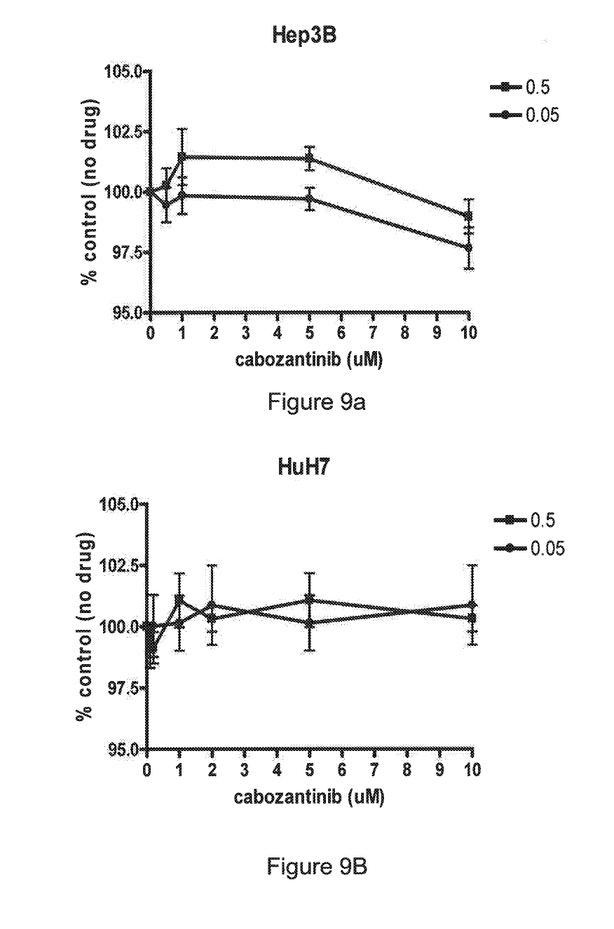

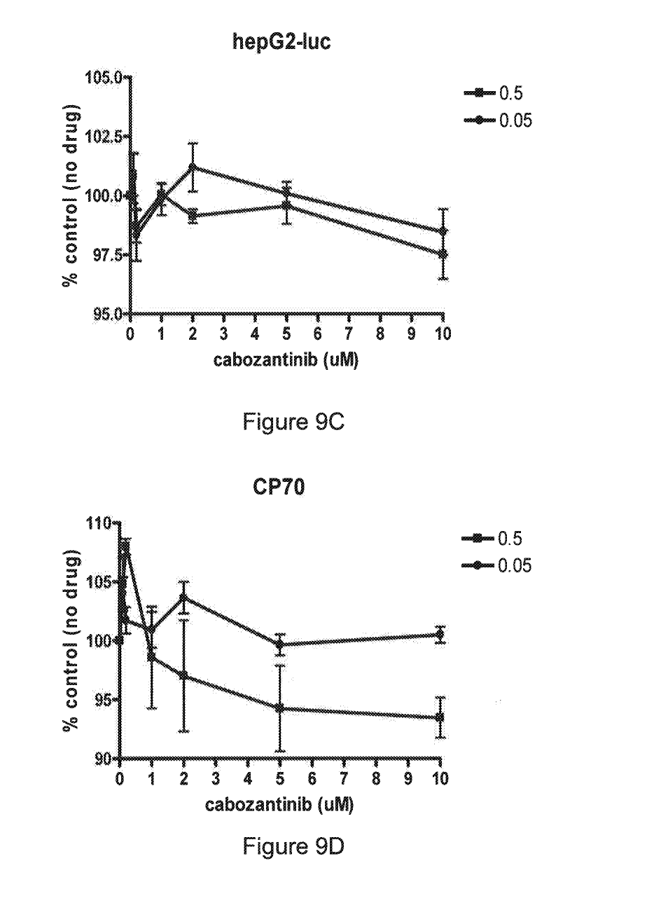

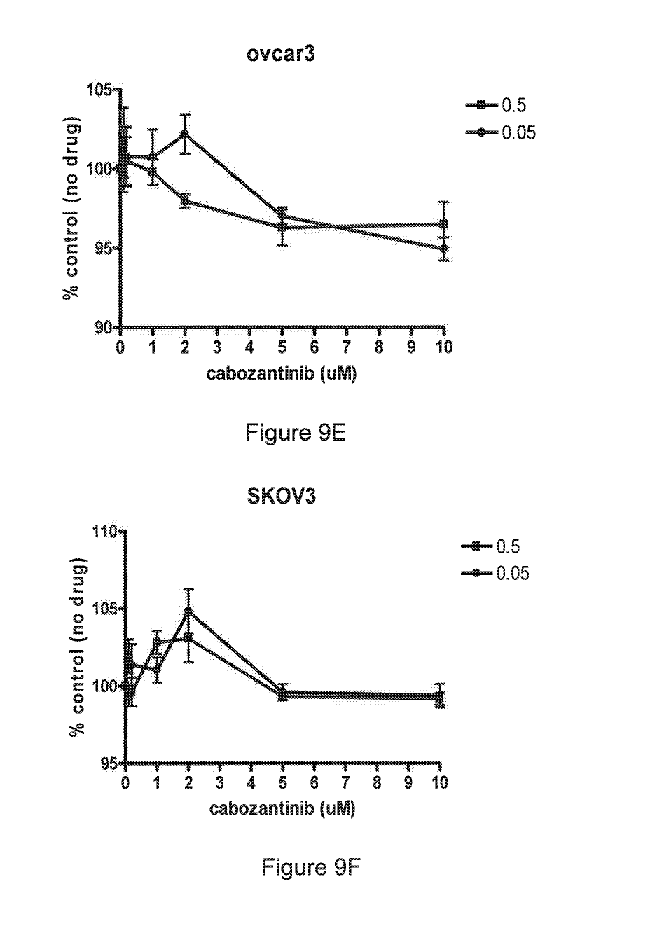

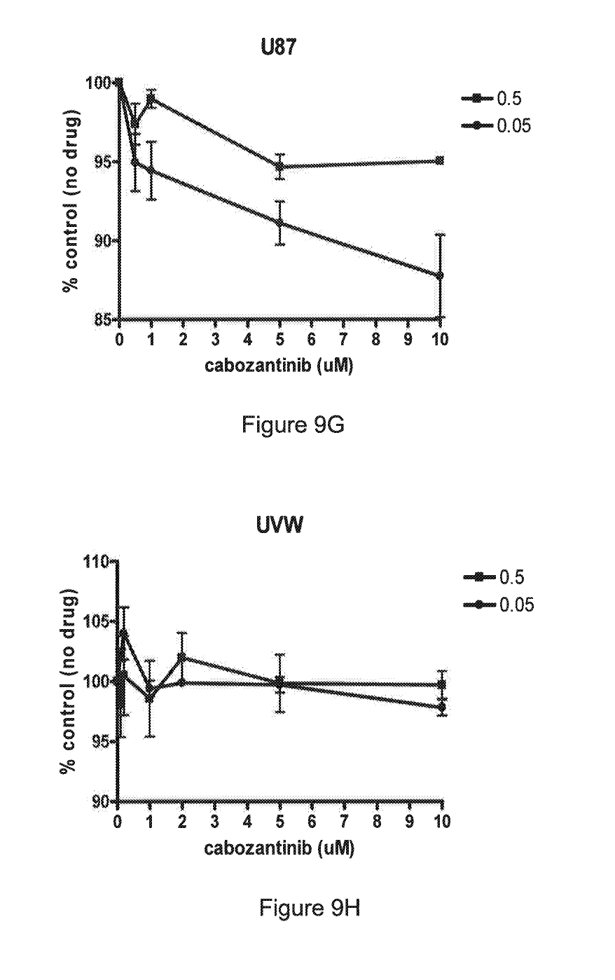

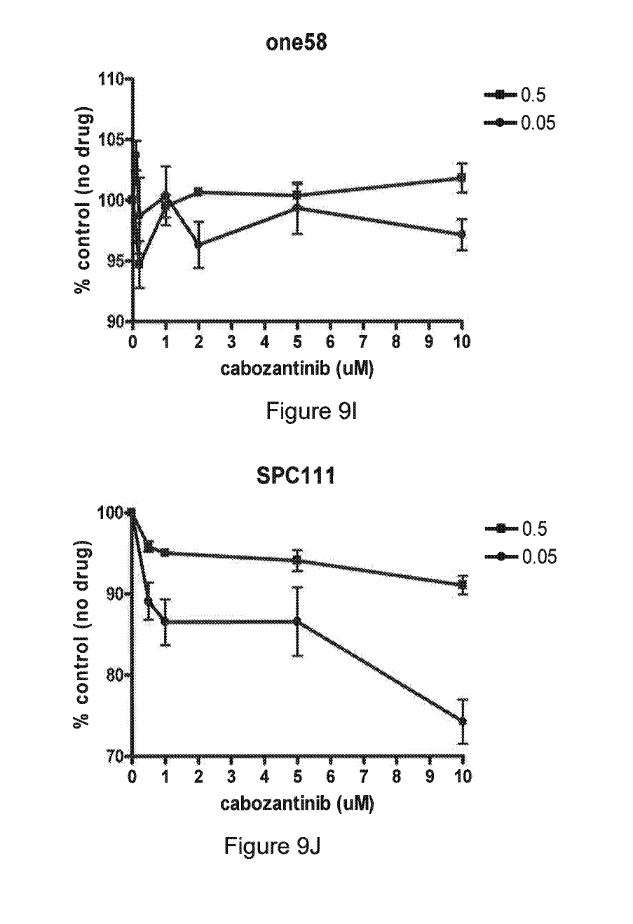

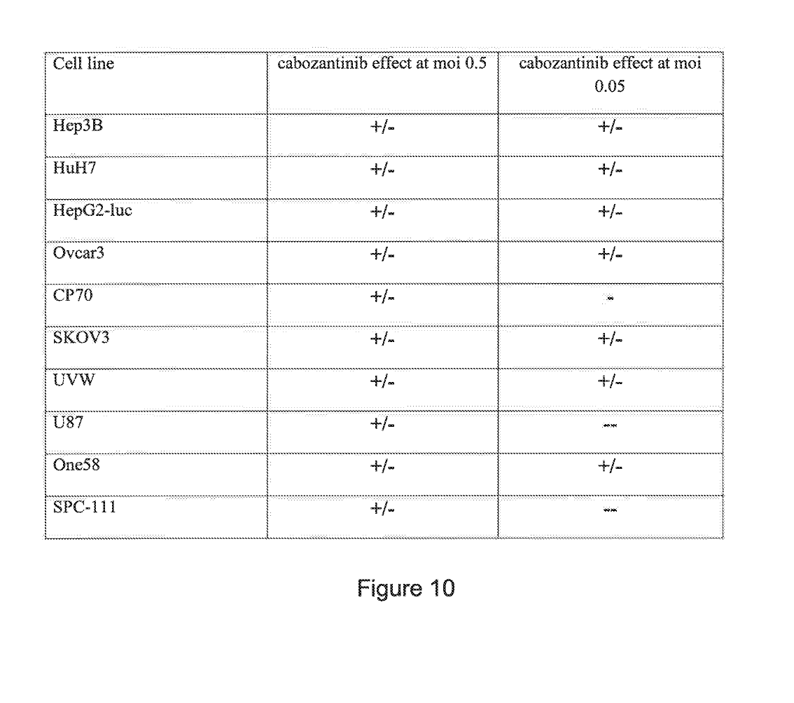

Cabozantinib had very limited toxicity with respect to HSV1716 as assessed by GFP expression during infection of the cell lines with HSV1716gfp. Cabozantinib was incubated with cells plus virus at 10 μM, 5 μM, 2 μM, 1 μM, 0.2 μM and 0.1 μM for 72 hrs and GFP fluorescence measured. Drug-mediated virus toxicities are presented in the graphs in FIG. 9 as percentage of control cells (virus alone and no drug) in Hep3B, HuH7, HepG2-luc, CP70, Ovcar3, SKOV3, U87, UM, One58 and SPC-111. Virus was used at moi 0.5 or moi 0.05. In all 10 of the cell lines tested, Cabozantinib had little effect on GFP expression during HSV1716gfp infection at moi 0.5 (<+/−5% variation from controls. Table 5 (FIG. 10)). At the lower moi 0.05, Cabozantinib had no effect on GFP expression during HSV1716gfp infection in all cells except CP70, U87 and SPC111. There was a small inhibitory effect at moi 0.05 in CP70 cells (>5% but <10% of control values, Table 5 (FIG. 10)) and cabozantinib had a more marked inhibitory effect on GFP expression during HSV1716gfp infection at moi 0.05 in U87 and SPC111 cells (>10% of controls, Table 2). Therefore cabozantinib had very limited toxic effects on HSV1716 and the results are summarised in Table 5 (FIG. 10).

### Combination Analysis

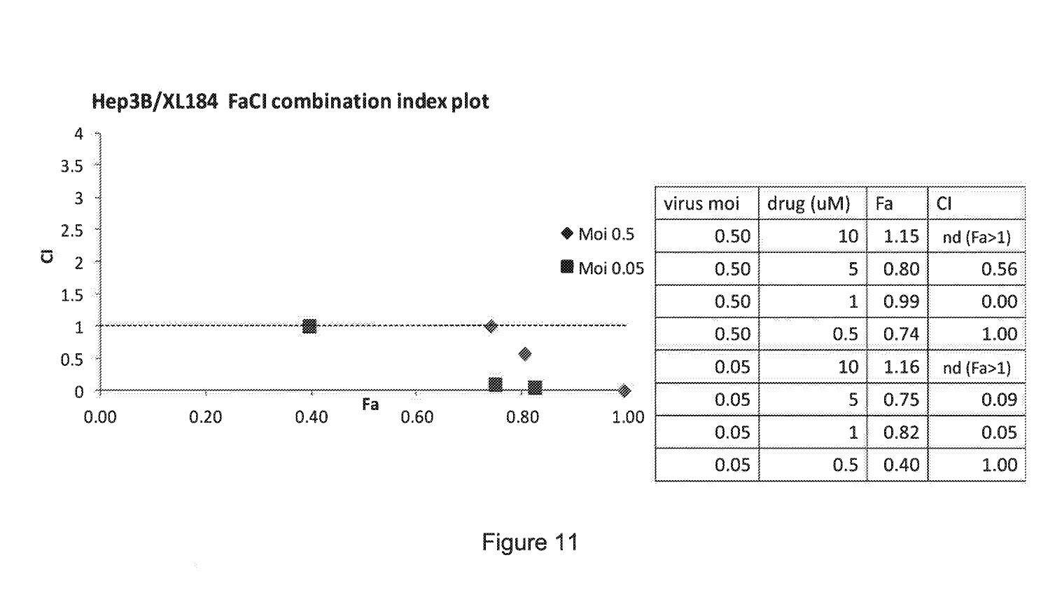

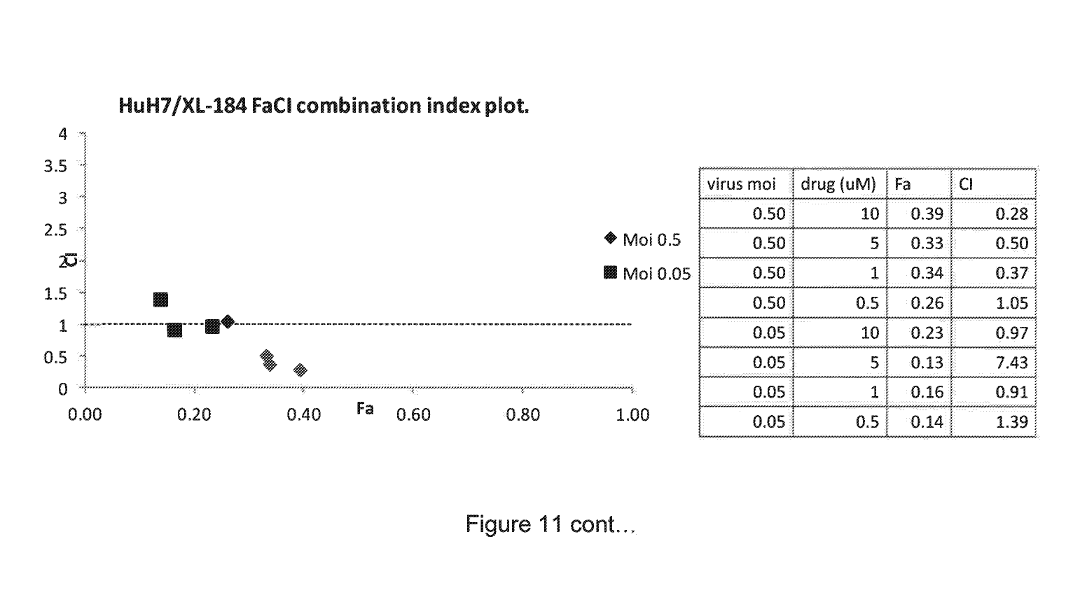

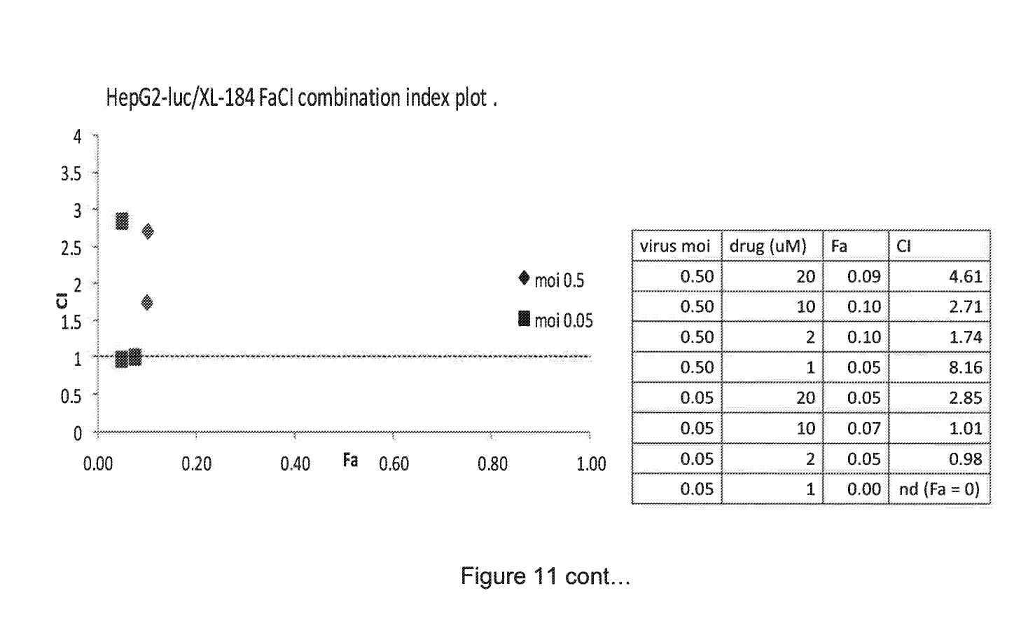

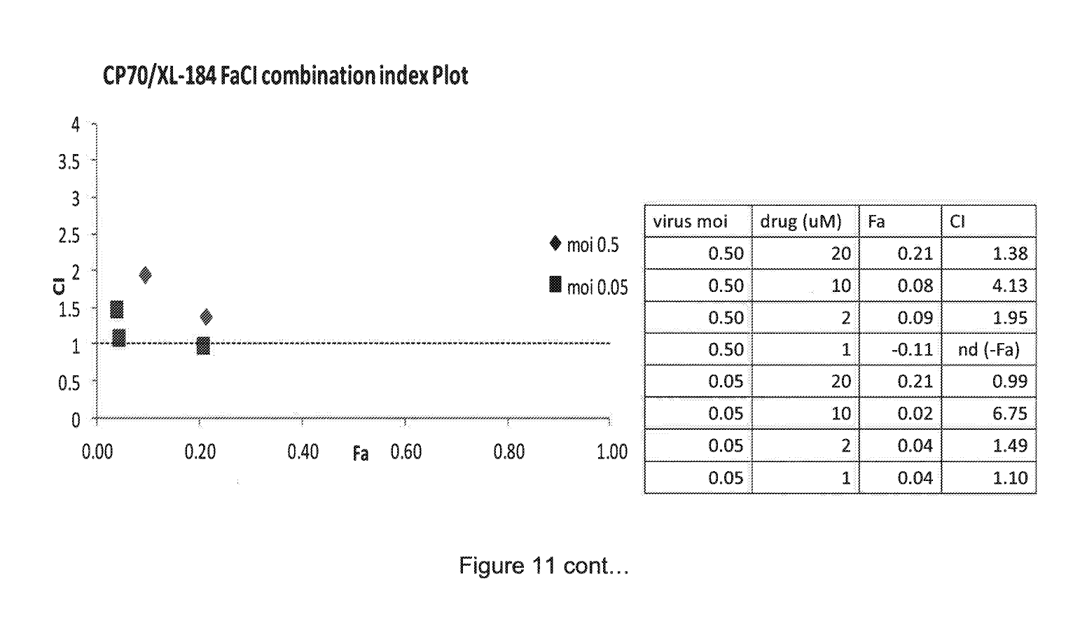

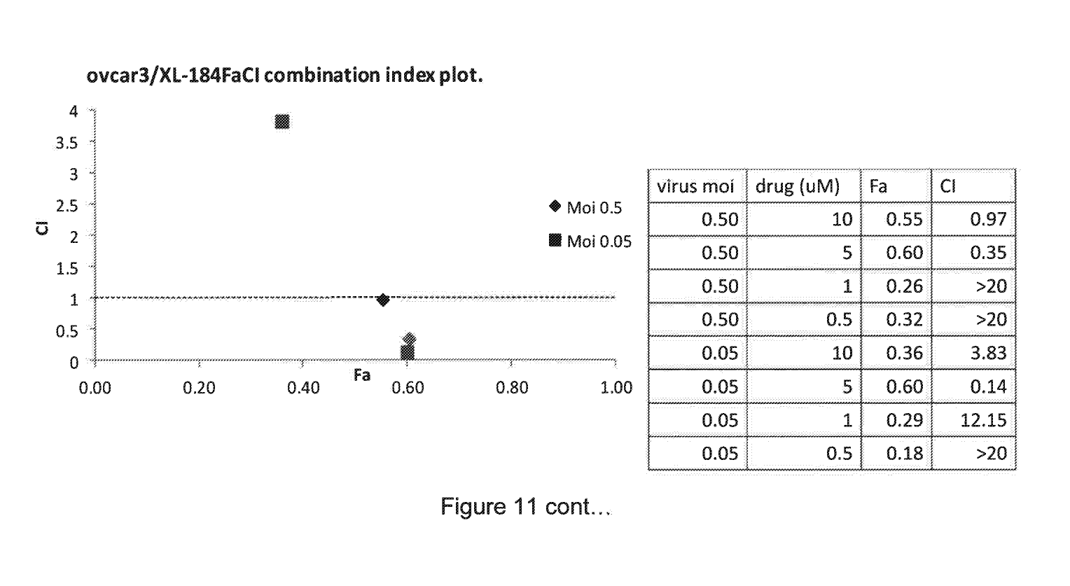

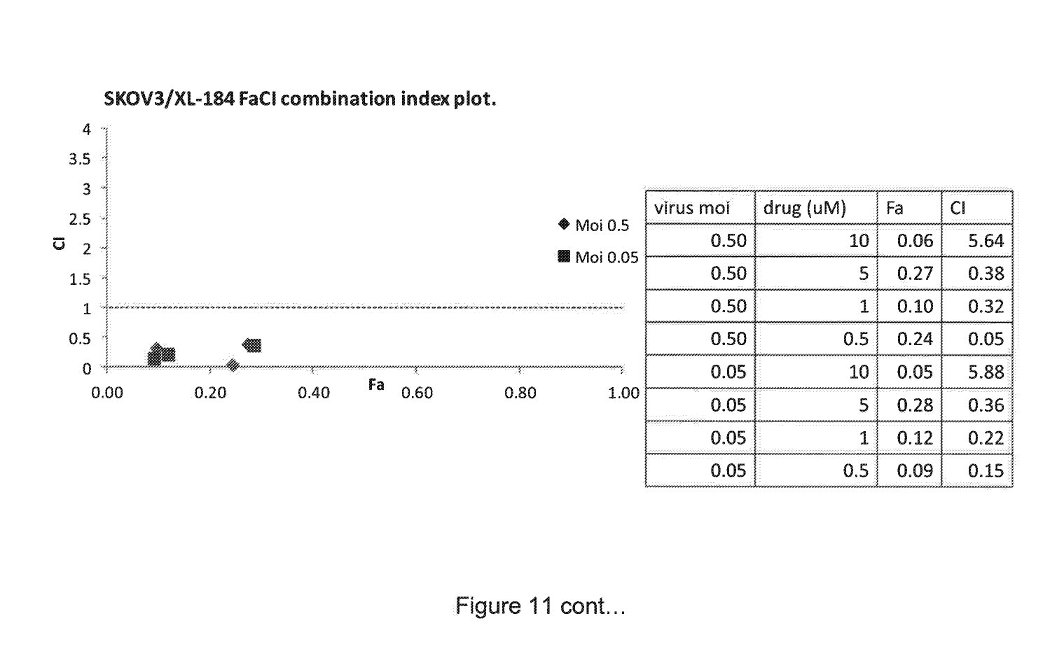

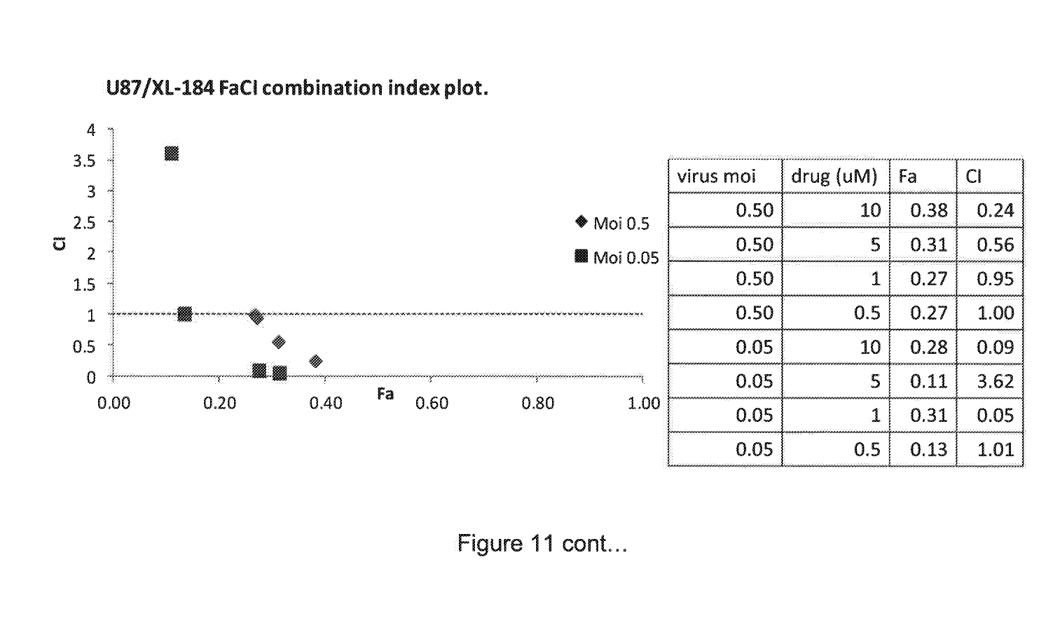

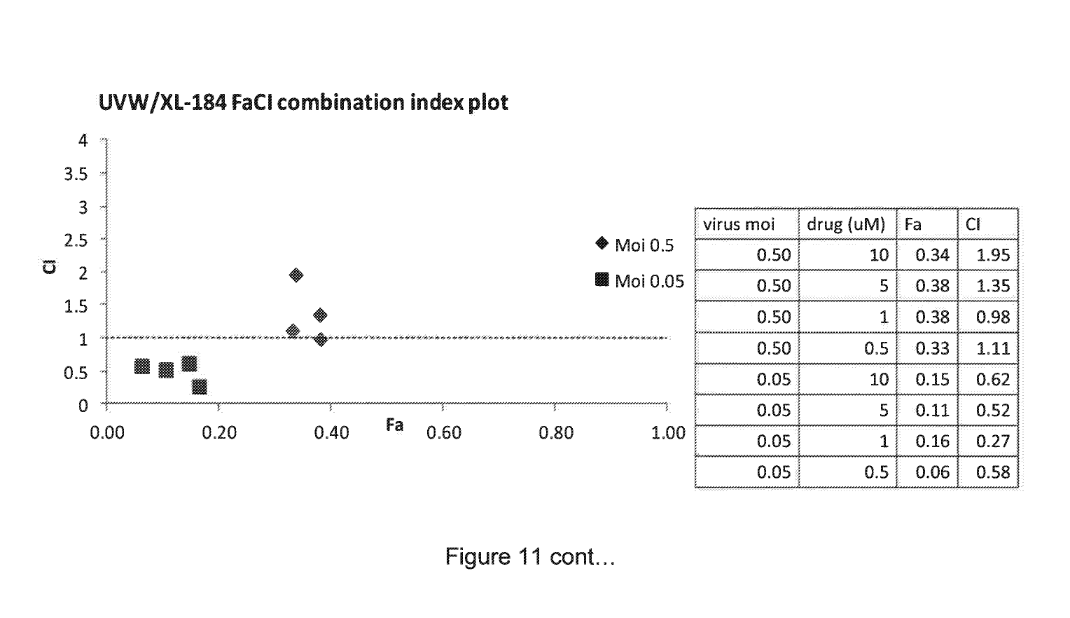

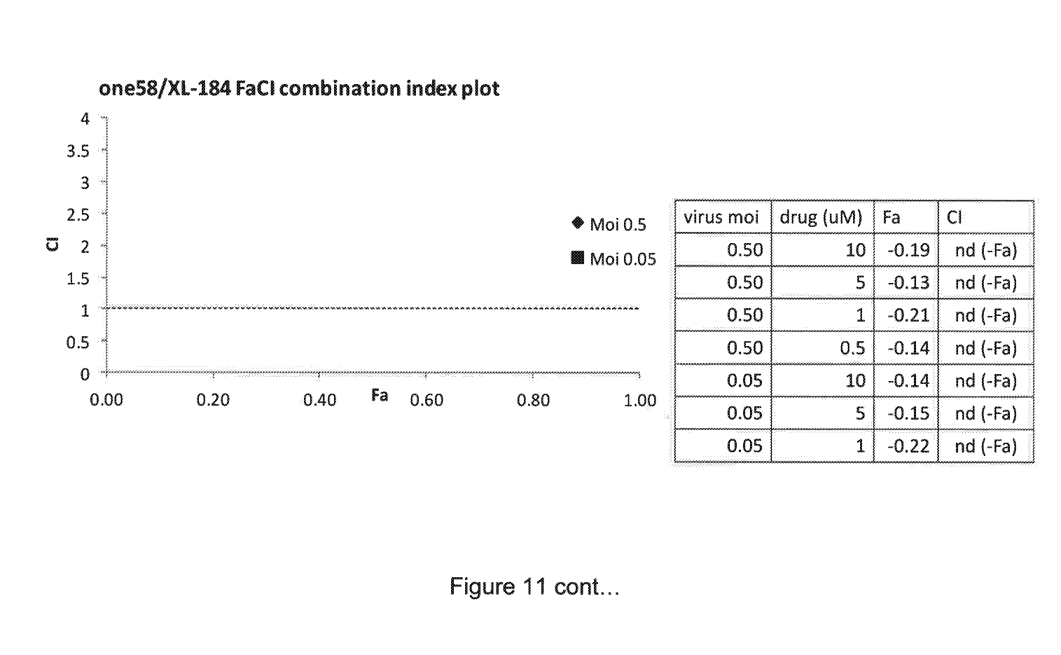

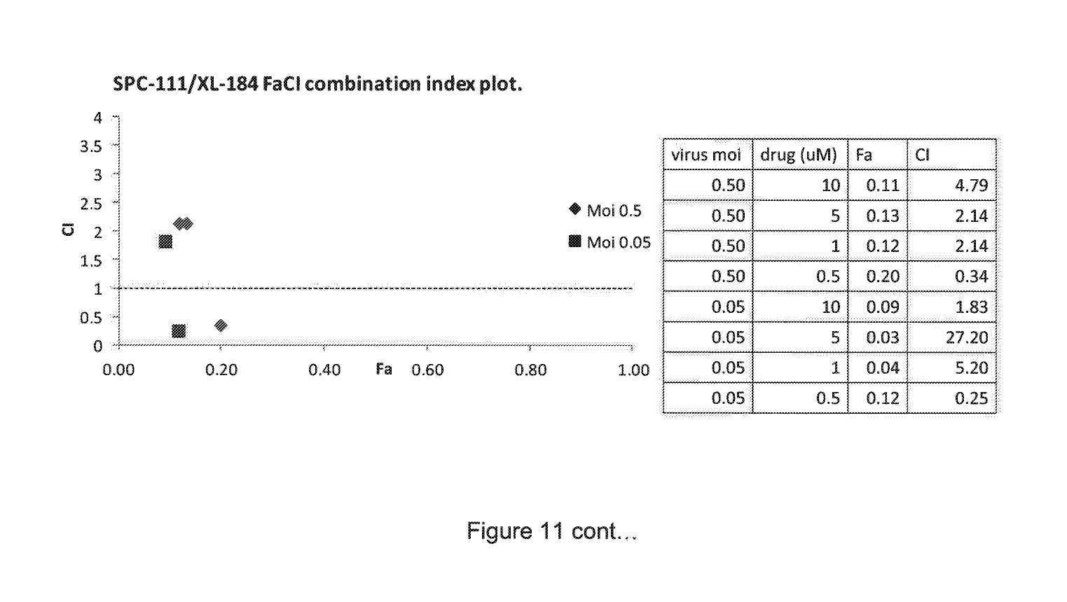

Cabozantinib was tested at 4 different doses (10 μM, 5 μM, 1 μM and 0.5 μM in Hep3B, HuH7, Ovcar3, SKOV3, U87, UM, One58 and SPC111 and 20, 10, 2 and 1 μM in HepG2-luc and CP70) in combination with HSV1716 at moi 0.5 and 0.05 and cell death after 72 hours exposure was assessed by DCP assay. Data were analysed for synergy/antagonism by calculating combination indices and graphing them against Fa (FaCl/Chou-Talalay plots) using a spreadsheet designed to automatically calculate Fa and Cl and graph the corresponding results from the raw DCP readings. The analysis was repeated on three separate plates and representative individual plots along with their respective Fa and Cl values are presented in FIG. 11 with synergies/antagonisms summarised in Table 6 (FIG. 12). The Cl values for the 4 drug concentrations at the two HSV1716 moi were determined and if less than 1 then the combination of virus and drug must act synergistically to enhance cell death. If more than 4 out of the 8 available combinations resulted in Cl values<1 then the drug was identified as combining synergistically with HSV1716 in that cell line. If more than 4/8 available combinations resulted in Cl values>1 then the drug was seen as antagonistic with HSV1716. A negative Fa value occurs when the test dcp value is less than the control (i.e. indicative of improved survival) and is therefore scored as antagonistic.

### Conclusions

Cabozantinib is toxic in most of the cell lines tested.
There are clear synergy signals in 5/10 cell lines tested (limited synergy in HepG2-luc, CP70 and One58, Ovcar3 and SPC111 cells).
Cabozantinib was strongly synergistic with HSV1716 in 2/2 HGG cell lines.
Synergies occurred at both moi.
Cabozantinib had very little direct HSV1716 toxicity.

### Example 4

### Pazopanib

Pazopanib was analysed in combination with HSV1716 in vitro using the cell lines described. Toxicity was assessed in each well using DCP cell death assays. Toxic effects of the virus on the drug were also assessed by using HSV1716gfp.

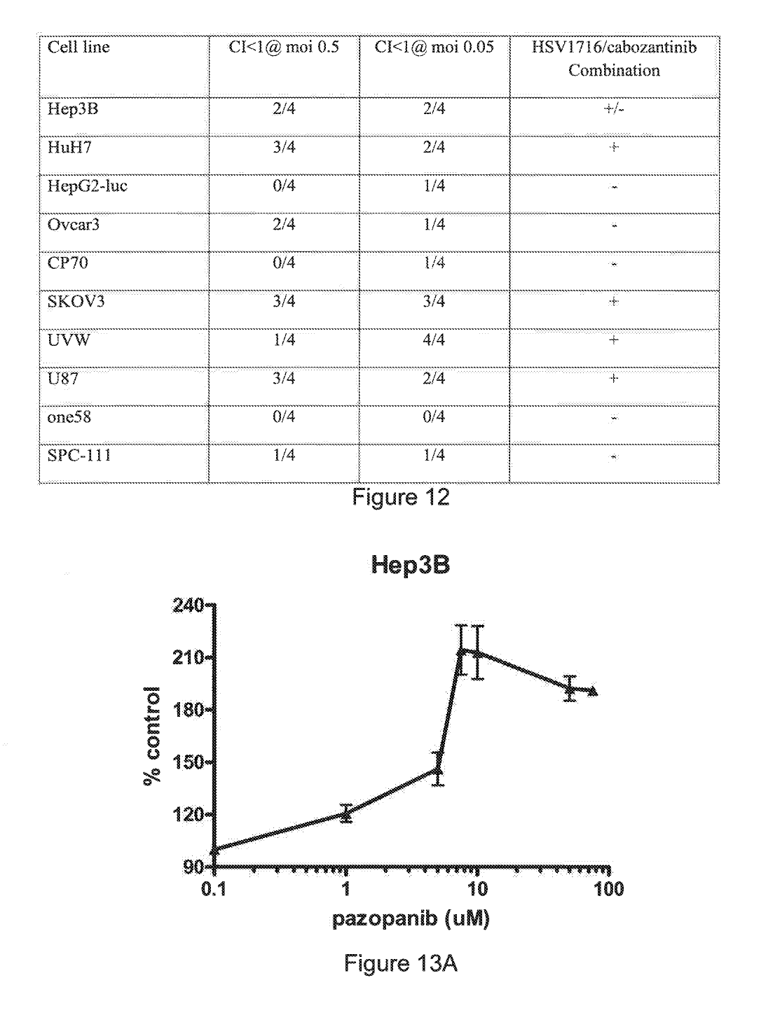

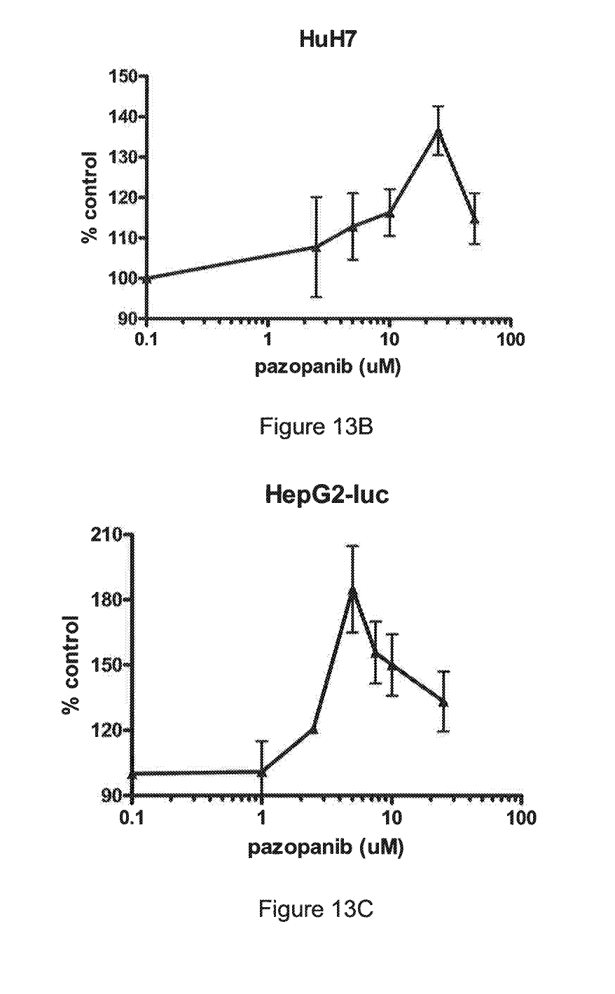

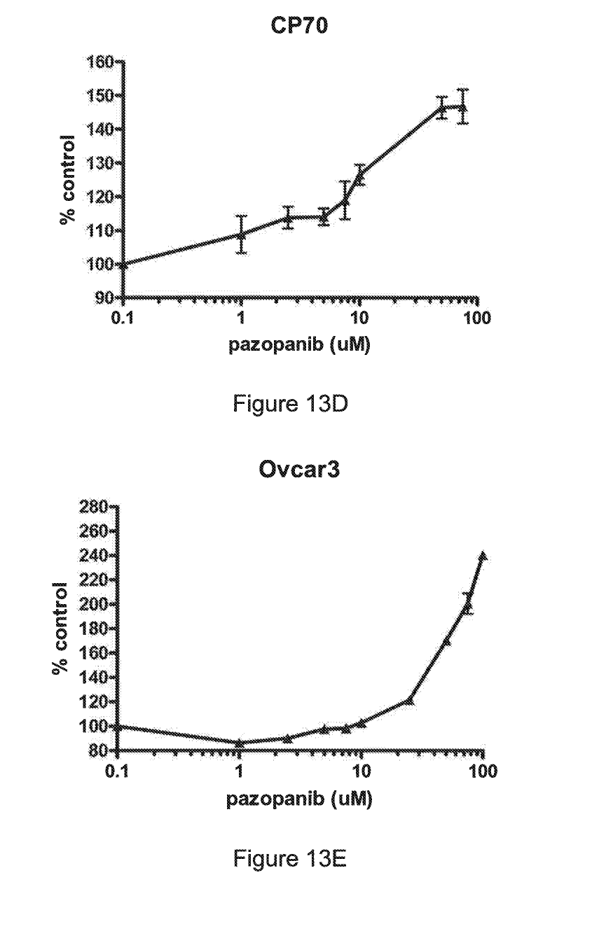

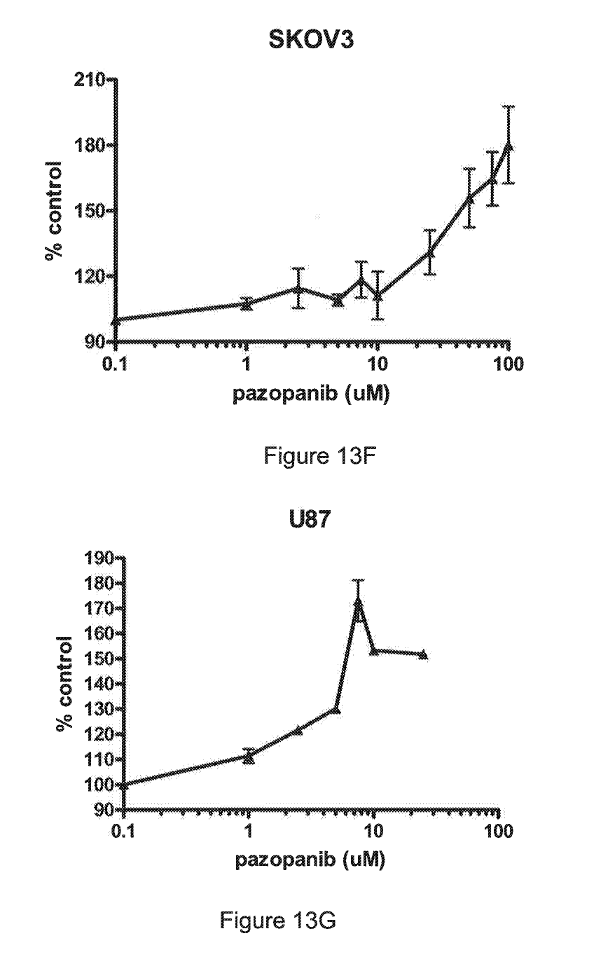

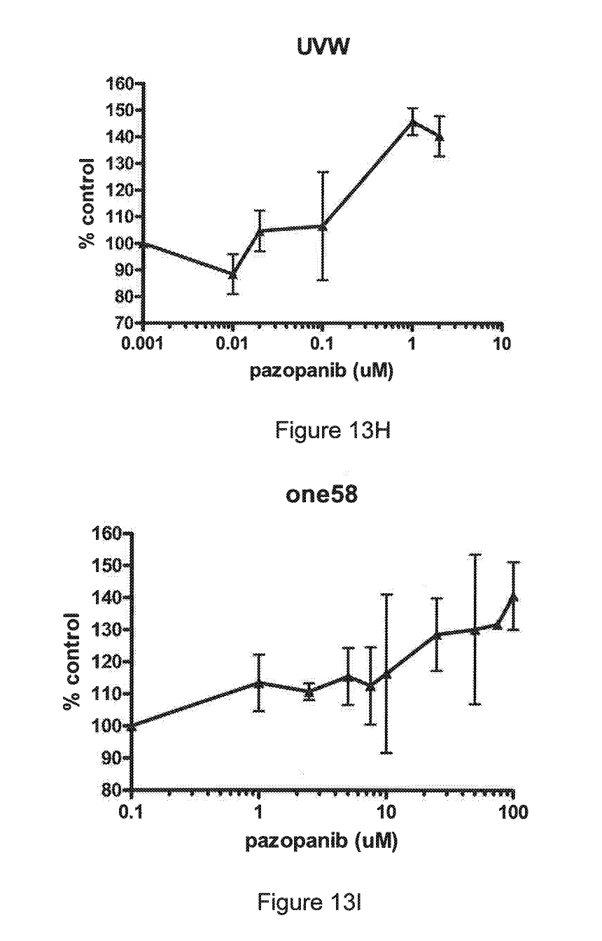

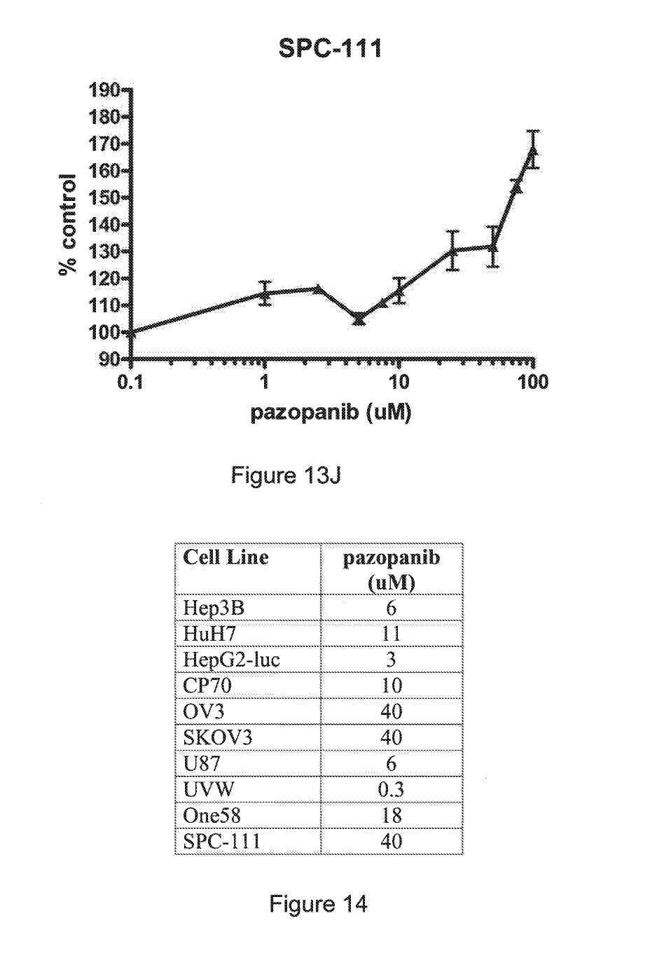

IC50 curves for each cell line tested are shown in FIG. 13 and the estimated IC50s are shown in Table 7 (FIG. 14). Pazopanib was toxic in all cell lines with IC50s for Hep3B, HuH7, HepG2-luc, CP70, U87 and One58 in the narrow range 3-18 μM. UVW cells were the most sensitive to this drug with an IC50 of 0.3 μM and Ovcar3, SKOV3 and SPC-111 were the most resistant with an IC50 of 40 μM in each cell line (Table 7; FIG. 14).

### HSV1716 Toxicity

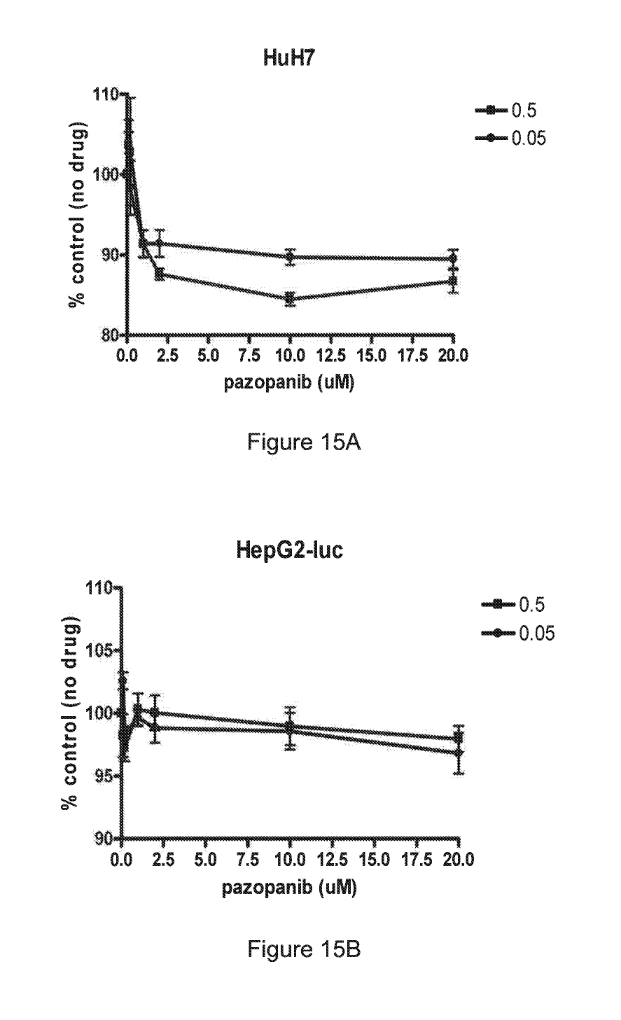

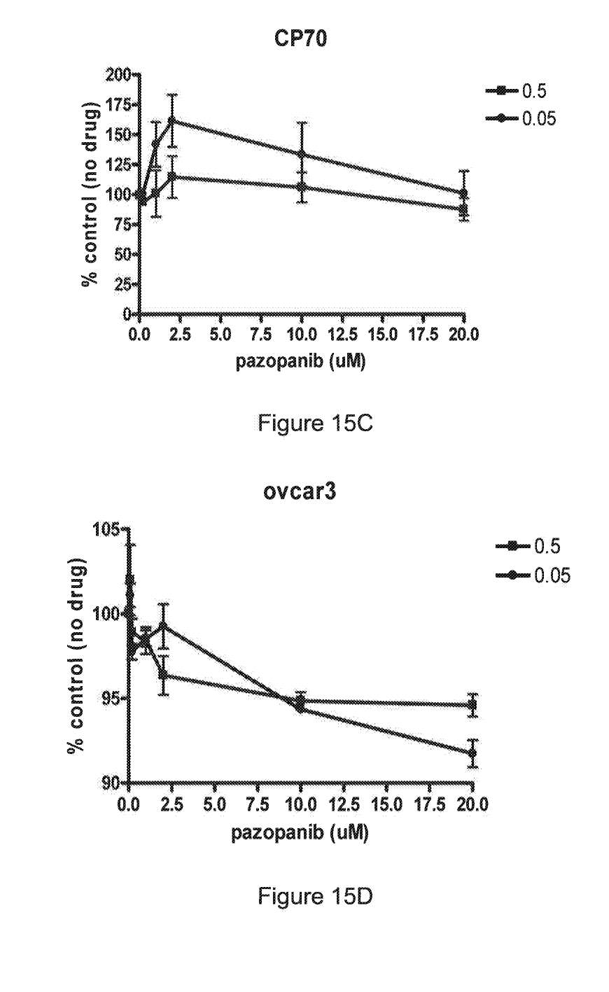

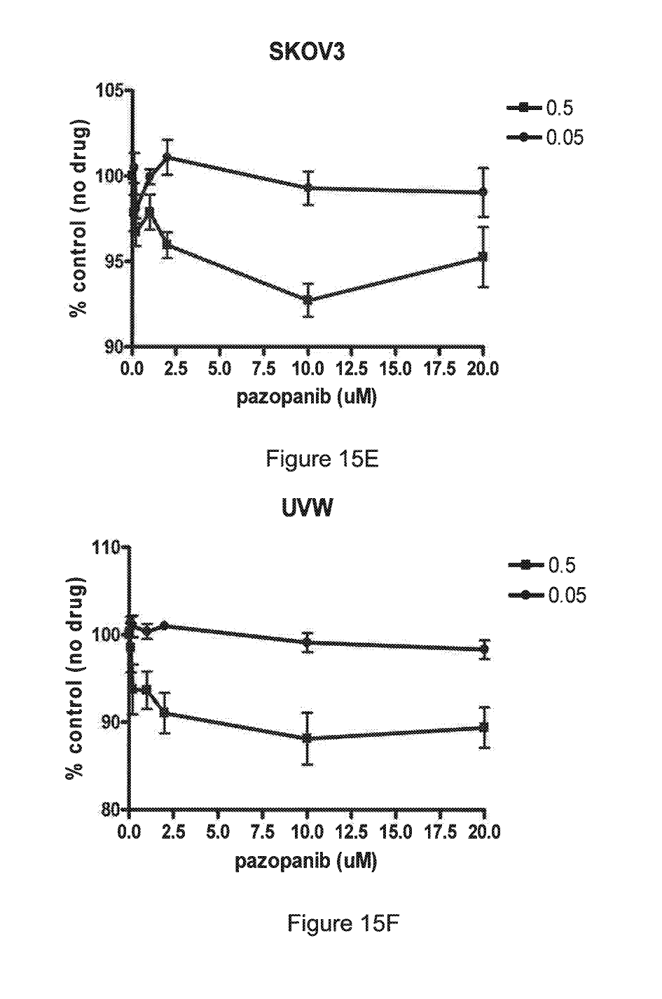

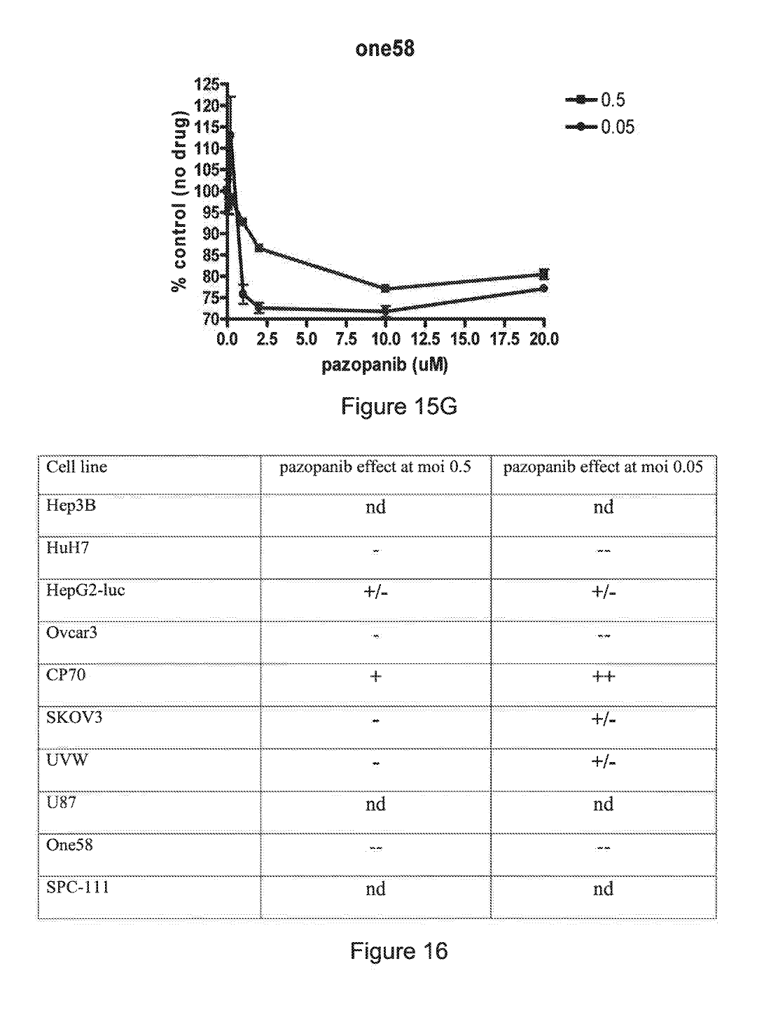

Pazopanib had some toxicity with respect to HSV1716 as assessed by GFP expression during infection of the cell lines with HSV1716gfp. Hep3B, U87 and SPC111 cells were not available for this analysis. Pazopanib was incubated with cells plus virus at 20 μM, 10 μM, 2 μM, 1 μM, 0.2 μM and 0.1 μM for 72 hrs and GFP fluorescence measured. Drug-mediated virus toxicities are presented in the graphs in FIG. 15 as percentage of control cells (virus alone and no drug) in HuH7, HepG2-luc, CP70, Ovcar3, SKOV3, UVW and One58. Virus was used at moi 0.5 or moi 0.05. In CP70 cells low dose Pazopanib stimulated GFP expression during HSV1716gfp infection but higher doses had no effect. In HepG2-luc cells, Pazopanib had little effect on GFP expression during HSV1716gfp infection at both moi (<+/−5% variation from control, Table 8; FIG. 16). At moi 0.5, Pazopanib inhibited GFP expression during HSV1716gfp infection in HuH7, Ovcar3, SKOV3, UVW and One58 (Table 8; FIG. 16) and there was a more potent inhibitory effect at the lower moi in HuH7, Ovcar3 and particularly One58 cells (Table 8; FIG. 16). There were no inhibitory effects of Pazopanib at the lower moi in SKOV3 and UVW cells. Therefore Pazopanib had some marked toxic effects on HSV1716 in most cell lines tested and the results are summarised in Table 8 (FIG. 16).

### Combination Analysis

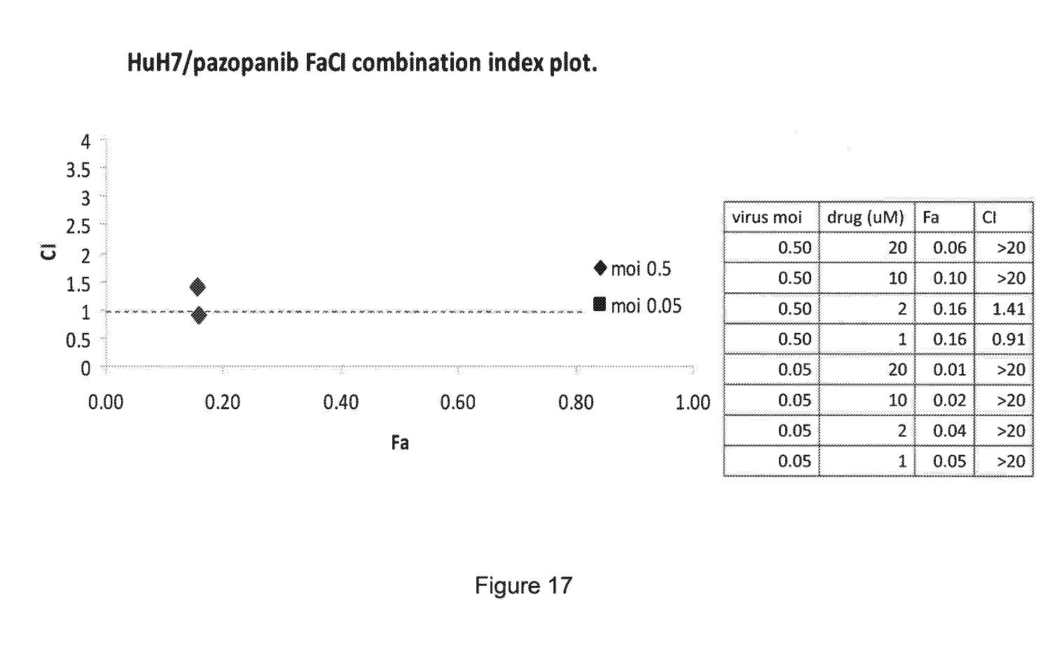

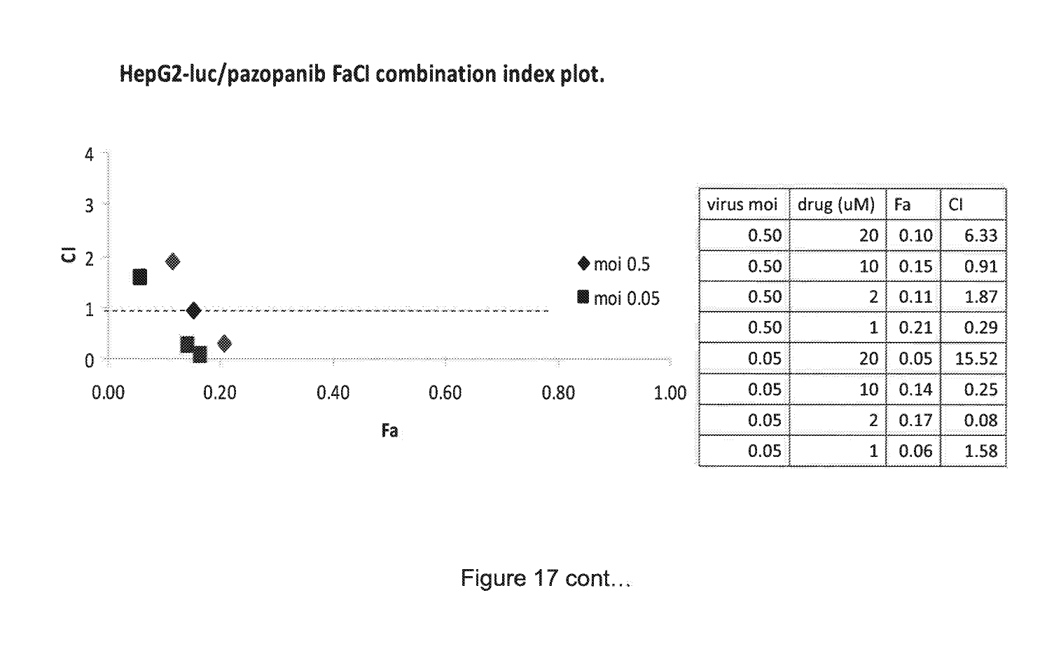

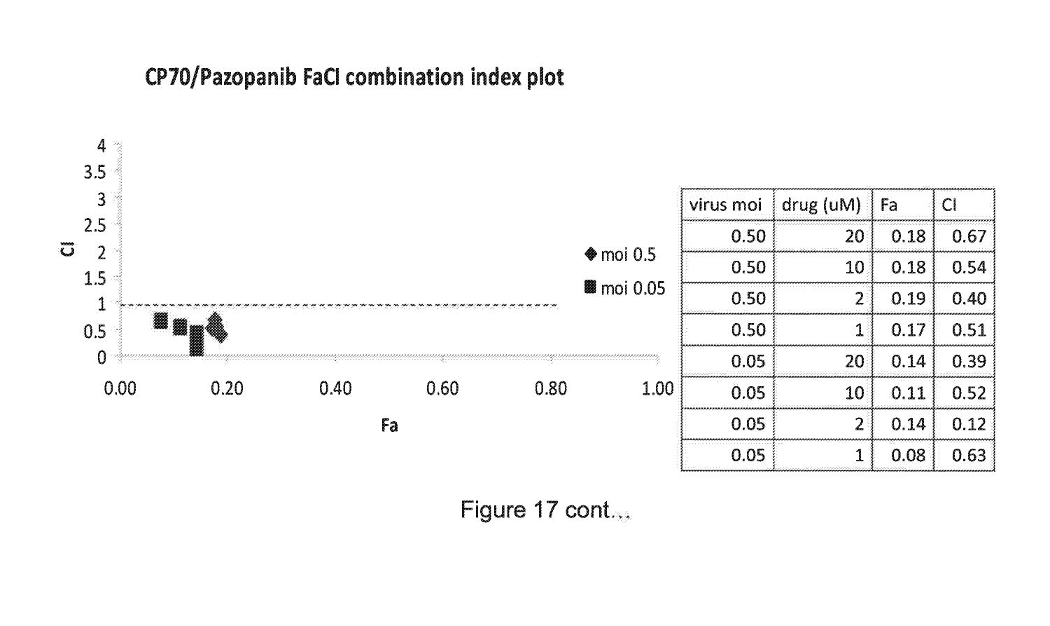

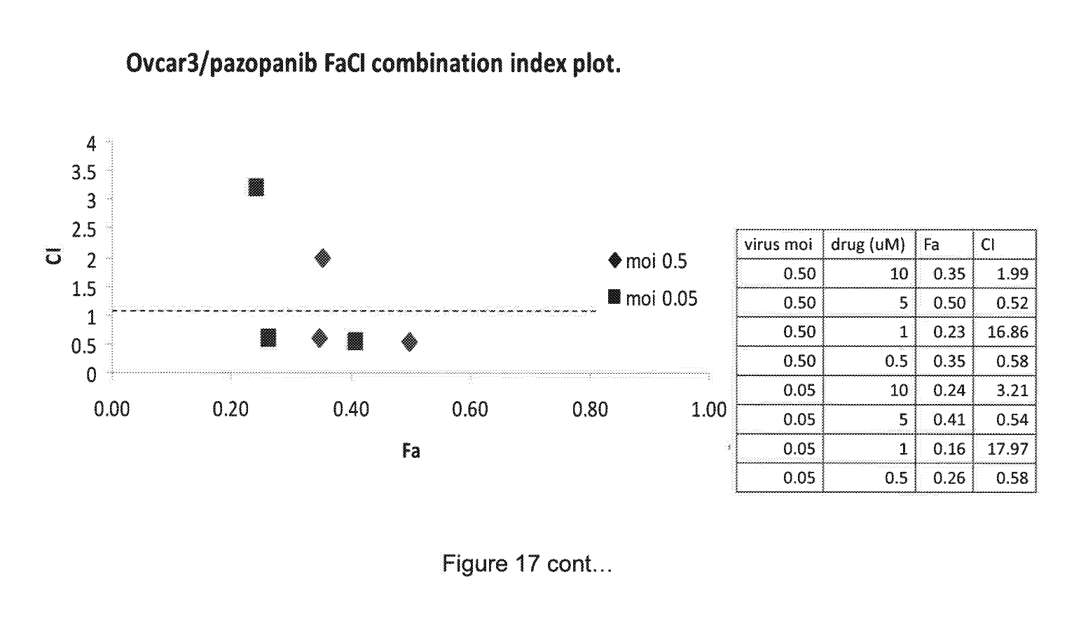

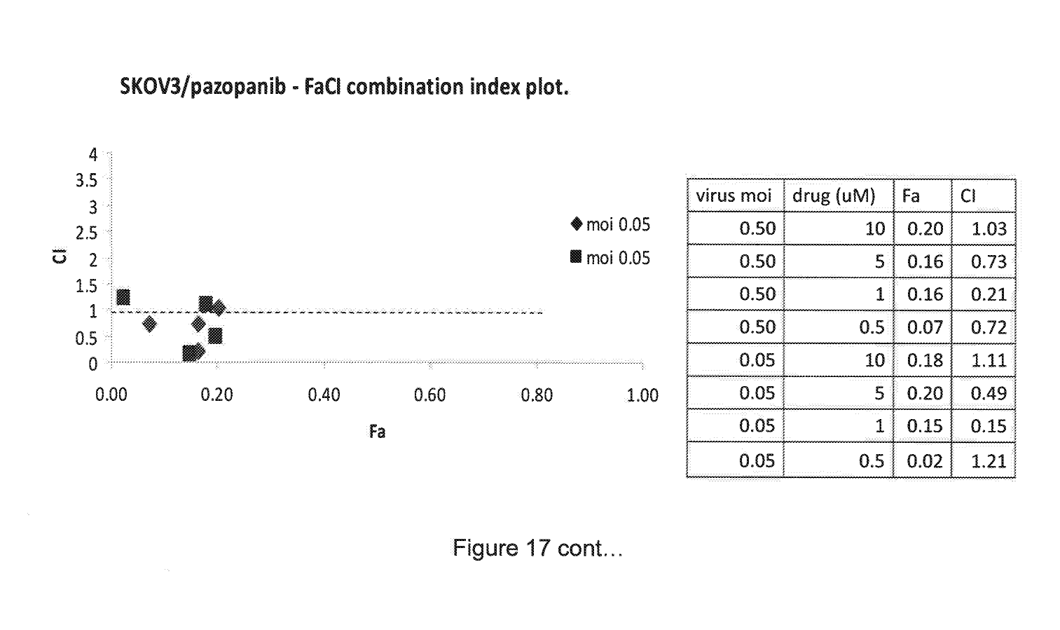

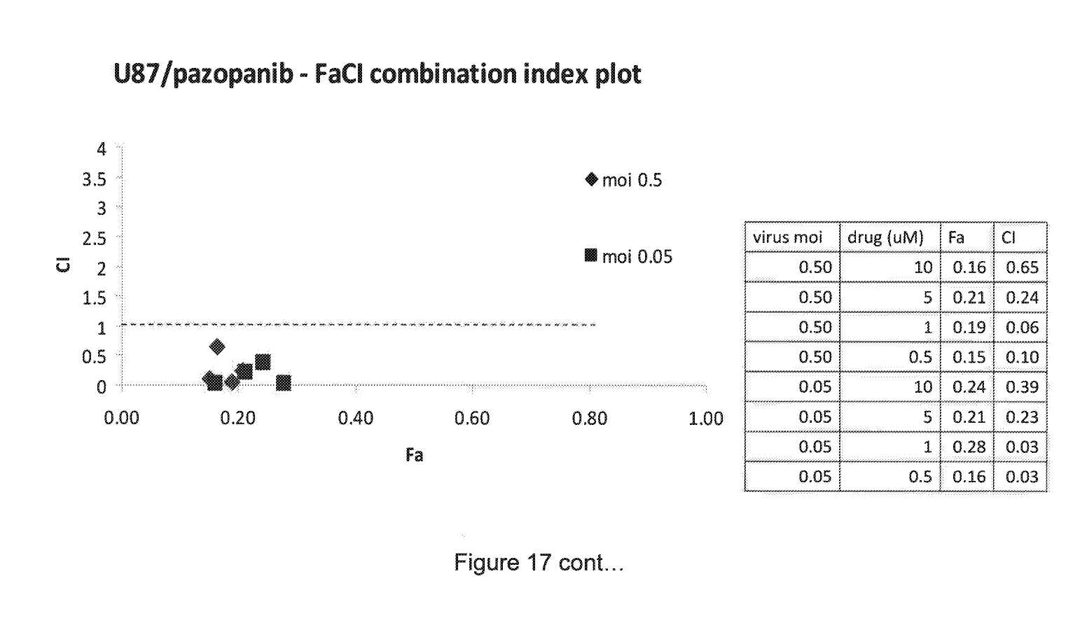

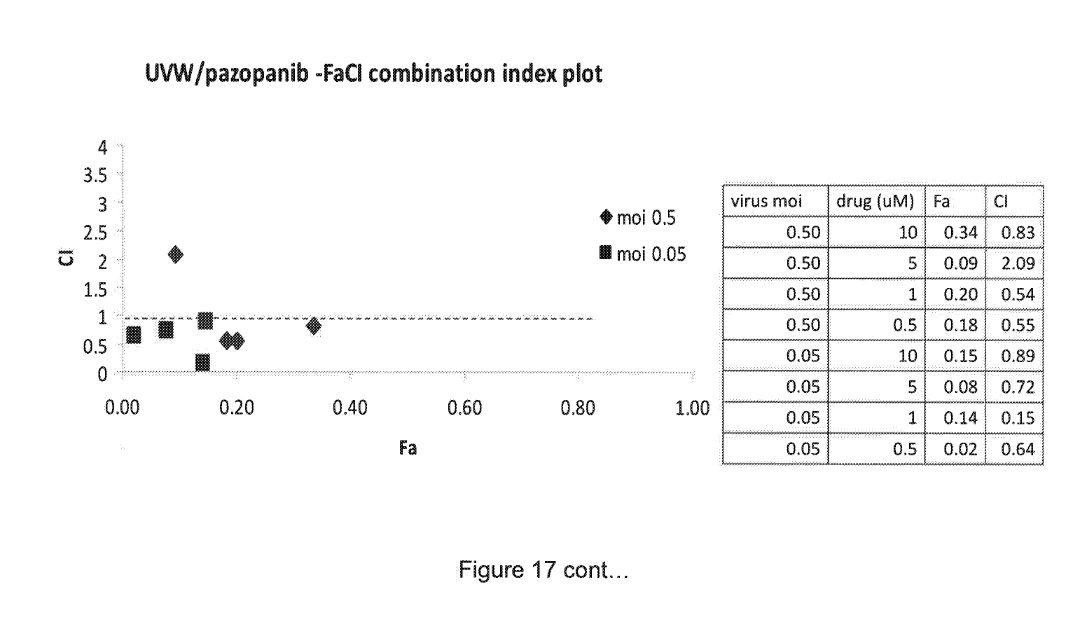

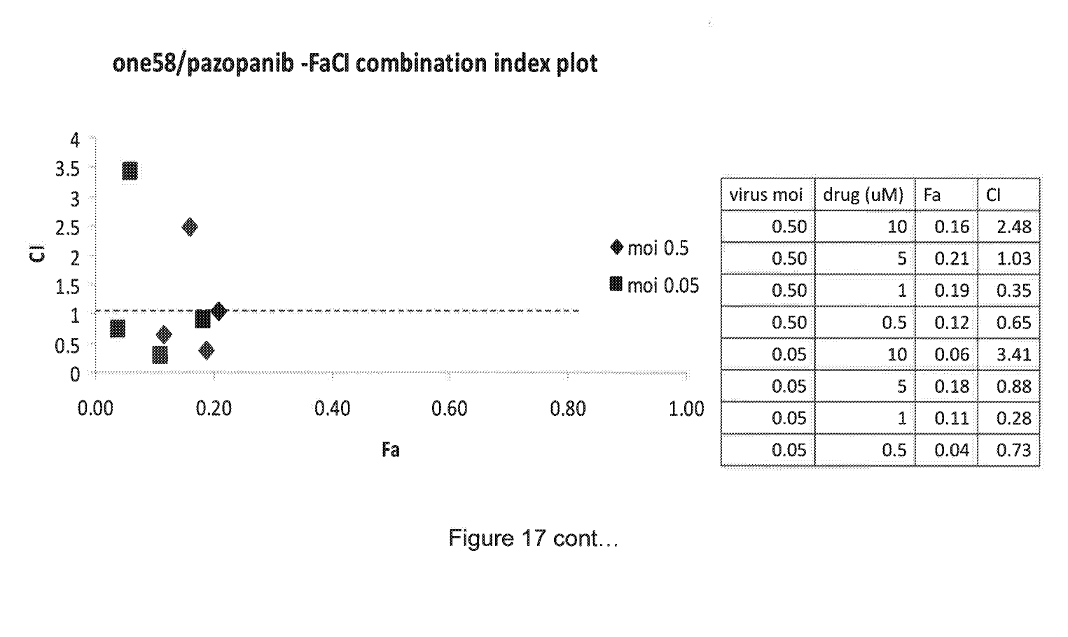

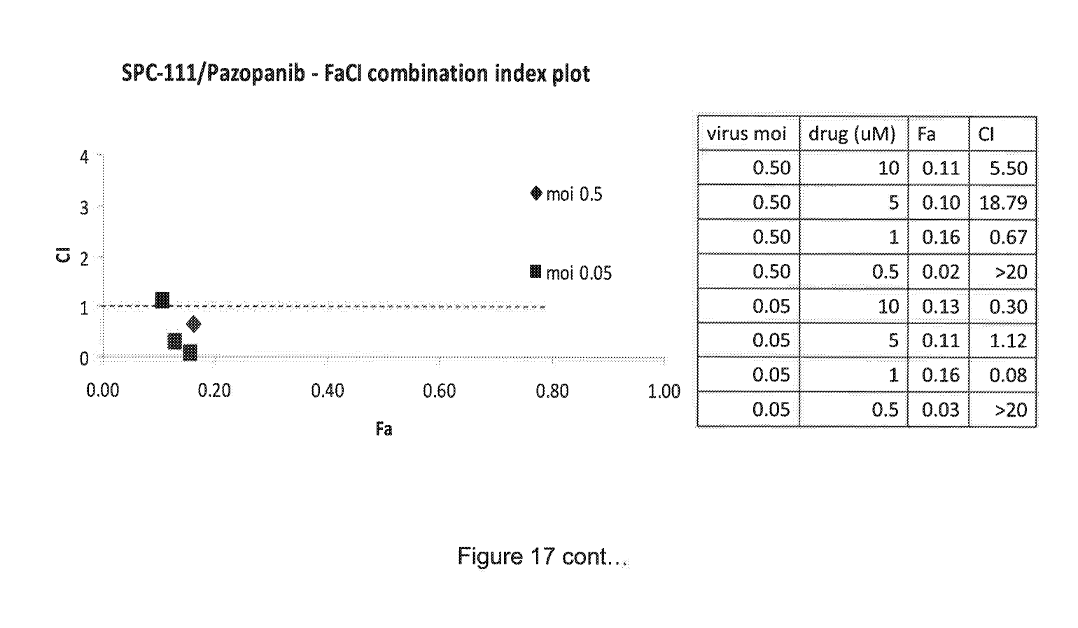

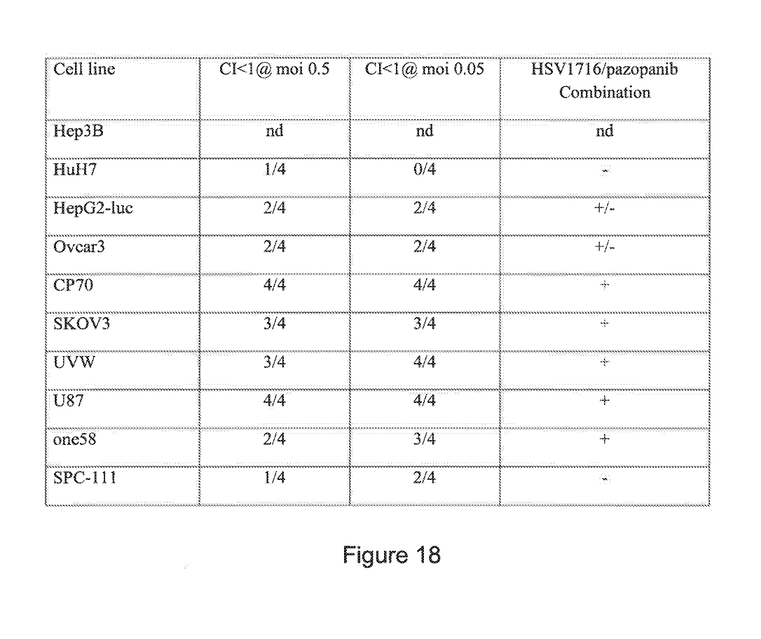

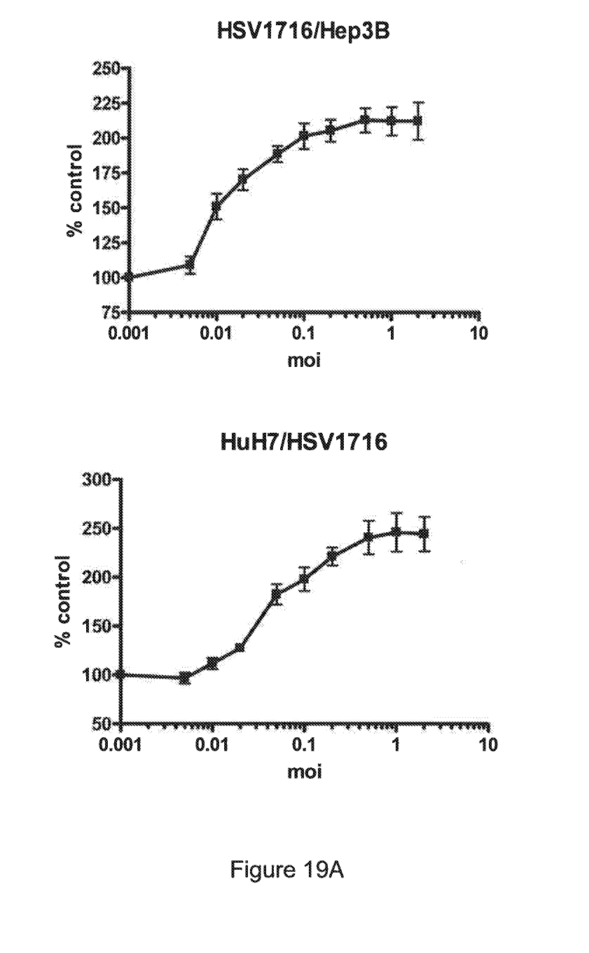

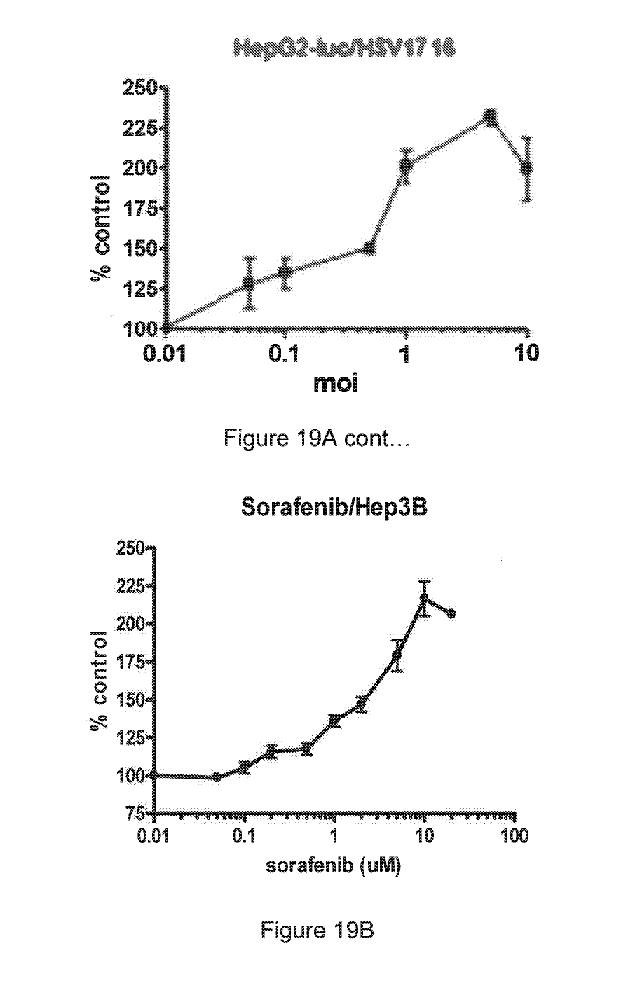

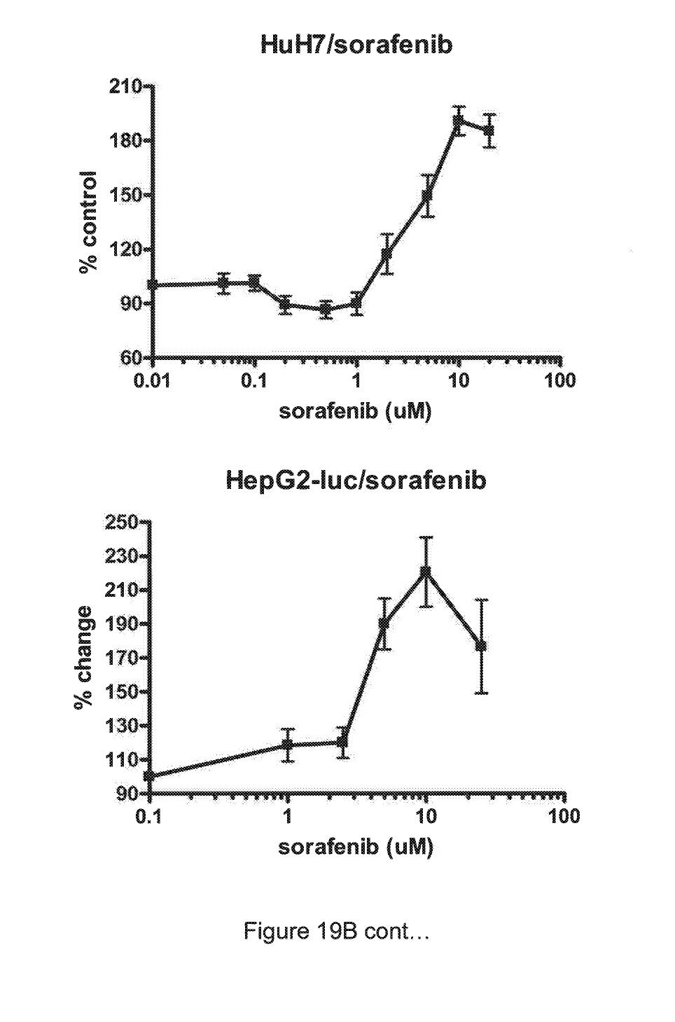

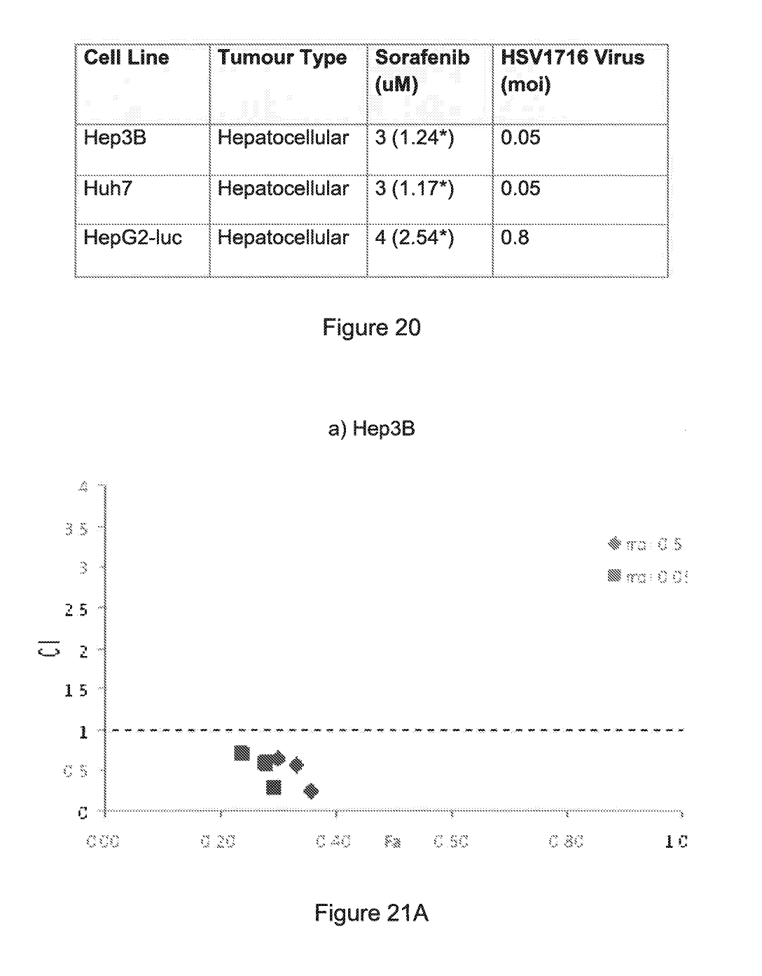

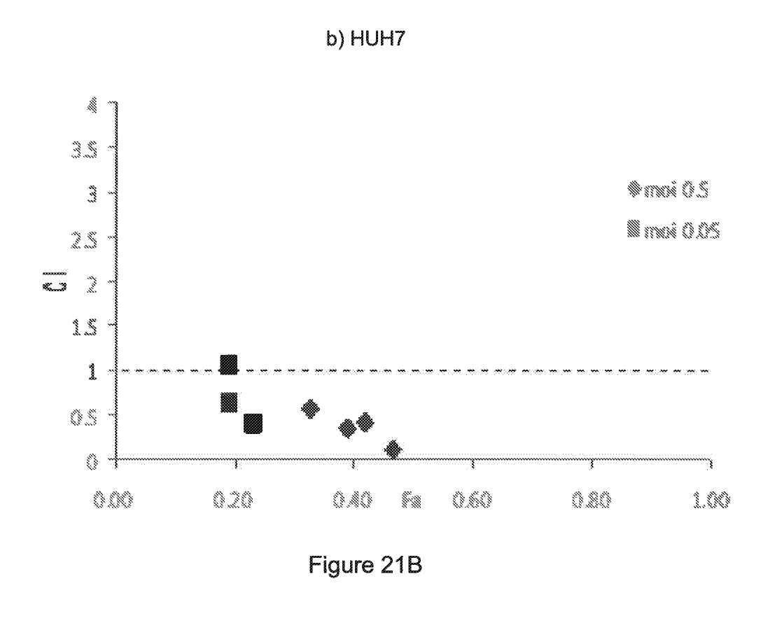

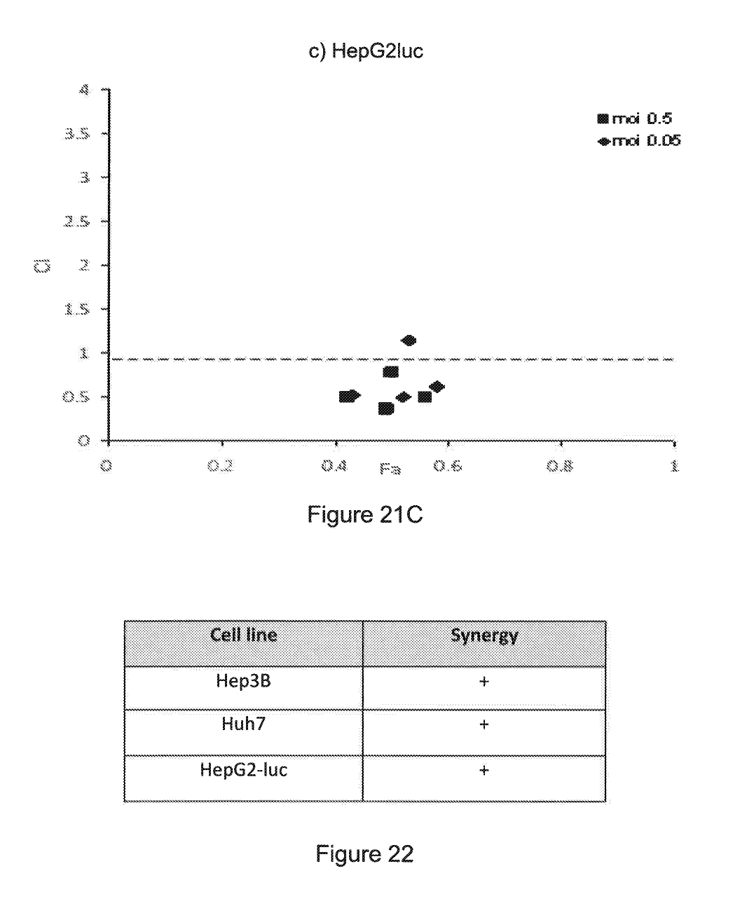

Pazopanib was tested at 4 different doses (20, 10, 2 and 1 μM in HuH7, HepG2-luc and CP70 cells and 10, 5, 1 and 0.5 μM in Ovcar3, SKOV3, U87, UVW, One58 and SPC111) in combination with HSV1716 at moi 0.5 and 0.05 and cell death after 72 hours exposure was assessed by DCP assay. Hep3B cells were not available for this analysis. Data were analysed for synergy/antagonism by calculating combination indices and graphing them against Fa (FaCl/Chou-Talalay plots) using a spreadsheet designed to automatically calculate Fa and Cl and graph the corresponding results from the raw DCP readings. The analysis was repeated on three separate plates and representative individual plots along with their respective Fa and Cl tables are presented in FIG. 17 with synergies/antagonisms summarised in Table 9 (FIG. 18). The Cl values for the 4 drug concentrations at the two HSV1716 moi were determined and if less than 1 then the combination of virus and drug must act synergistically to enhance cell death. If more than 4 out of the 8 available combinations resulted in Cl values<1 then the drug was identified as combining synergistically with HSV1716 in that cell line. If more than 4/8 available combinations resulted in Cl values>1 then the drug was seen as antagonistic with HSV1716. A negative Fa value occurs when the test DCP value is less than the control (i.e. indicative of improved survival) and is therefore scored as antagonistic.

### Conclusions

Pazopanib is toxic in most of the cell lines tested.
There are clear synergy signals in 7/9 cell lines tested (limited synergy in HuH7 and SPC111 cells).
Pazopanib was strongly synergistic with HSV1716 in 3/3 ovarian and 2/2 HGG cell lines.
Synergies occurred at both moi,
Pazopanib inhibited HSV1716 in most of the cell lines tested although the drug had no effect in HepG2-luc and was stimulatory in CP70. The effects of Pazopanib on HSV1716 replication did not correlate with virus toxicity as, for example, the drug was synergistic in both CP70 and One58 cells despite stimulating replication in the former and inhibiting it in the latter.

### Example 5

### Assays and Analysis

The following approach was used for the experiments reported in Examples 6 to 11.

### HSV1716 Toxicity

Toxicity of drugs with respect to HSV1716 was assessed by incubating cells plus HSV1716gfp virus with various concentrations of drugs, and measuring GFP fluorescence after 72 hours.

### Combination Analysis

Drugs were tested in combination with HSV1716, and cell death after 72 hours exposure was measured by DCP assay. Data were analysed for synergy/antagonism by calculating combination indices and graphing them against Fa (FaCl/Chou-Talalay plots) using a spreadsheet designed to automatically calculate Fa and Cl and graph the corresponding results from the raw DCP readings. Analysis was repeated on three separate plates.

Cl values were determined for the various drug/virus combinations. Cl values<1 indicate that the combination of virus and drug acts synergistically to enhance cell death. If 4 or more out of the 8 available combinations resulted in Cl values<1, then the drug was identified as combining synergistically with HSV1716 in that cell line. If 1-3 out of the 8 combinations resulted in Cl values<1, this was suggestive of some synergy with HSV1716. If 0 of the 8 available combinations resulted in Cl values<1 the drug was considered antagonistic with HSV1716. A negative Fa value occurred when the test DCP value was less than the control (i.e. indicative of improved survival) and was therefore scored as antagonistic.

### Example 6

### Crizotinib

Crizotinib was analysed in combination with HSV1716 in vitro as described, using the cell lines described. Toxicity was assessed in each well using DCP cell death assays. Toxic effects of Crizotinib on the virus were also assessed using HSV1716gfp, as described.

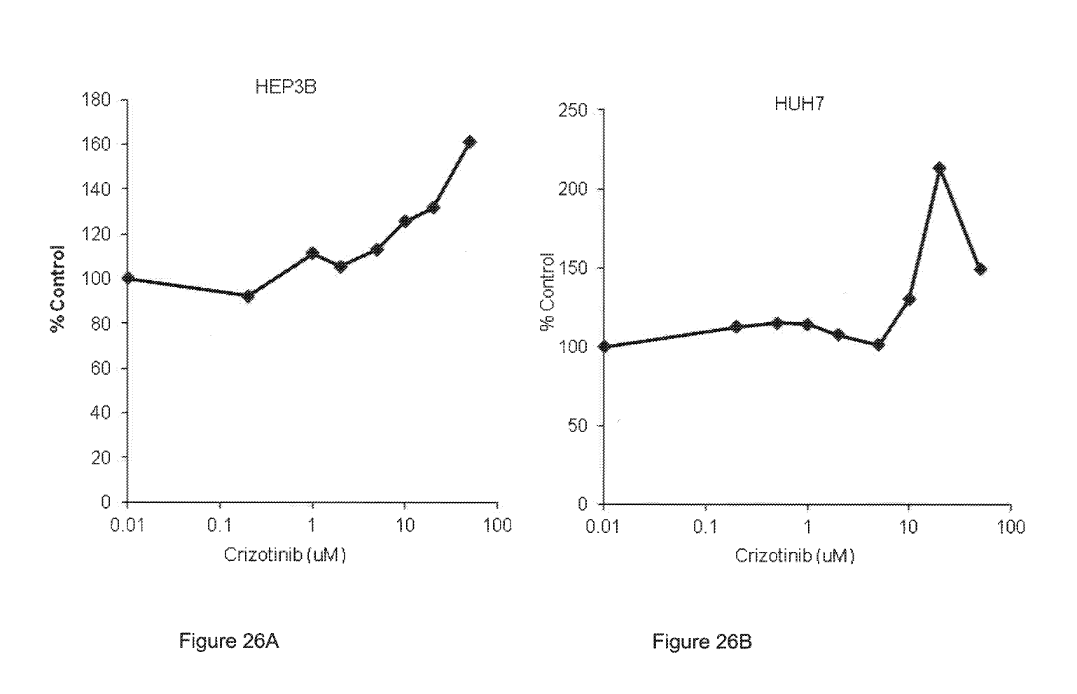

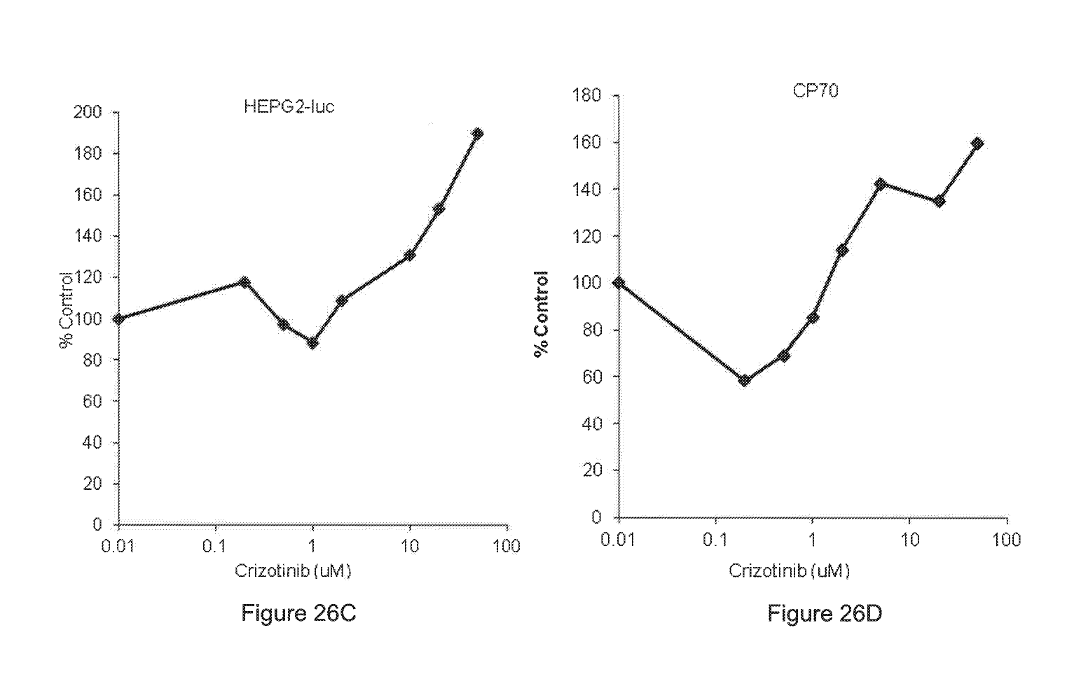

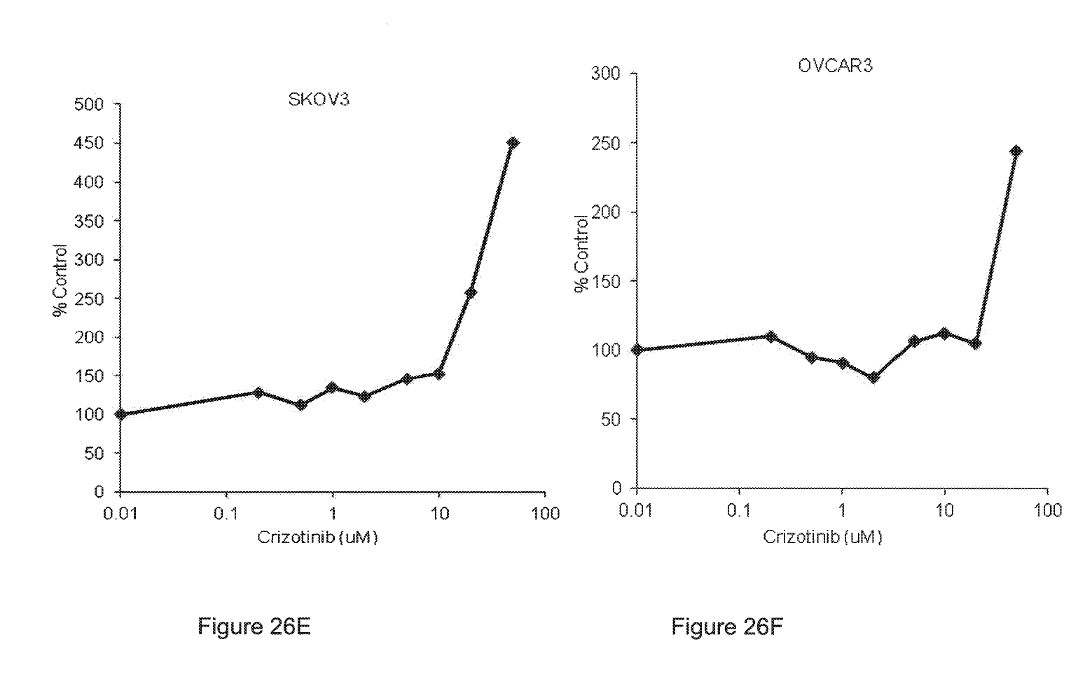

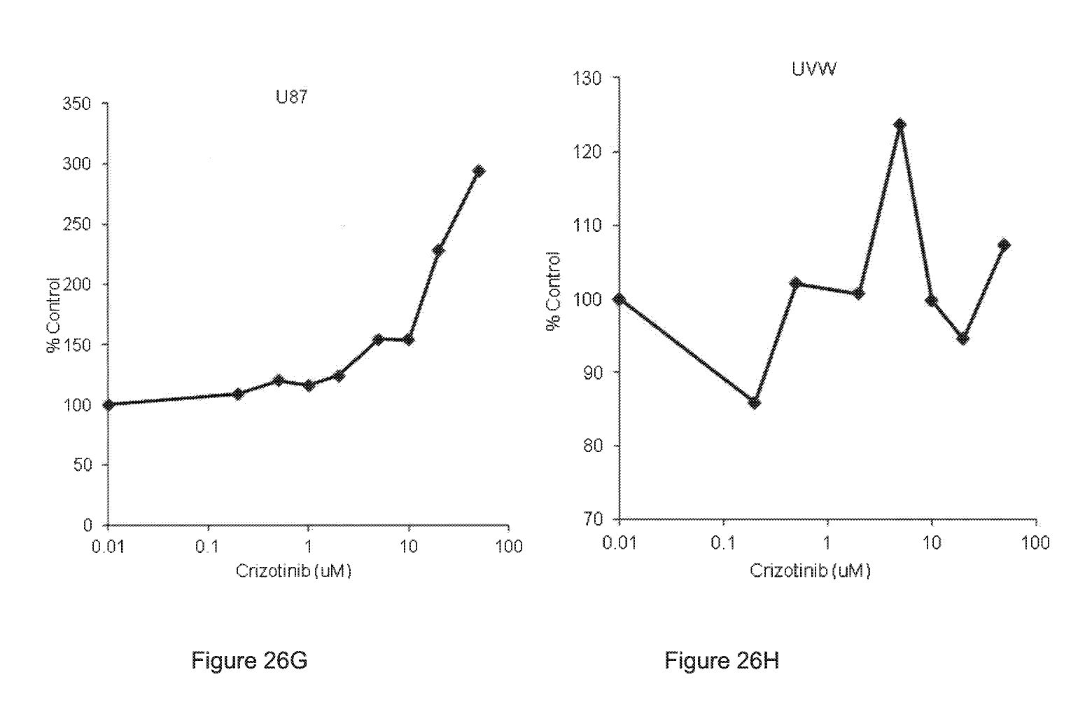

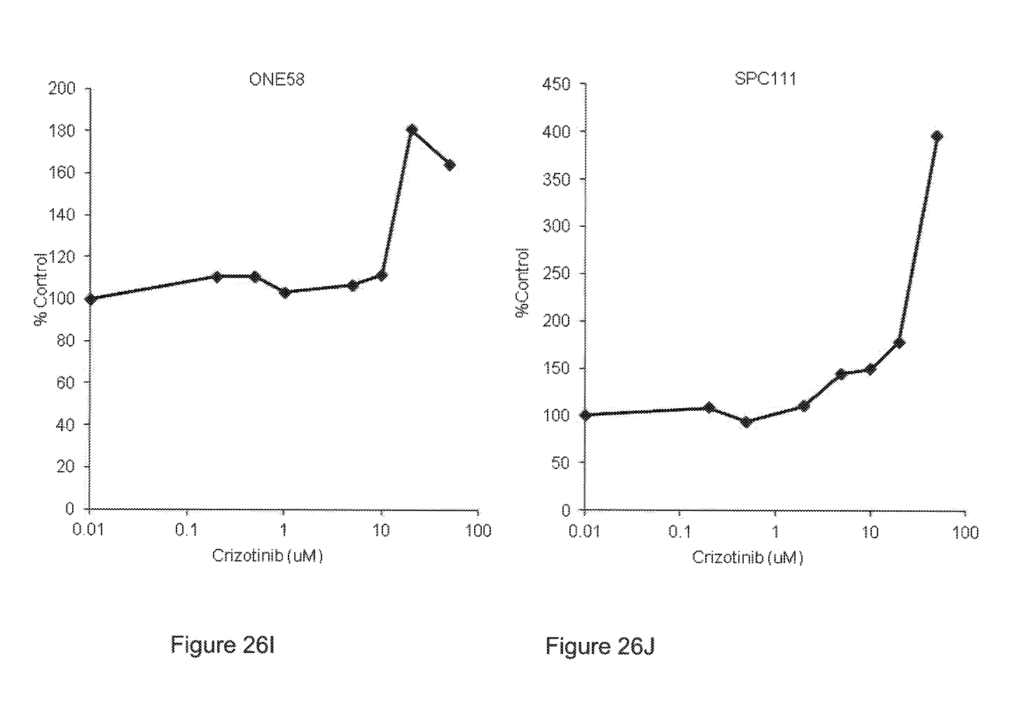

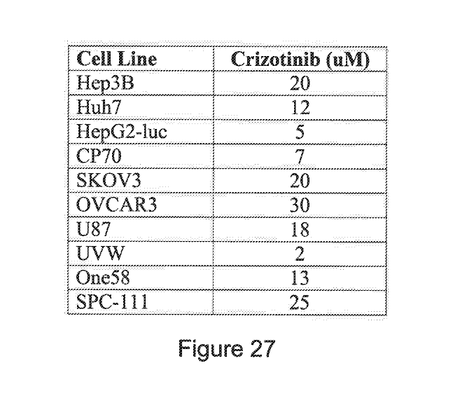

IC50 curves for each cell line tested are shown in FIG. 26 and the estimated IC50s are shown in Table 12 (FIG. 27). Crizotinib was toxic in most cell lines with IC50s in the range 2-30 μM with limited cell line variability in toxicity (Table 12; FIG. 27).

### HSV1716 Toxicity

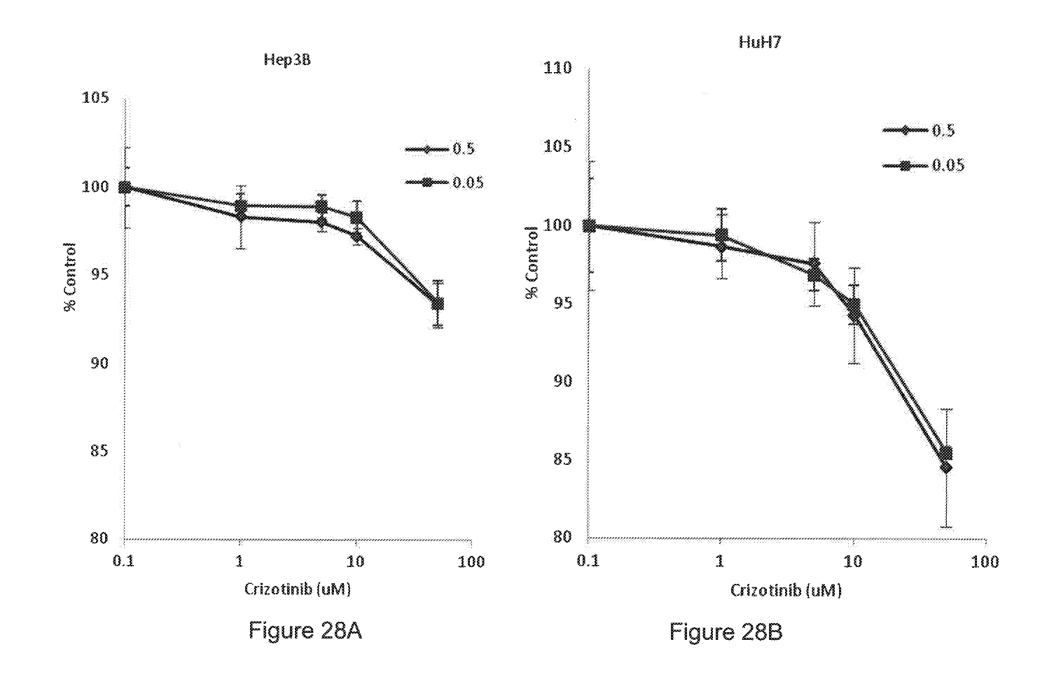

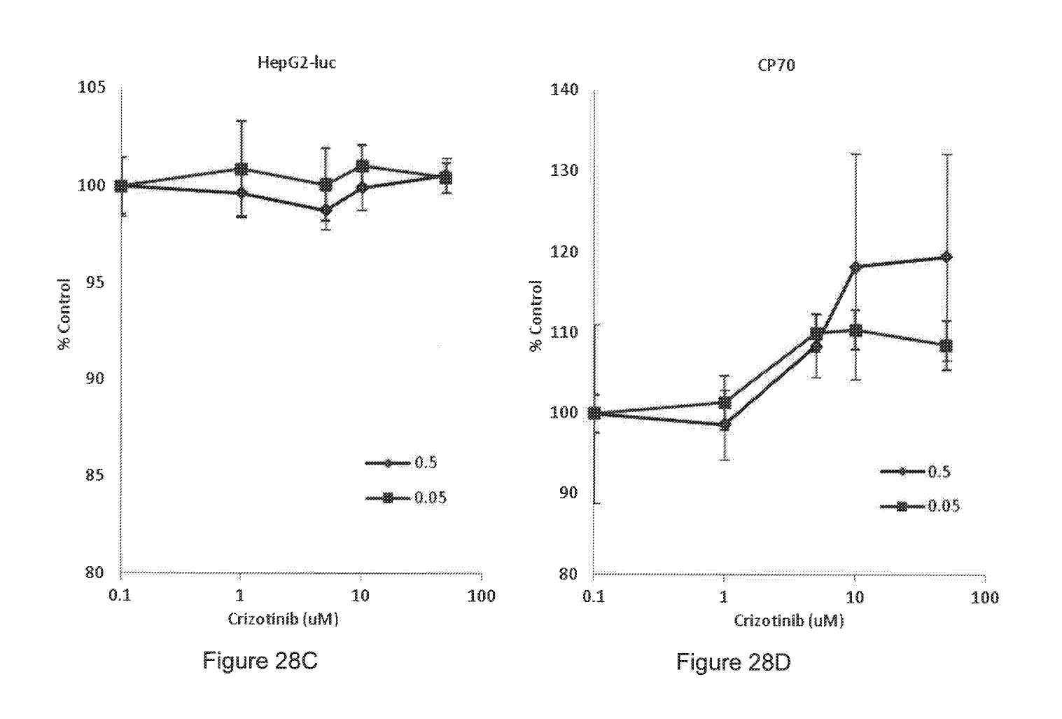

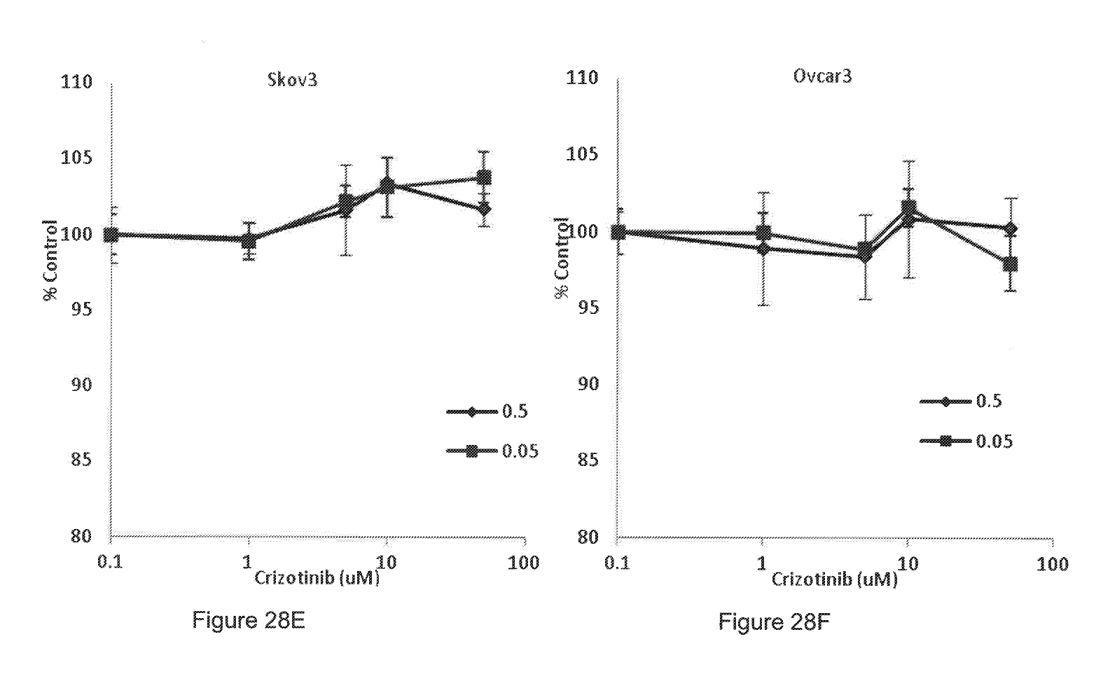

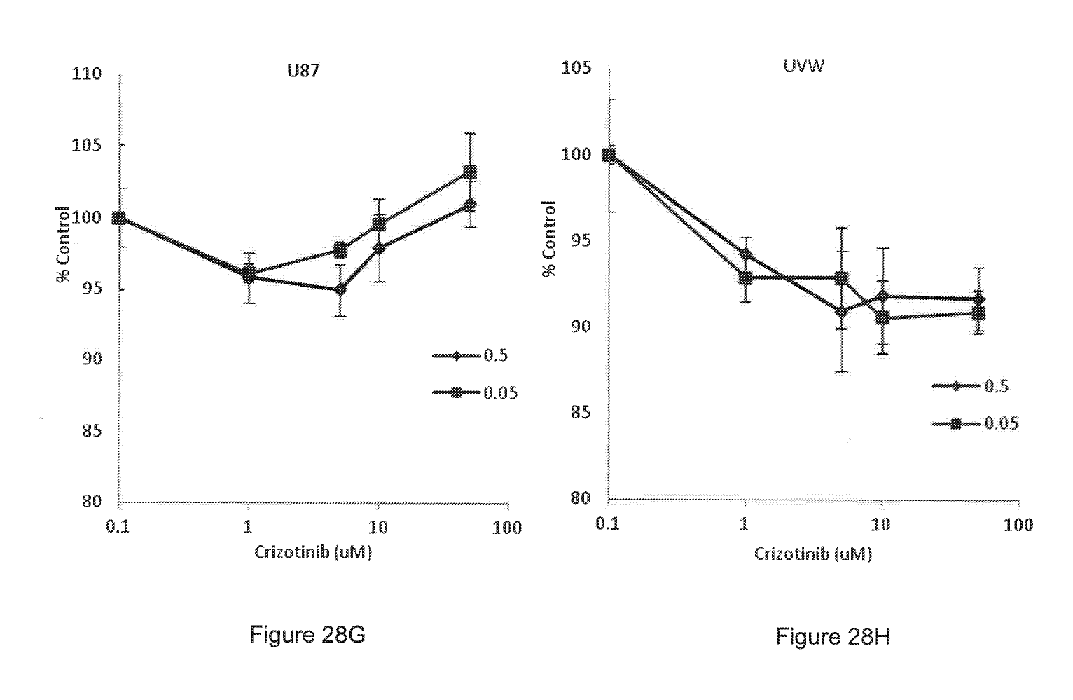

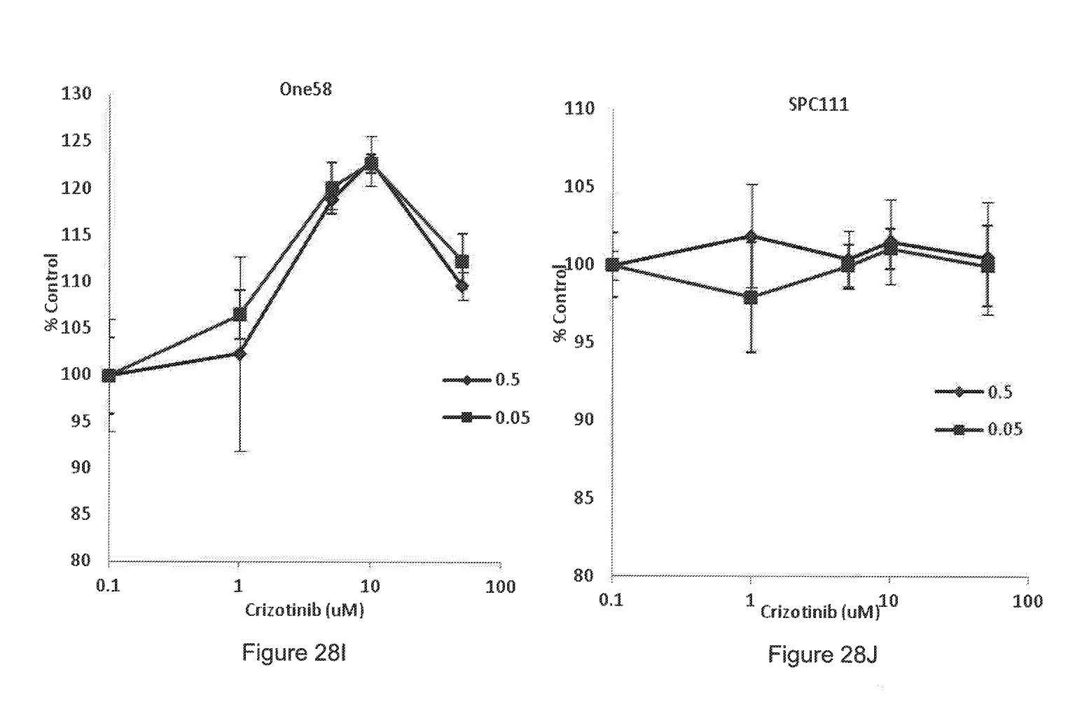

Drug-mediated virus toxicities are presented in the graphs in FIG. 28 as percentage of control cells (virus alone and no drug) in Hep3B, HuH7, HepG2-luc, CP70, Skov3, Ovcar3, U87, UVW, One58 and SPC-111 cells. Virus was used at moi 0.5 or moi 0.05.

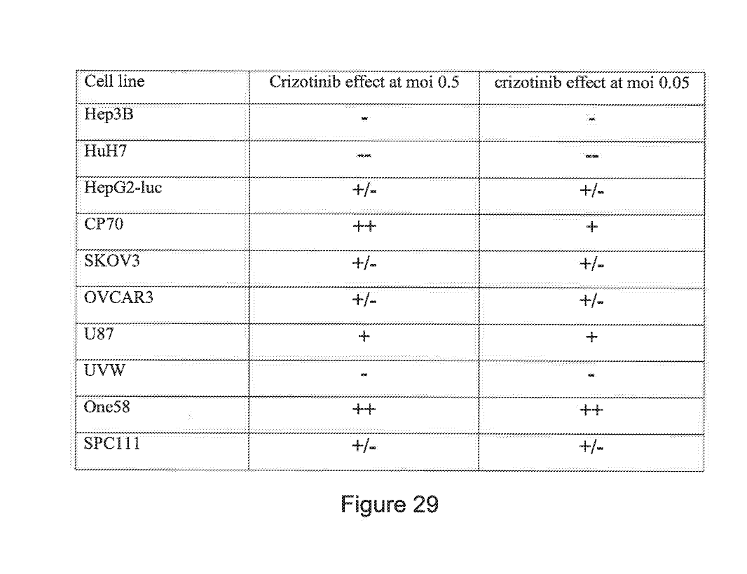

In 3 of the 10 cell lines tested, CP70, U87 and One58, Crizotinib increased GFP expression during HSV1716gfp infection at moi 0.5 and moi 0.05 (>5% variation from controls, Table 13; FIG. 29). Crizotinib had little effect on GFP expression during HSV1716gfp infection in HepG2-luc, Skov3, Ovcar3, SPC-111 lines at all drug doses. Crizotinib decreased GFP expression during HSV1716gfp infection at moi 0.5 and moi 0.05 in 3 cell lines, Hep3B, HuH7 and UVW (>5% variation from controls, Table 13; FIG. 29).

### Combination Analysis

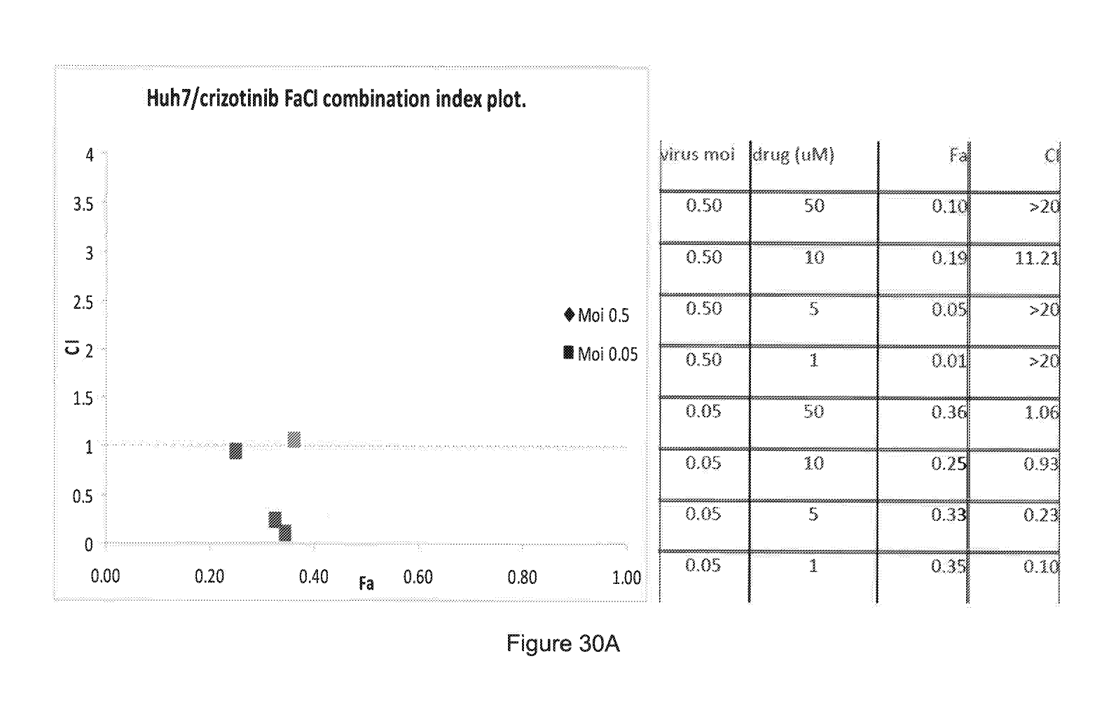

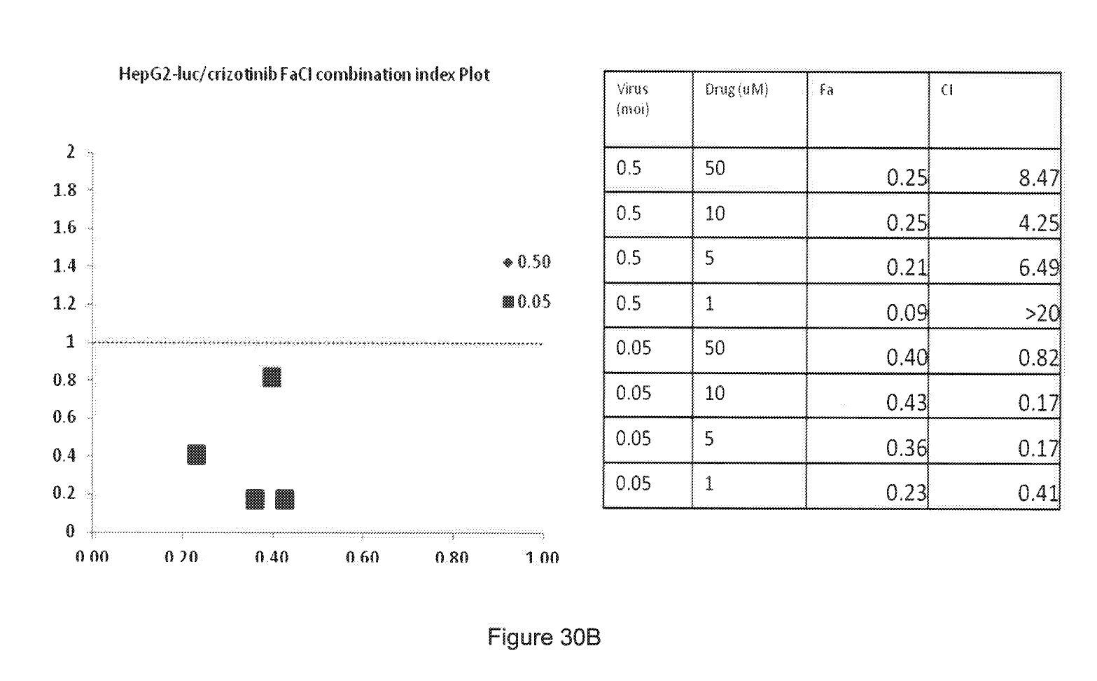

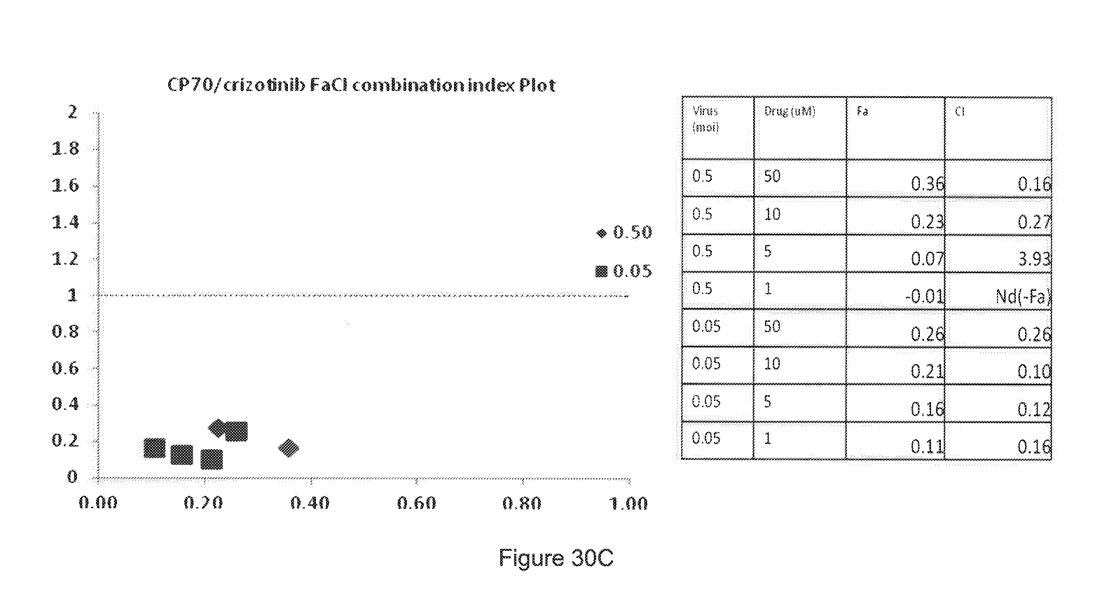

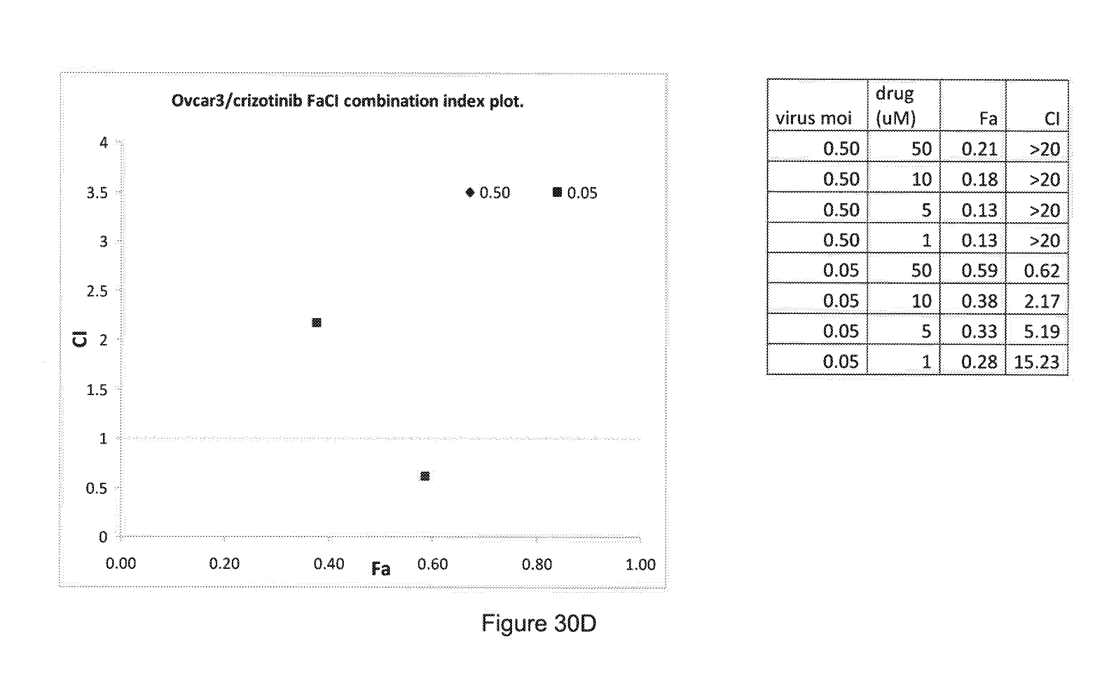

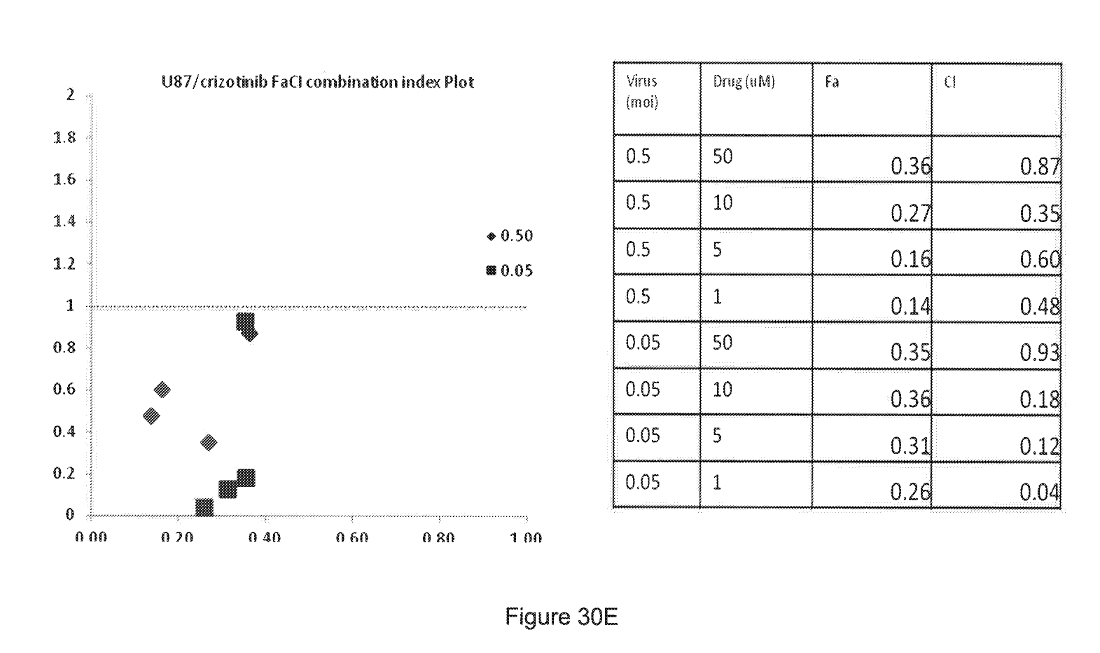

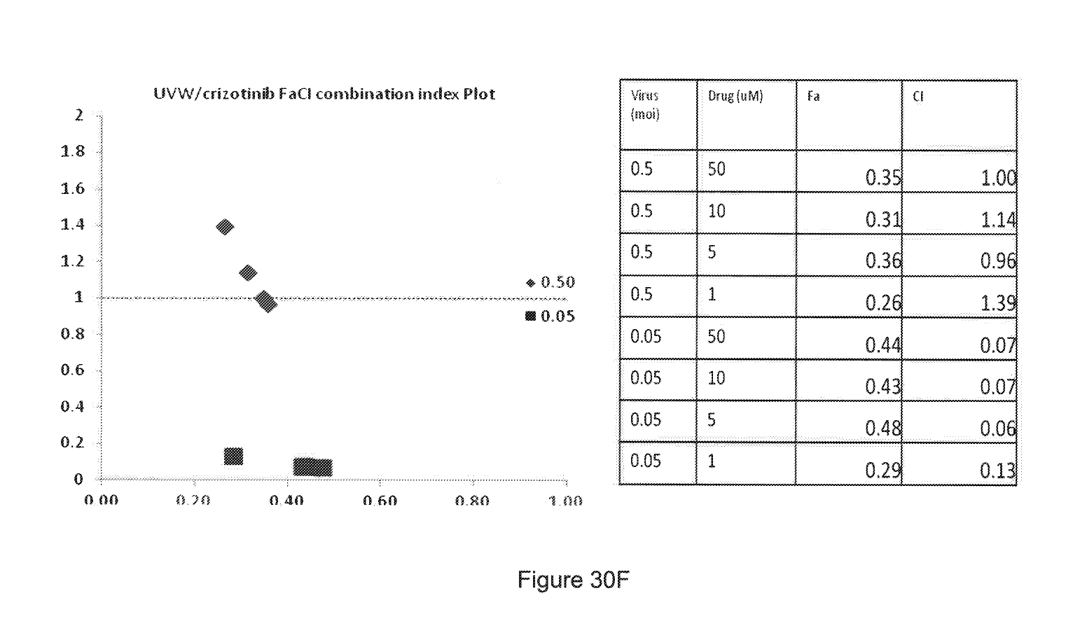

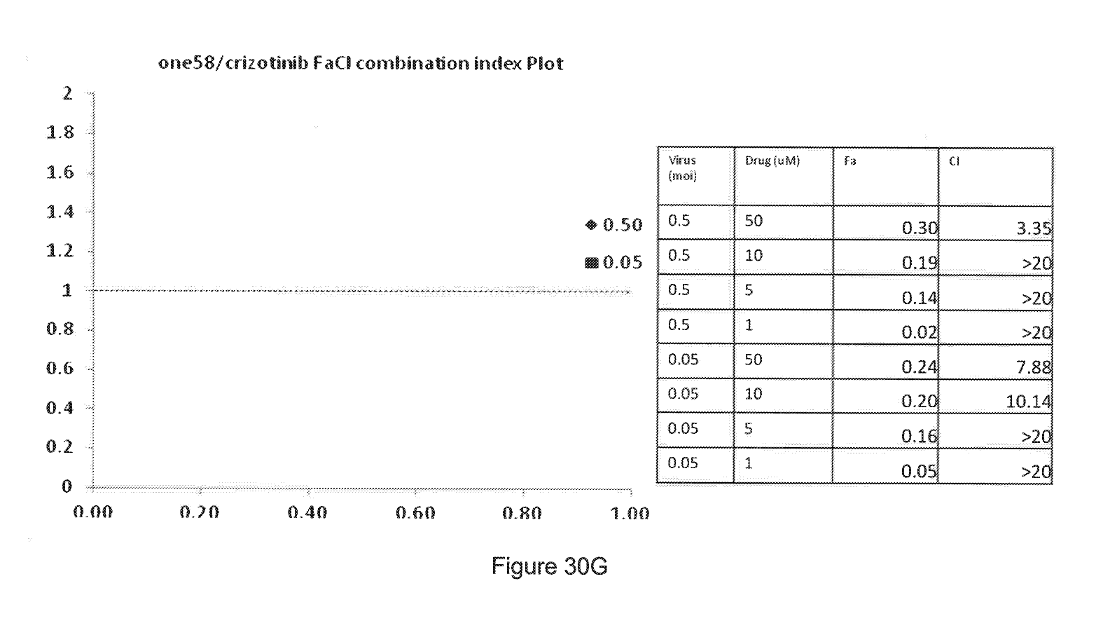

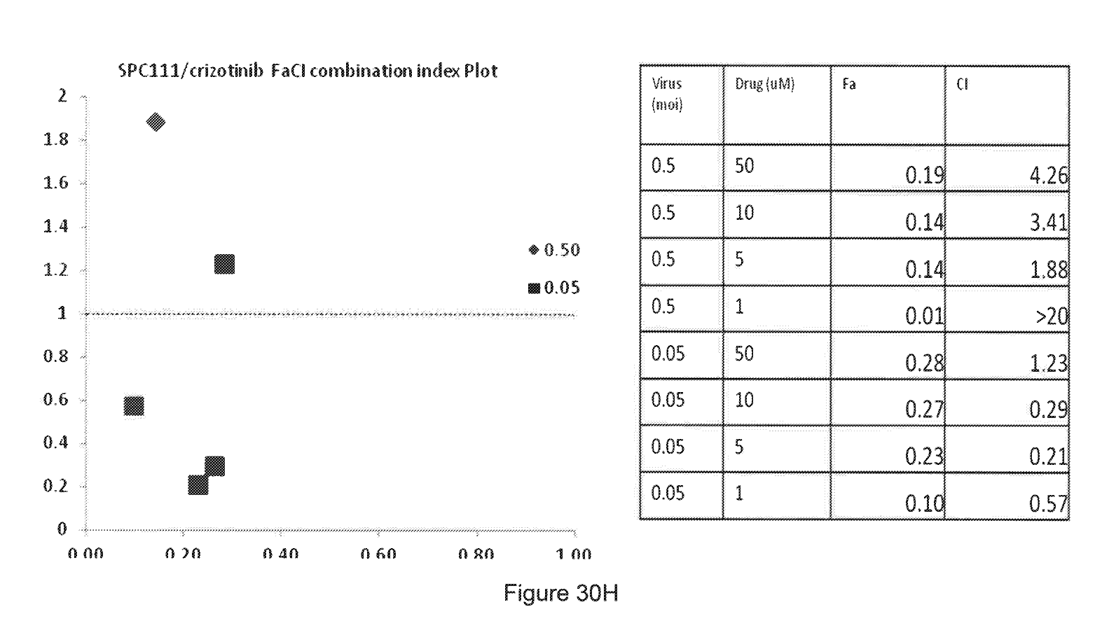

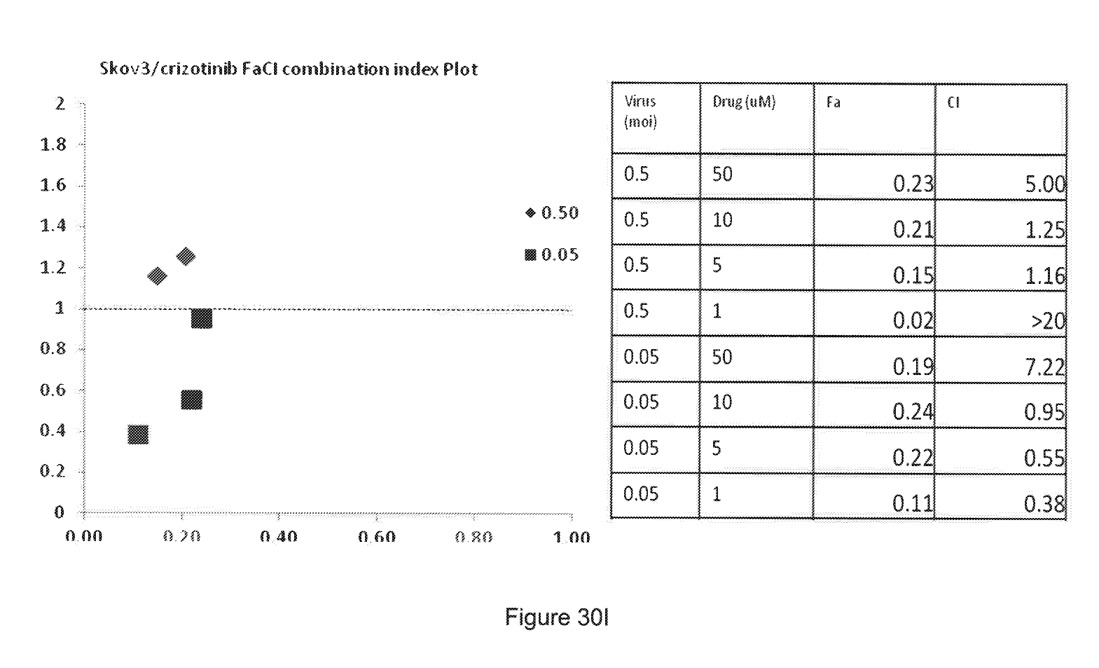

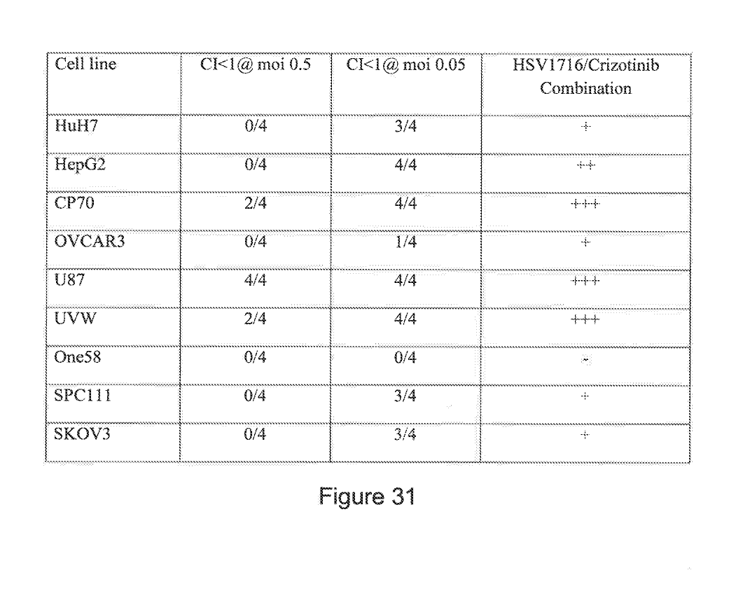

Crizotinib was tested at 4 different doses (50, 10, 5 and 1 μM) in HuH7, HepG2, CP70, Ovcar3, U87, UVW, One58, SPC111 and Skov3 cells, in combination with HSV1716 at moi 0.5 and 0.05. Representative individual plots along with their respective Fa and Cl tables are presented in FIG. 30, with synergies/antagonisms summarised in Table 14 (FIG. 31).

### Conclusions

Crizotinib is toxic in most cell lines tested at high doses.
Crizotinib was strongly synergistic with HSV1716 in U87, CP70 and UVW and synergistic in HepG2-luc cells.
Crizotinib displayed some limited synergy in HuH7, Ovcar3, SPC111 and SKOV3 cells principally at moi 0.05.
Crizotinib was antagonistic with HSV1716 in One58 cells at both moi.
Synergies occurred at both moi, moi 0.5 generated synergy in 3 of the 9 cell lines and moi 0.05 generated synergy in 8 of the 9 cell lines tested.
Crizotinib increased GFP expression in CP70, One58 and U87 cells.

### Example 7

### Erlotinib

Erlotinib was analysed in combination with HSV1716 in vitro as described, using the cell lines described and A431 cells, a human squamous carcinoma cell line that over-expresses EGFR and are sensitive to EGFR inhibitors (Emanuel et al. (2008) Mol Pharm. 73(2): 338-348). Toxicity was assessed in each well using DCP cell death assays.

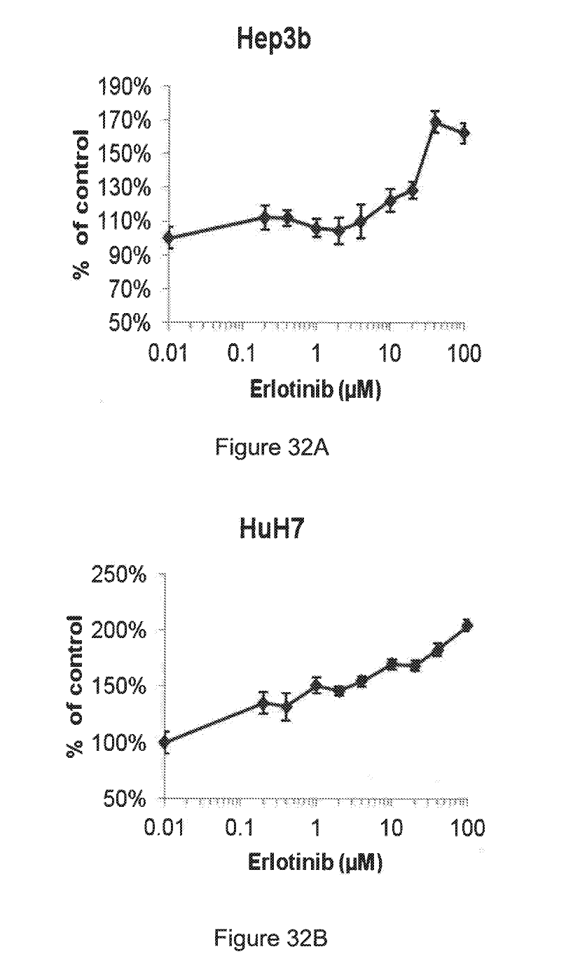

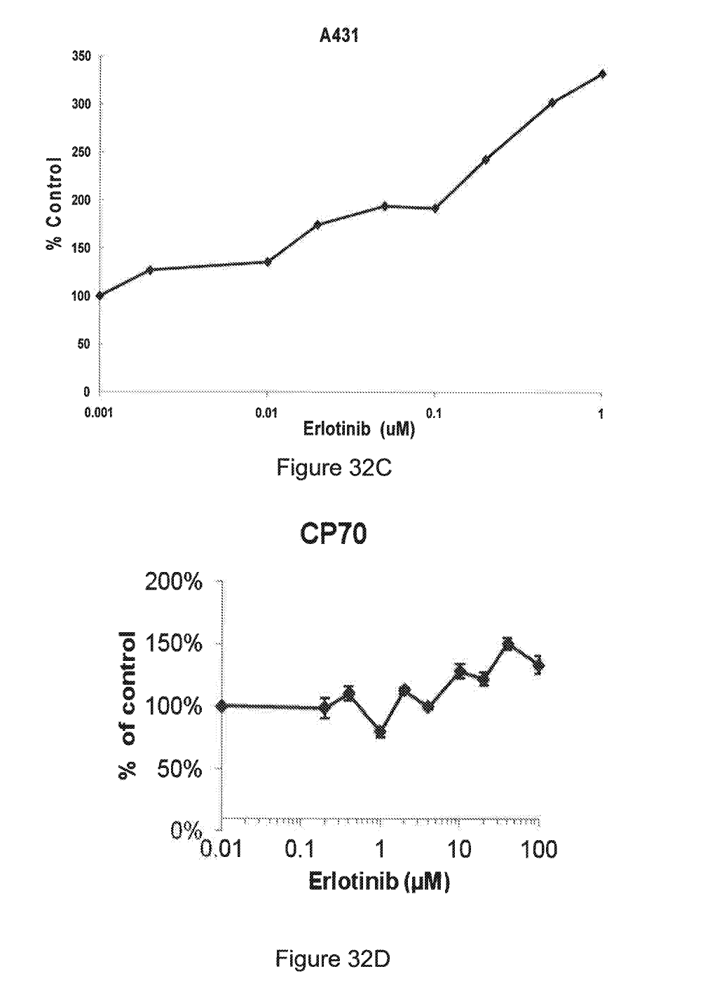

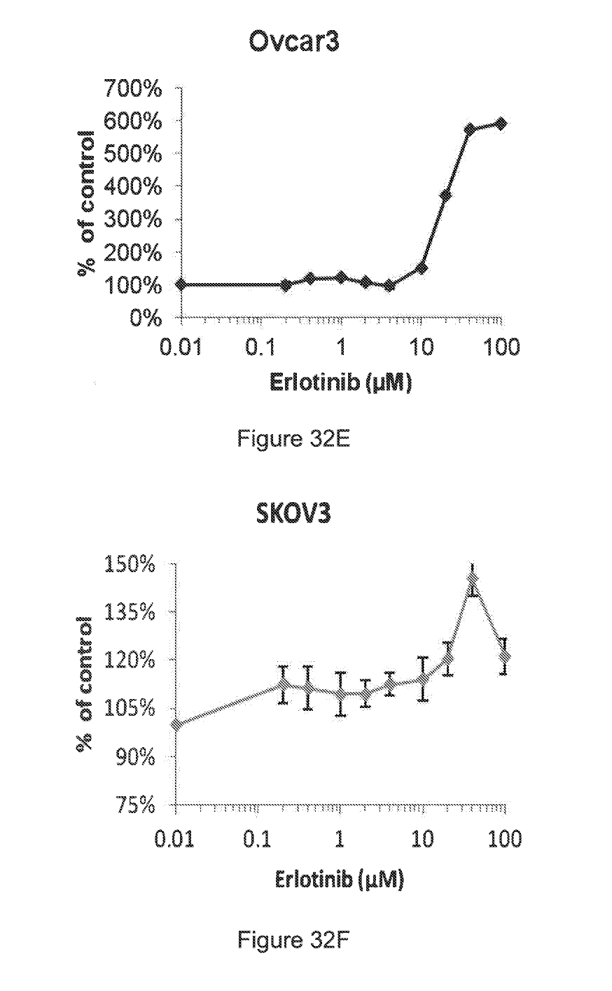

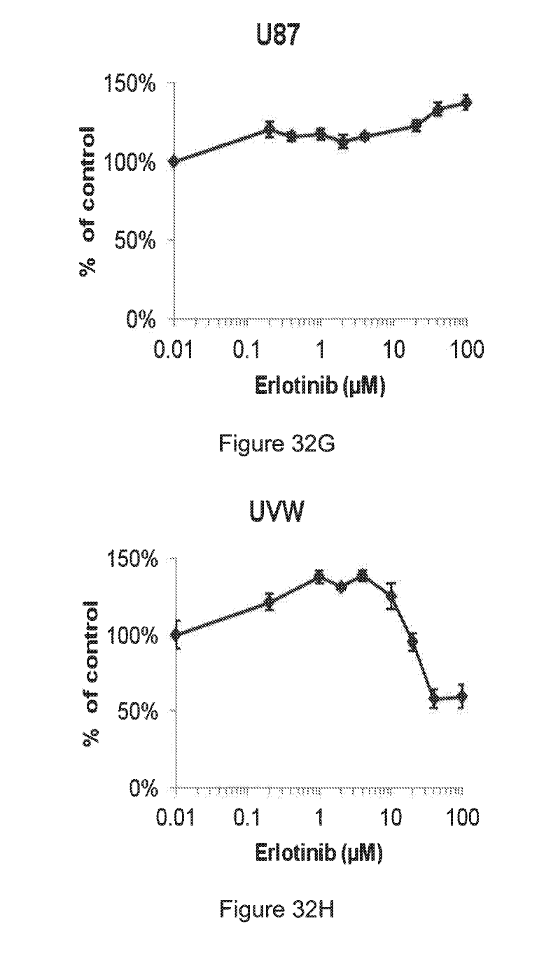

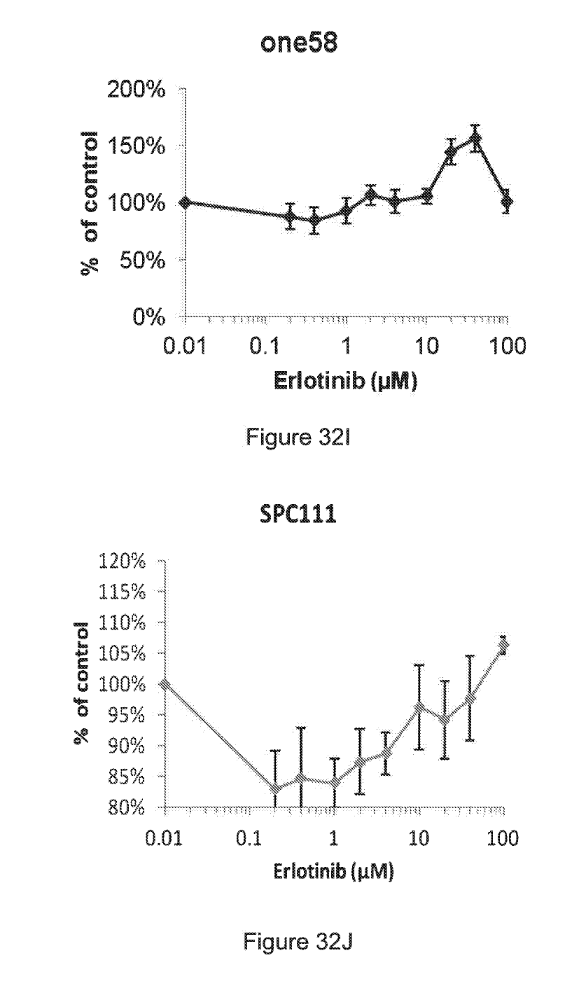

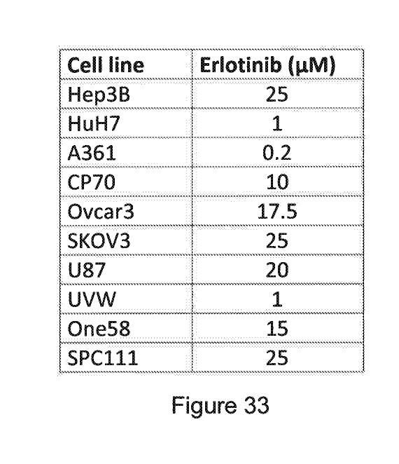

IC50 curves for each cell line tested are shown in FIG. 32 and the estimated IC50s are shown in Table 15 (FIG. 33). Only A431 cells were sensitive to Erlotinib with an IC50 of 0.2 μM. Erlotinib at higher concentrations was toxic in all other cell lines with IC50s in the range of 1-25 μM (Table 15; FIG. 33). The IC50 for Erlotinib in A431 cells has been previously reported to be 0.650 μM and the IC50 for non-sensitive cells is >10 μM (Emanuel et al. (2008) Mol Pharm. 73(2): 338-348).

### Combination Analysis

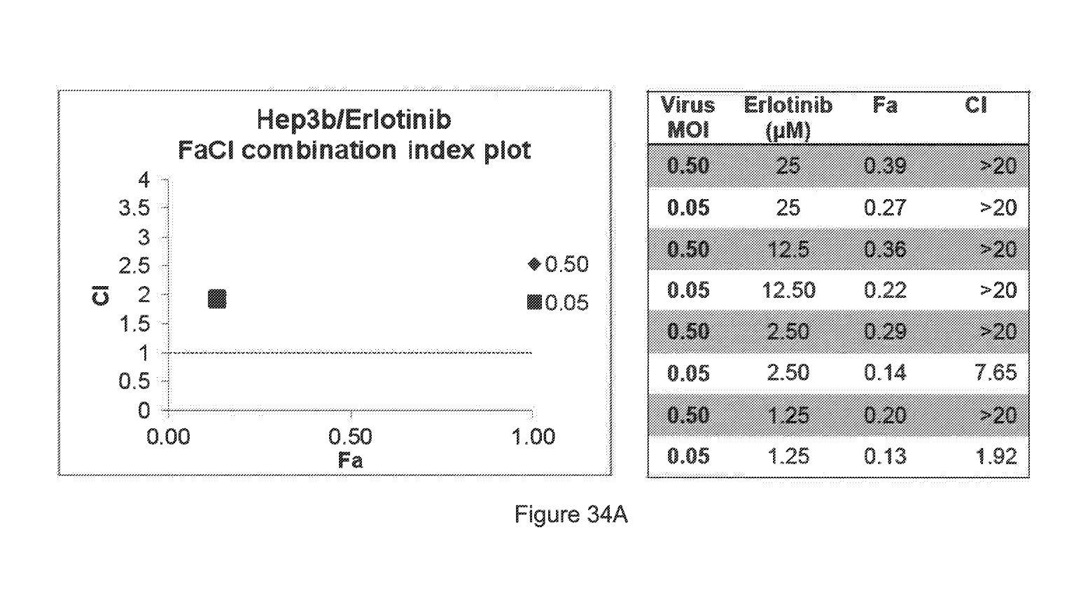

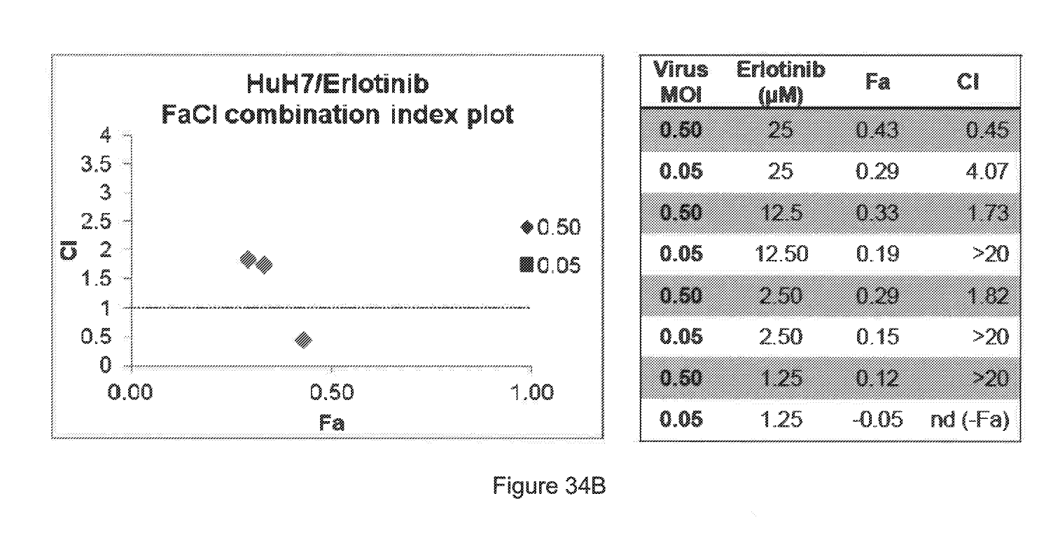

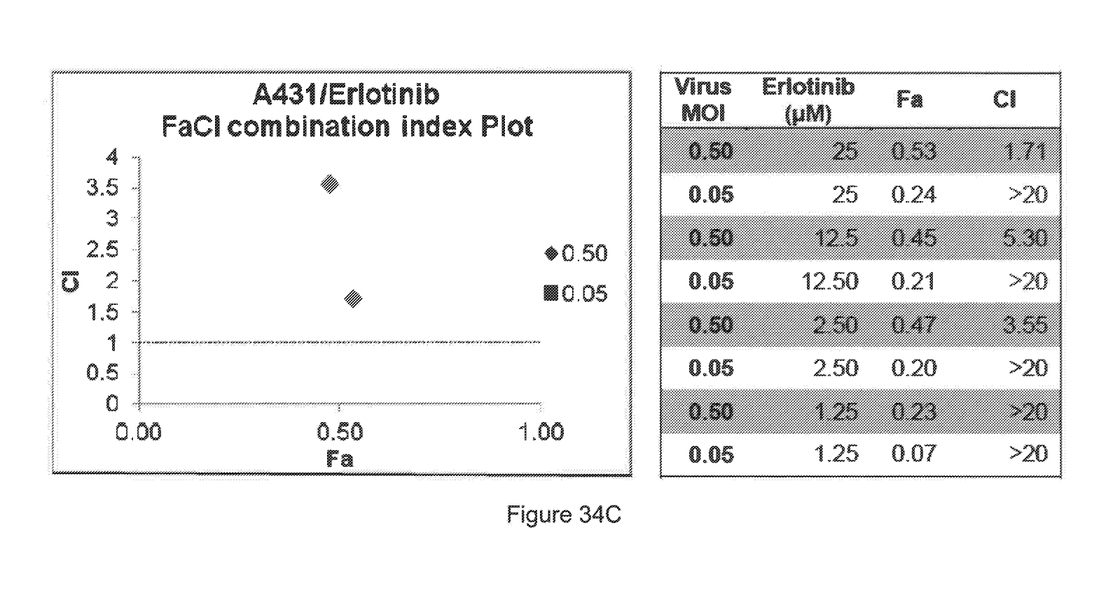

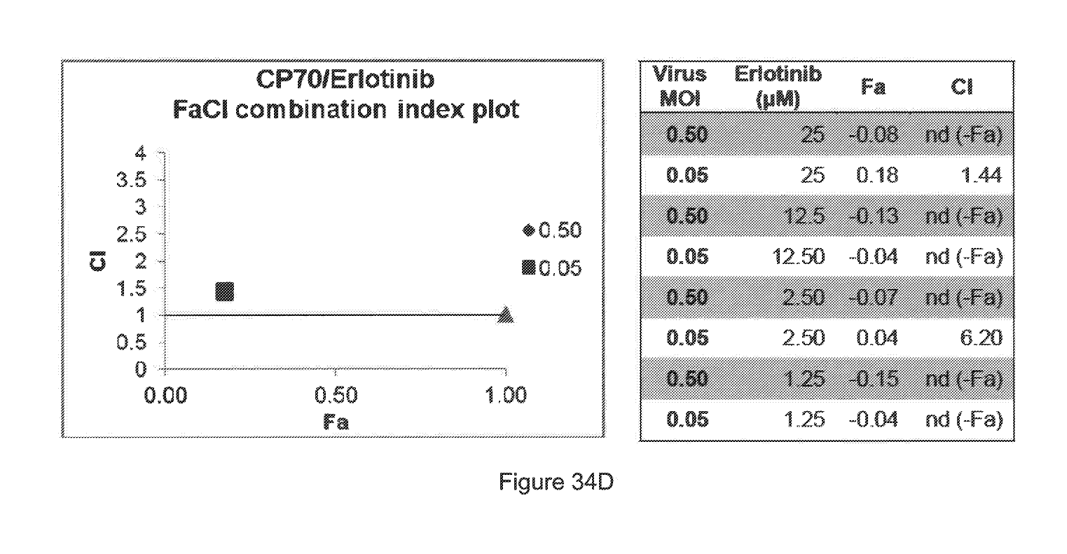

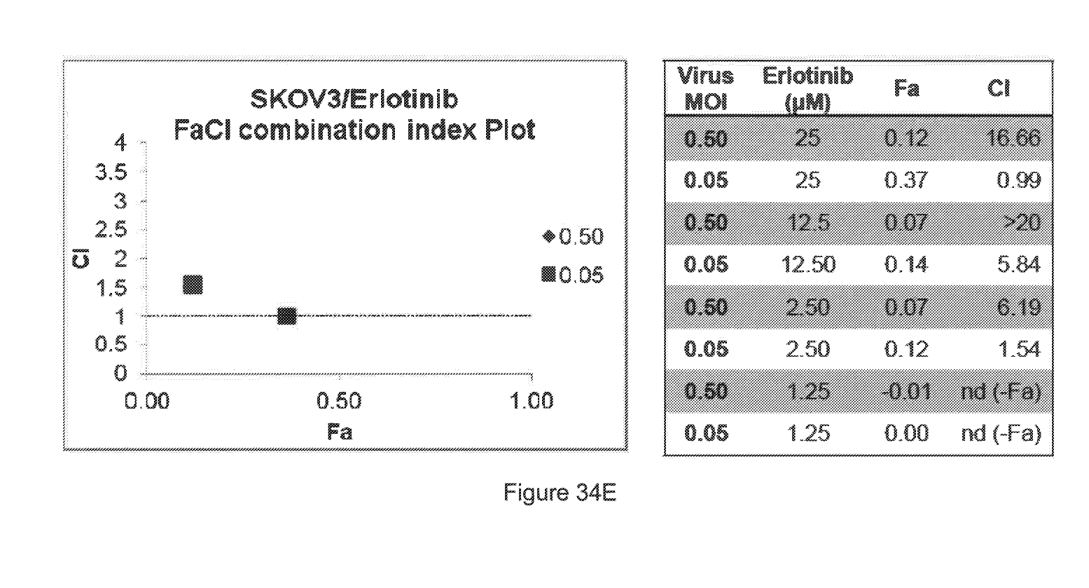

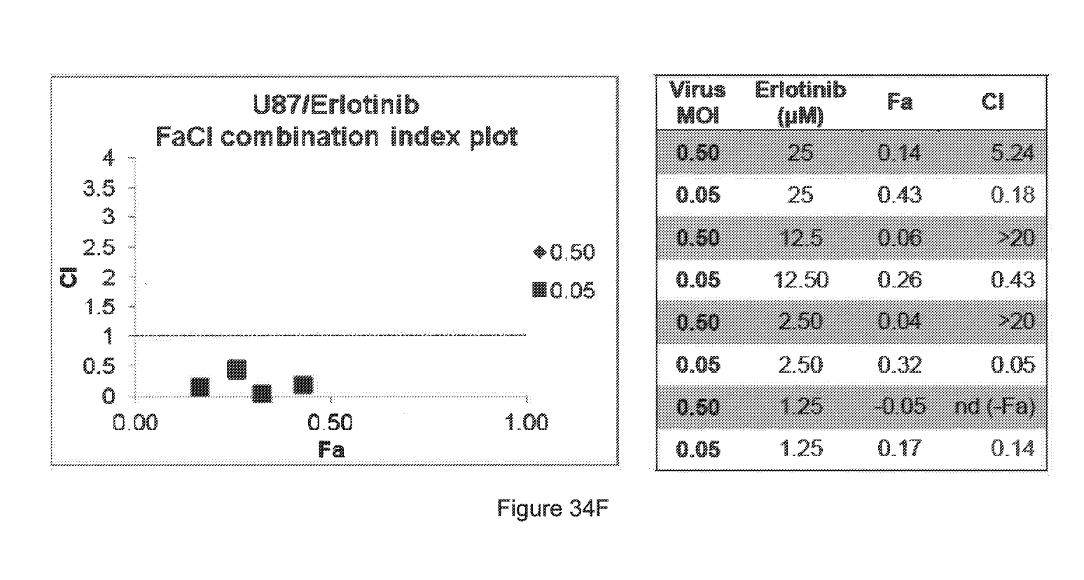

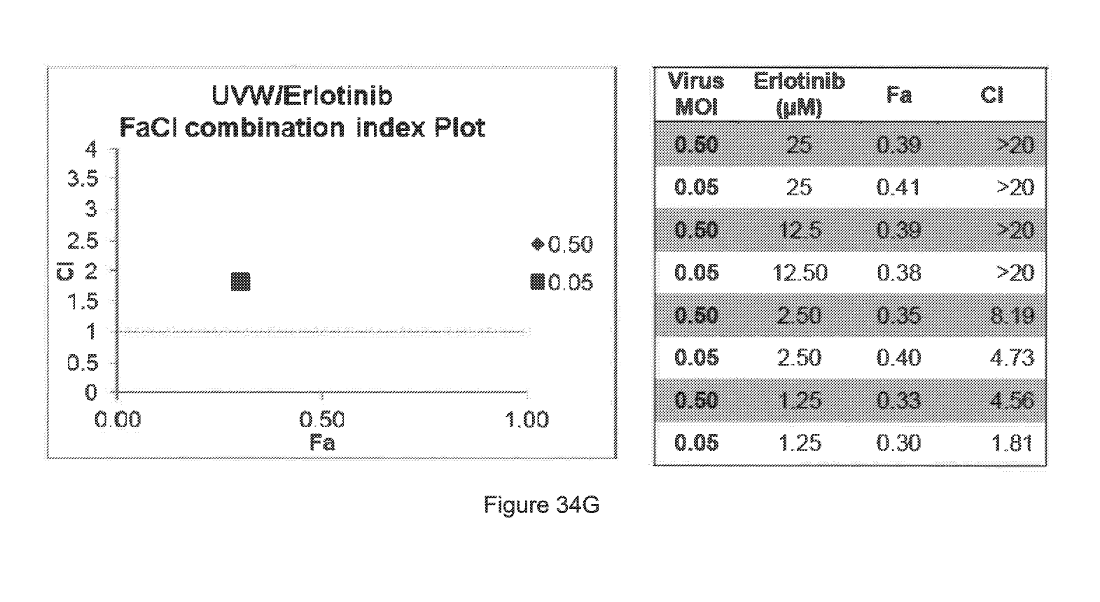

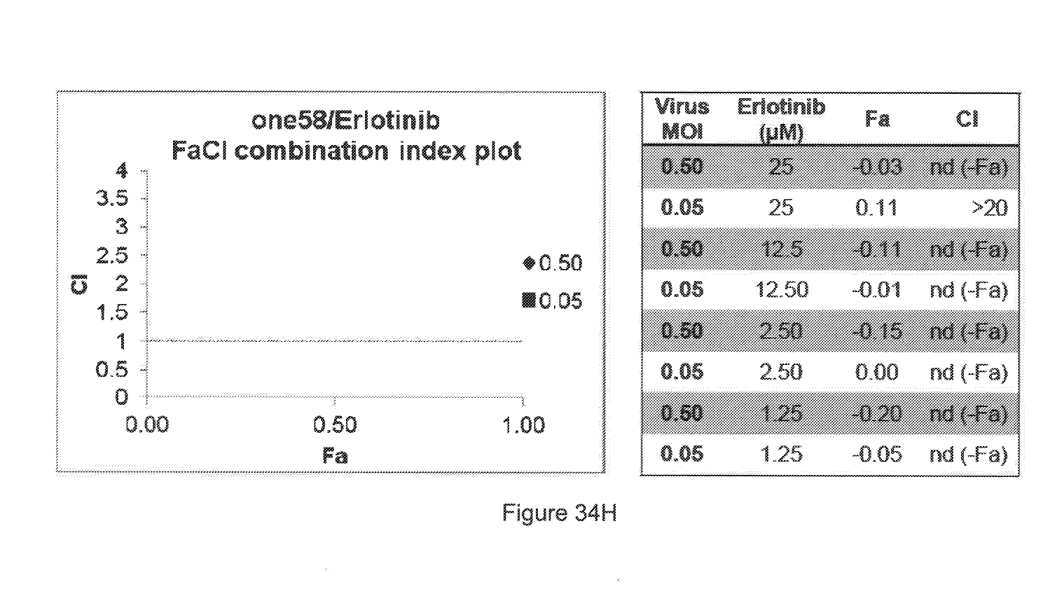

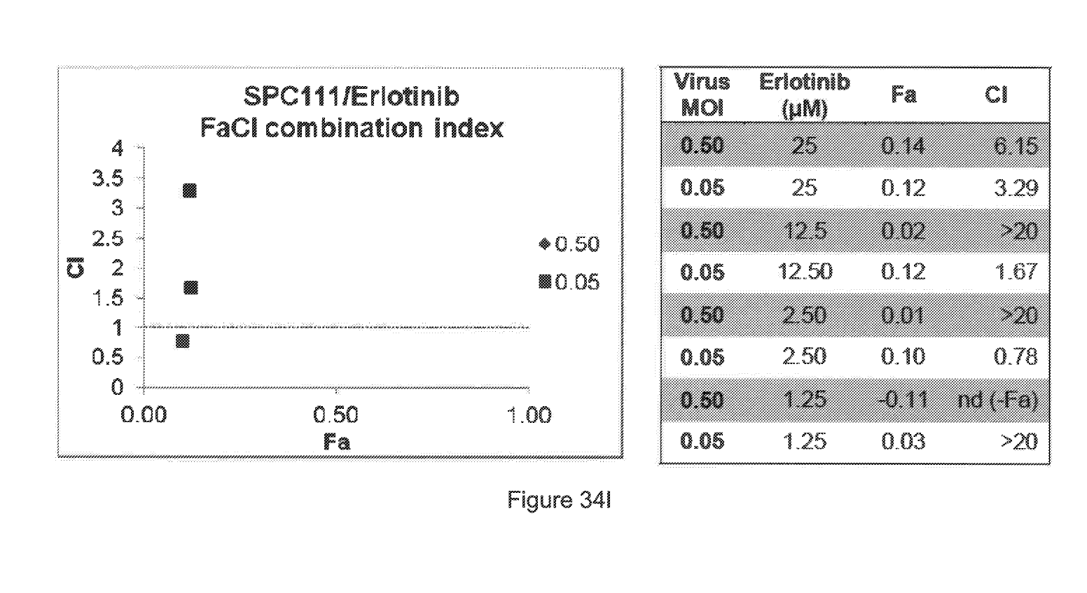

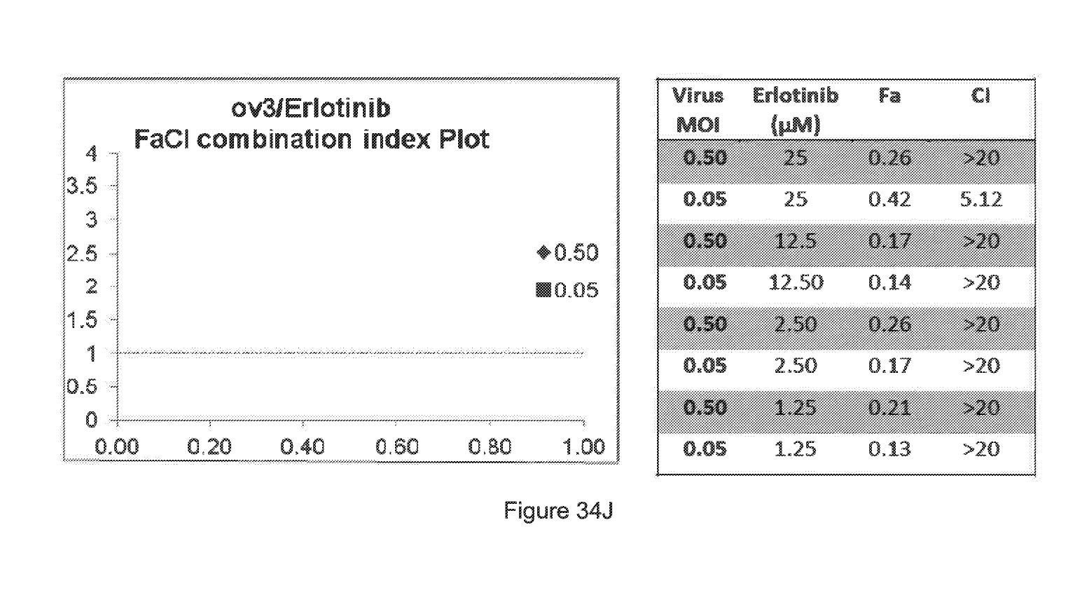

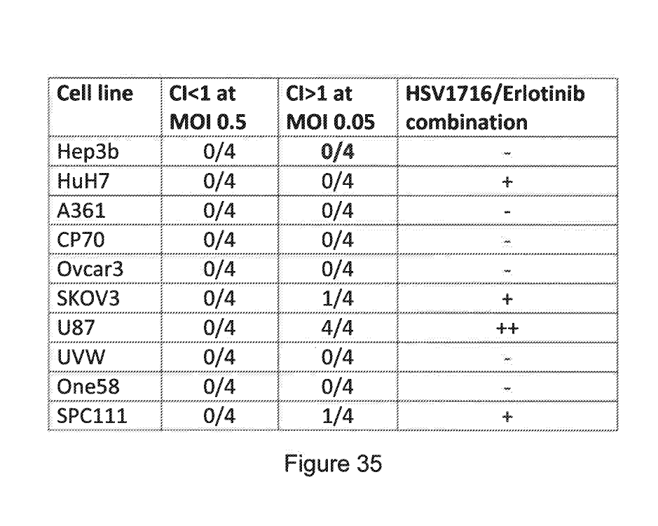

Erlotinib was tested at 4 different doses (25, 12.5, 2.5 and 1.25 μM) in A431, CP70, Hep3B, HuH7, One58, Ovcar3, SKOV3, SPC111, U87 and UVW cells, in combination with HSV1716 at moi 0.5 and 0.05. Representative individual plots along with their respective Fa and Cl table of values are presented in FIG. 34 with synergies/antagonism summarised in Table 16 (FIG. 35).

### Conclusions

Erlotinib exhibited toxicity in all the cell lines, with A431 most sensitive.
Antagonism was detected in 9 out of 10 cell lines tested.
Erlotinib was synergistic with HSV1716 at 0.05 moi in U87 cells.
Erlotinib displayed limited synergy with HSV1716 in HuH7 (1/8, moi 0.5), Skov3 (1/8, moi 0.05) and SPC111 (1/8, moi 0.05) cells.

### Example 8

### Gefitinib

Gefitinib was analysed in combination with HSV1716 in vitro as described, using the cell lines described. Toxicity was assessed in each well using DCP cell death assays.

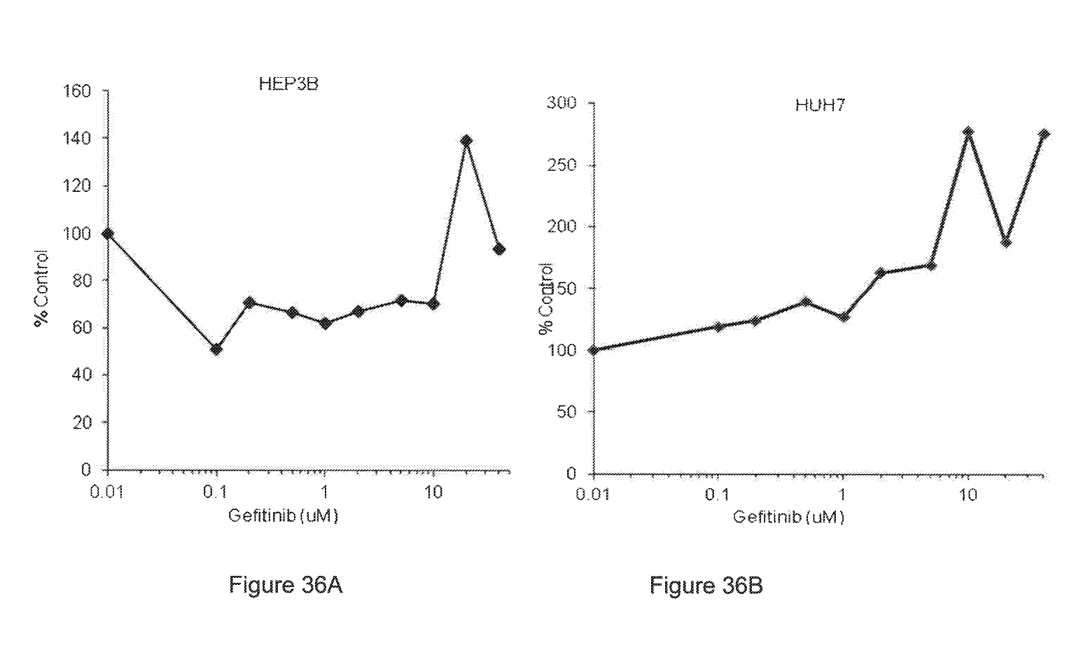

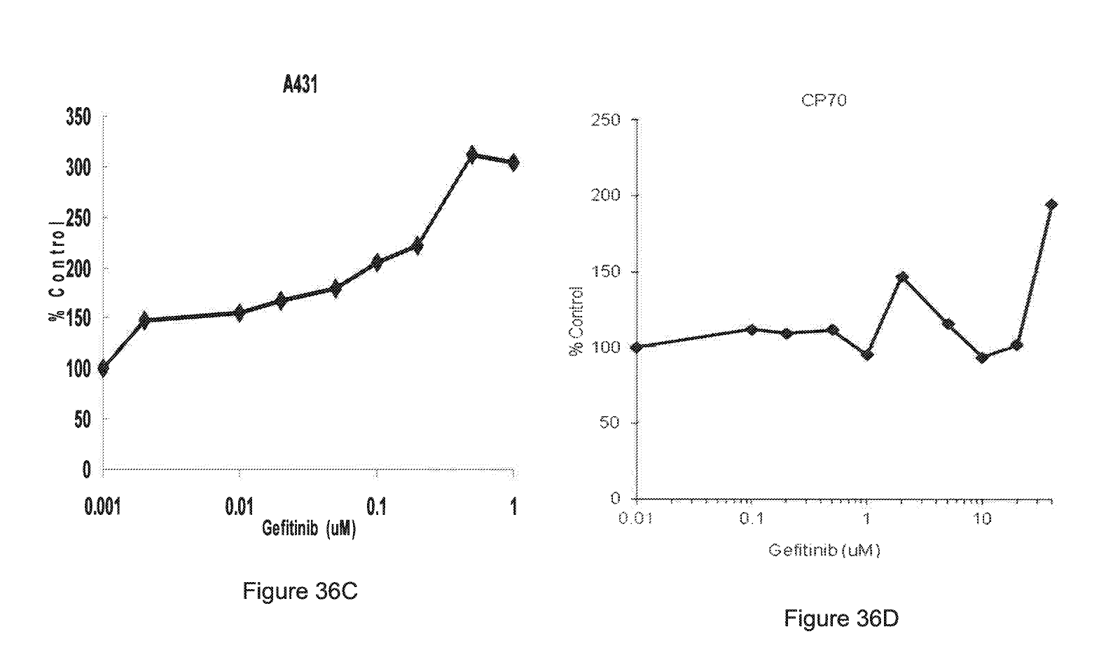

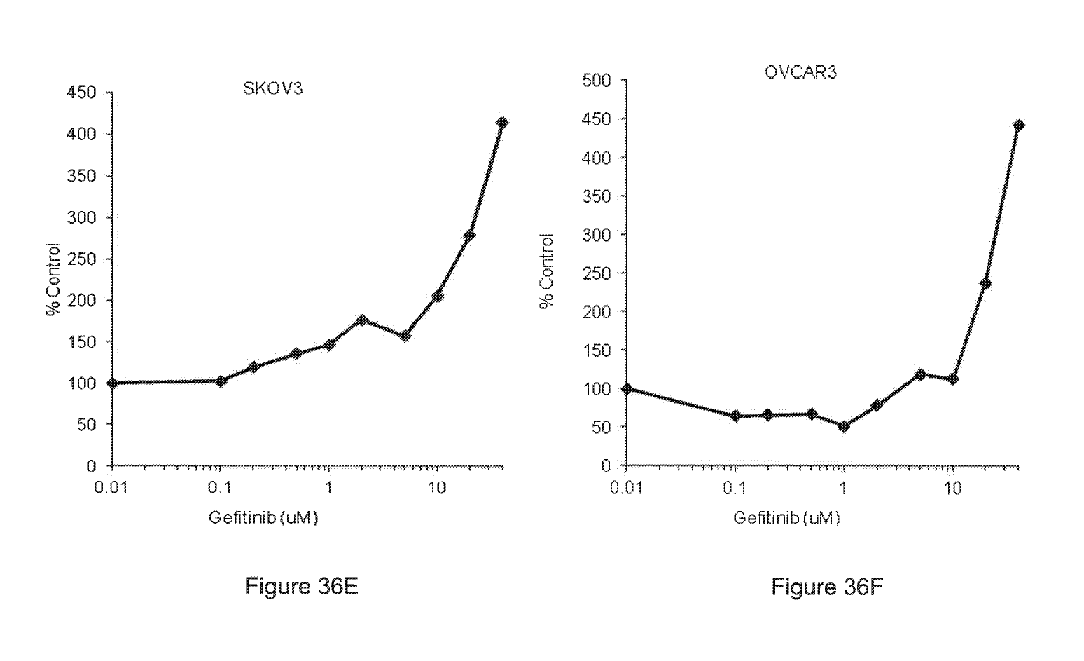

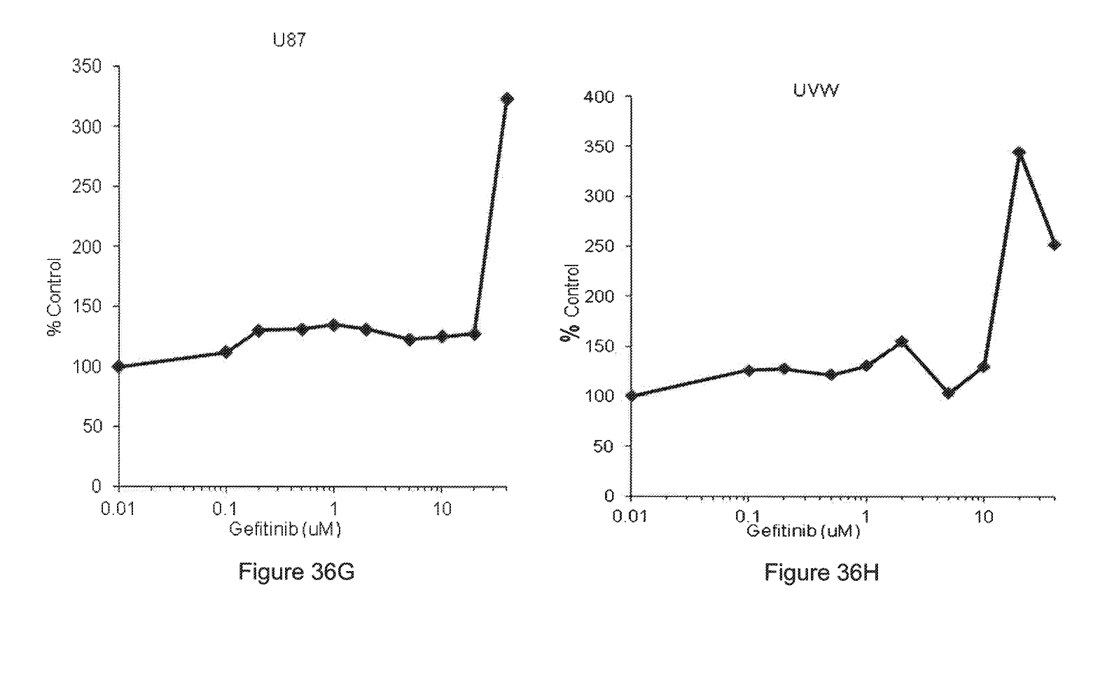

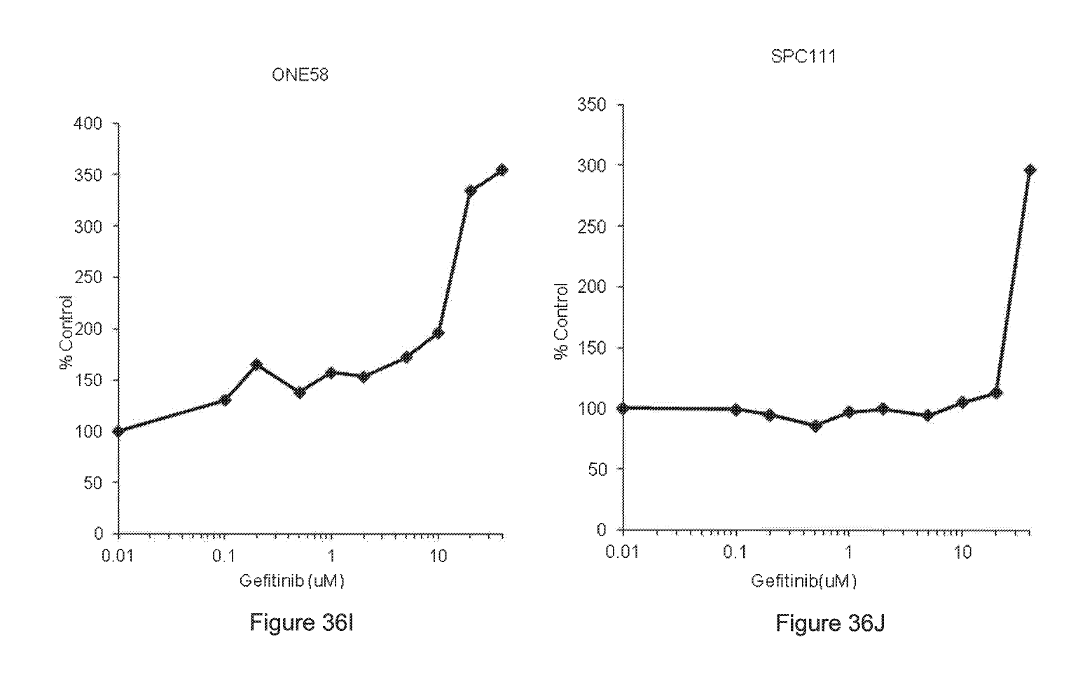

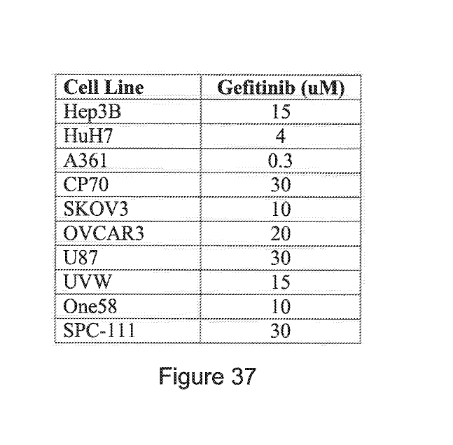

IC50 curves for each cell line tested are shown in FIG. 36, and the estimated IC50s are shown in Table 17 (FIG. 37). Only A431 cells were sensitive to Gefitinib, with an IC50 of 0.3 μM. Gefitinib at higher concentrations was toxic in all other cell lines, with IC50s in the range of 4-30 μM (Table 17; FIG. 37). The IC50 for Gefitinib in A431 cells has been previously reported to be 0.825 μM and the IC50 for non-sensitive cells is >10 μM (Emanuel et al. (2008) Mol Pharm. 73(2): 338-348).

### Combination Analysis

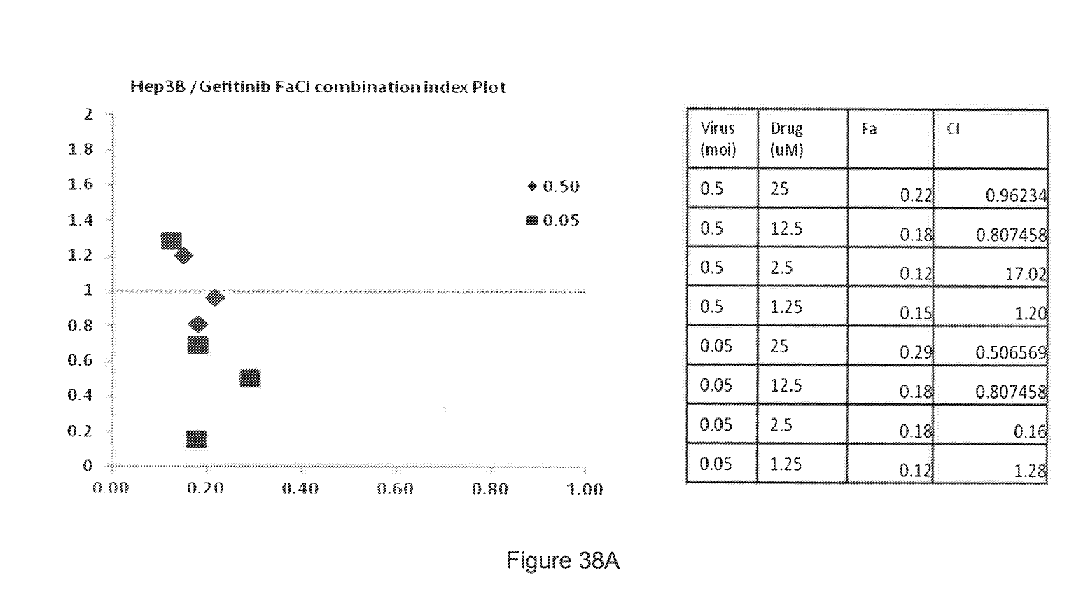

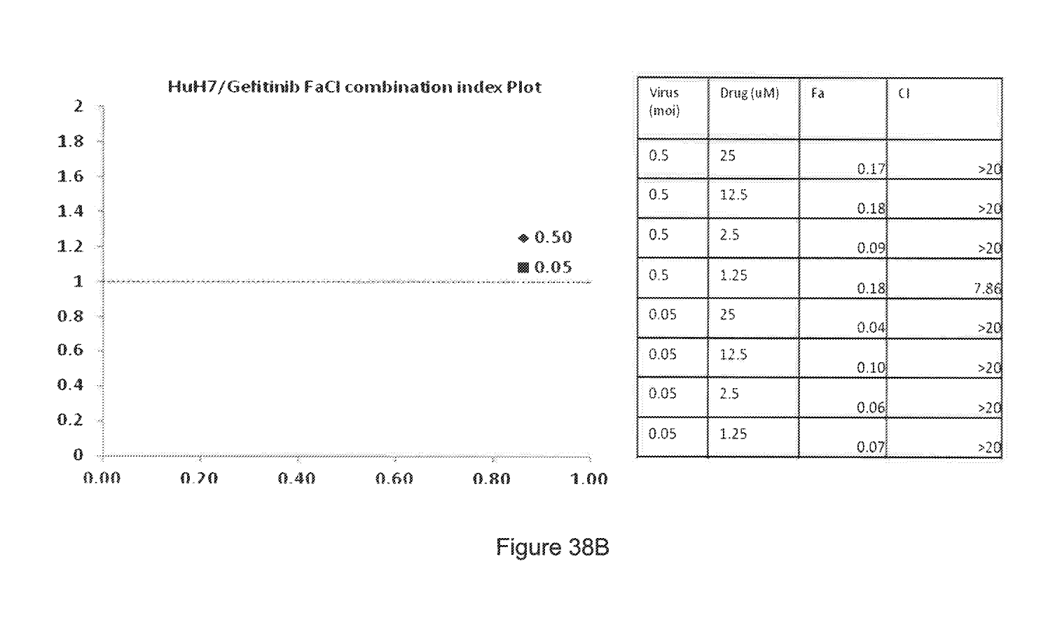

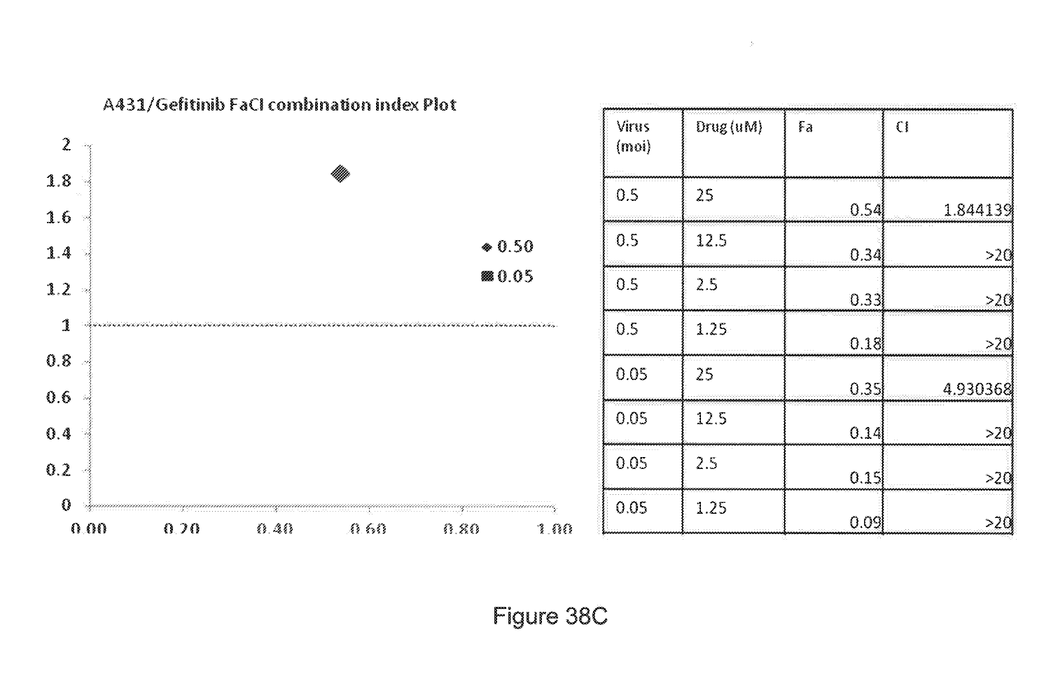

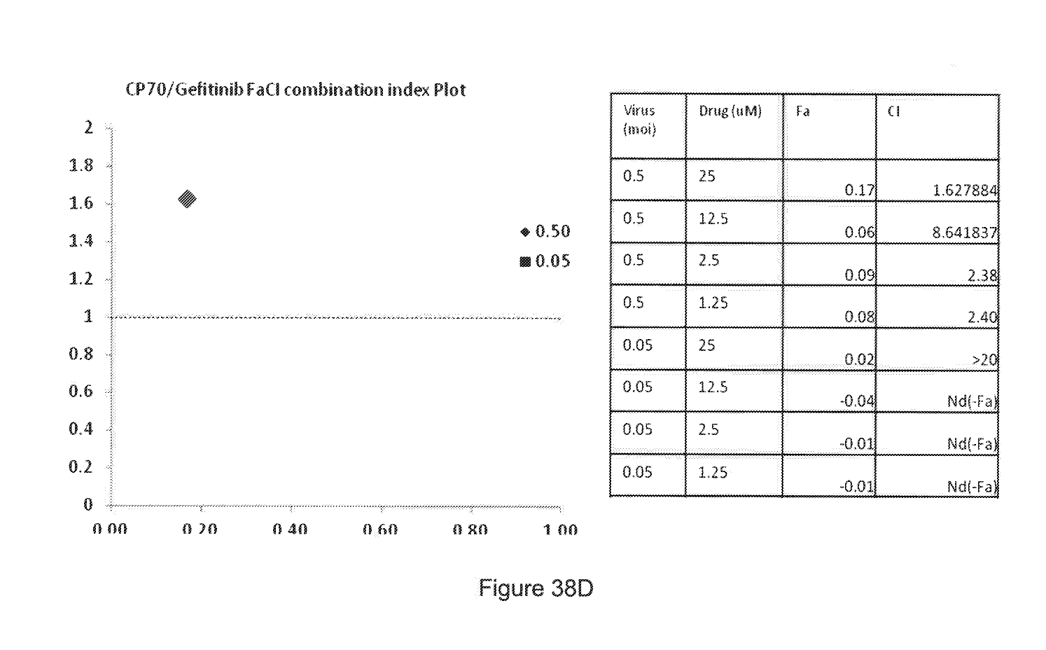

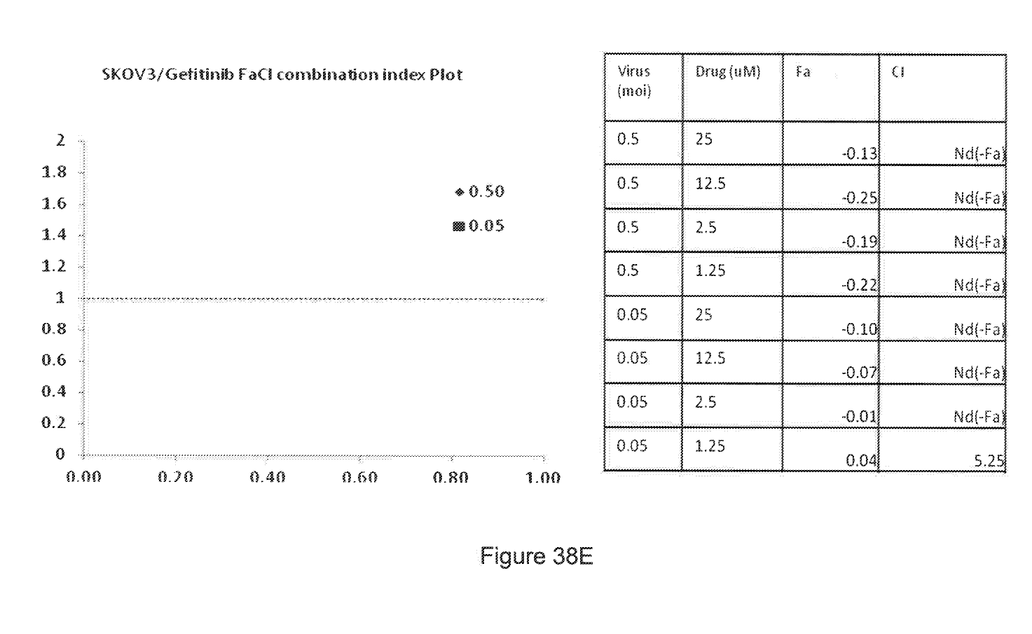

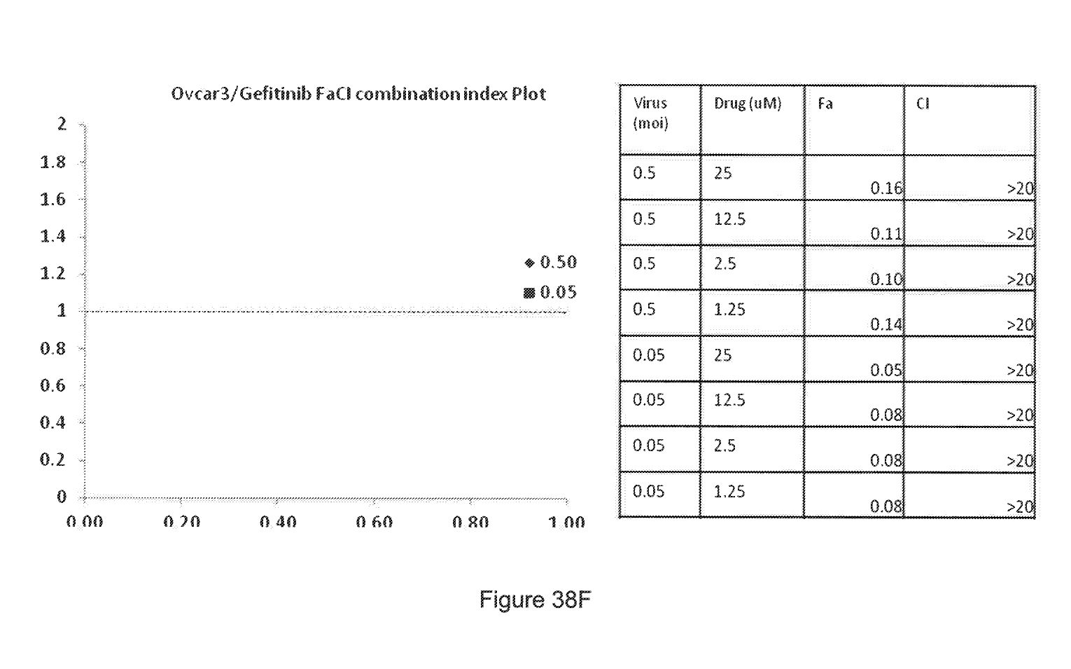

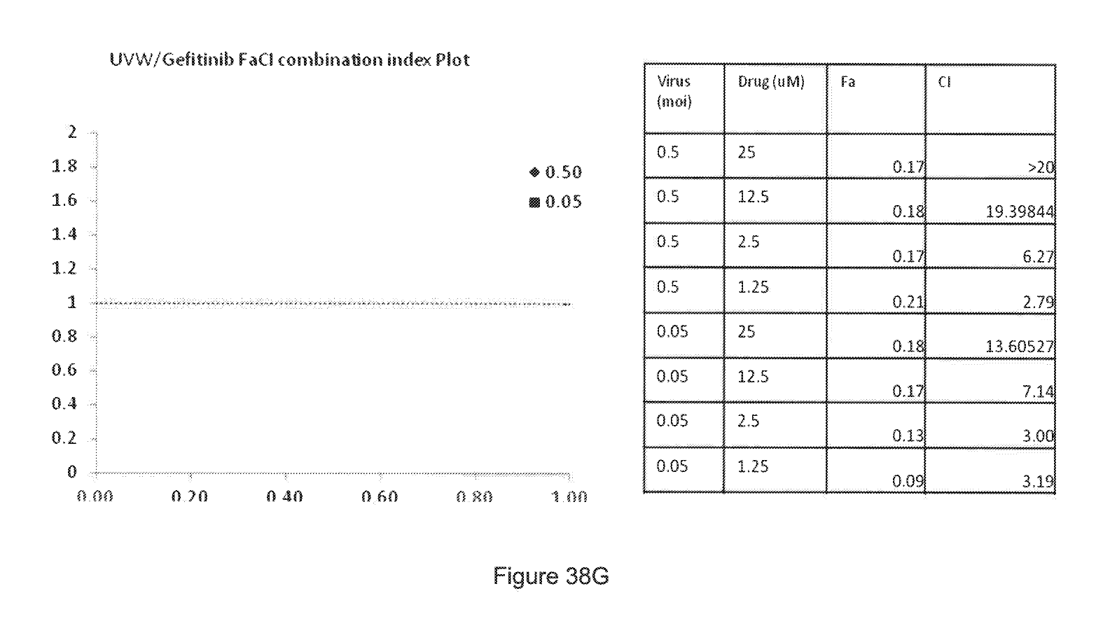

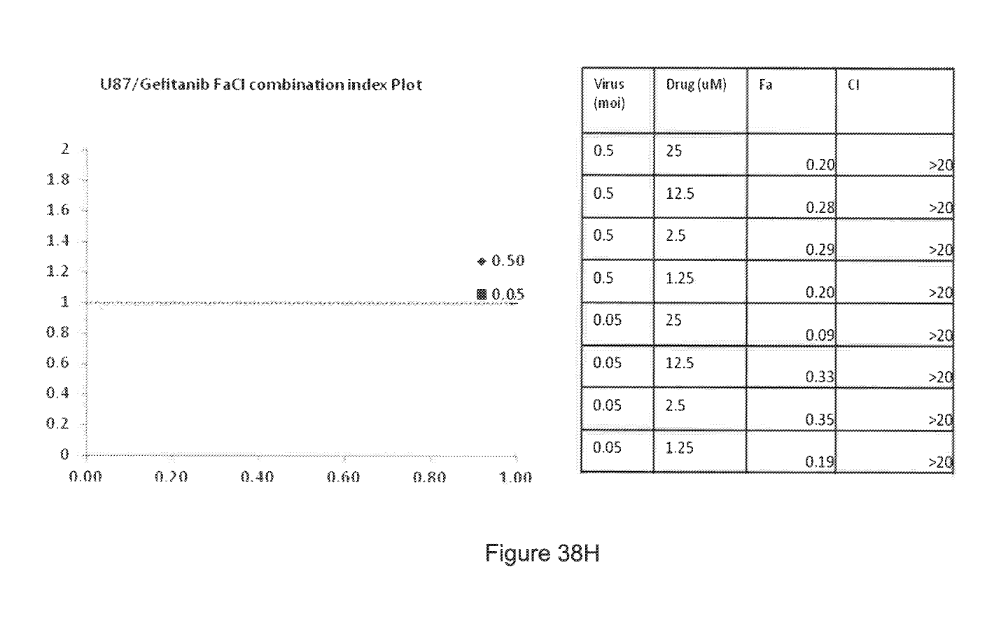

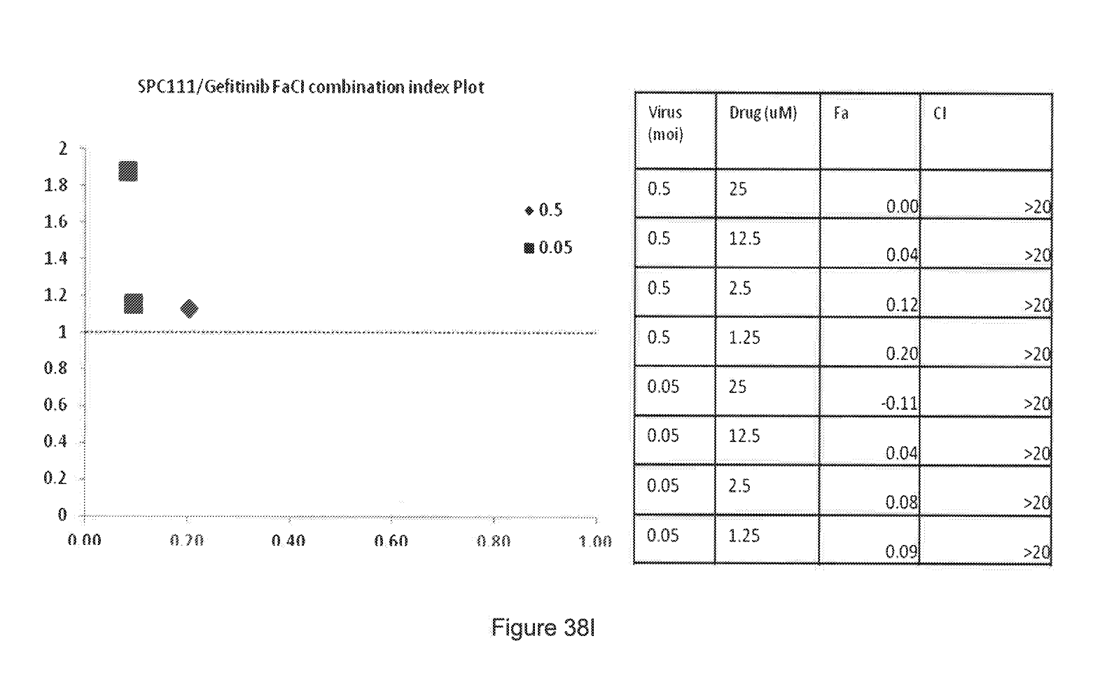

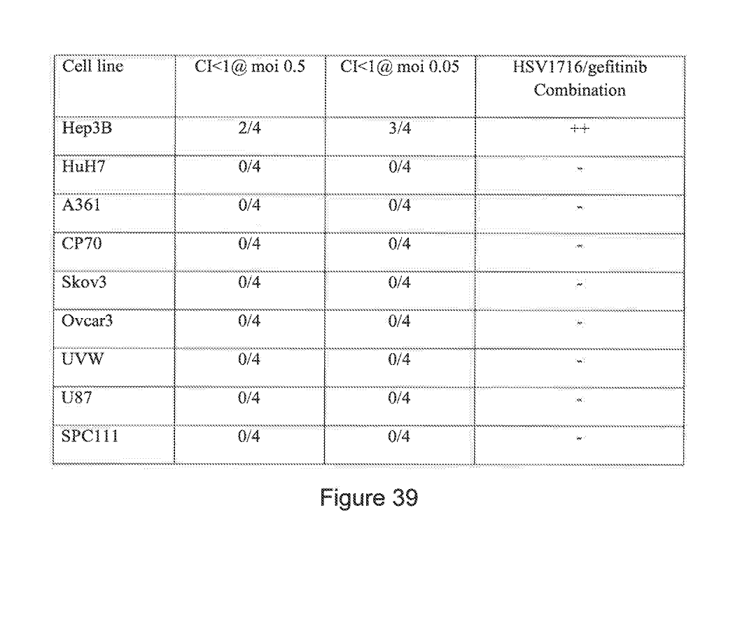

Gefitinib was tested at 4 different doses (25, 12.5, 2.5 and 1.25 μM) in Hep3B, HuH7, A431, CP70, Skov3, Ovcar3, UVW, U87 and SPC111 cells, in combination with HSV1716 at moi 0.5 and 0.05. Representative individual plots along with their respective Fa and Cl values are presented in FIG. 38 with synergies/antagonisms summarised in Table 18 (FIG. 39).

### Conclusions

Apart from in A431 cells, Gefitinib is only toxic at high doses in the cell lines tested.
Gefitinib was antagonistic with HSV1716 in all cell lines, with the exception of Hep3B.
Synergy of Gefitinib in combination with HSV1716 was detected in Hep3B cells at both moi.

### Example 9

### Lapatinib

Lapatinib was analysed in combination with HSV1716 in vitro as described, using the cell lines described. Toxicity was assessed in each well using DCP cell death assays.

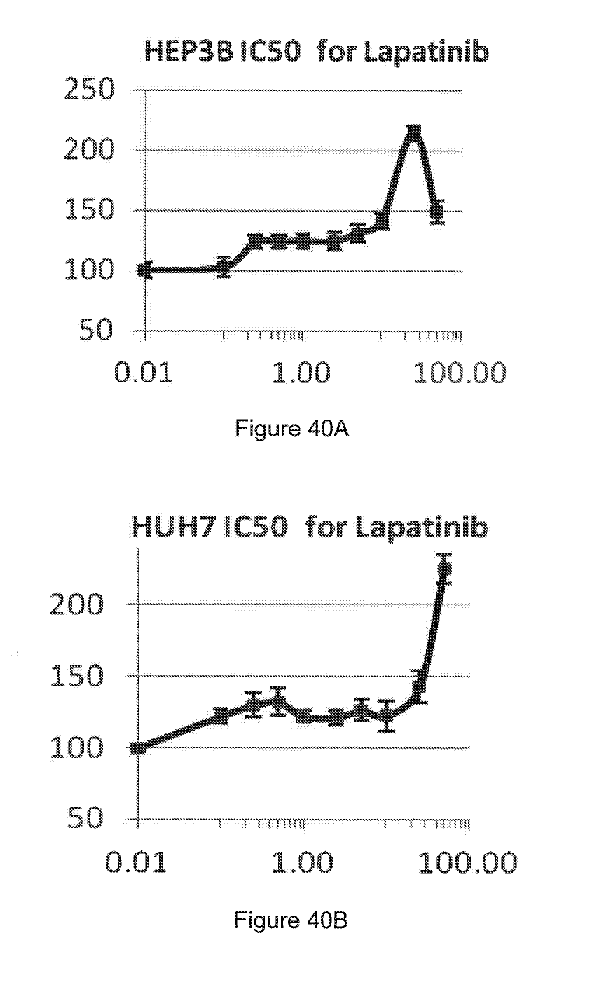

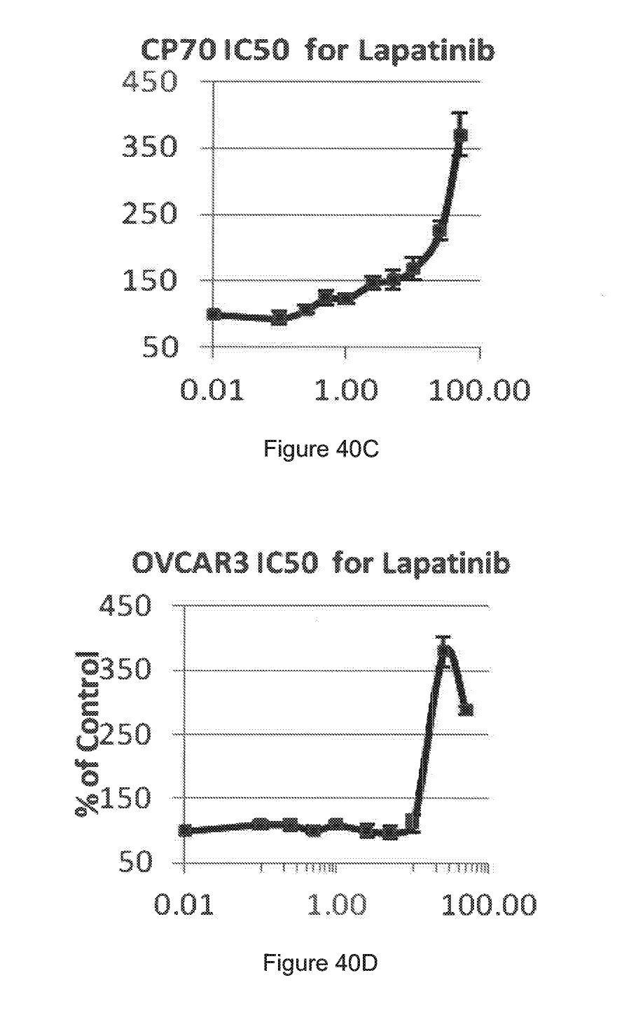

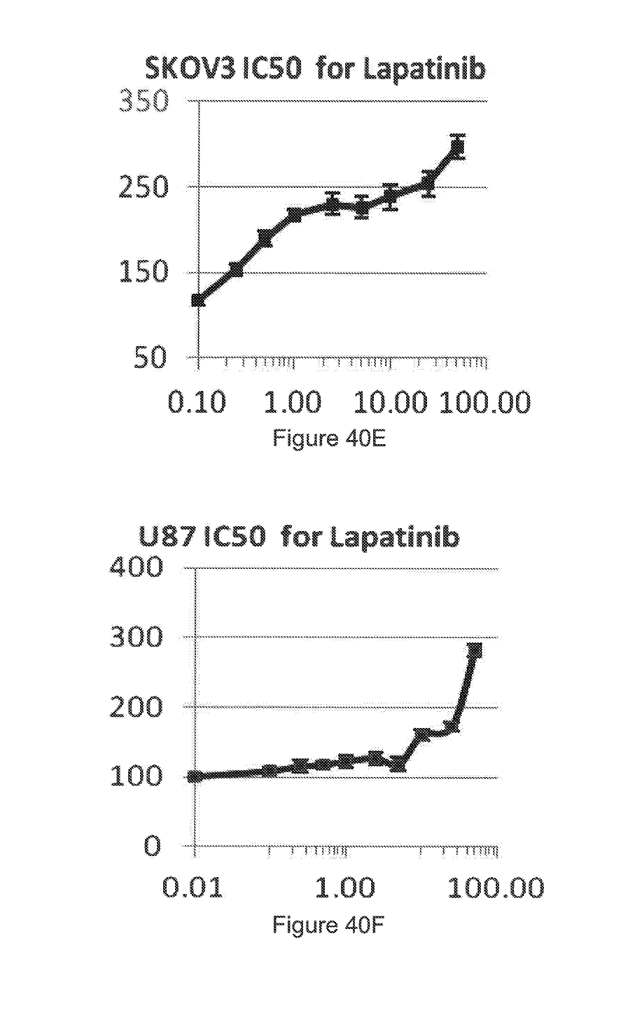

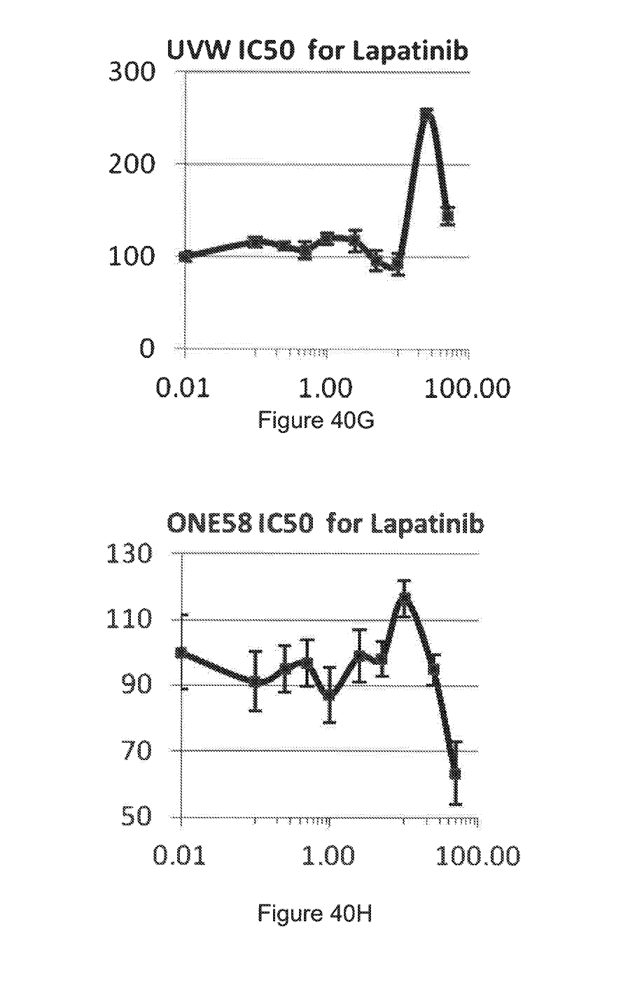

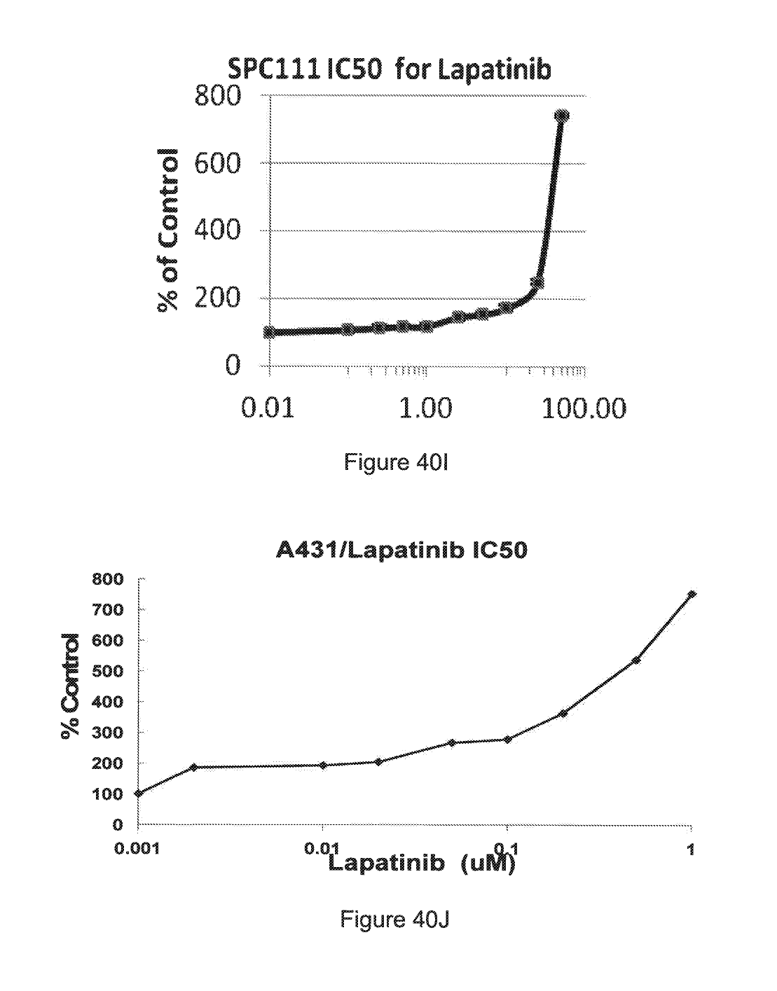

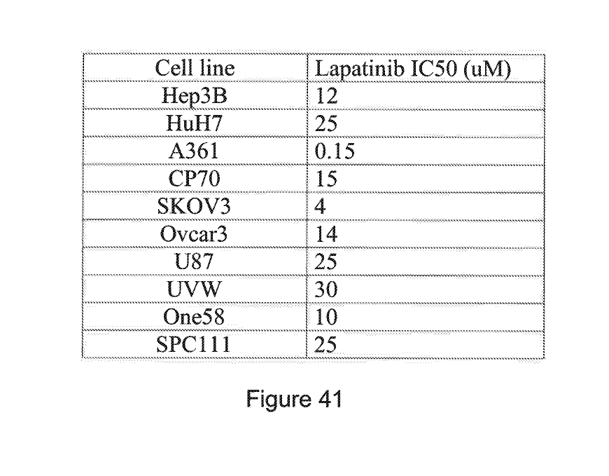

IC50 curves for each cell line tested are shown in FIG. 40 and the estimated IC50s are shown in Table 19 (FIG. 41). A431 cells displayed sensitivity to Lapatinib, with an IC50 of 0.15 μM. Lapatinib was toxic in all other cell lines at higher concentrations, with IC50s in the range of 4-30 μM (Table 19; FIG. 41). Lapatinib inhibits the growth of both EGFR- and ErbB2-overexpressing cells, with higher inhibitory activity against EGFR- or ErbB2-overexpressing cells (IC50 of 0.09-0.21 μM, including A431 with IC50=0.16 μM), compared with cells expressing low levels of EGFR or ErbB2 with IC50 of 3-12 μM (Rusnack et al. (2001) Mol Cancer Ther. 1:85-94).

### Combination Analysis

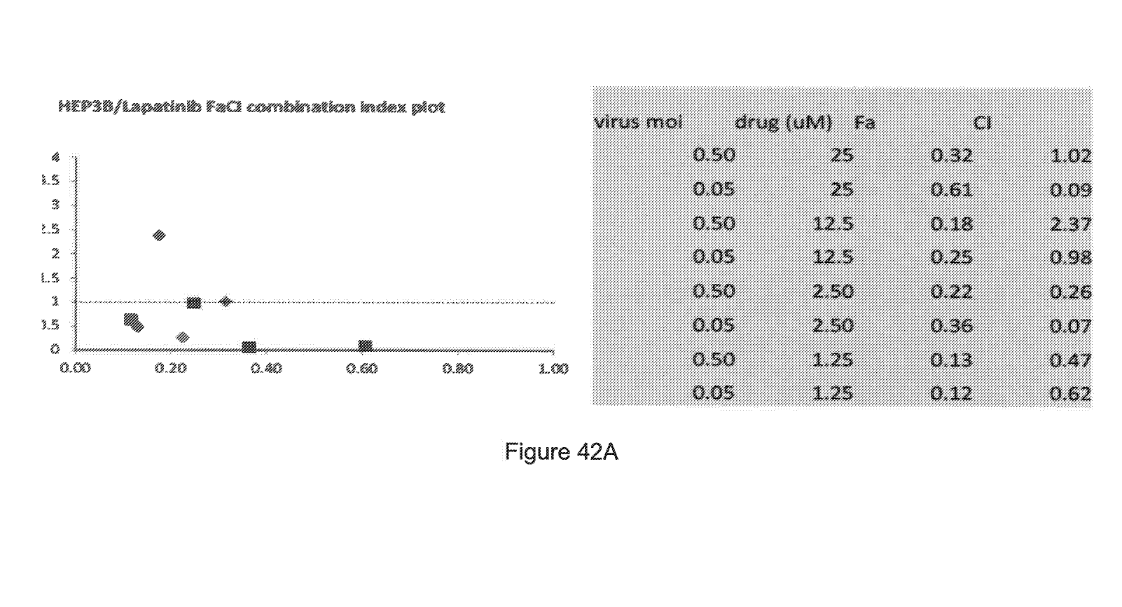

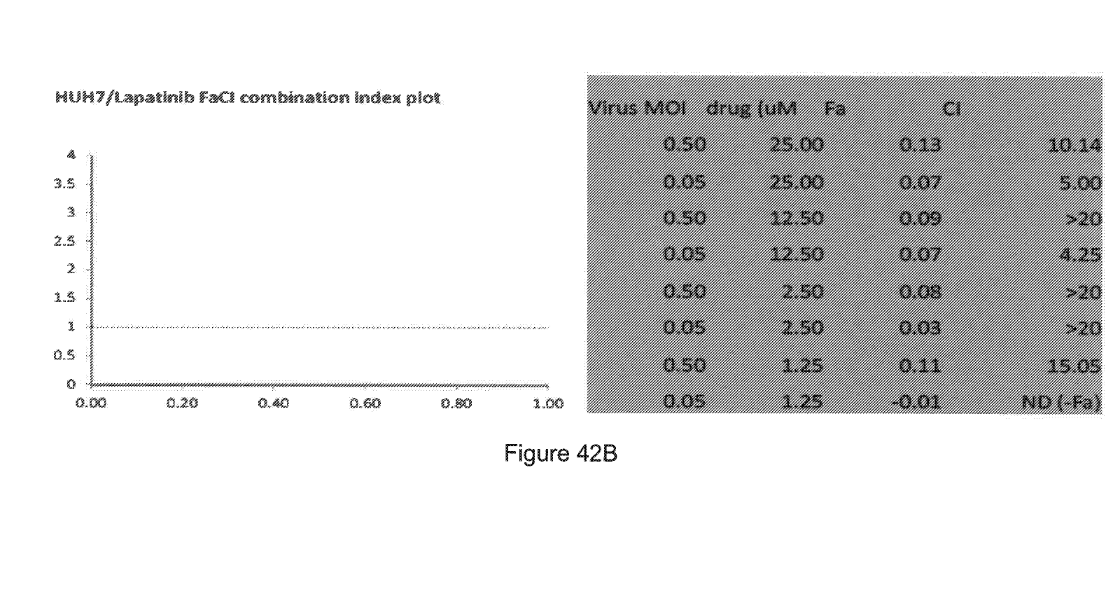

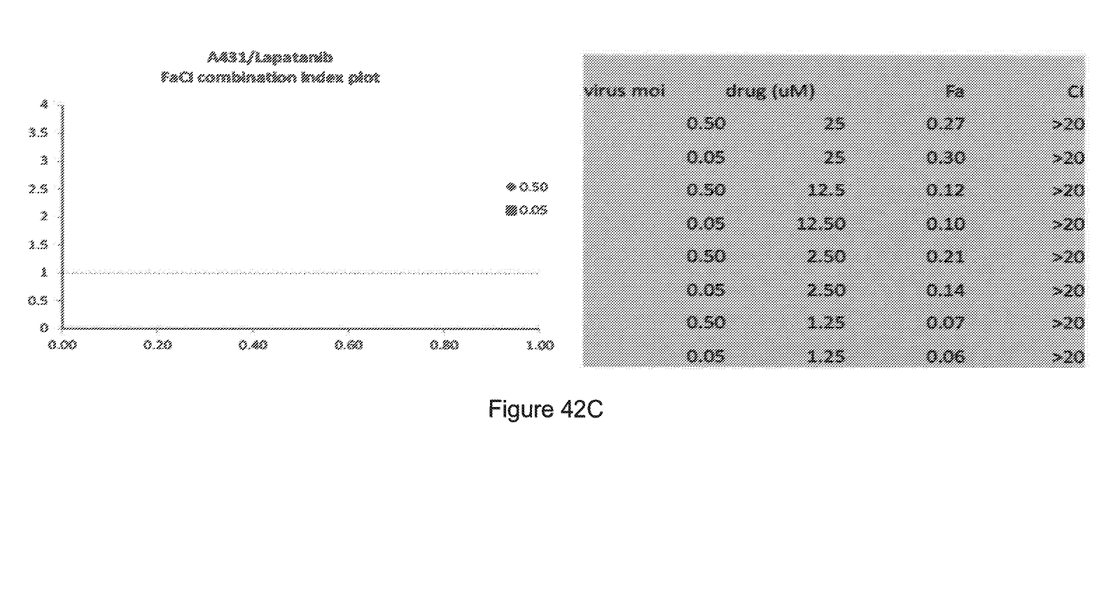

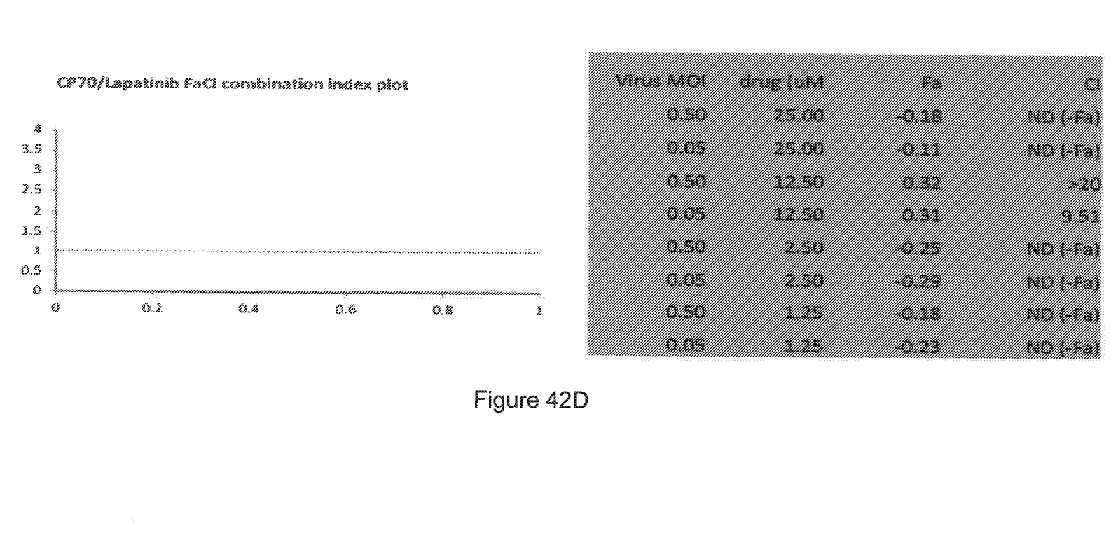

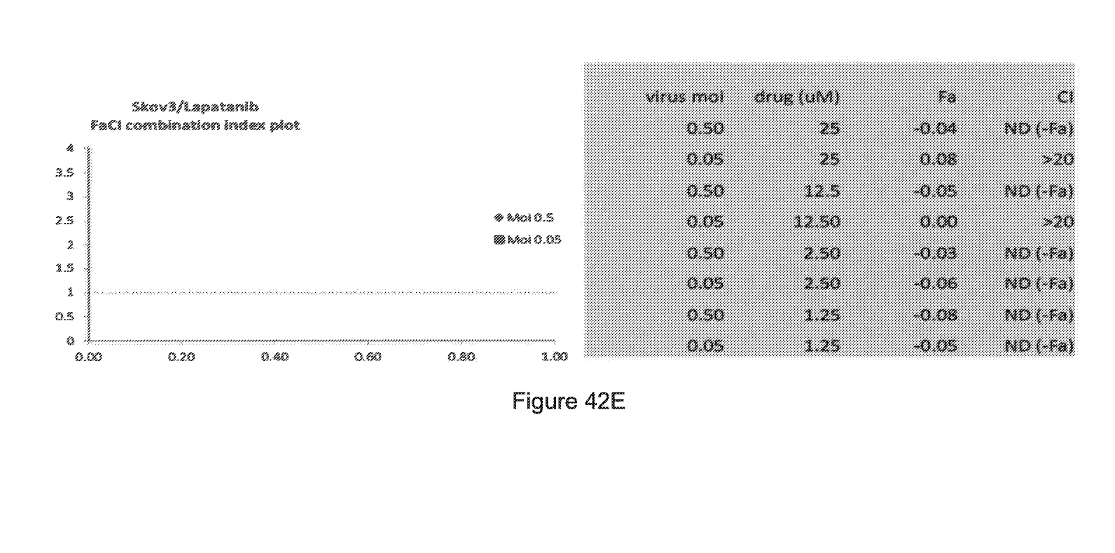

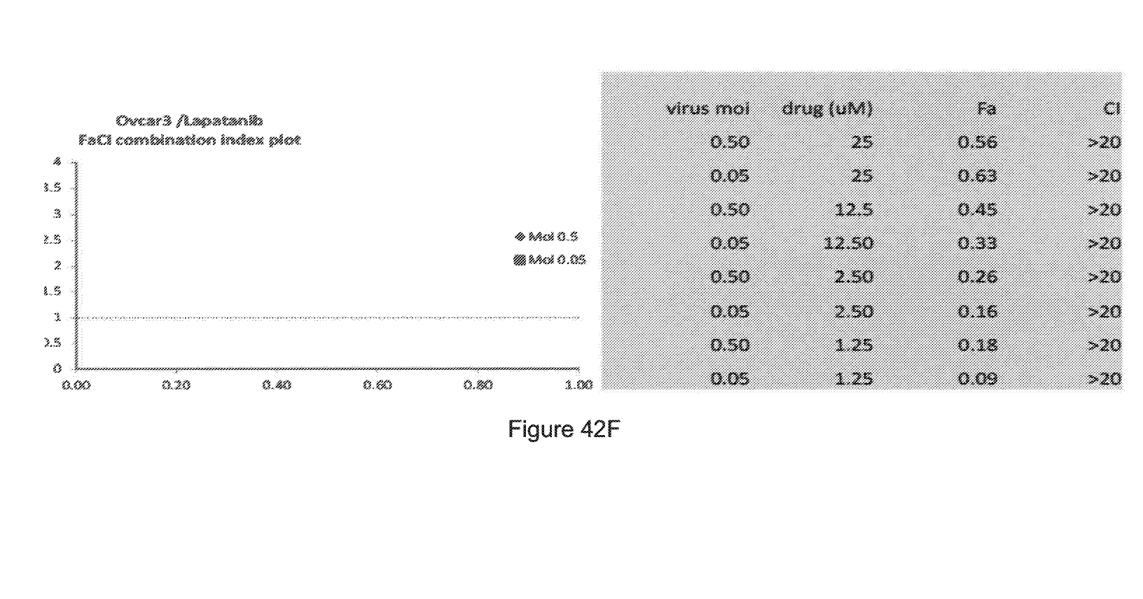

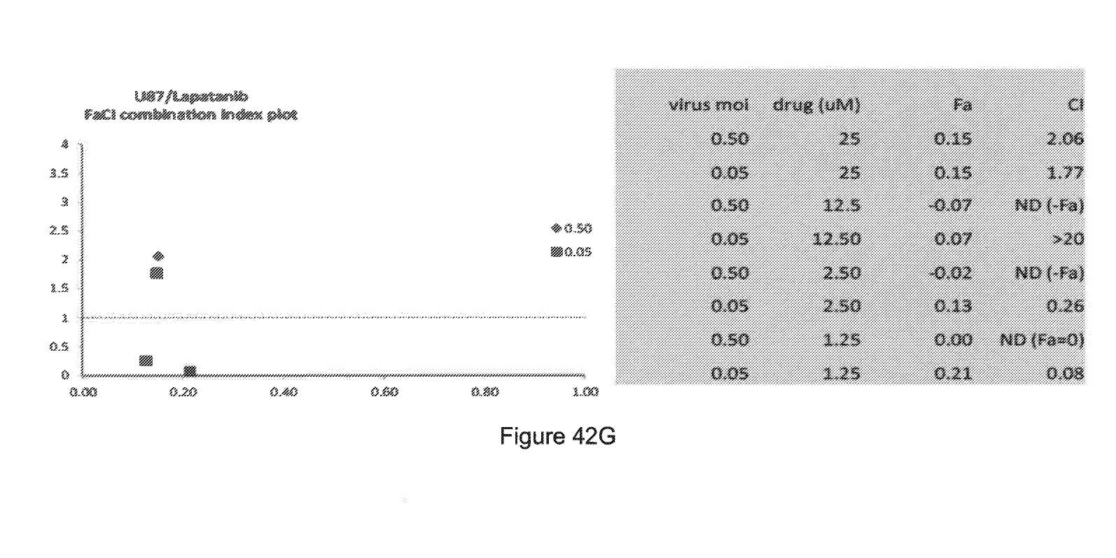

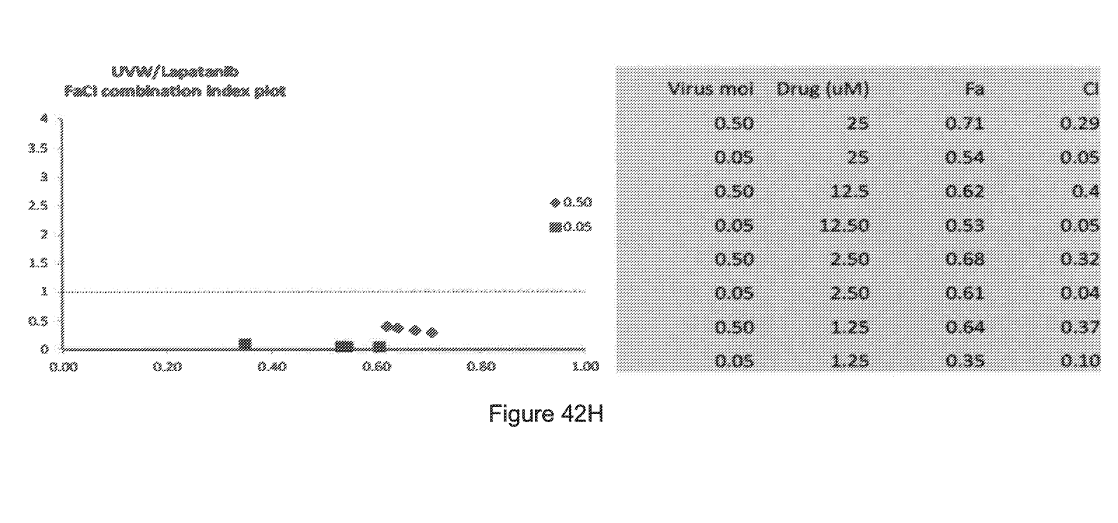

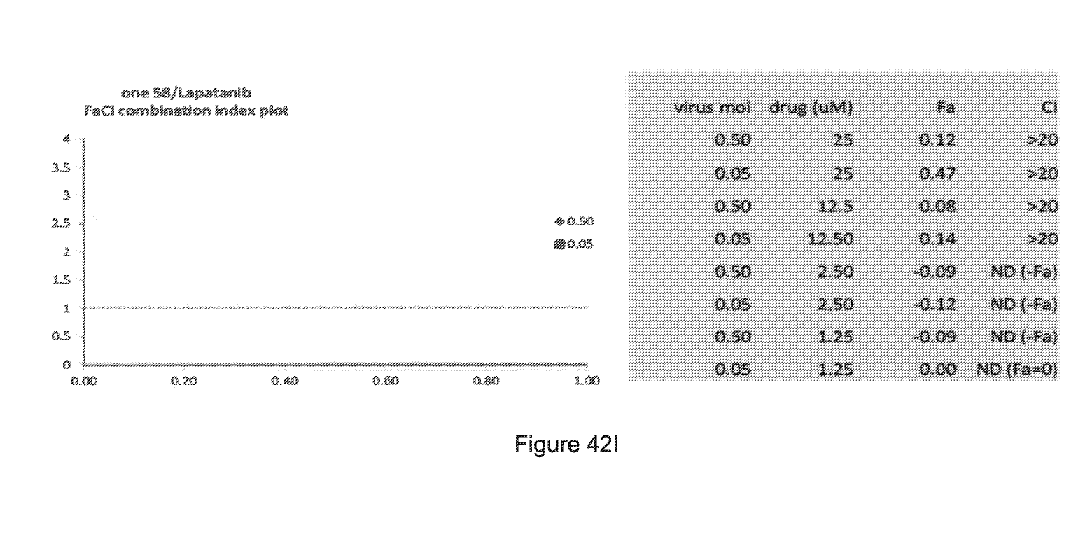

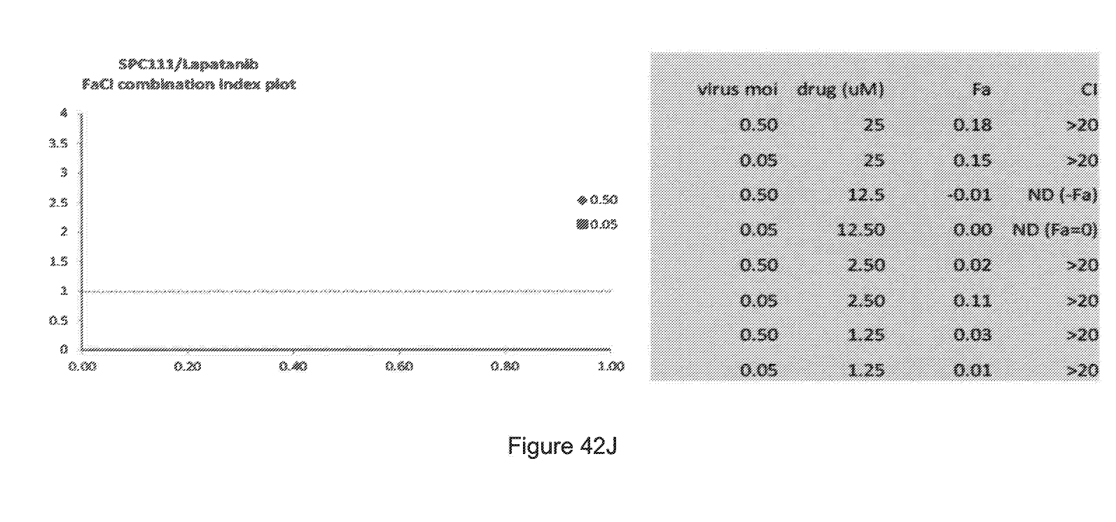

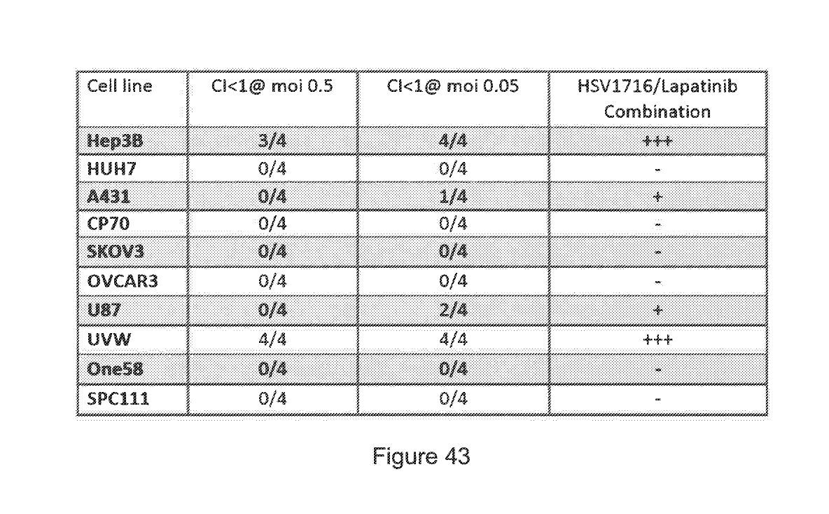

Lapatinib was tested at 4 different doses (25, 12.5, 2.5 and 1.25 μM) in Hep3B, HuH7, A431, CP70, Ovcar3, Skov3, U87, UVW, One58 and SPC111 cells, in combination with HSV1716 at moi 0.5 and 0.05. Representative average plots along with their respective Fa and Cl values are presented in FIG. 42 with synergies/antagonisms summarised in Table 20 (FIG. 43).

### Conclusions

Lapatanib was toxic in all of the cell lines, with A431 cells most sensitive.
Antagonism was detected in 8 of the 10 cell lines tested.
Lapatanib was strongly synergistic with HSV1716 at both moi in Hep3B and UVW cells.
Lapatanib displayed some synergy with HSV1716 in A431 (1/8, moi 0.05) and U87 (2/8, moi 0.05) cells.

### Example 10

### Sunitinib

Sunitinib was analysed in combination with HSV1716 in vitro as described, using the cell lines described. Toxicity was assessed in each well using DCP cell death assays. Toxic effects of Sunitinib on the virus were also assessed using HSV1716gfp, as described.

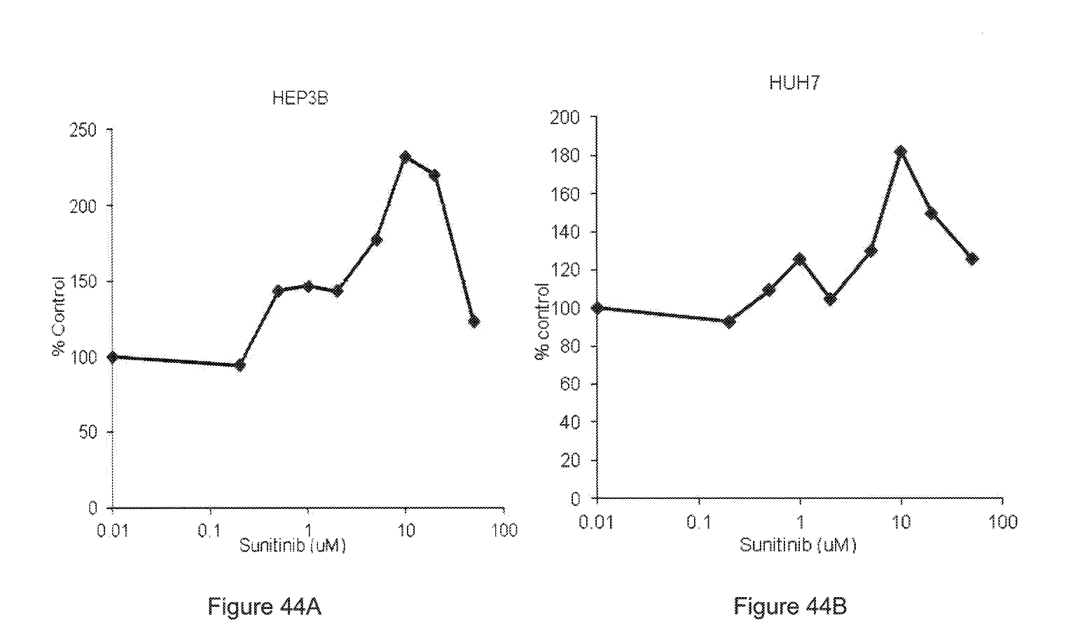

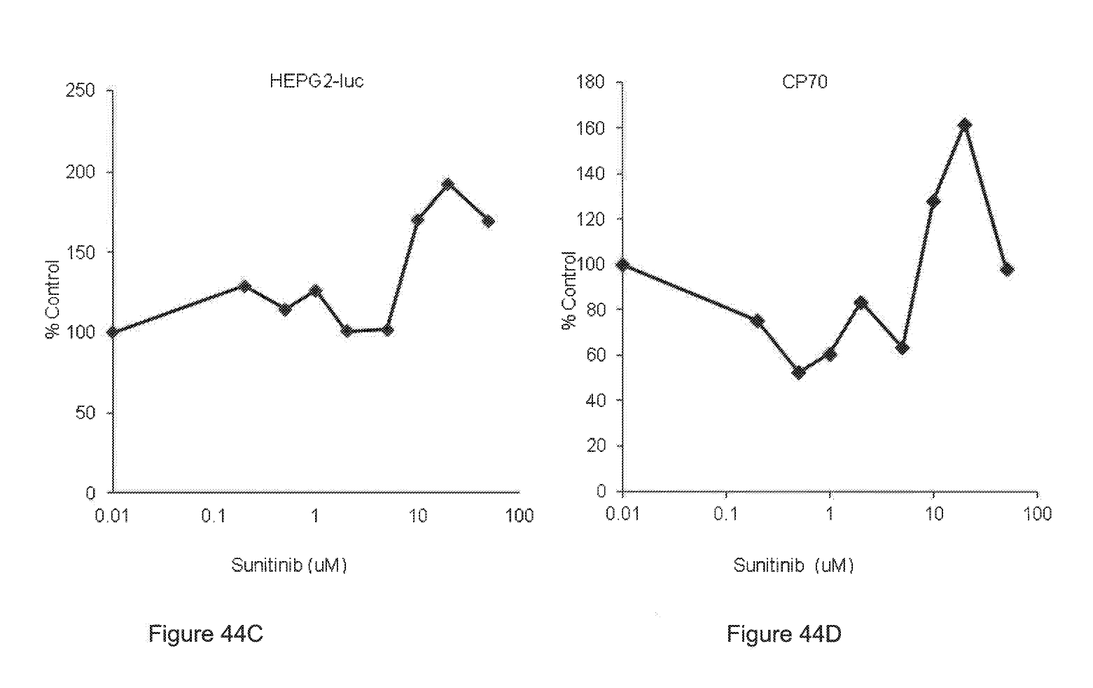

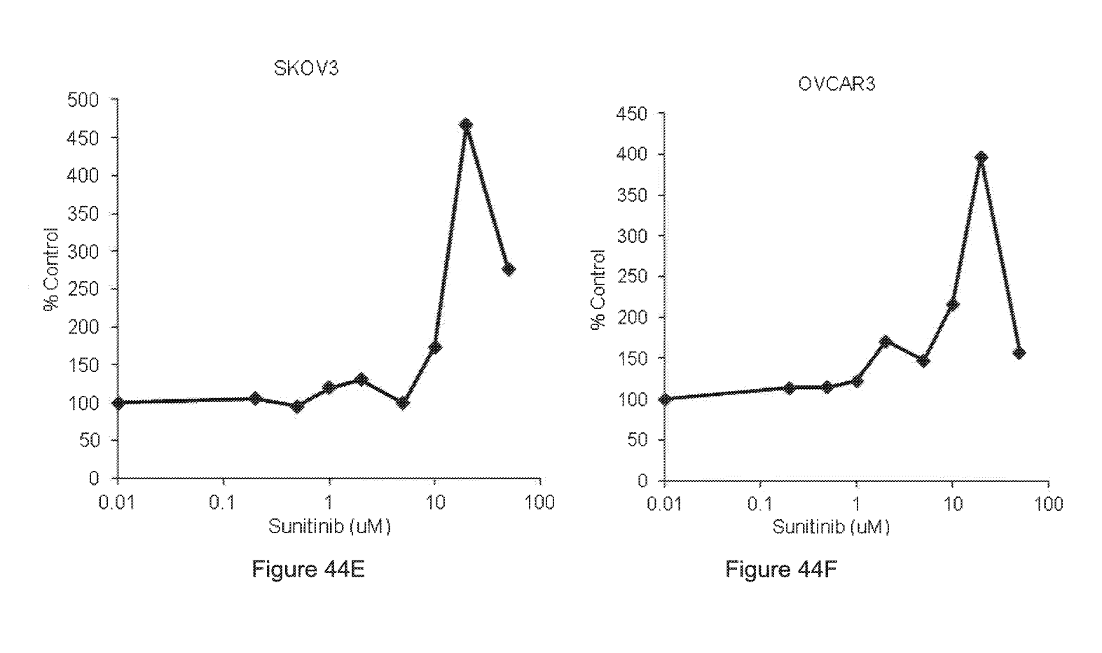

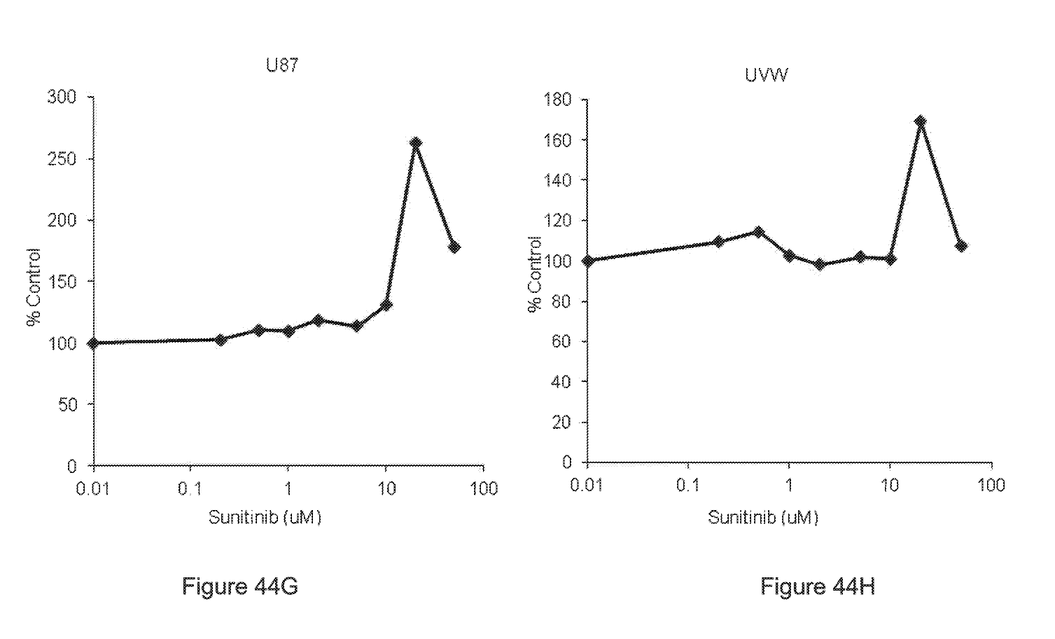

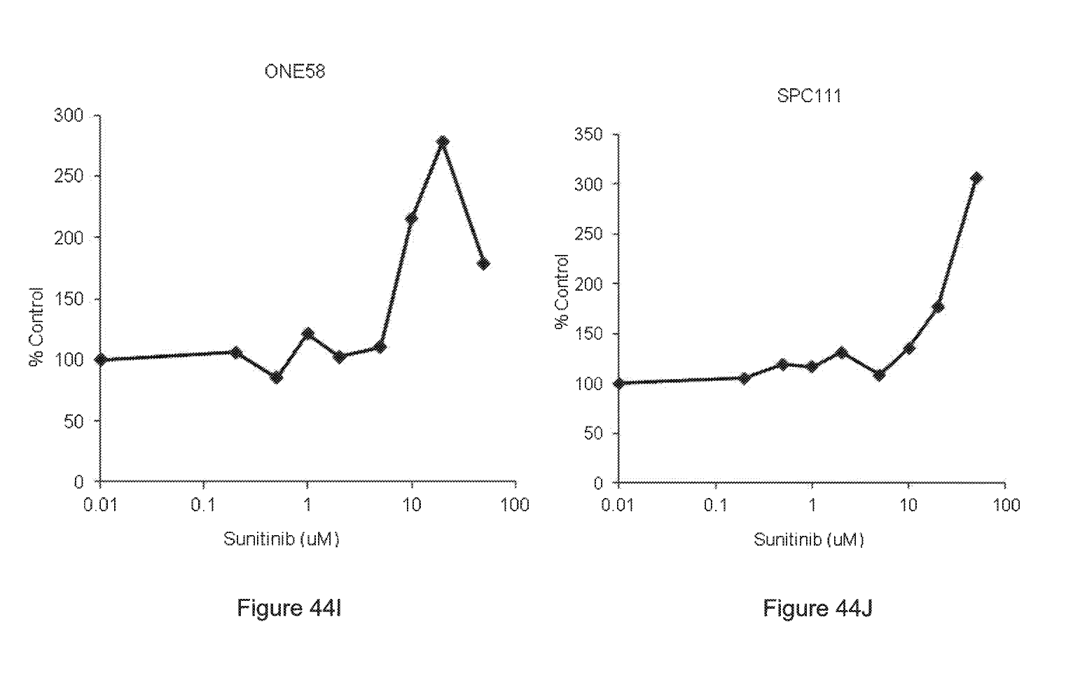

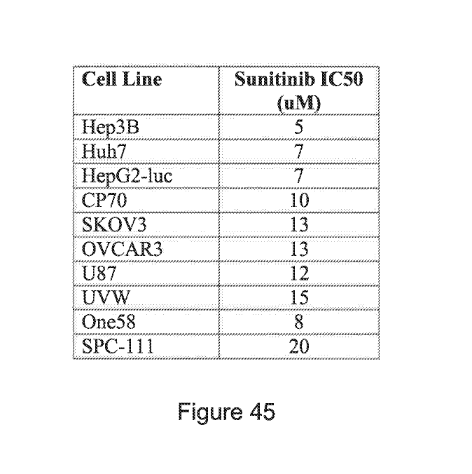

IC50 curves for each cell line tested are shown in FIG. 44 and the estimated IC50s are shown in Table 21 (FIG. 45). Sunitinib was toxic in all cell lines with IC50s in the range 0.5-15 μM with limited cell line variability in toxicity (Table 21; FIG. 42). The Sunitinib IC50 in several RCC cell lines is between 4 and 10 μM (Huang et al. (2010) Cancer Res 70(3); 1053-1062).

### HSV1716 Toxicity

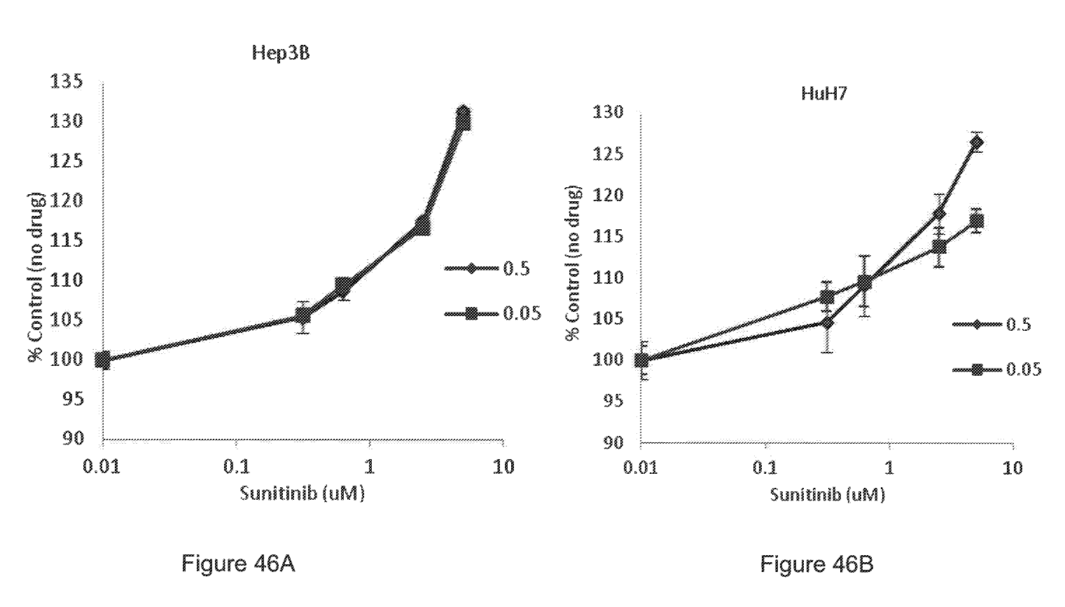

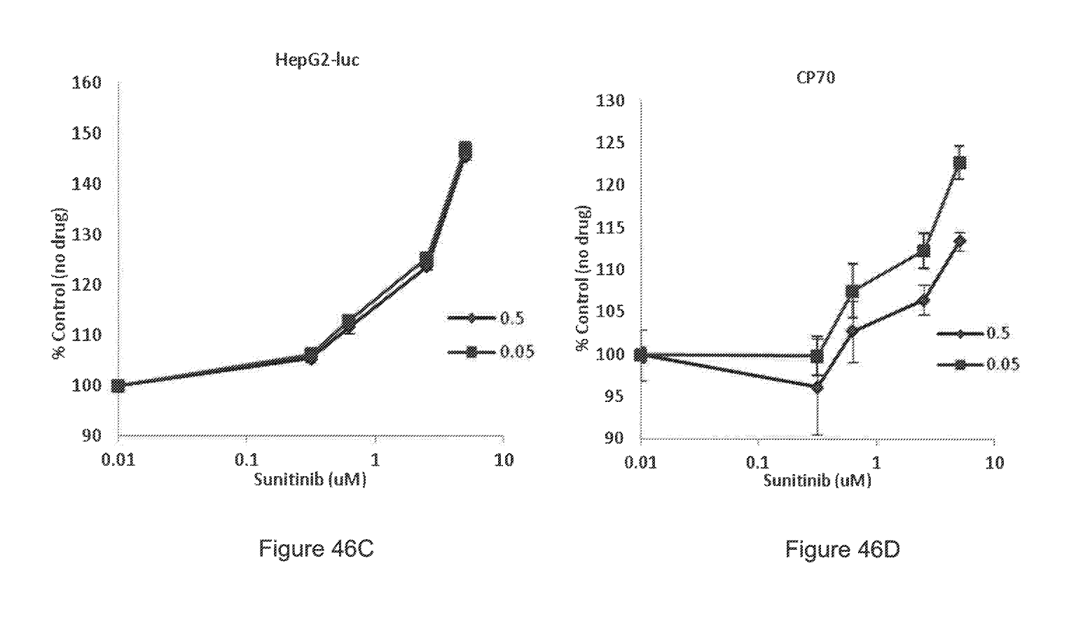

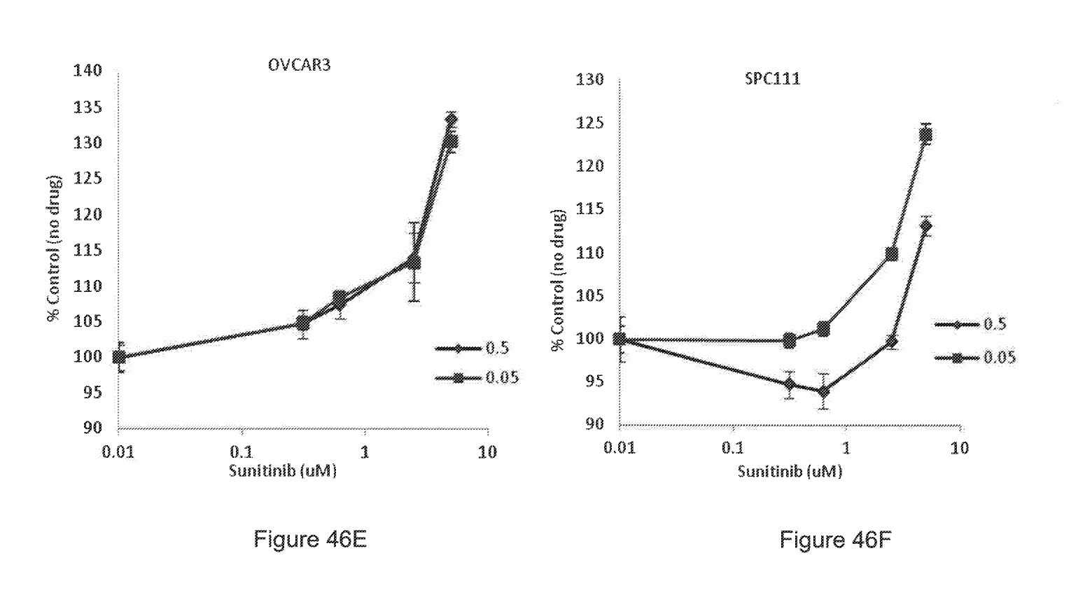

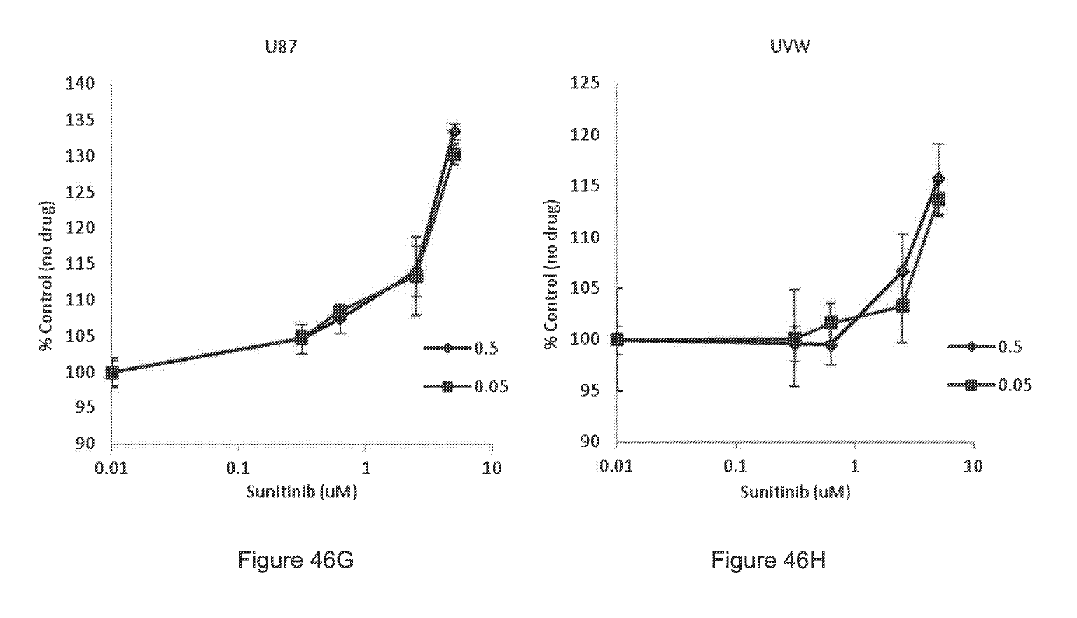

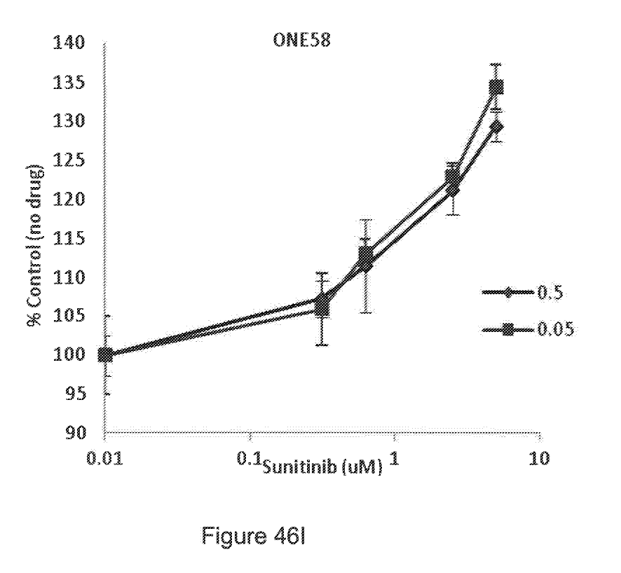

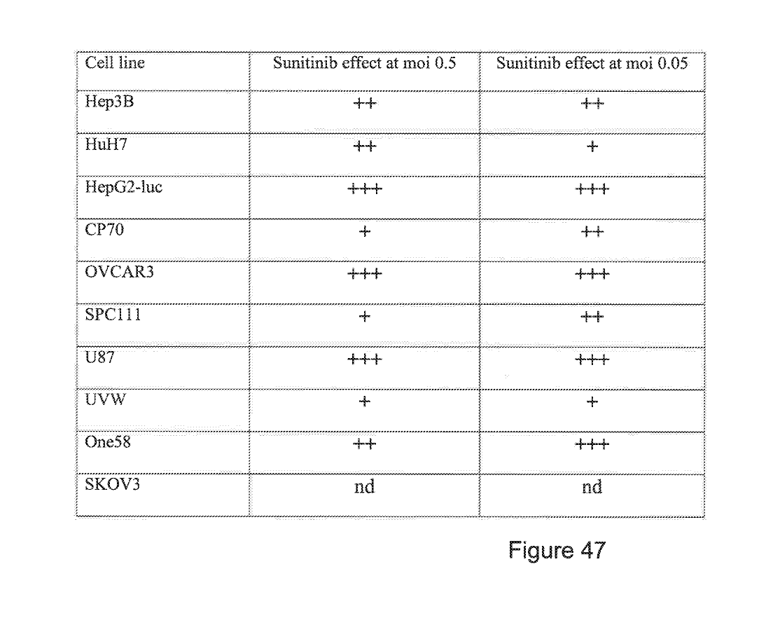

Sunitinib displayed no toxicity with respect to HSV1716. Sunitinib was incubated with cells plus virus at 5, 2.5, 0.625 and 0.312 μM. Drug-mediated virus toxicities are presented in the graphs in FIG. 46 as percentage of control cells (virus alone and no drug) in Hep3B, HuH7, HepG2-luc, CP70, Ovcar3, U87, UVW, One58 and SPC-111 cells. Virus was used at moi 0.5 or moi 0.05. In all 9 of the cell lines tested. Sunitinib increased GFP expression during HSV1716gfp infection at moi 0.5 and moi 0.05 (>15% variation from controls. Table 22; FIG. 47). There was little or no variation in GFP expression between moi 0.5 and moi 0.05 in Hep3B, HuH7, HepG2, Ovcar3, U87, and UVW cell lines at all Sunitinib concentrations. At the lower moi 0.05, the increase in GFP expression in CP70 and SPC111 cells was more pronounced than at moi 0.5. Increase in GFP expression in all cell lines suggests Sunitinib enhanced viral replication.

### Combination Analysis

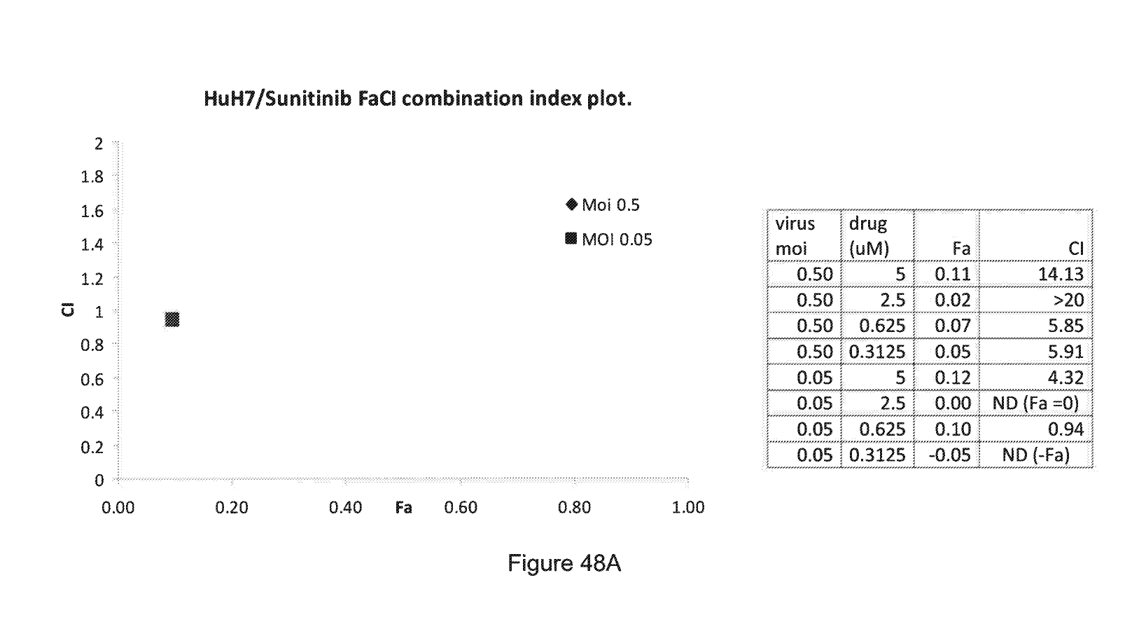

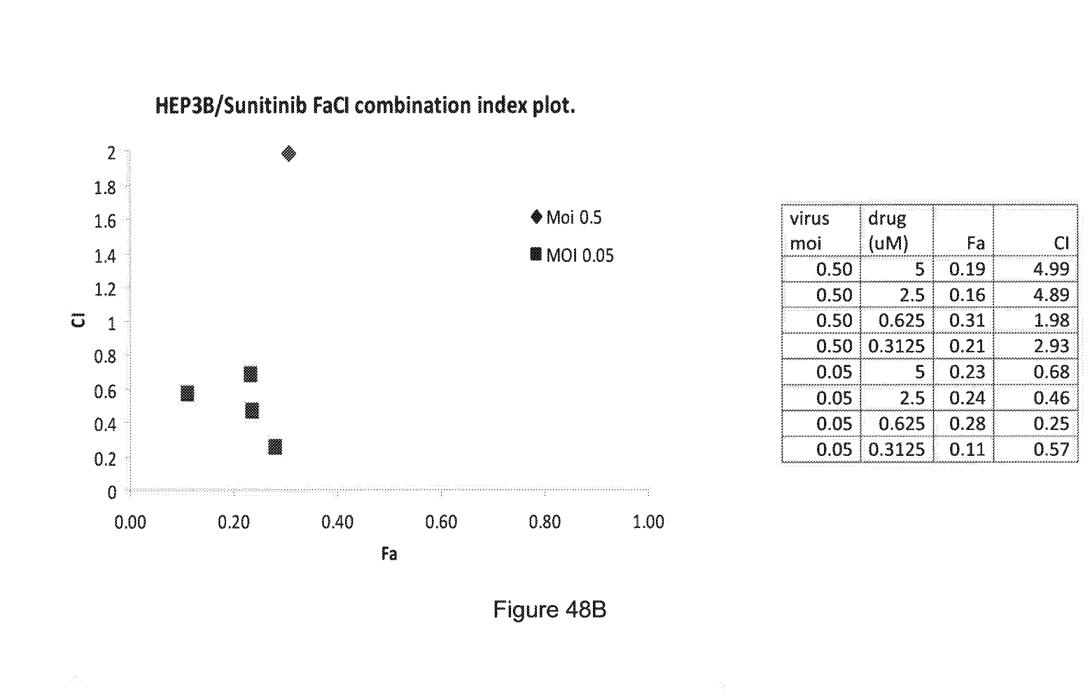

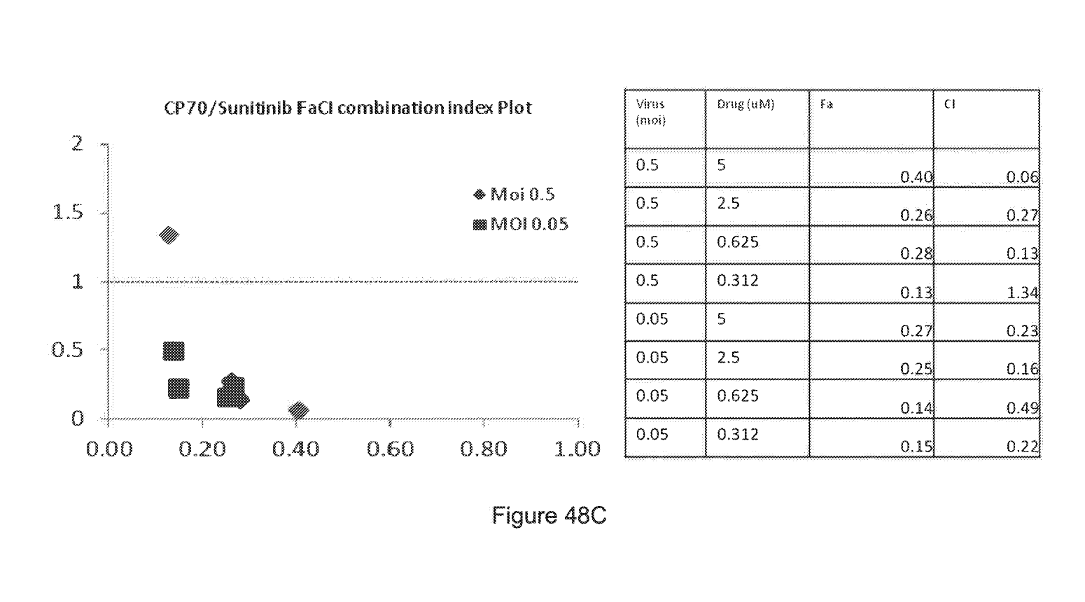

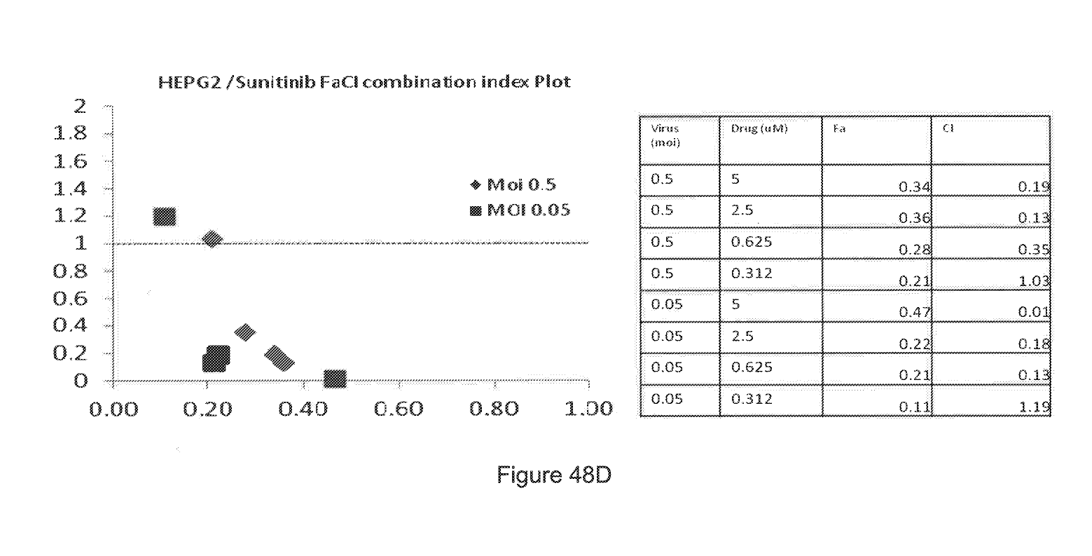

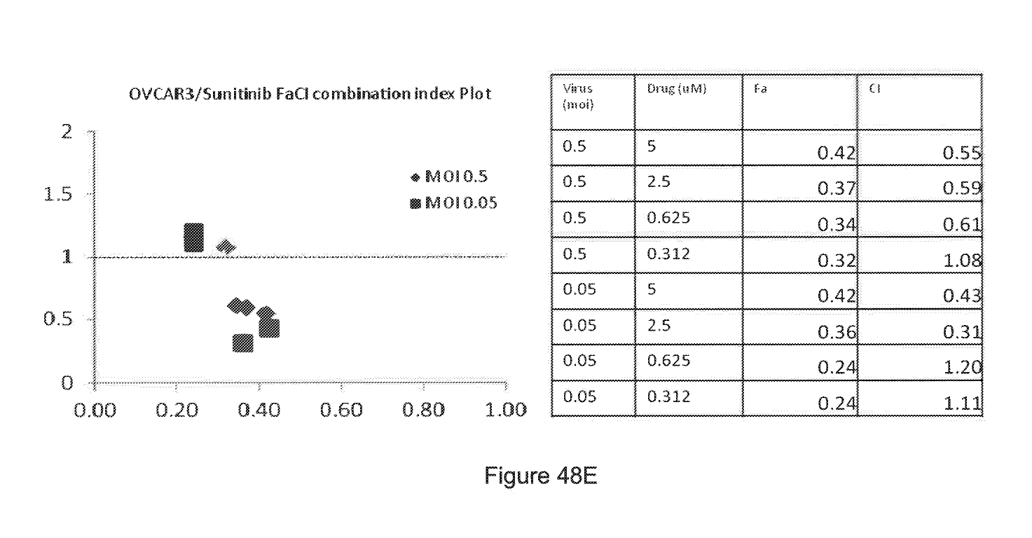

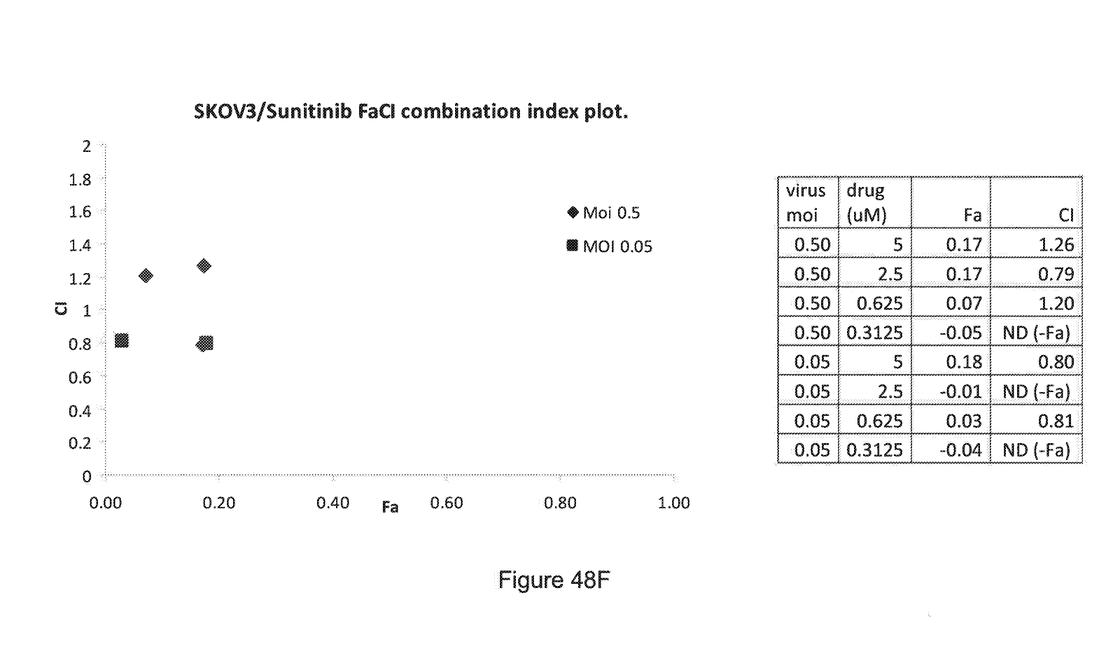

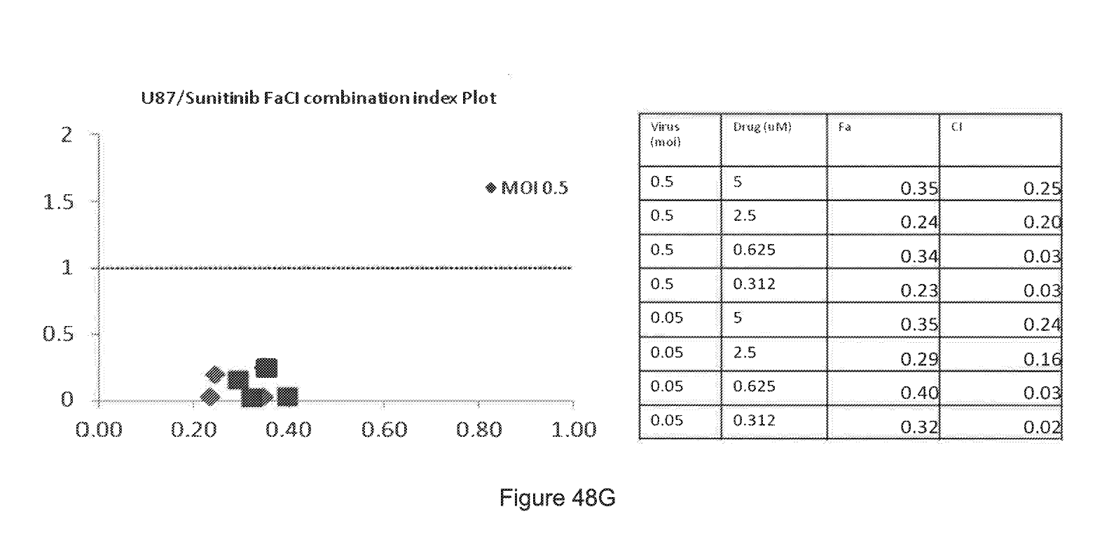

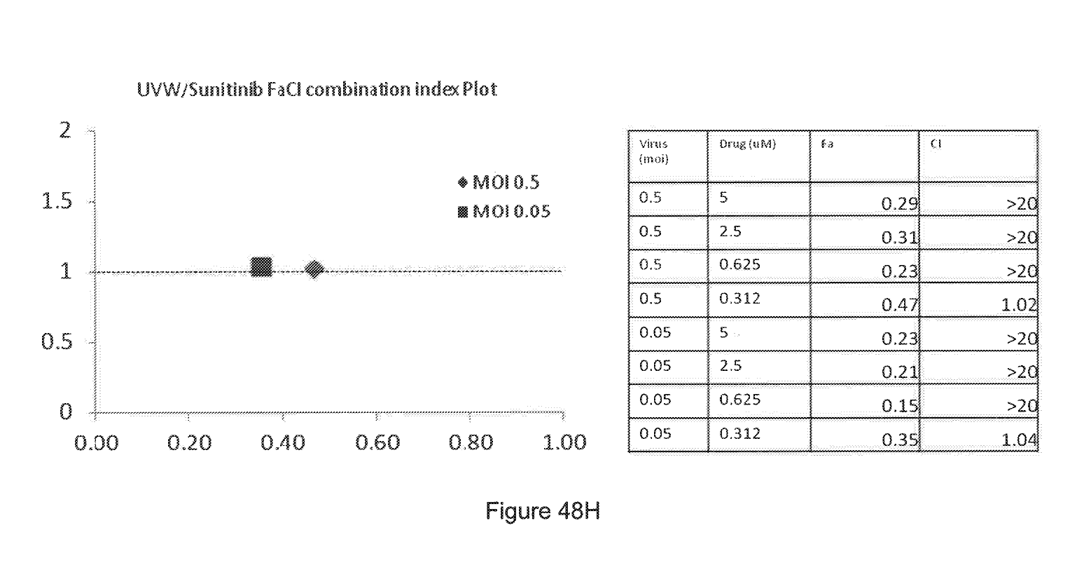

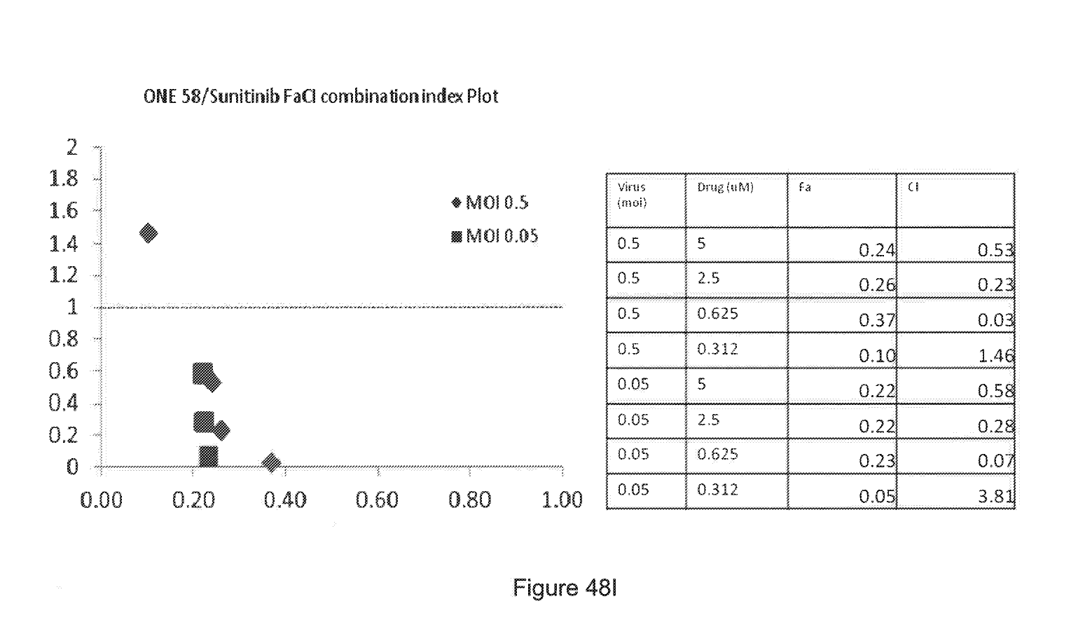

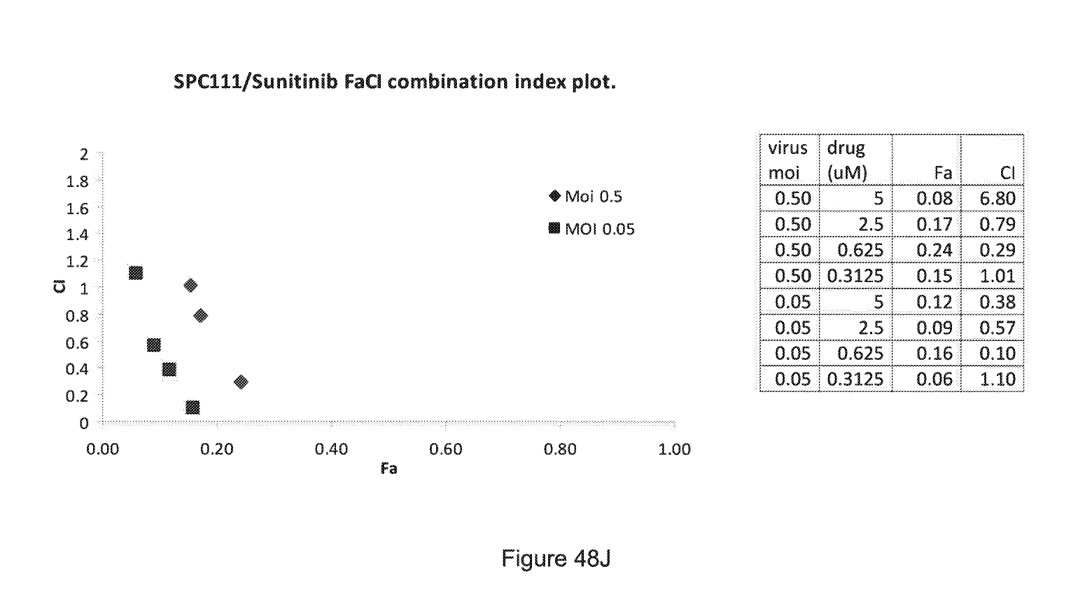

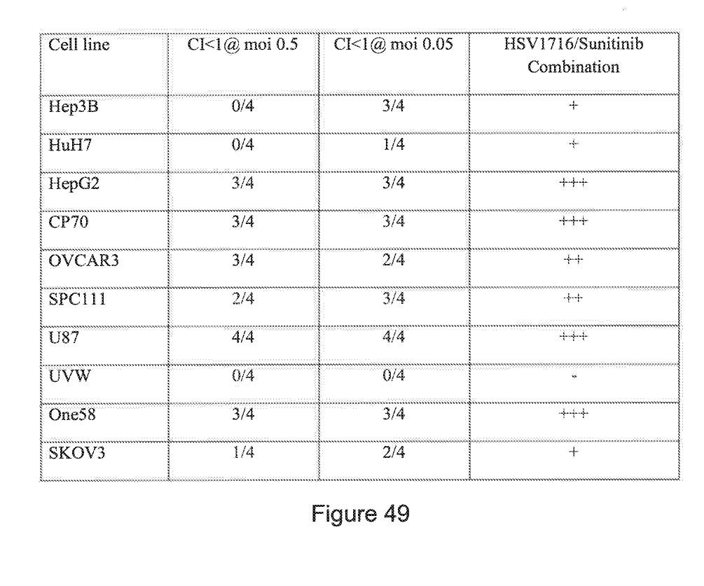

Sunitinib was tested at 4 different doses (5, 2.5, 0.625 and 0.312 μM) in Hep3B. HuH7, HepG2, Ovcar3, U87, UVW, One58, CP70 and SPC111 cells, in combination with HSV1716 at moi 0.5 and 0.05. Representative individual plots along with their respective Fa and Cl values are presented in FIG. 48, with synergies/antagonisms summarised in Table 23 (FIG. 49).

### Conclusions

Sunitinib is toxic in most of the cell lines tested.
Synergism of Sunitinib with HSV1716 was detected in 9 of the 10 cell lines tested.
Sunitinib was strongly synergistic with HSV1716 in U87, One58, SPC111, HepG2-luc and CP70 cells.
There was some evidence of synergy in Hep3B, HuH7 and SKOV3 cells.
Sunitinib was antagonistic with HSV1716 in UVW cells at both moi, and in Hep3B and HuH7 cells at moi 0.5.
Synergies occurred at both moi, moi 0.5 generated synergy in 7 of the 10 cell lines and moi 0.05 generated synergy in 9 of the 10 cell lines tested.
Sunitinib displayed no HSV1716 toxicity, and increased viral replication in all cell lines tested.

### Example 11

### Caspase Studies

### Methods

The Caspase-Glo 3/7 and 9 (Promega, UK) assay formats were used to measure caspase 3/7 and 9 activities in HSV1716-, TKi- or HSV1716+TKi-treated cells.

The Caspase-Glo 3/7 Assay is a homogeneous, luminescent assay that measures caspase-3 and -7 activities. The assay provides a luminogenic caspase-3/7 substrate, which contains the tetrapeptide sequence DEVD, in a reagent optimized for caspase activity, luciferase activity and cell lysis. Adding a single Caspase-GloR 3/7 Reagent in an “add-mix-measure” format results in cell lysis, followed by caspase cleavage of the substrate and generation of a “glow-type” luminescent signal produced by luciferase, and luminescence is proportional to the amount of caspase activity present. The Caspase-GloR 3/7 Reagent relies on the properties of a proprietary thermostable luciferase (Ultra-Glo™ Recombinant Luciferase), which is formulated to generate a stable “glow-type” luminescent signal and improve performance across a wide range of assay conditions.

The Caspase-Glo 9 Assay is a homogeneous luminescent assay that measures caspase-9 activity. The assay provides a luminogenic caspase-9 substrate in a buffer system optimized for caspase activity, luciferase activity and cell lysis. The addition of a single Caspase-Glo 9 Reagent in an “add-mix-measure” format results in cell lysis, followed by caspase cleavage of the substrate, and generation of a “glow-type” luminescent signal (produced by the luciferase reaction). The signal generated is proportional to the amount of caspase activity present. The Caspase-Glo 9 Reagent relies on the properties of a proprietary thermostable luciferase (Ultra-Glo™ Recombinant Luciferase), which generates the stable “glow-type” luminescent signal and improves performance across a wide range of assay conditions.

The Caspase-GloR 3/7 and 9 assays are designed for use with multiwell 96-, 384- and 1536-well formats. Cell washing, removing medium and multiple pipetting steps are not required.

The effects of HSV1716, TKi and HSV1716+TKi were tested using the cells from the cell lines described. 96-well plates were set up with two rows of each cell type at c.5000 cells/well. After 24 hours in culture, cells were treated in quadruplicate with HSV1716 at moi 1, TKi at 2.5 μM or HSV1716 at moi 1+2.5 μM TKi and incubated for 48 hours. Control cells received an equivalent volume of medium and 1 μM docetaxel was used as a positive control for apoptosis. Caspase 3/7 and caspase 9 activities were then measured as indicated above.

Following caspase 3/7 or caspase 9 activity measurement in each well, the values were corrected for the total number of cells in each well by assaying DCP using the CytoTox-Glo Cytotoxicity kit. The assay selectively detects dead cells but, with the addition of the Lysis Reagent provided with the kit, the CytoTox-Glo Cytotoxicity Assay also deliver the luminescent signal associated with the total number of cells in each assay well.

For analysis, the caspase 3/7 or caspase 9 light output readings were divided by the total DCP value for the respective well to correct for the number of cells in each well. GraphPad Prism 4.00 was used to graph the relative caspase 3/7 or caspase 9 activities and were then analysed in GraphPad Prism 4.00 using ANOVA with Tukey's multiple comparison test run as post-run test.

### Synergistic Combinations of HSV1716 and TKi Enhance Caspase 3/7 Activity

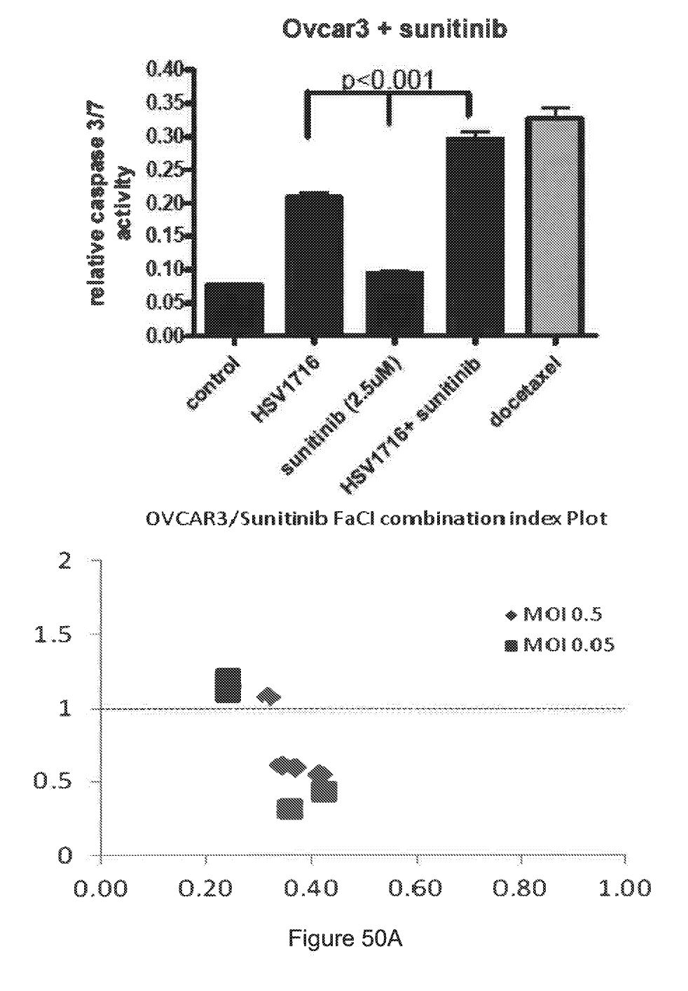

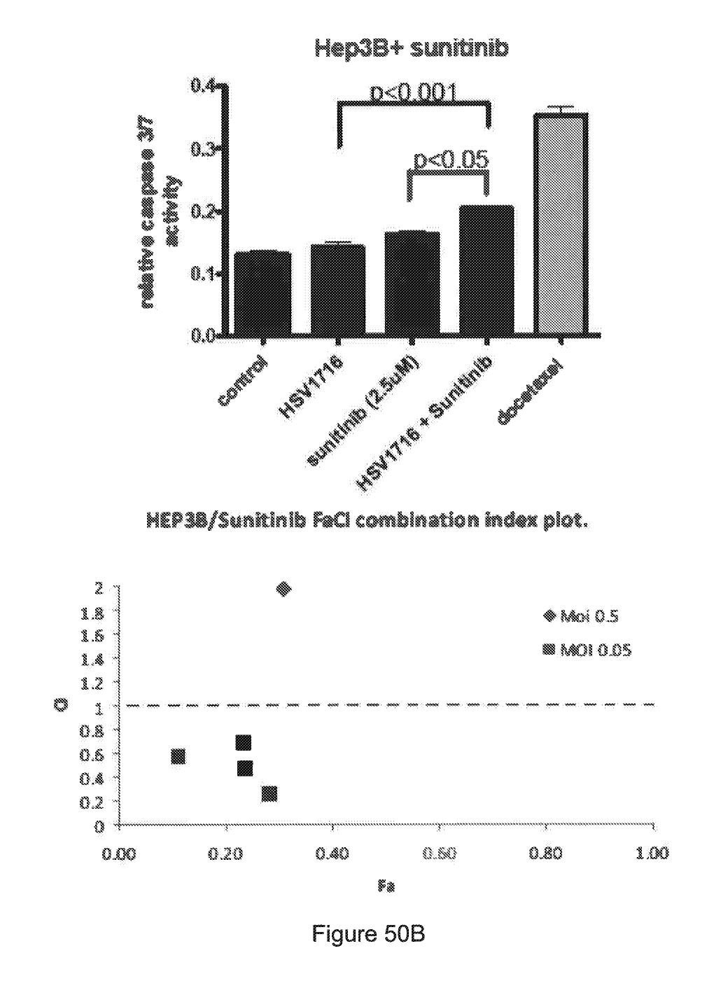

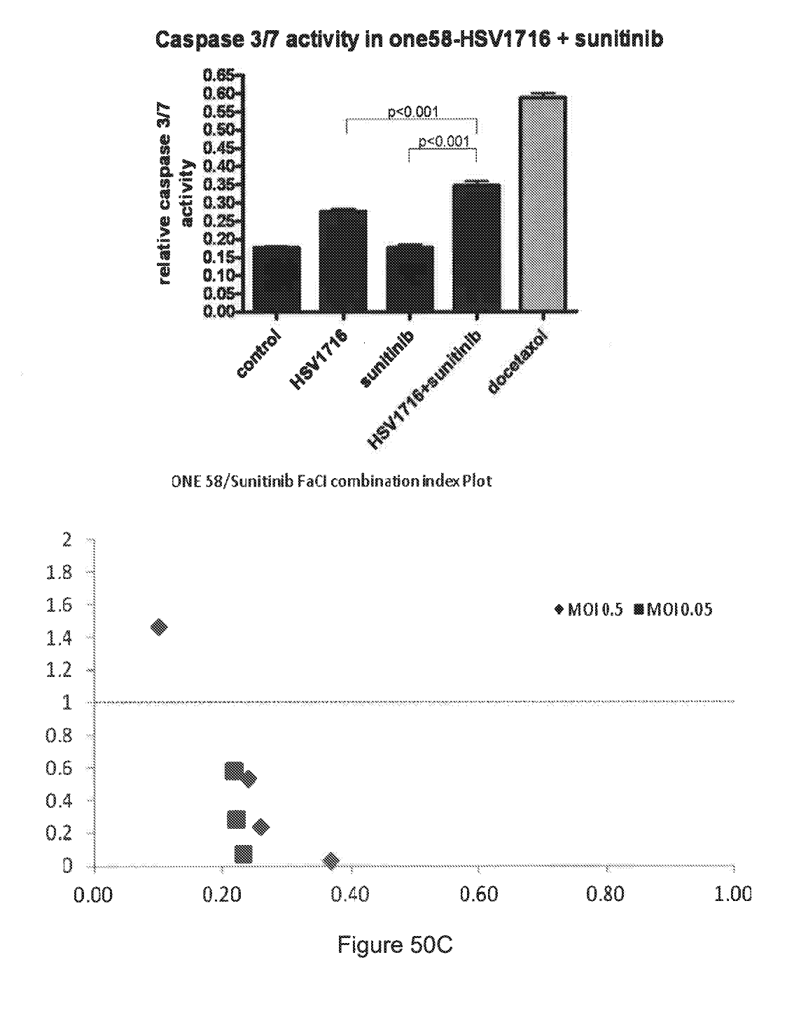

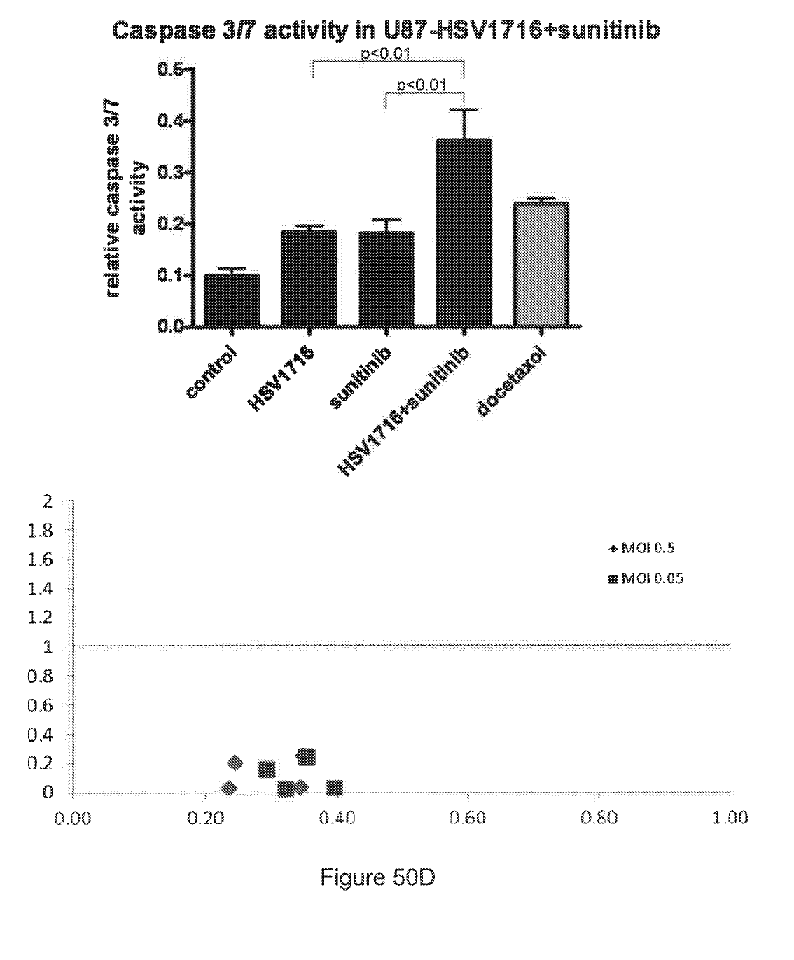

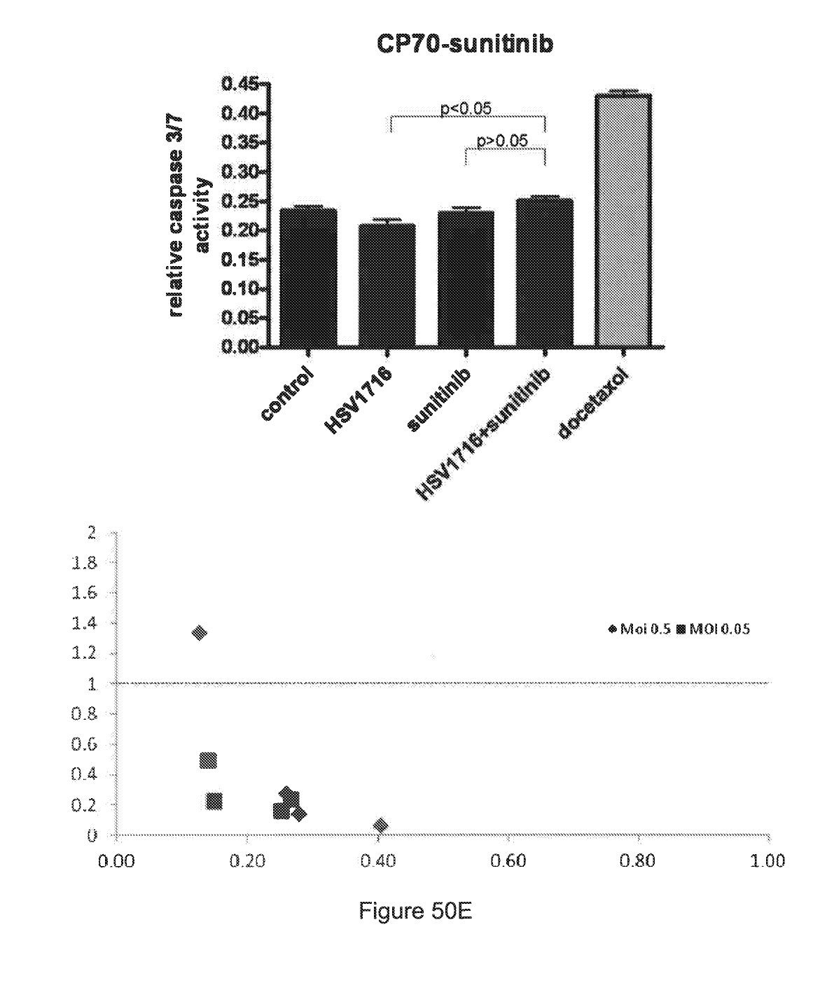

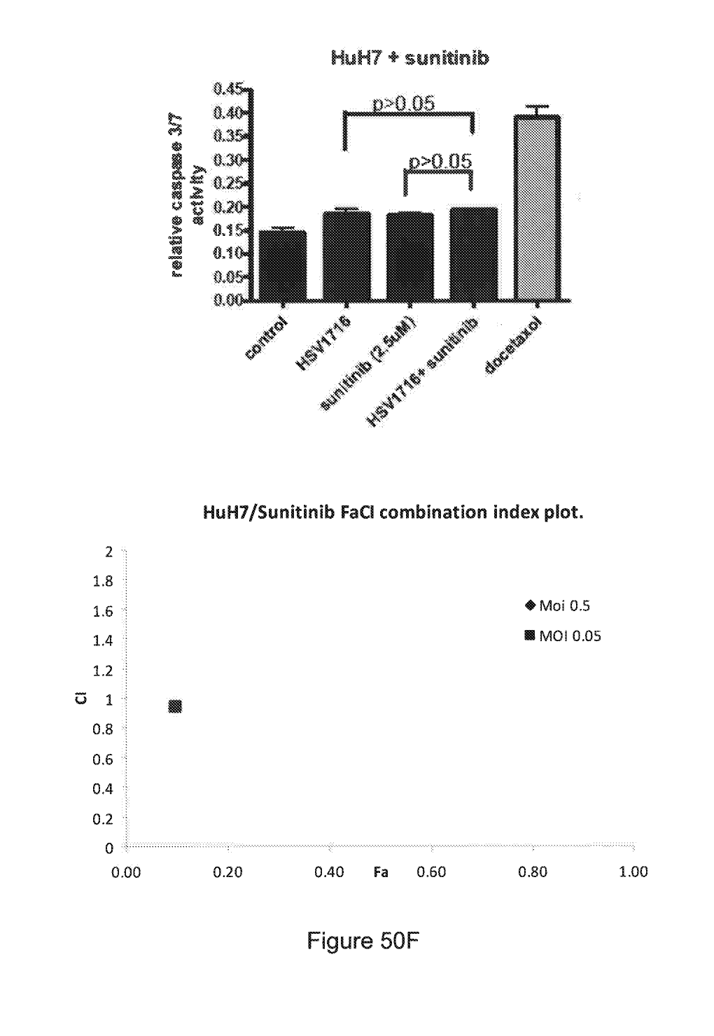

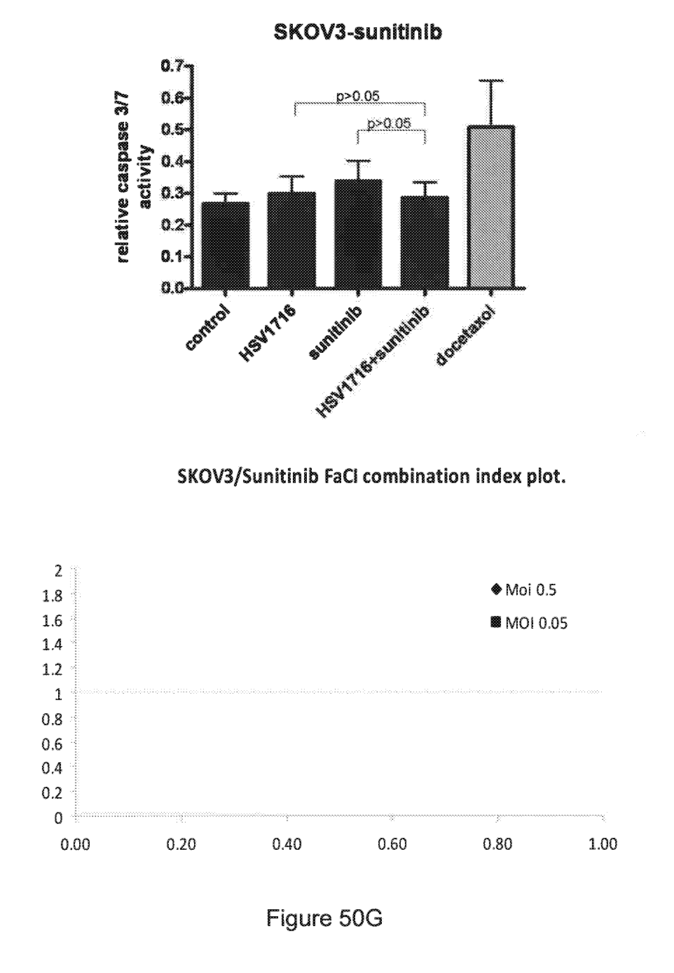

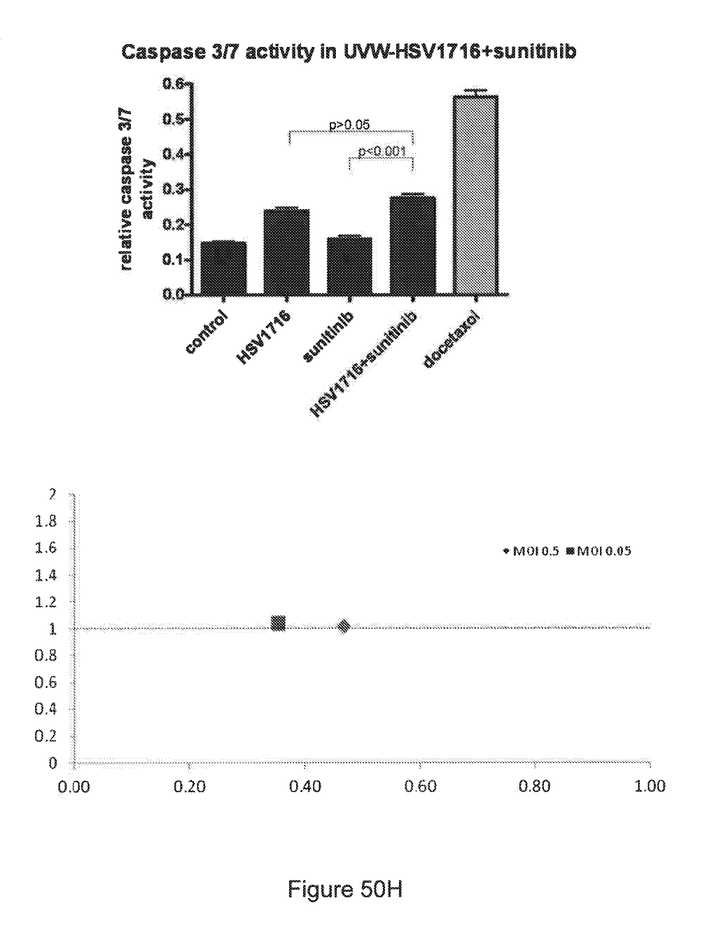

HSV1716 and Sunitinib combined synergistically to significantly enhance caspase 3/7 activity in Ovcar3, Hep3B, One58, U87 and CP70 cells over activity observed with HSV1716 or Sunitinib alone (FIGS. 50A-50E). By contrast, HSV1716 Sunitinib in HuH7, SKOV3 and UVW cells did not combine synergistically. For HuH7 and SKOV3 cells, the combination failed to enhance caspase 3/7 activity compared to HSV1716 or Sunitinib alone, whilst for UVW cells, the combination failed enhance activity compared to HSV1716 alone (FIGS. 50E-50H).

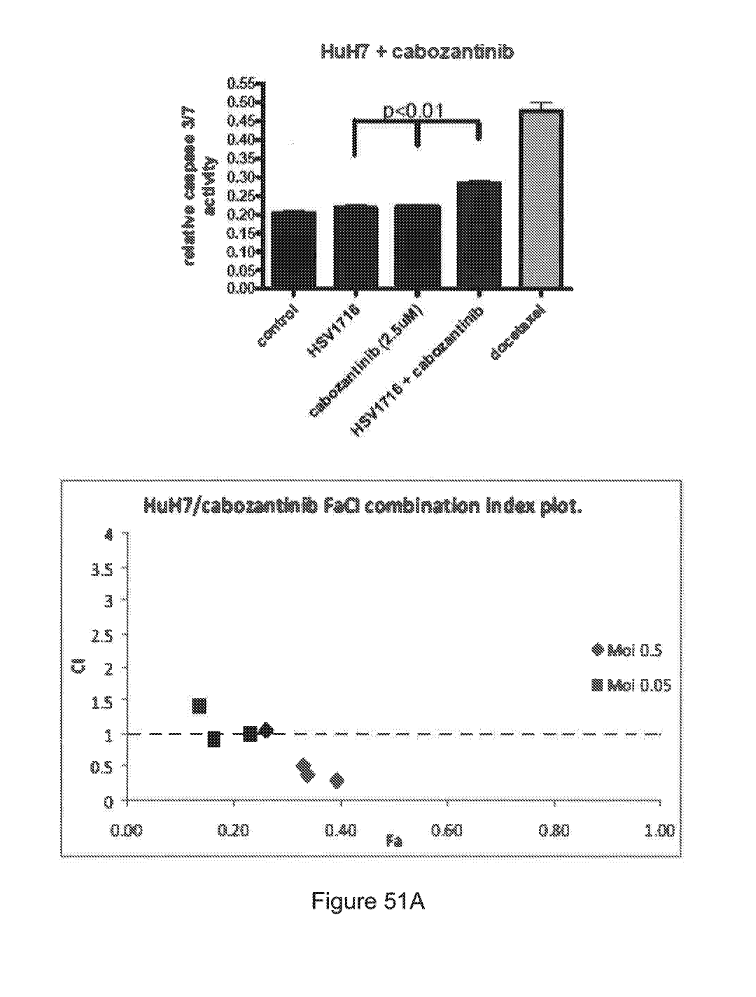

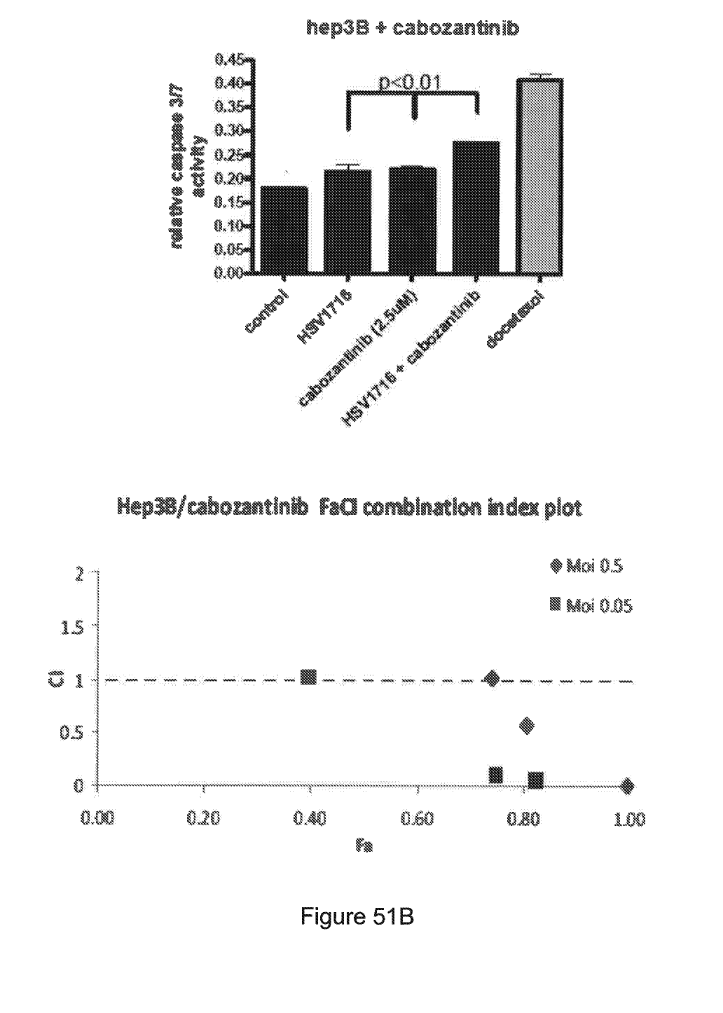

Synergistic combinations of HSV1716 and cabozantinb significantly enhanced caspase 3/7 activity (FIGS. 51A and 51B). In HuH7 and Hep3B cells, HSV1716 combined synergistically with cabozantinib and the combination significantly enhanced caspase 3/7 activity compared to either HSV1716 or cabozantinib alone.

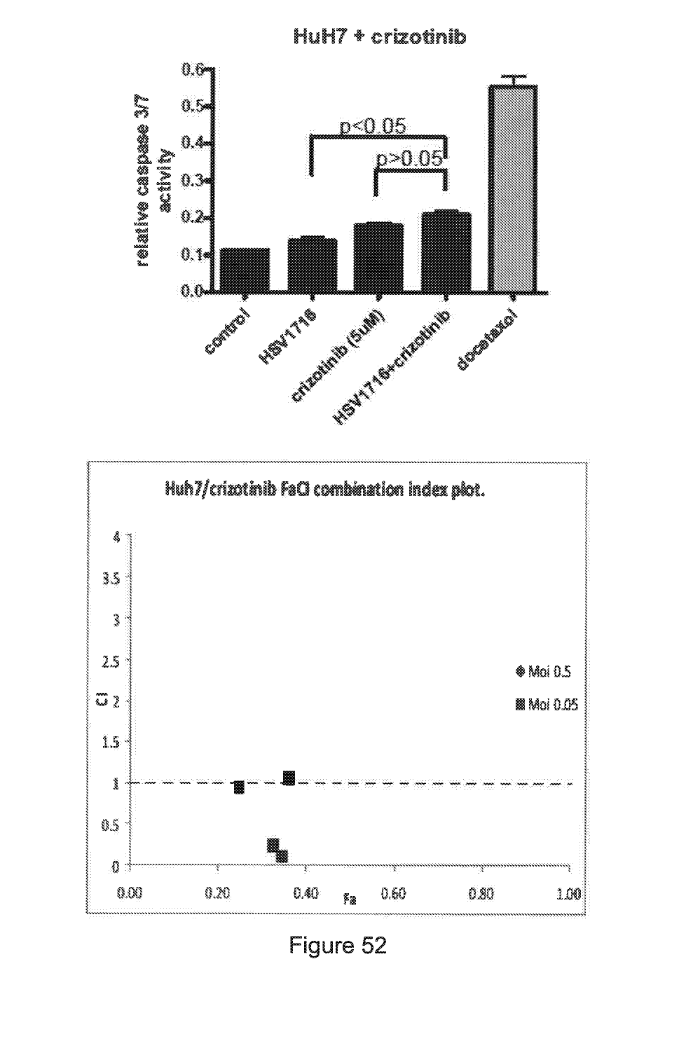

Similarly, HSV1716 combines synergistically with Crizotinib in HuH7 cells to significantly enhances caspase 3/7 activity (FIG. 52).

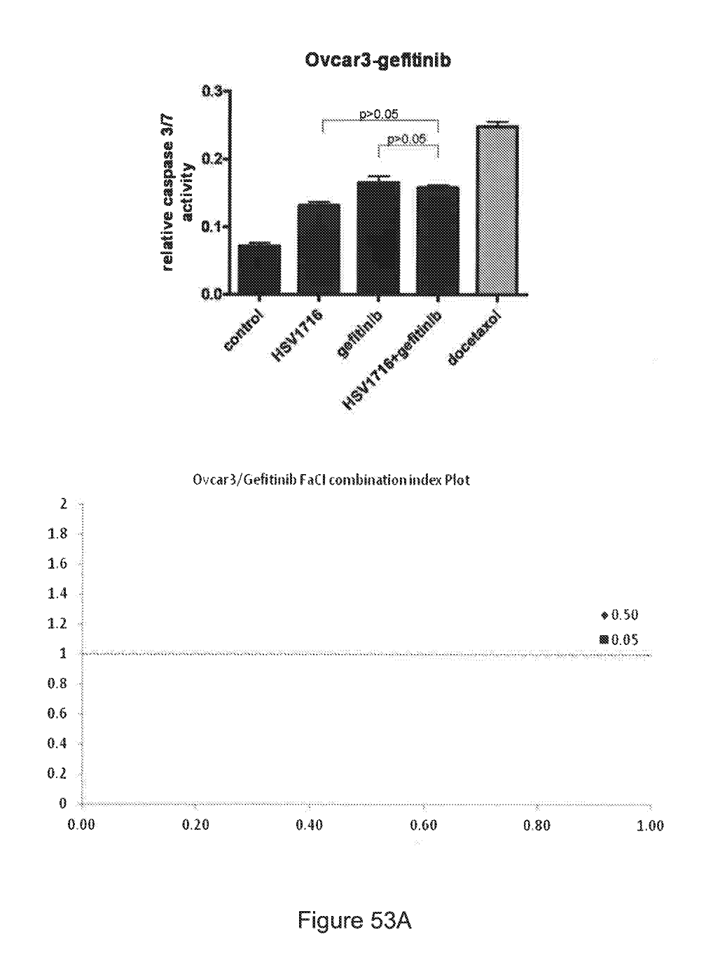

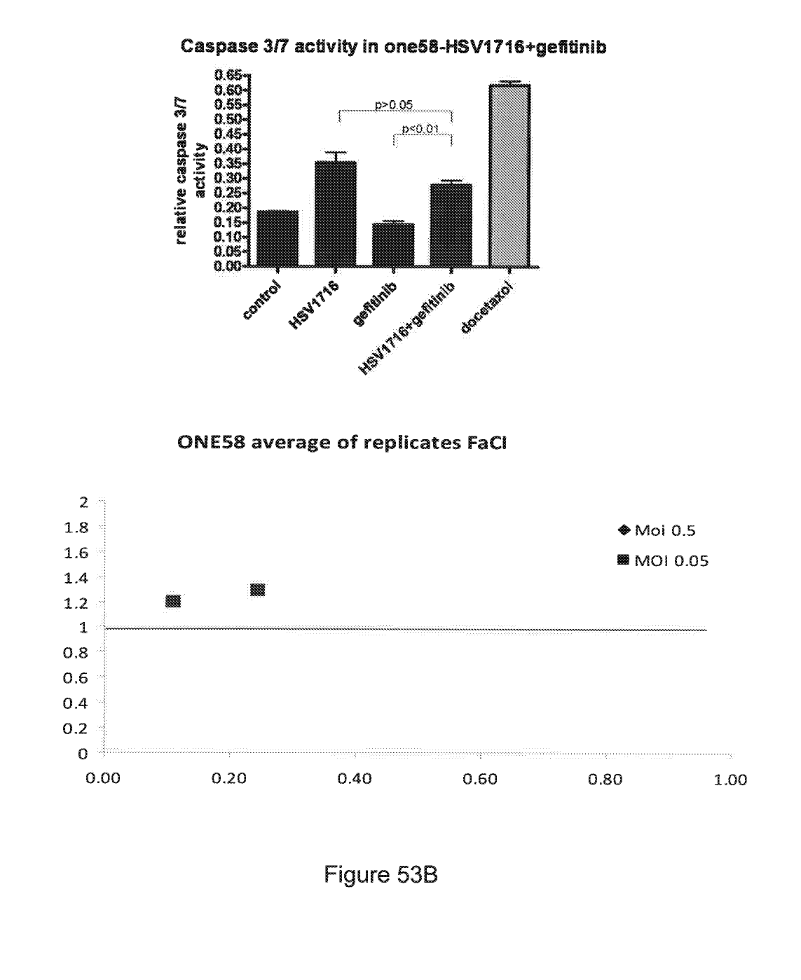

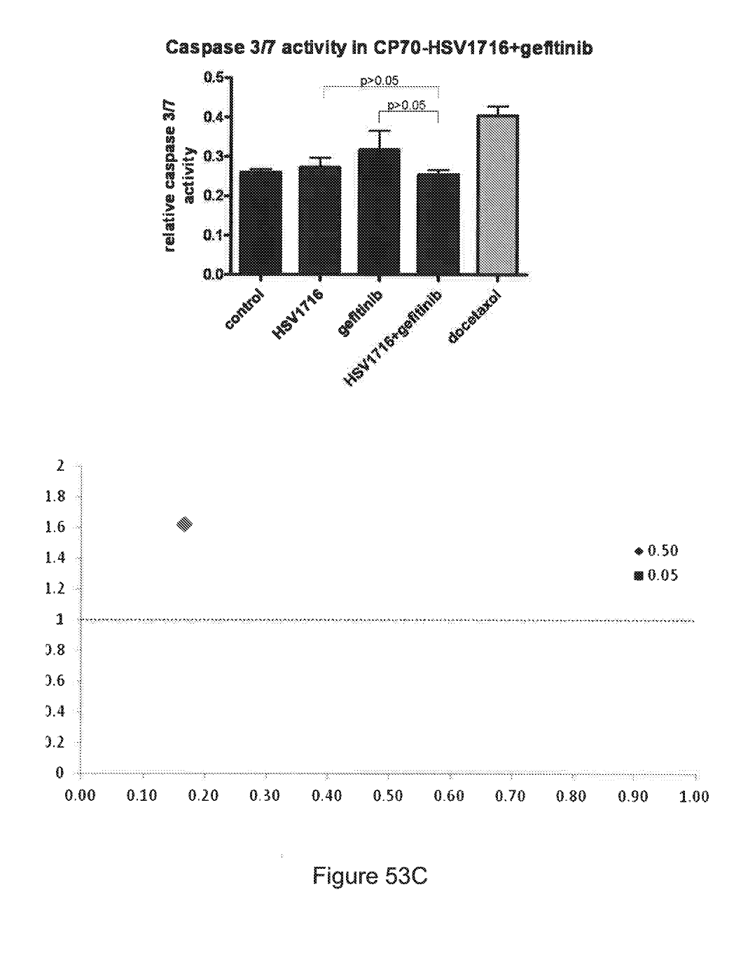

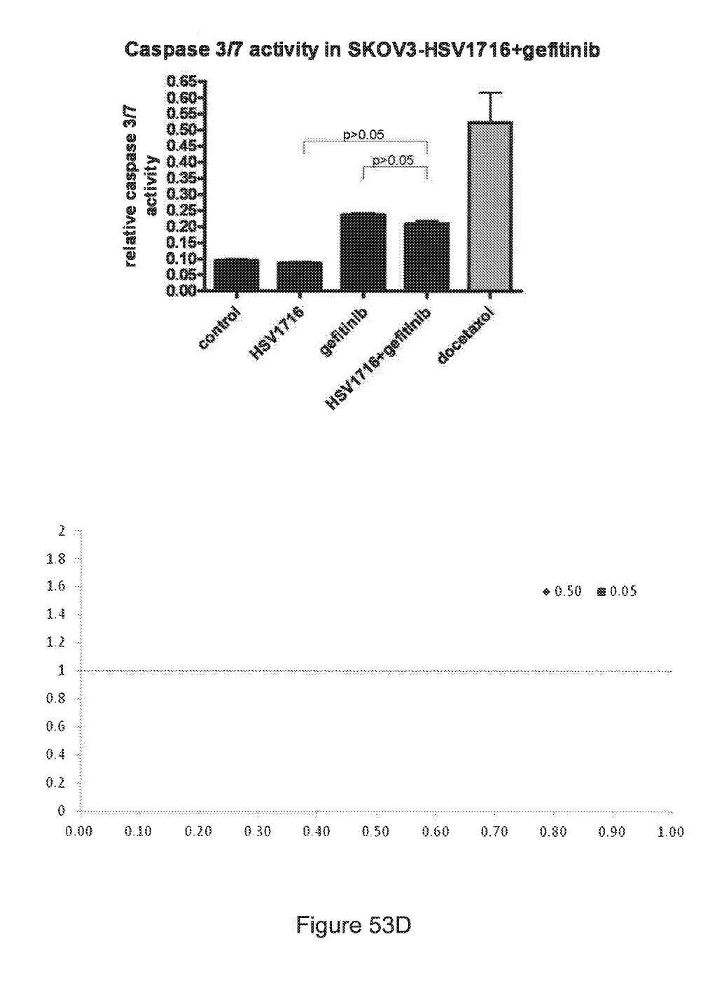

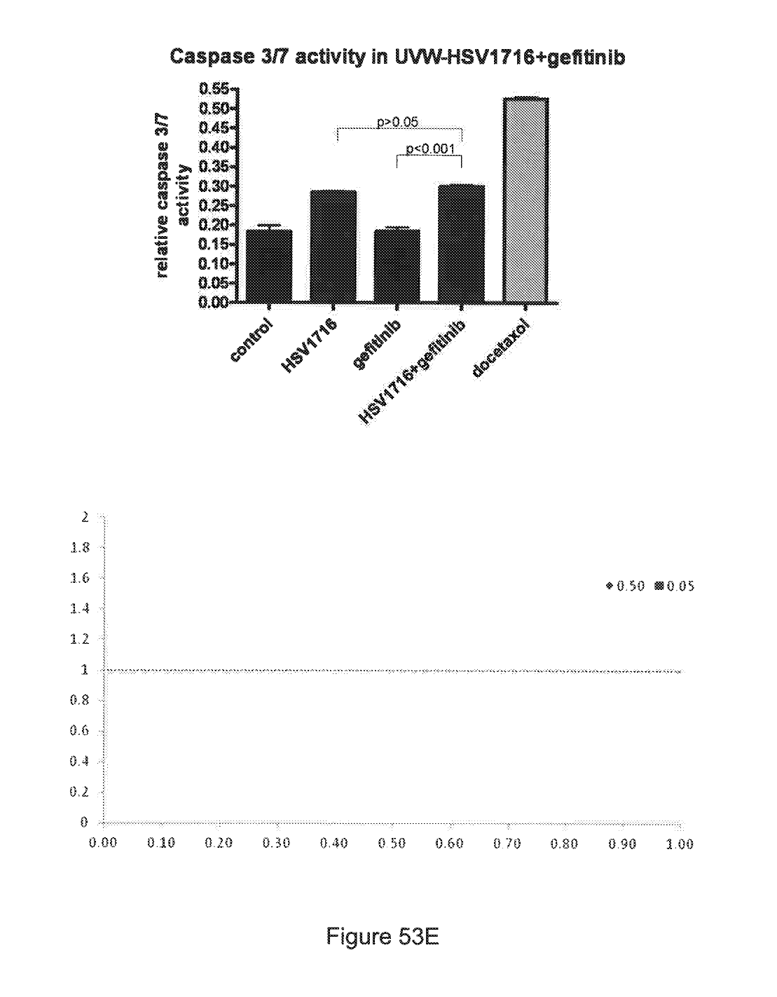

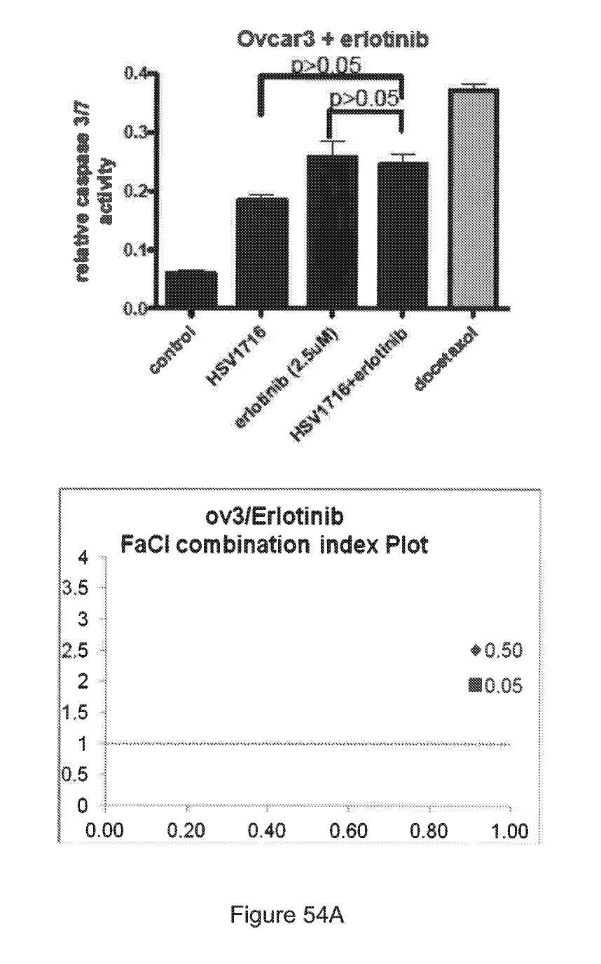

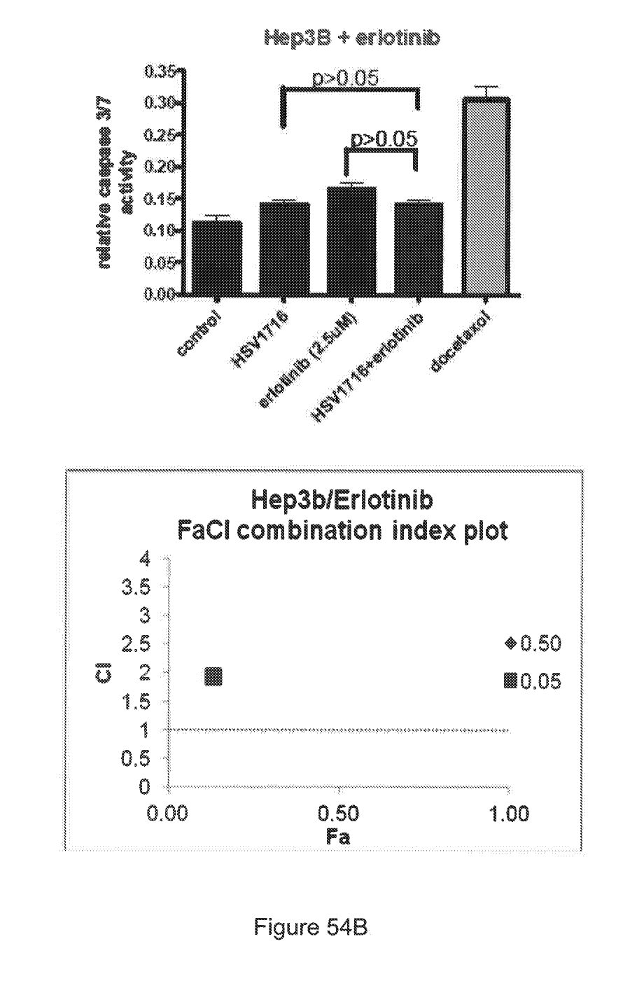

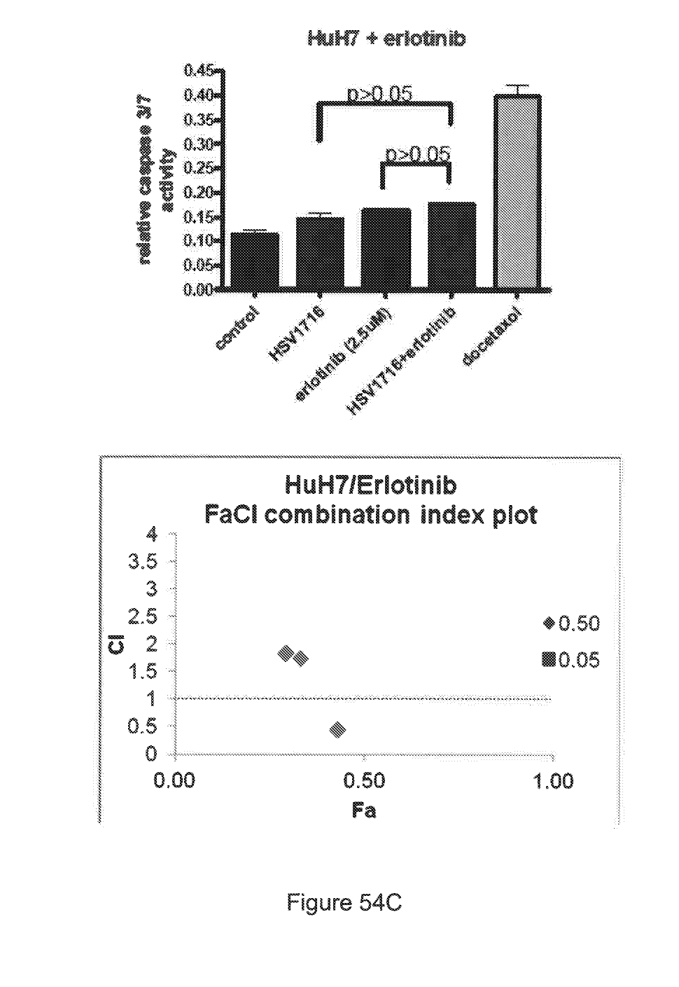

The EGFR TK inhibitors Gefitinib and Erlotinib are mostly antagonistic with HSV1716, and neither activated caspase 3/7 when combined with the virus. In Ovcar3, One58, CP70, SKOV3 and UVW cells, Gefitinib did not combine synergistically with HSV1716. For Ovcar3, CP70 and SKOV3 cells, the combination failed to significantly enhance caspase 3/7 activity compared to HSV1716 or Gefitinib alone, whilst for One58 and UVW cells, the combination failed to enhance activity compared to HSV1716 alone (FIGS. 53A-53E). Similarly, in Ovcar3. Hep3B or HuH7 cells, Erlotinib did not combine synergistically with HSV1716, and the combination failed to enhance caspase 3/7 activity compared to drug alone or virus alone (FIGS. 54A-54C).

### Synergistic Combinations of HSV1716 and TKi Enhance Caspase 9 Activity

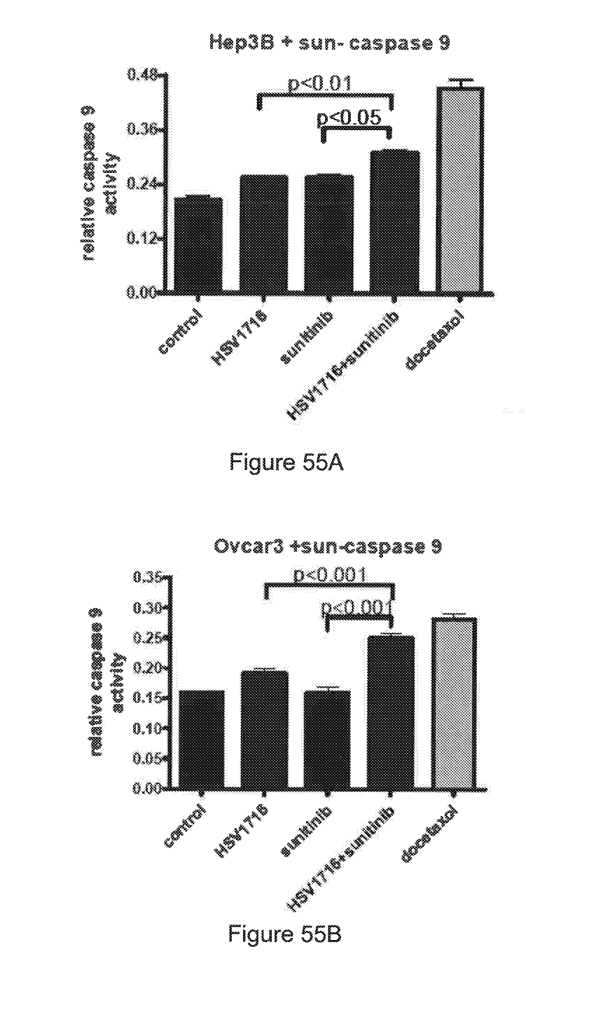

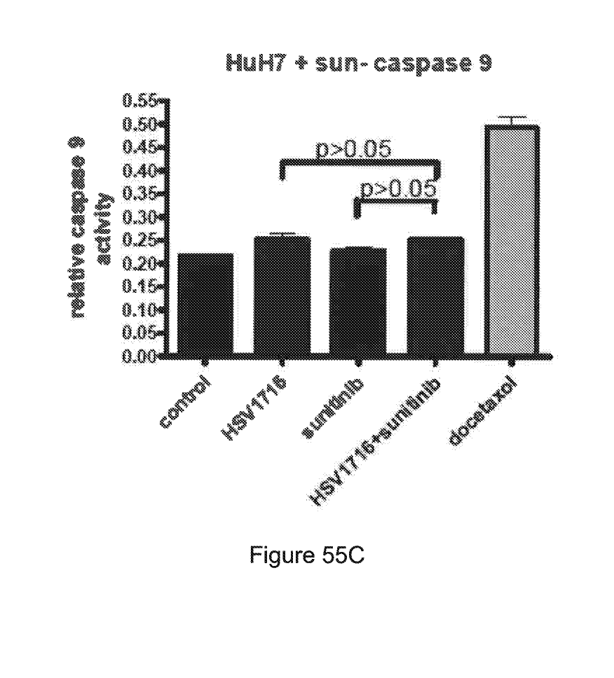

In Hep3B and Ovcar3 cells, Sunitinib and HSV1716 combine synergistically to significantly enhance caspase 9 activity (FIGS. 55A and 55B). By contrast, in HuH7 cells, HSV1716 and Sunitinib displayed no significant increase in caspase 9 activity (FIG. 55C).

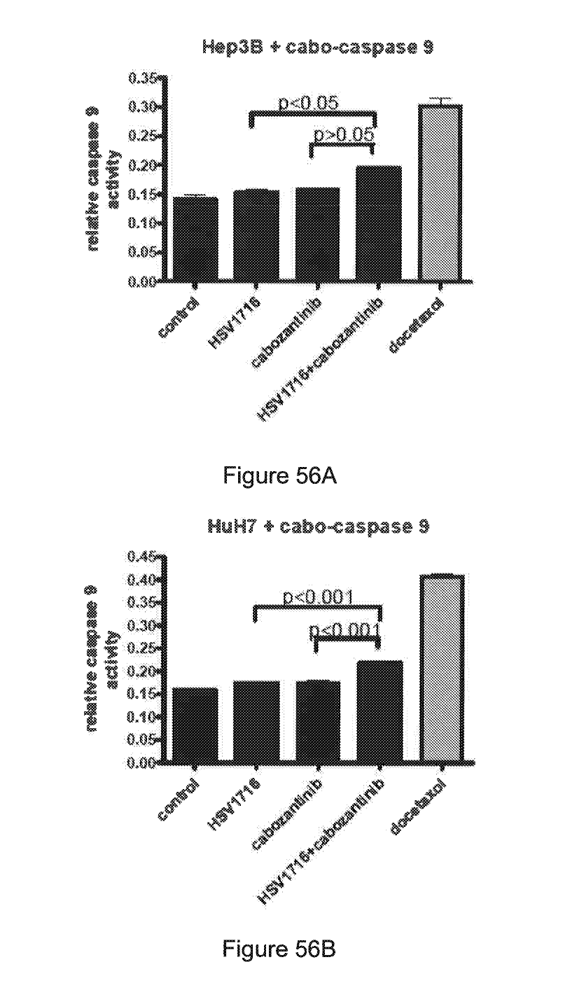

In Hep3B and HuH7 cells, Cabozantinib and HSV1716 combine synergistically to enhance caspase 3/7 activity and this is accompanied by a significant enhancement of caspase 9 activity (FIGS. 56A and 56B).

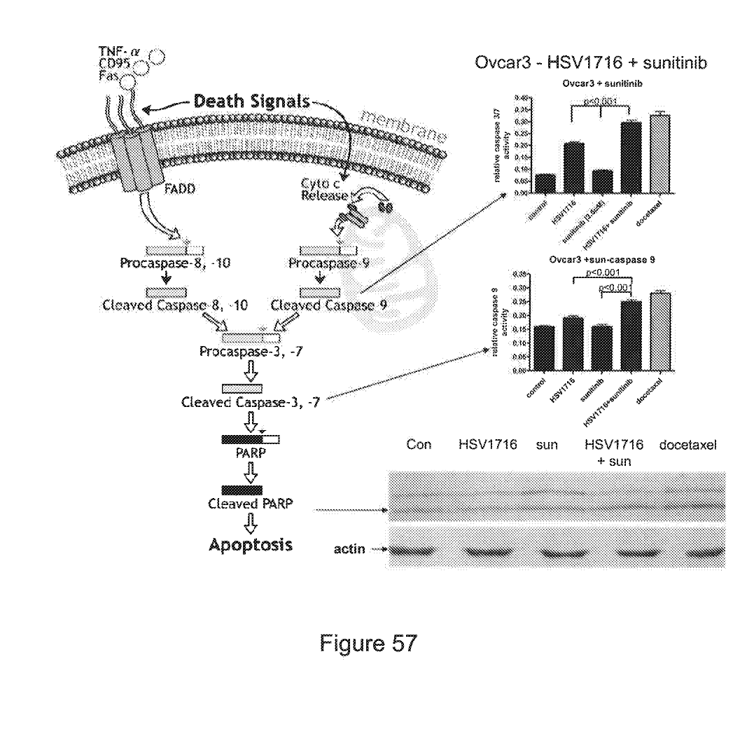

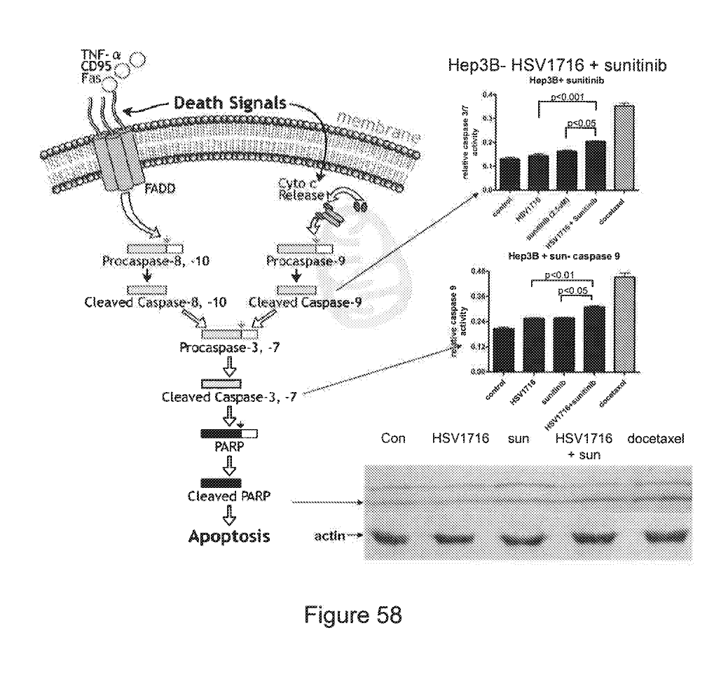

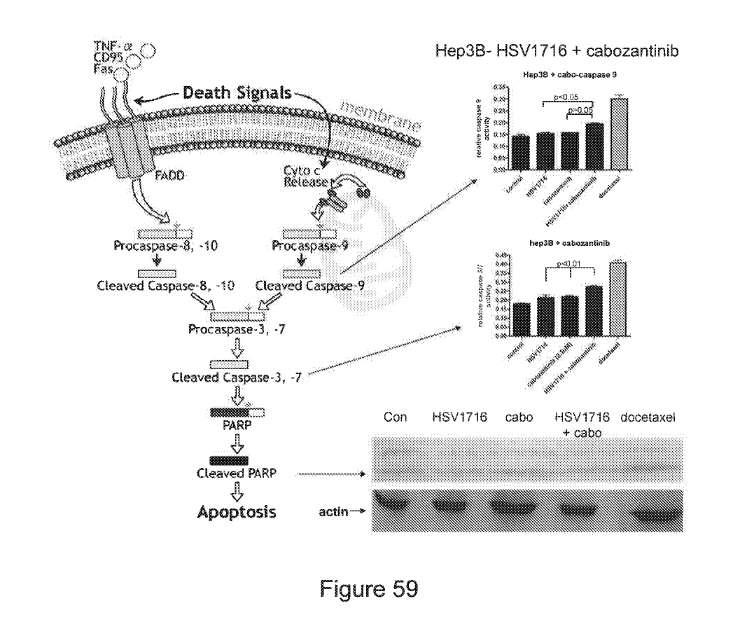

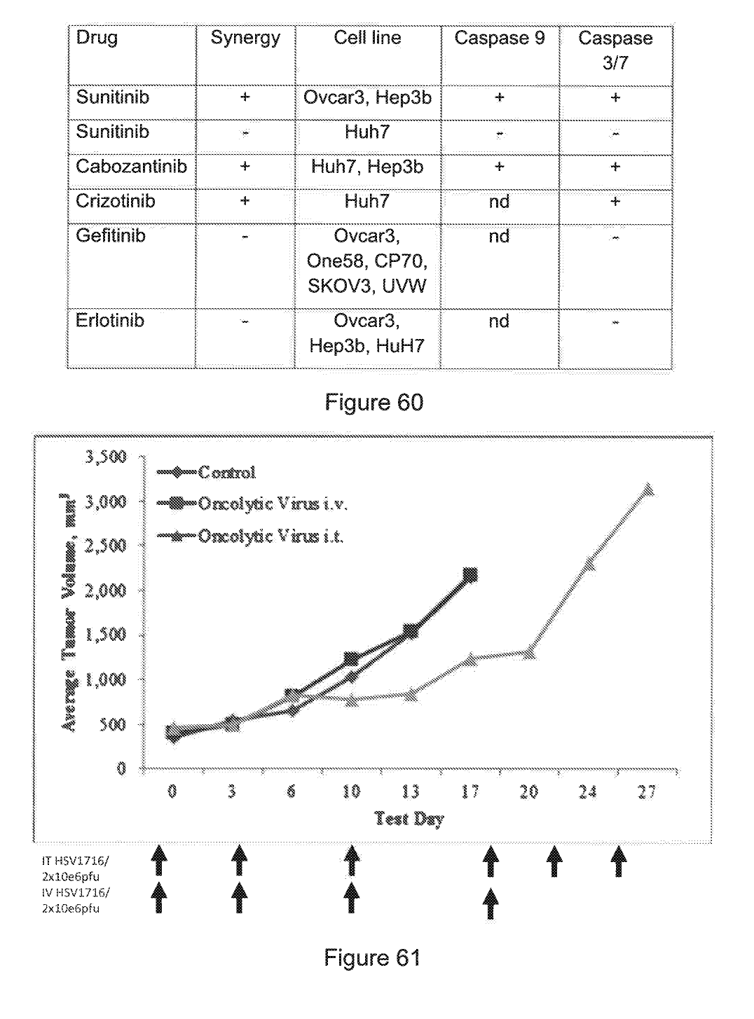

HSV1716 combines synergistically with tyrosine kinase inhibitors such as Sunitinib (FIGS. 57 and 58) or Cabozantinib (FIG. 59) in cell lines such as Ovcar3 or Hep3B and the intrinsic apoptosis cascade is activated. Thus. HSV1716/TKi synergies significantly enhance caspase 9 activity resulting in increased caspase 3/7 activity (Table 24; FIG. 60) with subsequent activation of the apoptotic cell death cascade as exemplified by cleavage of PARP (FIGS. 57-59), In cell lines in which HSV1716 does not combine synergistically with TKi, there was no significant increase of caspase 3/7 activity.

### Summary of Results

In cell lines in which HSV1716 combines synergistically with a tyrosine kinase inhibitor there is activation of caspases 9 and 3/7. In cell lines in which there are no combinatorial synergies, neither caspase 9 nor caspase 3/7 are activated.

### Example 12

### In Vivo Studies

HSV1716 (Seprehvir) was used in a personalised oncology study. Patient-derived tumour samples were obtained from a gastric carcinoma. There have been no previous in vitro or in vivo pre-clinical studies with Seprehvir in human gastric carcinoma cells/xenografts. Seprehvir was stored at −70° C., and multiple vials were provided to avoid repeated freeze/thawing.

### Study 1

Two groups of 3 mice with gastric carcinoma implants were treated with 2×10⁶ pfu Seprehvir by either intratumoural (IT) or intravenous (IV) routes of administration. Control mice (n=3) received no treatment and in these mice the implants grew rapidly and all were sacrificed within 18 days (FIG. 61). For IV injection, Seprehvir was administered on days 0, 3, 10 and 18, at which point the tumors were too large and the mice were sacrificed. IV injection of 2×10⁶ pfu Seprehvir had little or no effect on tumour growth inhibition and there was no prolongation of survival (FIG. 61).

For IT injection of Seprehvir, 2×10⁶ pfu was administered on days 0, 3, 10, 18, 21 and 25. There was a marked inhibition of tumour growth between days 6 and 20 but thereafter the xenografts grew very rapidly and subsequent doses of virus had little effect. All IT treated mice were sacrificed on day 27 therefore the tumour growth inhibition was able to prolong survival.

Three groups of 3 mice with gastric carcinoma implants were treated daily for 21 days with Lapatinib, Cabozantinib or Regorafenib by oral gavage. Lapatinib is dual tyrosine kinase inhibitor which inhibits the epidermal growth factor receptors (EGFR) and HER2/ERB2 and is most commonly used to treat breast cancer. Cabozantinib is a potent, orally bioavailable tyrosine kinase inhibitor that targets multiple pathways including VEGFR2, the rearranged during transfection (RET) and mesenchymal-epithelial transition factor (MET) receptor. Regorafenib (Fluoro-Sorafenib) is a multi-target inhibitor for VEGFR1, VEGFR2, VEGFR3, PDGFRβ, Kit, RET and Raf-1.

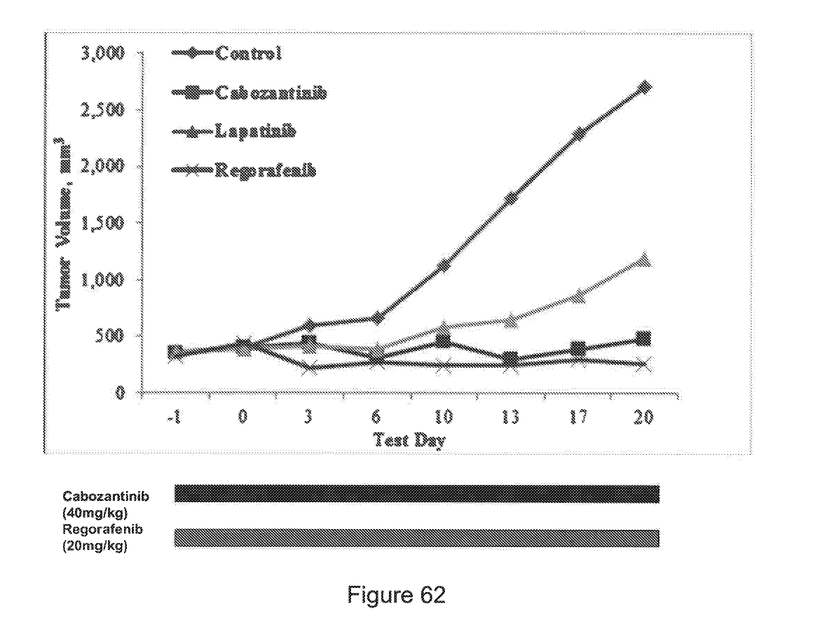

Control mice (n=3) received no treatment and in these mice the implants grew rapidly and all were sacrificed within 18 days (FIG. 62). Cabozantinib at 40 mg/kg or Regorafenib at 20 mg/kg were highly effective at tumour growth inhibition with little or no growth of the xenografts during the 21 days observation (FIG. 62). Initially, Lapatinib had some tumour growth inhibition until day 6, but there was marked tumour growth thereafter.

### Study 2

A follow-on study with HSV1716 administering more frequent by IV dosing at the higher concentration of 10⁷ pfu was performed using the same human gastric carcinoma xenograft model. The study included Seprehvir in combination with Cabozantinib at 40 mg/kg and regorafenib at 20 mg/kg administered daily by oral gavage.

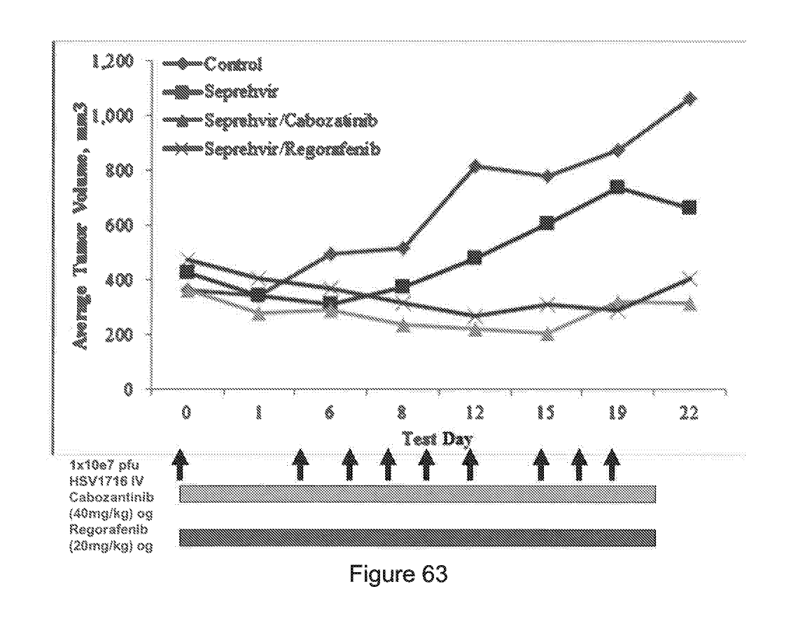

Groups of 3 mice received IV injection of HSV1716 at 1×10⁷ pfu on days 1, 3, 5, 8, 10, 12, 15, 17 and 19. There is an initial period of tumour growth inhibition for up to 10 days which is then followed by tumour growth thereafter although the xenografts are still smaller than controls throughout and at the end of the experiment on day 21 (FIG. 63). This pattern is similar to that seen in the initial experiment with IT HSV1716 at 2×10⁶ pfu/dose (FIG. 61). In the combination treated mice, Seprehvir Cabozantinib and Seprehvir Regorafenib were highly efficient at preventing tumour growth and throughout the observation period there is clear evidence of tumour shrinkage in both combination cohorts (FIG. 63).

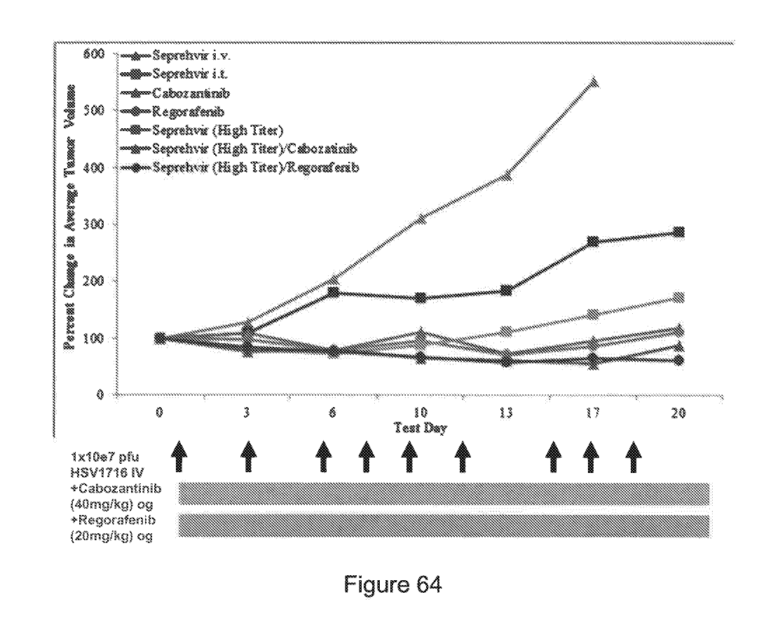

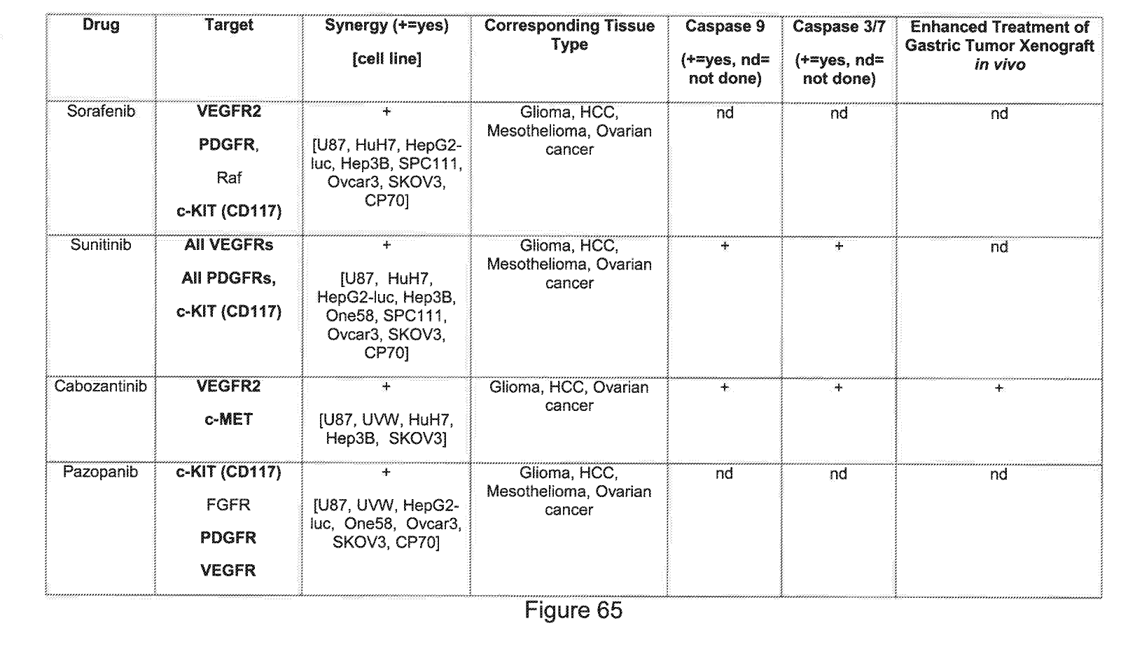

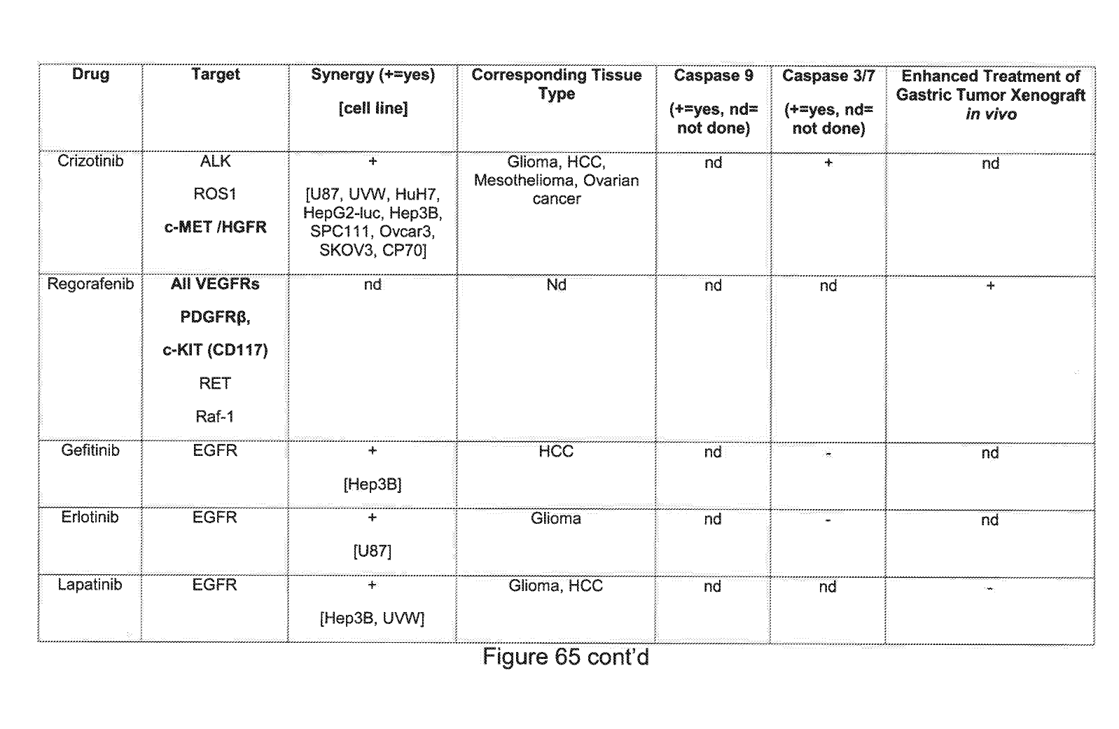

There were no mice treated with Cabozantinib or Regorafenib alone in this follow-up study and the data from both studies were combined in a graph of percentage change in average tumour volumes (FIG. 64). Study 1 data from Cabozantinib and Regorafenib alone and from IT HSV1716 and 2×10⁶ pfu IV HSV1716 are also presented in this graph as percentage change in average tumour volumes.

In the first cohort (Serpehvir IV), mice received HSV1716 IV at 2×10⁷ pfu but there was no tumour growth inhibition and the xenograft growth was equivalent to control mice (FIG. 61). There was some tumour growth inhibition by IT HSV1716 at this dose (Seprehvir IT).

Both Cabozantinib and Regorafenib were effective at inhibition of tumour growth over the 21 days although in both there is evidence of tumour growth in the latter stages of the observation period after day 13. In the 2^{nd} cohort, mice received IV injections of HSV1716 at 1×10⁷ pfu on days 1, 3, 5, 8, 10, 12, 15, 17 and 19 (Seprehvir (High Titer)) and there is effective tumour growth inhibition although not as potent as either Cabozantinib or Regorafenib alone. However, in combination with both Cabozantinib and Regorafenib, HSV1716 was highly efficient at inhibiting tumour growth and, particularly in the later stages of the experiment beyond day 13, the combination was apparently more effective than either agent alone.

### SUMMARY

Oncology studies have demonstrated tumour growth inhibition by intratumoural HSV1716 at 2×10⁶ pfu and intravenous HSV1716 at 1×10⁷ pfu in a challenging patient-derived gastric carcinoma xenograft model. Results clearly demonstrated compatibility of HSV1716 in vivo with two tyrosine kinase inhibitors, cabozantinib and regorafenib and suggest that tumour growth inhibition by both drugs was enhanced in combination with IV HSV1716. In total 9 IV injections of HSV1716 at 1×10⁷ pfu were administered per mouse over 21 days with no reported toxicity.

1. A method of treating cancer in a subject, the method comprising simultaneous or sequential administration of an oncolytic herpes simplex virus and a receptor tyrosine kinase (RTK) inhibitor, wherein the RTK inhibitor is a small molecule inhibitor, and is an inhibitor of a vascular endothelial growth factor receptor (VEGFR), wherein the combination of oncolytic herpes simplex virus and RTK inhibitor induces apoptosis in cells of the cancer, with the proviso that when the RTK inhibitor is Sorafenib, the cancer is not a liver cancer.

2. The method of treating cancer according to claim 1, wherein the combination of oncolytic herpes simplex virus and RTK inhibitor provides a synergistic level of cell killing.

3. The method of treating cancer according to claim 1, wherein the subject is selected for treatment by a method comprising:
infecting, in vitro, at least one cell of a subject's cancer with an oncolytic herpes simplex virus,
contacting said at least one cell with a small molecule RTK inhibitor that is an inhibitor of a vascular endothelial growth factor receptor (VEGFR),
determining whether (i) the combined action of the oncolytic herpes simplex virus and RTK inhibitor induces apoptosis in said at least one cell and/or (ii) the combined action of the oncolytic herpes simplex virus and RTK inhibitor provides a synergistic level of cell killing.

4. (canceled)

5. The method of treating cancer according to claim 1, wherein the RTK inhibitor is selected from the group consisting of Sorafenib, Pazopanib, Cabozantinib, Sunitinib, and Regorafenib.

6. The method of treating cancer according to claim 1, wherein the RTK inhibitor is selected from the group consisting of ZM 306416, Regorafenib, Cabozantinib, Foretinib, Golvatinib, ZM 323881 HCl, Apatinib, BMS 794833, RAF265, Ponatinib, Vandetanib, Ki8751, OSI-930, TSU-68, Brivanib, SAR131675, Ninetedinib, Dovitinib, Telatinib, KRN 633, MGCD-265, Tivozanib, ENMD-2076, Levantinib, Brivanib alaninate, Motesanib diphosphate, and Linifanib.

7. The method of treating cancer according to claim 1, wherein:
(a) the cancer is a glioma and the RTK inhibitor is selected from the group consisting of Sorafenib, Pazopanib, Cabozantinib, Sunitinib, and Regorafenib; or
(b) the cancer is a liver cancer, e.g. hepatocellular carcinoma, and the RTK inhibitor is selected from the group consisting of Pazopanib, Cabozantinib, Sunitinib, Crizitonib, and Regorafenib; or
(c) the cancer is a lung cancer, e.g. mesothelioma, and the RTK inhibitor is selected from the group consisting of Sorafenib, Pazopanib, Sunitinib, and Regorafenib; or
(d) the cancer is an ovarian cancer and the RTK inhibitor is selected from the group consisting of Sorafenib, Pazopanib, Cabozantinib, Sunitinib, and Regorafenib.

8. The method of treating cancer according to claim 1, wherein administration of the RTK inhibitor is direct administration to the cancer.

9. The method of treating cancer according to claim 1, wherein all copies of the ICP34.5 gene in the genome of the oncolytic herpes simplex virus are modified such that the ICP34.5 gene is incapable of expressing a functional ICP34.5 gene product.

10. The method of treating cancer according to claim 1, wherein the oncolytic herpes simplex virus is a mutant of HSV-1 strain 17.

11. The method of treating cancer according to claim 1, wherein the oncolytic herpes simplex virus is HSV1716.

12. (canceled)

13. A kit comprising a predetermined amount of an oncolytic herpes simplex virus and a predetermined amount of chemotherapeutic agent, wherein the chemotherapeutic agent is a receptor tyrosine kinase (RTK) inhibitor selected from the group consisting of ZM 306416, Regorafenib, Cabozantinib, Foretinib, Golvatinib, ZM 323881 HCl, Apatinib, BMS 794833, RAF265, Ponatinib, Vandetanib, Ki8751, OSI-930, TSU-68, Brivanib, SAR131675, Ninetedinib, Dovitinib, Telatinib, KRN 633, MGCD-265, Tivozanib, ENMD-2076, Levantinib, Brivanib alaninate, Motesanib diphosphate, and Linifanib.

14. A kit according to claim 13, wherein the oncolytic herpes simplex virus is a mutant of HSV-1 strain 17 or is HSV1716.

15. A method of selecting a subject for treatment with an oncolytic herpes simplex virus and a receptor tyrosine kinase (RTK) inhibitor, the method comprising:
infecting, in vitro, at least one cell of a subject's cancer with an oncolytic herpes simplex virus,
contacting said at least one cell with a small molecule RTK inhibitor that is an inhibitor of a vascular endothelial growth factor receptor (VEGFR), and
determining whether (i) the combined action of the oncolytic herpes simplex virus and RTK inhibitor induces apoptosis in said at least one cell and/or (ii) the combined action of the oncolytic herpes simplex virus and RTK inhibitor provides a synergistic level of cell killing in said at least one cell.

16. (canceled)
